SEQUENCE LISTING (47 sequences)

SEQ ID NO: 1 - protein, 120 aa, synthetic construct [sequence omitted]

SEQ ID NO: 2 - protein, 107 aa, synthetic construct [sequence omitted]

SEQ ID NO: 3 - protein, 119 aa, synthetic construct [sequence omitted]

SEQ ID NO: 4 - protein, 107 aa, synthetic construct [sequence omitted]

SEQ ID NO: 5 - protein, 7 aa, synthetic construct
  source 1..7: note = Trastuzumab Chothia CDR H1
GFNIKDT

SEQ ID NO: 6 - protein, 6 aa, synthetic construct
  source 1..6: note = Trastuzumab Chothia CDR H2
YPTNGY

SEQ ID NO: 7 - protein, 11 aa, synthetic construct
  source 1..11: note = Trastuzumab Chothia/kabat/AbM CDR H3
WGGDGFYAMDY

SEQ ID NO: 8 - protein, 11 aa, synthetic construct
  source 1..11: note = Trastuzumab Chothia/Kabat/AbM CDR L1
RASQDVNTAVA

SEQ ID NO: 9 - protein, 7 aa, synthetic construct
  source 1..7: note = Trastuzumab Chothia/Kabat/AbM CDR L2
SASFLYS

SEQ ID NO: 10 - protein, 9 aa, synthetic construct
  source 1..9: note = Trastuzumab Chothia/Kabat/IMGT/AbM CDR L3
QQHYTTPPT

SEQ ID NO: 11 - protein, 5 aa, synthetic construct
  source 1..5: note = Trastuzumab Kabat CDR H1
DTYIH

SEQ ID NO: 12 - protein, 17 aa, synthetic construct
  source 1..17: note = Trastuzumab Kabat CDR H2
RIYPTNGYTRYADSVKG

SEQ ID NO: 13 - protein, 8 aa, synthetic construct
  source 1..8: note = Trastuzumab IMGT CDR H1
GFNIKDTY

SEQ ID NO: 14 - protein, 8 aa, synthetic construct
  source 1..8: note = Trastuzumab IMGT CDR H2
IYPTNGYT

SEQ ID NO: 15 - protein, 13 aa, synthetic construct
  source 1..13: note = Trastuzumab IMGT CDR H3
SRWGGDGFYAMDY

SEQ ID NO: 16 - protein, 6 aa, synthetic construct
  source 1..6: note = Trastuzumab IMGT CDR L1
QDVNTA

SEQ ID NO: 17 - skipped

SEQ ID NO: 18 - protein, 10 aa, synthetic construct
  source 1..10: note = Trastuzumab AbM CDR H1
GFNIKDTYIH

SEQ ID NO: 19 - protein, 10 aa, synthetic construct
  source 1..10: note = Trastuzumab AbM CDR H2
RIYPTNGYTR

SEQ ID NO: 20 - protein, 7 aa, synthetic construct
  source 1..7: note = Pertuzumab Chothia CDR H1
GFTFTDY

SEQ ID NO: 21 - protein, 6 aa, synthetic construct
  source 1..6: note = Pertuzumab Chothia CDR H2
NPNSGG

SEQ ID NO: 22 - protein, 10 aa, synthetic construct
  source 1..10: note = Pertuzumab Chothia/Kabat/AbM CDR H3
NLGPSFYFDY

SEQ ID NO: 23 - protein, 11 aa, synthetic construct
  source 1..11: note = Pertuzumab Chothia/Kabat/AbM CDR L1
KASQDVSIGVA

SEQ ID NO: 24 - protein, 7 aa, synthetic construct
  source 1..7: note = Pertuzumab Chothia/Kabat/AbM CDR L2
SASYRYT

SEQ ID NO: 25 - protein, 9 aa, synthetic construct
  source 1..9: note = Pertuzumab Chothia/Kabat/IMGT/AbM CDR L3
QQYYIYPYT

SEQ ID NO: 26 - protein, 5 aa, synthetic construct
  source 1..5: note = Pertuzumab Kabat CDR H1
DYTMD

SEQ ID NO: 27 - protein, 17 aa, synthetic construct
  source 1..17: note = Pertuzumab Kabat CDR H2
DVNPNSGGSIYNQRFKG

SEQ ID NO: 28 - protein, 8 aa, synthetic construct
  source 1..8: note = Pertuzumab IMGT CDR H1
GFTFTDYT

SEQ ID NO: 29 - protein, 8 aa, synthetic construct
  source 1..8: note = Pertuzumab IMGT CDR H2
VNPNSGGS

SEQ ID NO: 30 - protein, 12 aa, synthetic construct
  source 1..12: note = Pertuzumab IMGT CDR H3
ARNLGPSFYFDY

SEQ ID NO: 31 - protein, 6 aa, synthetic construct
  source 1..6: note = Pertuzumab IMGT CDR L1
QDVSIG

SEQ ID NO: 32 - skipped

SEQ ID NO: 33 - protein, 10 aa, synthetic construct
  source 1..10: note = Pertuzumab AbM CDR H1
GFTFTDYTMD

SEQ ID NO: 34 - protein, 10 aa, synthetic construct
  source 1..10: note = Pertuzumab AbM CDR H2
DVNPNSGGSI

SEQ ID NO: 35 - protein, 330 aa, synthetic construct
  source 1..330: note = CH of Trastuzumab/Pertuzumab
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 107 aa, synthetic construct [sequence omitted]

SEQ ID NO: 37 - protein, 450 aa, synthetic construct
  source 1..450: note = heavy chain of Trastuzumab
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, synthetic construct
  source 1..214: note = light chain of Trastuzumab
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 449 aa, synthetic construct
  source 1..449: note = heavy chain of Pertuzumab
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct
  source 1..214: note = light chain of Pertuzumab
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 4 aa, synthetic construct
  source 1..4: note = short peptide
GGFG

SEQ ID NO: 42 - protein, 4 aa, synthetic construct
  source 1..4: note = short peptide
GGVA

SEQ ID NO: 43 - protein, 4 aa, synthetic construct
  source 1..4: note = short peptide
GFLG

SEQ ID NO: 44 - protein, 4 aa, synthetic construct
  source 1..4: note = short peptide
EAAA

SEQ ID NO: 45 - protein, 5 aa, synthetic construct
  source 1..5: note = short peptide
GGGGG

SEQ ID NO: 46 - protein, 5 aa, synthetic construct
  source 1..5: note = linker subunit
GGGGS

SEQ ID NO: 47 - protein, 20 aa, synthetic construct
  source 1..20: note = (GGGGS)4
GGGGSGGGGSGGGGSGGGGS

(19) United States
(12) Patent Application Publication
(10) Pub. No.: US 2026/0241020 A1
(43) Pub. Date: Aug. 20, 2026

(54) Antibody-drug conjugate, and preparation method therefor and use thereof

(71) Applicant: SICHUAN KELUN-BIOTECH BIOPHARMACEUTICAL CO., LTD., Chengdu, Sichuan (CN)

(72) Inventors: Qiang Tian, Chengdu, Sichuan (CN); Yitao Zhang, Chengdu, Sichuan (CN); Jian Ye, Chengdu, Sichuan (CN); Yile Li, Chengdu, Sichuan (CN); Pan Song, Chengdu, Sichuan (CN); Qin Zhou, Chengdu, Sichuan (CN); Hu Long, Chengdu, Sichuan (CN); Yu Yang, Chengdu, Sichuan (CN); Xiaoxi Yuan, Chengdu, Sichuan (CN); Hongmei Song, Chengdu, Sichuan (CN); Junyou Ge, Chengdu, Sichuan (CN)

(21) Appl. No.: 19/469,740

(22) PCT Filed: May 10, 2024
(86) PCT No.: PCT/CN2024/092371

§ 371 Date: Sep. 26, 2025

(30) Foreign Application Priority Data
May 12, 2023 (CN) 202310538792.7

(51) Int. Cl. A61K 47/68; A61P 35/00; C07K 16/32

(52) U.S. Cl. CPC A61K 47/68031; A61K 47/6855; A61K 47/6889; A61P 35/00; C07K 16/32

(57) ABSTRACT

Provided are an antibody-drug conjugate, and a preparation method therefor and use thereof. The antibody-drug conjugate has a structure as shown in formula Ab-[M-L-E-D]ₓ, the drug is selected from a tubulin inhibitor, and the prepared antibody-drug conjugate has a better drug antibody coupling ratio and has a good targeted killing effect on solid tumors such as gastric cancer, breast cancer, lung cancer and urothelial carcinoma.

The present application is based on and claims priority to the Chinese application with application number CN 202310538792.7, filed on May 12, 2023. The disclosure of the Chinese application is hereby incorporated into the present application in its entirety.

### TECHNICAL FIELD

The present application relates to the field of targeted therapy, and specifically to an antibody-drug conjugate, and preparation method therefor and use thereof.

### BACKGROUND ART

Antibody-drug conjugate (ADC) for cancer treatment is typically composed of a monoclonal antibody, a bioactive molecule (primarily a tumor-killing cytotoxin) and a linker. The bioactive molecule is covalently coupled to the antibody via the linker. The antibody recognizes a specific target on the surface of tumor cells, directing the ADC to the tumor microenvironment and cancer cell surfaces. Through endocytosis, the ADC enters the cancer cells, where the bioactive molecule is released. By inhibiting the tubulin of the cancer cells or damaging the DNA of the cancer cells, the bioactive molecule kills the cancer cells while minimizing damage to normal tissue cells.

The ErbB family of receptor tyrosine kinases are important mediators of cell growth, differentiation, and survival. This family includes four members: epidermal growth factor receptor (EGFR or ErbB1), Her2 (ErbB2), Her3 (ErbB3) and Her4 (ErbB4). In clinical practice, the anti-ErbB2 antibody trastuzumab (trade name Herceptin) is often used to treat breast cancer with high expression of ErbB2, but the clinical response rate remains low. In order to improve the therapeutic efficacy, in recent years, conjugates (e.g., Trastuzumab emtansine, Disitamab vedotin, Trastuzumab deruxtecan) combining anti-ErbB2 antibodies with tubulin inhibitors such as maytansines (e.g., DM1), auristatins (e.g., MMAE) or DNA topoisomerase I inhibitors (e.g., D×d) have been used for clinical treatment.

With the widespread clinical application of the aforementioned ADC drugs in the treatment of Her2-expressing tumors, safety issues including neurotoxicity, hematotoxicity, hepatotoxicity, and interstitial pneumonia and drug resistance issues have gradually emerged (Pharmacology & Therapeutics 2019, 200, 110-125; Breast Cancer Research and Treatment 2020, 183, 23-39; JAMA Oncol. 2021, 7, 1873-1881; Drug Deliv. 2022, 29, 1335-1344; Cancers 2023, 15, 1130; Cancers 2023, 15, 1278.)

Specifically, both of the linker moieties of Disitamab vedotin and Trastuzumab deruxtecan adopt maleimide linker MC, which has been reported to be susceptible to reversible Michael addition and thiol exchange reactions under physiological conditions, resulting in reduced efficacy and increased toxicity (Nat Biotechnol. 2012, 32, 184-189; Bioconjugate Chem. 2015, 26, 145-152). In terms of the hydrophilicity of the linker, Disitamab vedotin and Trastuzumab deruxtecan incorporate the more hydrophobic linkers of valine-citrulline (Val-Cit) and glycine-glycine-phenylalanine-glycine (Gly-Gly-Phe-Gly), respectively. Literature reports that the hydrophilicity of linkers significantly affects the hydrophilicity of ADCs, thereby affecting the aggregation, pharmacokinetic properties and toxicity of ADCs (Chemical Linkers in Antibody-Drug Conjugates (ADCs), Drug Discovery Series No. 81, Chapter 3.). As for the bioactive molecule moieties that play a role in killing cancer cells, Trastuzumab emtansine utilizes the microtubule inhibitor DM1 as a cytotoxin, which leads to a weak bystander effect after being matched with a non-cleavable linker; Disitamab vedotin employs the auristatin toxin MMAE as its bioactive molecule, which may associates with problems such as cumulative neurotoxicity upon repeated administration; and both of the aforementioned ADC drugs utilize non-site-specific random conjugation, resulting in drug-to-antibody ratio (DAR) of about 4 and relatively poor homogeneity.

### Contents of the Invention

The present invention aims to solve the above problems in the existing pharmaceutical technology, specifically to provide an anti-Her2 antibody-drug conjugate capable of being used for the treatment of Her2-expressing tumors. The antibody-drug conjugate has good stability, uniformity, hydrophilicity, effectiveness and safety.

The present application relates to an antibody-drug conjugate, and exemplarily discloses an antibody-drug conjugate having a structure represented by the general formula Ab-[M-L-E-D]ₓ with trastuzumab as a targeting moiety. The results show that the conjugate has better drug-to-antibody conjugation ratio (e.g., 6.5 to 8.5), and exerts excellent binding activity and proliferation inhibition on Her2-positive cells, and has good targeted killing effect on Her2-positive tumors (e.g., breast cancer, lung cancer or gastric cancer). Therefore, the present application provides an antibody-drug conjugate for treating Her2-expressing cancer, a pharmaceutical composition comprising the antibody-drug conjugate, and use thereof in treating Her2-expressing cancer.

### Antibody-Drug Conjugate

In one aspect, the present application provides an antibody-drug conjugate, which has the formula Ab-[M-L-E-D]ₓ, where:
Ab represents an antibody or antigen-binding fragment thereof capable of specifically binding to epidermal growth factor receptor 2 (Her2), a member of the ErbB family of receptor tyrosine kinases:
M represents a structural fragment connected to the antibody or antigen-binding fragment thereof;
L represents a structural fragment connecting the linking site M and E;
E represents a structural fragment connecting L and D;
D represents a cytotoxic drug fragment;
x is 1 to 10.

In the antibody-drug conjugate, the cytotoxic drug can be connected to the antibody or antigen-binding fragment thereof through the “M-L-E” fragment as shown in the present application.

In some embodiments, M is selected from the group consisting of the following structural fragments:

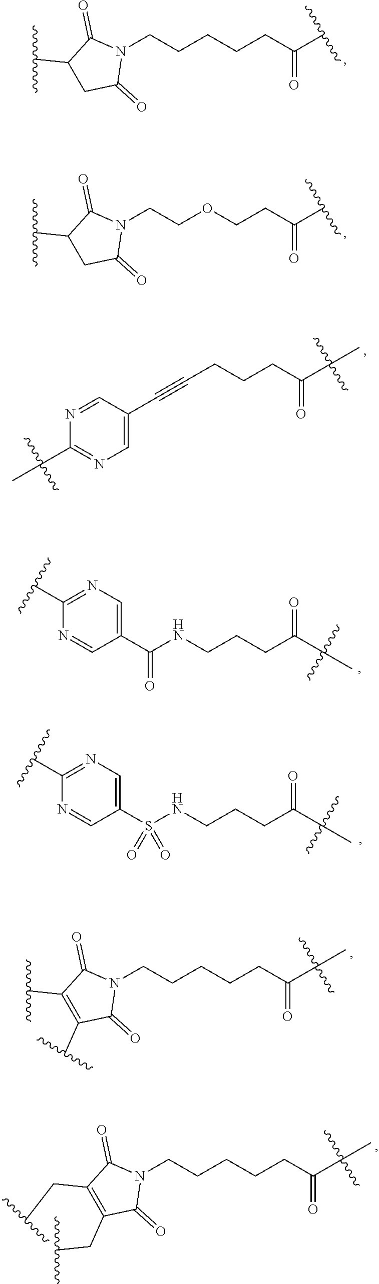

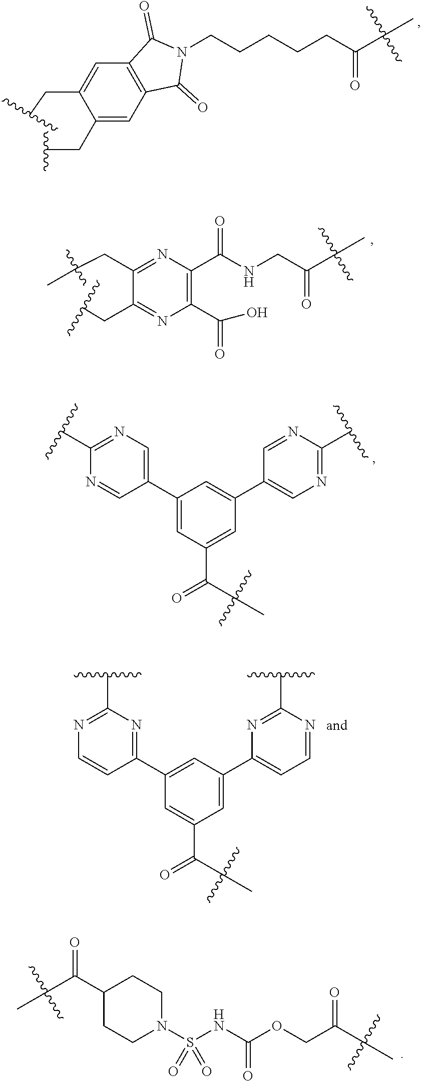 [SMILES: *Cc1cc2c(cc1C*)C(=O)N(CCCCCC(*)=O)C2=O ; *Cc1nc(C(=O)O)c(C(=O)NCC(*)=O)nc1C* ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; "and" ; *C(=O)COC(=O)NS(=O)(=O)N1CCC(C(*)=O)CC1]

In some embodiments, M is selected from the group consisting of the following structural fragments:

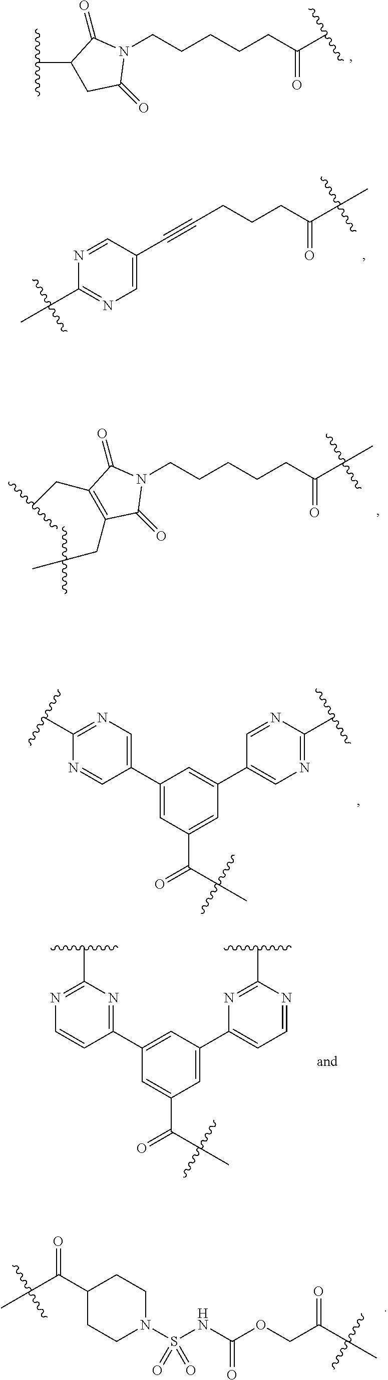 [SMILES: *C(=O)CCCCCN1C(=O)CC(*)C1=O ; *C(=O)CCCC#Cc1cnc(*)nc1 ; *CC1=C(C*)C(=O)N(CCCCCC(*)=O)C1=O ; *C(=O)c1cc(-c2cnc(*)nc2)cc(-c2cnc(*)nc2)c1 ; *C(=O)c1cc(-c2ccnc(*)n2)cc(-c2ccnc(*)n2)c1 ; *C(=O)COC(=O)NS(=O)(=O)N1CCC(C(*)=O)CC1]

In some embodiments, M is selected from the group consisting of the following structural fragments:

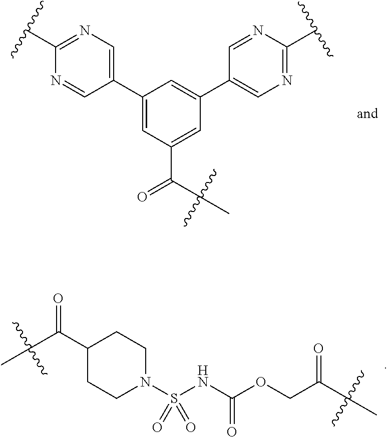 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

In some embodiments, M is the following structural fragment:

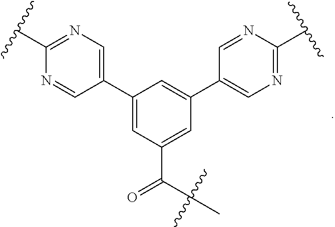 [SMILES: *C(=O)c1cc(-c2cnc(*)nc2)cc(-c2cnc(*)nc2)c1]

In some embodiments, M is the following structural fragment:

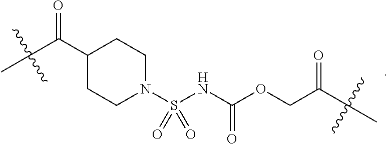 [SMILES: *C(=O)COC(=O)NS(=O)(=O)N1CCC(C(*)=O)CC1]

In some embodiments, L is selected from the group consisting of divalent structural fragments composed of one or more of the following groups: C₁₋₆ alkylene, 6- to 10-membered arylene, 5- to 6-membered heteroarylene, —N(R′)—, carbonyl group, —O—, a residue of an amino acid or an analog thereof (the amino acid is a natural amino acid or a non-natural amino acid, such as Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala or Lys (COCH₂CH₂ (OCH₂CH₂)ᵣOCH₃)), Lys (R′), a fragment of a short peptide consisting of two or more amino acids (the short peptide is, for example, selected from the group consisting of Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly (SEQ ID NO: 41), Gly-Gly-Val-Ala (SEQ ID NO: 42), Gly-Phe-Leu-Gly (SEQ ID NO: 43), Glu-Ala-Ala-Ala (SEQ ID NO: 44) and Gly-Gly-Gly-Gly-Gly (SEQ ID NO: 45),

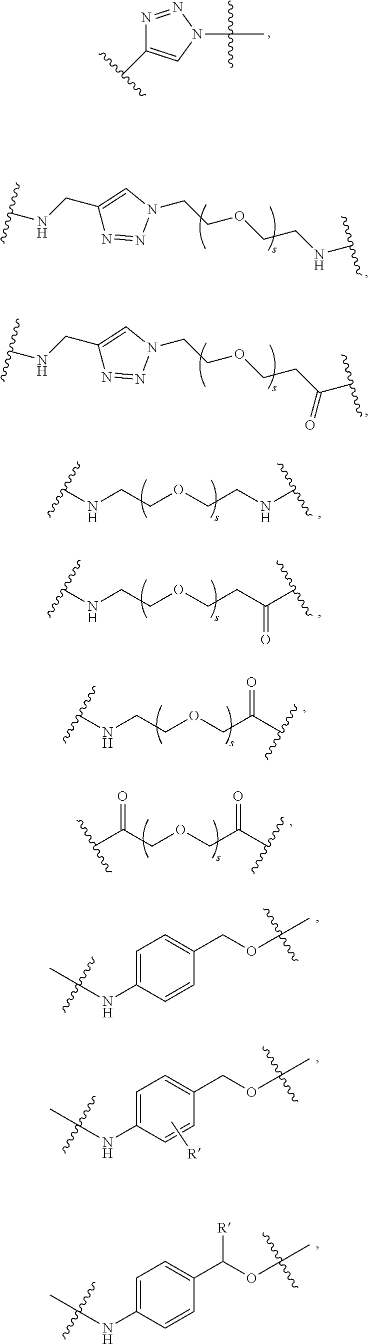

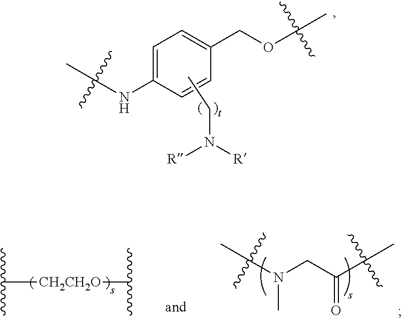

where R′ and R″ each independently represent hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—O—(CH₂CH₂O)ᵣ—C₁₋₆ alkyl, —(CH₂N(Me)-C(═O))ᵣ—C₁₋₆ alkyl, a polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), where r is an integer selected from 1 to 20, such as 1, 2, 3, 4, 5, 6, 7, 8, 9, 10, 11, 12, 13, 14, 15, 16, 17, 18, 19, 20; s is an integer selected from 1 to 20, such as 1, 2, 3, 4, 5, 6, 7, 8, 9, 10, 11, 12, 13, 14, 15, 16, 17, 18, 19, 20, preferably 5, 8 or 9; t is an integer selected from 1 to 5, such as 1, 2, 3, 4, 5; preferably, tis 1.

In some embodiments, “—NOTA” refers to

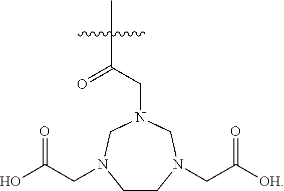 [SMILES: *C(=O)CN1CN(CC(=O)O)CCN(CC(=O)O)C1]

In some embodiments, “-DOTA” refers to

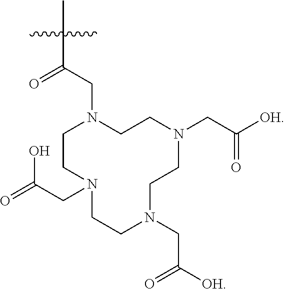 [SMILES: *C(=O)CN1CCN(CC(=O)O)CCN(CC(=O)O)CCN(CC(=O)O)CC1]

In some embodiments, “-DOTAGA” refers to

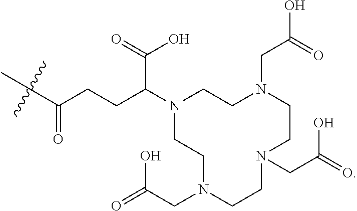 [SMILES: *C(=O)CCC(C(=O)O)N1CCN(CC(=O)O)CCN(CC(=O)O)CCN(CC(=O)O)CC1]

In some embodiments, r is an integer selected from 1 to 15, such as an integer of 1 to 12, 3 to 12, 1 to 10, 1 to 8, 3 to 8, 1 to 6, 1 to 4, 1 to 2, such as 1, 2, 3, 4, 5, 6, 7, 8, 9, 10, 11, 12, 13, 14, 15, 16, 17, 18, 19 or 20.

In some embodiments, s is an integer selected from 1 to 15, such as an integer of 1 to 12, 3 to 12, 5 to 10, such as 1, 2, 3, 4, 5, 6, 7, 8, 9, 10, 11, 12, 13, 14, 15, 16, 17, 18, 19 or 20.

In some embodiments, L is selected from the group consisting of substituted or unsubstituted structural fragments composed of one or more of the following groups: C₁₋₆ alkylene, 6- to 10-membered aryl, 5- to 6-membered heteroaryl, —N(R′)—, carbonyl group, —O—, a natural amino acid or an unnatural amino acid or an analog thereof (e.g., Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala, Lys(COCH₂CH₂(OCH₂CH₂)ᵣOCH₃)), a short peptide composed of amino acids (e.g., Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly),

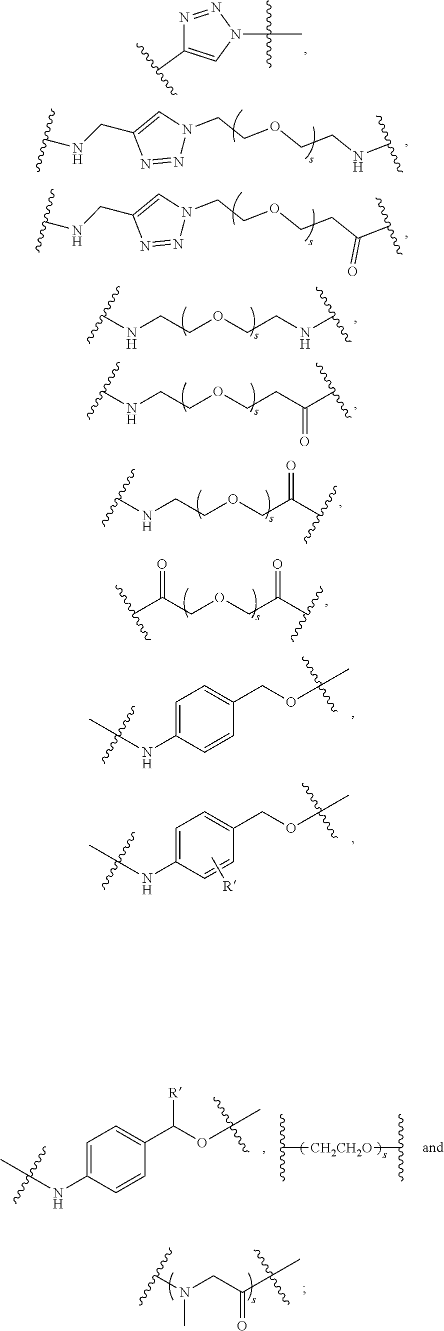

where R′ represents hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —(CH₂N(Me)-C(═O))ᵣ—C₁₋₆ alkyl, or polyethylene glycol fragment containing 1 to 10 EO units (i.e., —(CH₂CH₂O)ᵣ—C₁₋₆ alkyl), where r is an integer selected from 1 to 20; s is an integer selected from 1 to 20.

In some embodiments, L is selected from the group consisting of divalent structural fragments composed of one or more of the following groups: C₁₋₆ alkylene, carbonyl group, —NH—, Lys(R′), Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

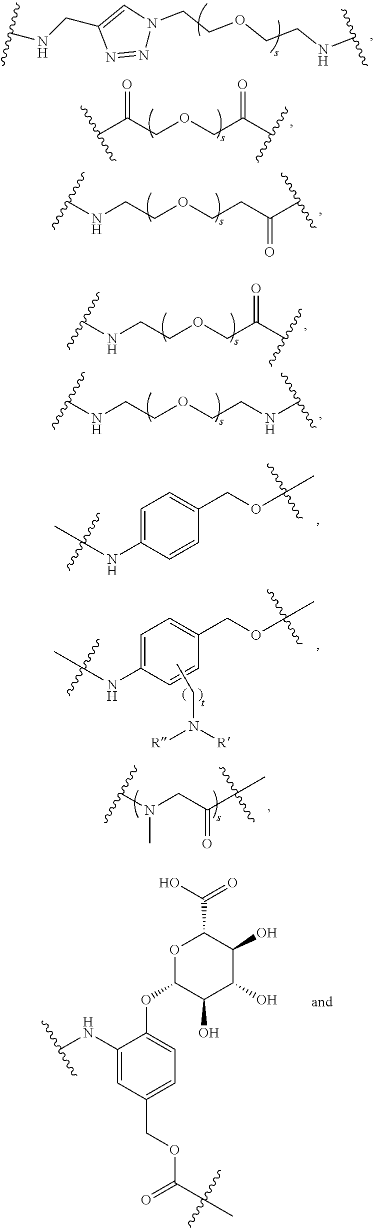

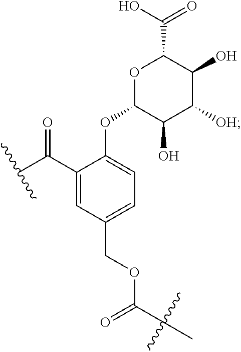

where R′ and R″ each independently represent hydrogen, C₁₋₆ alkyl, glycosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—O—(CH₂CH₂O)ᵣ—C₁₋₆ alkyl, —(CH₂N(Me)-C(═O))ᵣ—C₁₋₆ alkyl, polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), where r is an integer selected from 1 to 20; s is an integer selected from 1 to 20, preferably, s is 5, 8 or 9; t is an integer selected from 1 to 5; preferably, tis 1.

In some embodiments, L is selected from the group consisting of substituted or unsubstituted structural fragments composed of one or more of the following groups: C₁₋₆ alkylene, carbonyl group, —NH—, Ala-Ala, Ala-Lys, Ala-Pro, Gly-Glu, Gly-Gly, Phe-Lys, Val-Ala, Val-Cit, Val-Lys, Ala-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala, Gly-Gly-Gly-Gly-Gly,

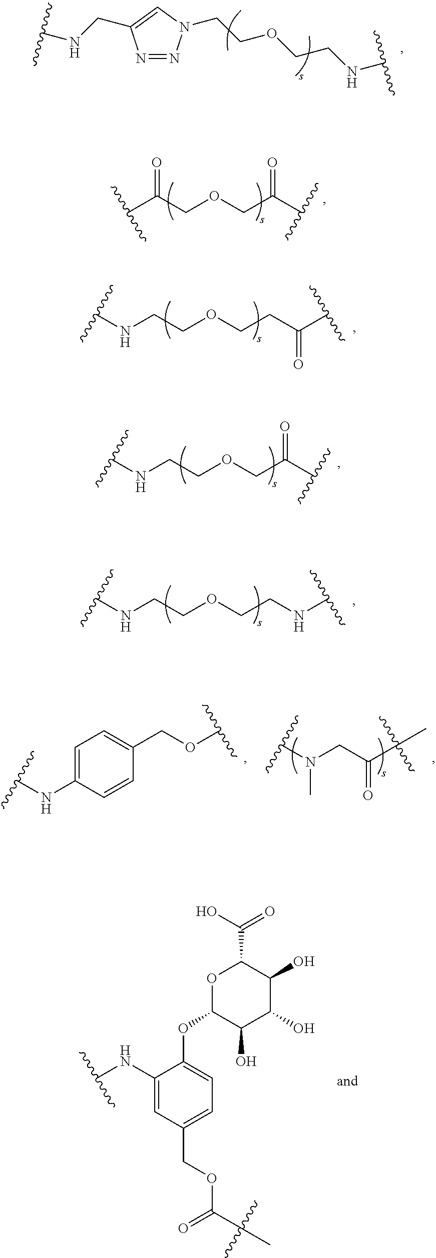

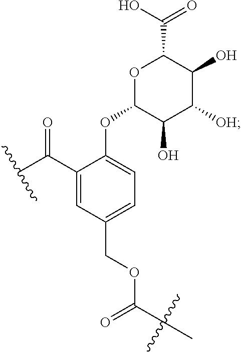

where s is an integer selected from 1 to 20.

In some embodiments, L is selected from the group consisting of divalent structural fragments composed of one or more of the following groups: carbonyl group, Lys(R′), Val-Ala, Val-Cit, Ala-Ala-Ala, D-Leu-Ala-Glu, Glu-Val-Ala, Ser-D-Ala-Pro, Val-Lys-Gly, Gly-Gly-Val-Ala,

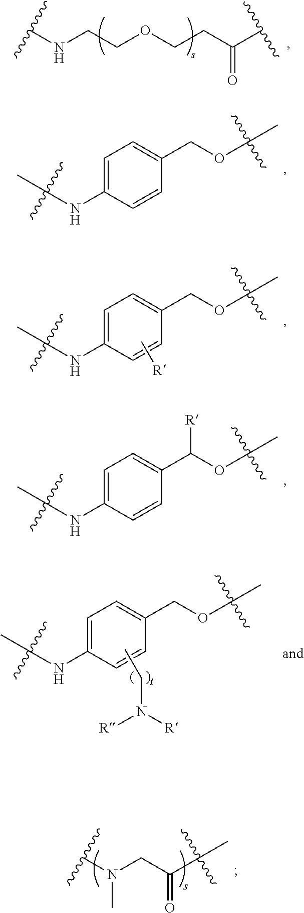

where R′ and R″ each independently represent hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CHEN(C₁₋₆ alkyl)-C(═O)—O— (CH₂CHO)ᵣ—C₁₋₆ alkyl, —(CH₂N(Me)-C(═O))ᵣ—C₁₋₆ alkyl, or polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), where r is an integer selected from 1 to 20; s is an integer selected from 1 to 20; tis an integer selected from 1 to 5; preferably, tis 1.

In some embodiments, L is selected from the group consisting of structural fragments composed of one or more of the following groups:

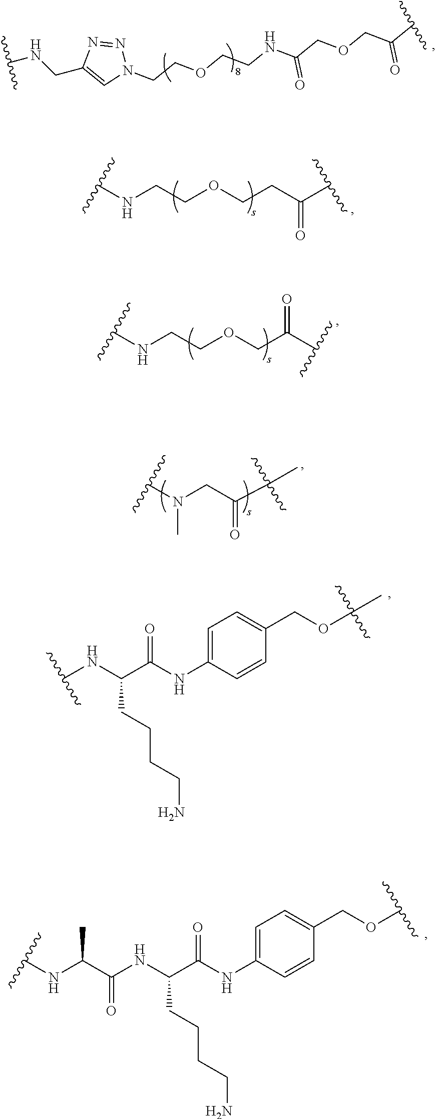

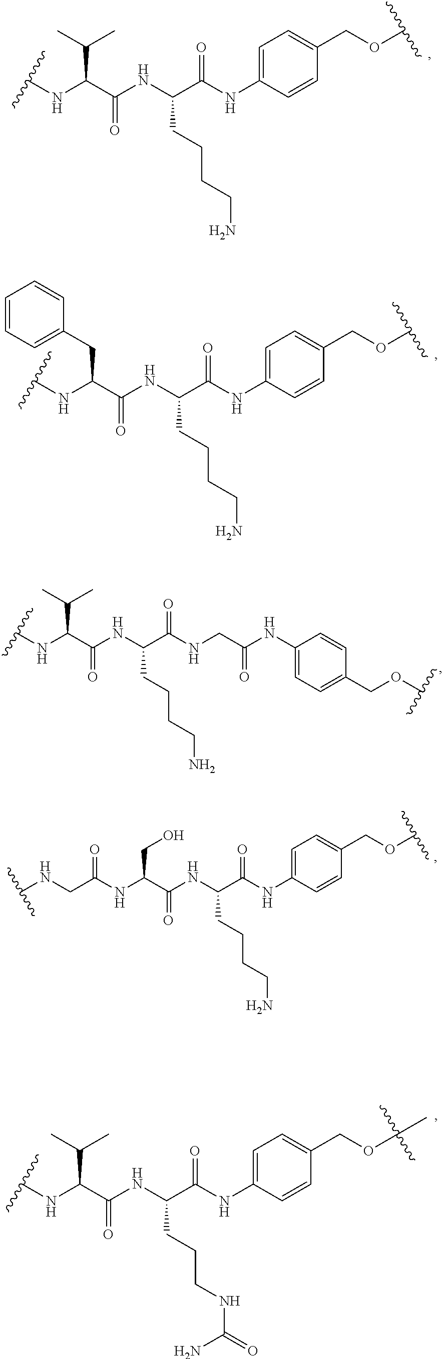 [SMILES: *N[C@H](C(=O)N[C@@H](CCCCN)C(=O)Nc1ccc(CO*)cc1)C(C)C ; *N[C@@H](Cc1ccccc1)C(=O)N[C@@H](CCCCN)C(=O)Nc1ccc(CO*)cc1 ; *N[C@H](C(=O)N[C@@H](CCCCN)C(=O)NCC(=O)Nc1ccc(CO*)cc1)C(C)C ; *NCC(=O)N[C@@H](CO)C(=O)N[C@@H](CCCCN)C(=O)Nc1ccc(CO*)cc1 ; *N[C@H](C(=O)N[C@@H](CCCNC(N)=O)C(=O)Nc1ccc(CO*)cc1)C(C)C]

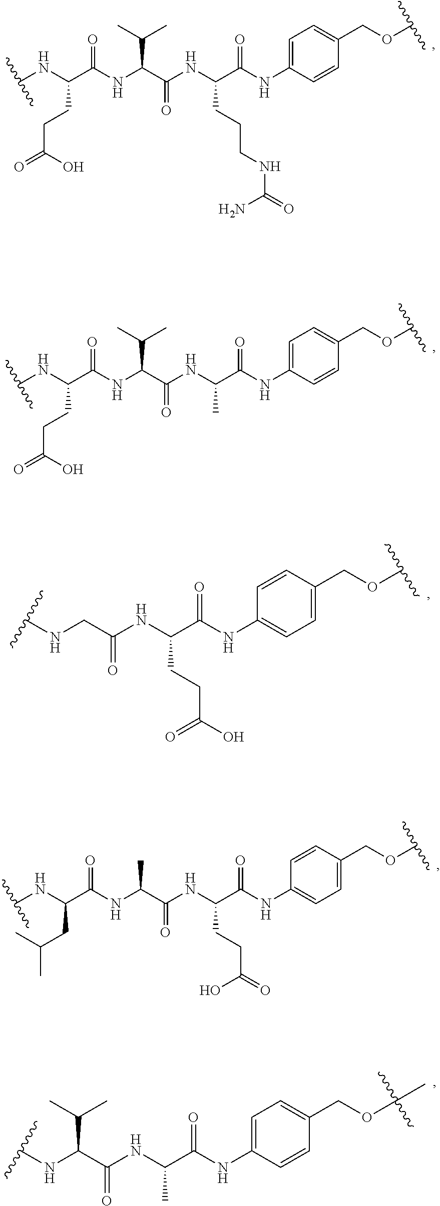 [SMILES: *N[C@@H](CCC(=O)O)C(=O)N[C@H](C(=O)N[C@@H](CCCNC(N)=O)C(=O)Nc1ccc(CO*)cc1)C(C)C ; *N[C@@H](CCC(=O)O)C(=O)N[C@H](C(=O)N[C@@H](C)C(=O)Nc1ccc(CO*)cc1)C(C)C ; *NCC(=O)N[C@@H](CCC(=O)O)C(=O)Nc1ccc(CO*)cc1 ; *N[C@H](CC(C)C)C(=O)N[C@@H](C)C(=O)N[C@@H](CCC(=O)O)C(=O)Nc1ccc(CO*)cc1 ; *N[C@H](C(=O)N[C@@H](C)C(=O)Nc1ccc(CO*)cc1)C(C)C]

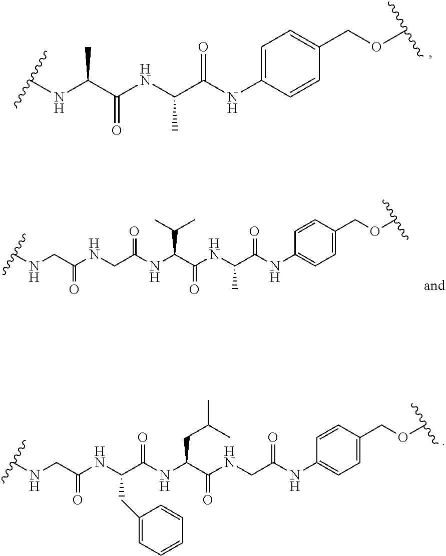 [SMILES: *N[C@@H](C)C(=O)N[C@@H](C)C(=O)Nc1ccc(CO*)cc1 ; *NCC(=O)NCC(=O)N[C@H](C(=O)N[C@@H](C)C(=O)Nc1ccc(CO*)cc1)C(C)C ; *NCC(=O)N[C@@H](Cc1ccccc1)C(=O)N[C@@H](CC(C)C)C(=O)NCC(=O)Nc1ccc(CO*)cc1]

In some embodiments, L is selected from the group consisting of structural fragments composed of one or more of the following groups:

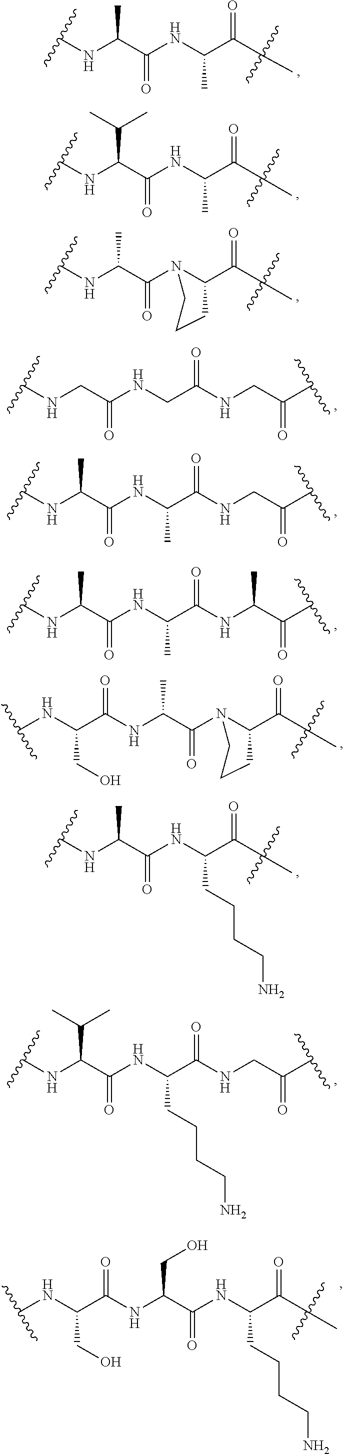

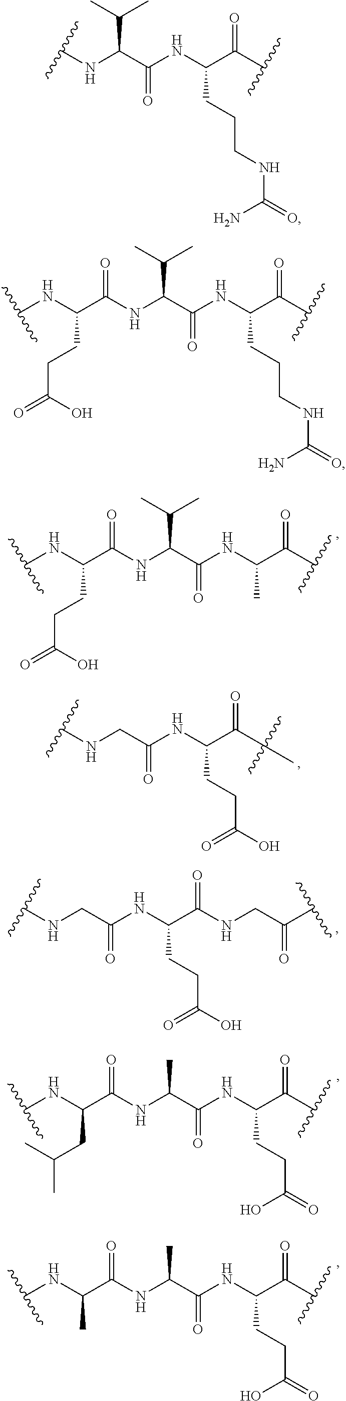

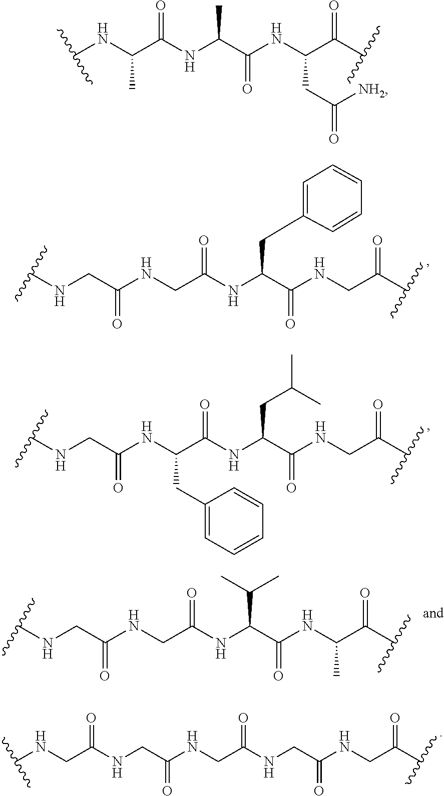

In some embodiments, L is selected from the group consisting of divalent structural fragments composed of one or more of the following groups:

[chemical structure]

s is an integer selected from 1 to 20.

In some embodiments, L is selected from the group consisting of the following substituted or unsubstituted structural fragments:

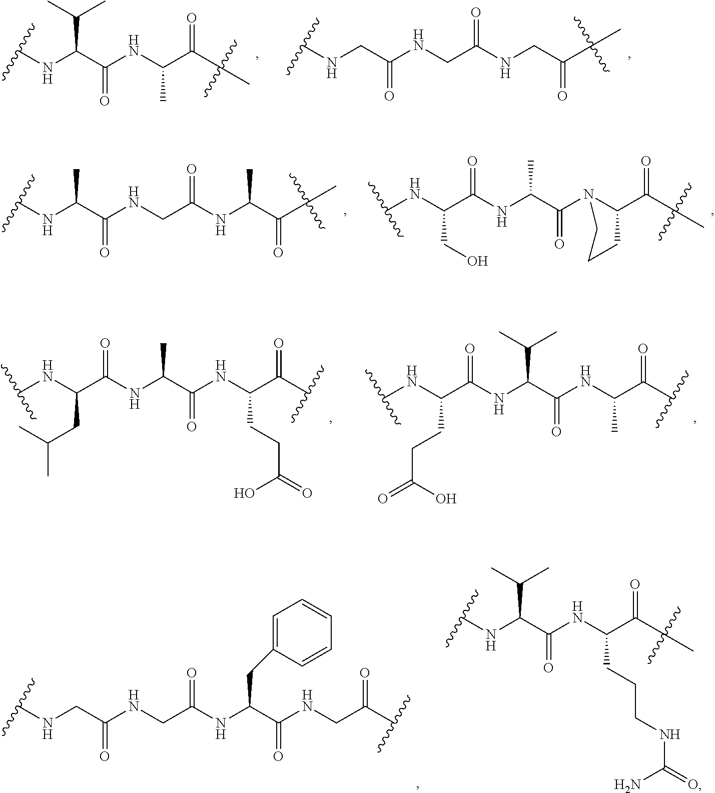

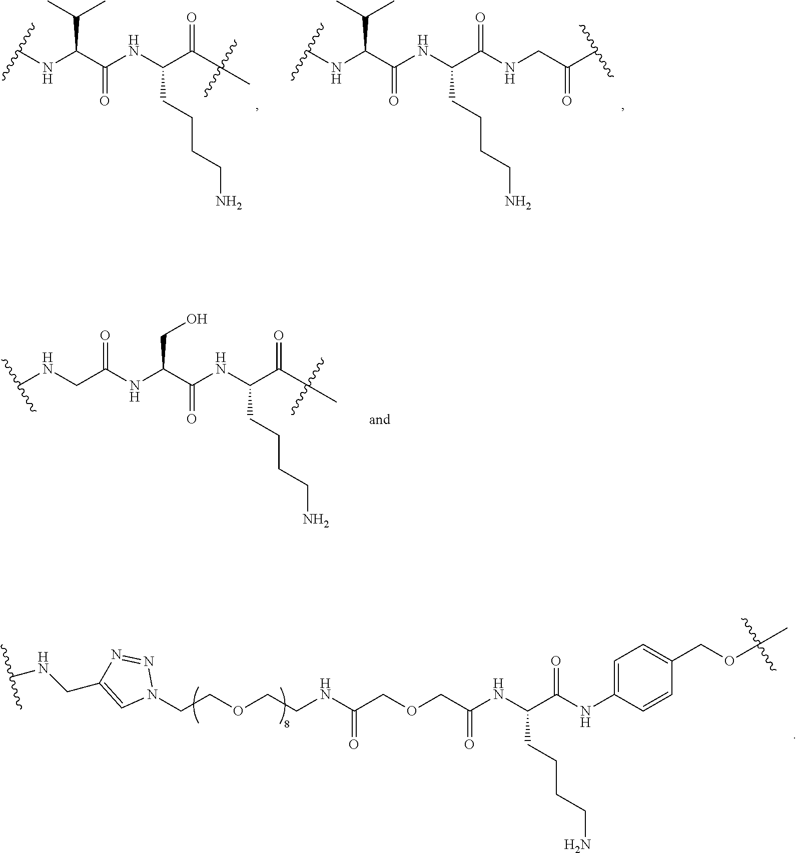

In some embodiments, L is selected from the group consisting of:

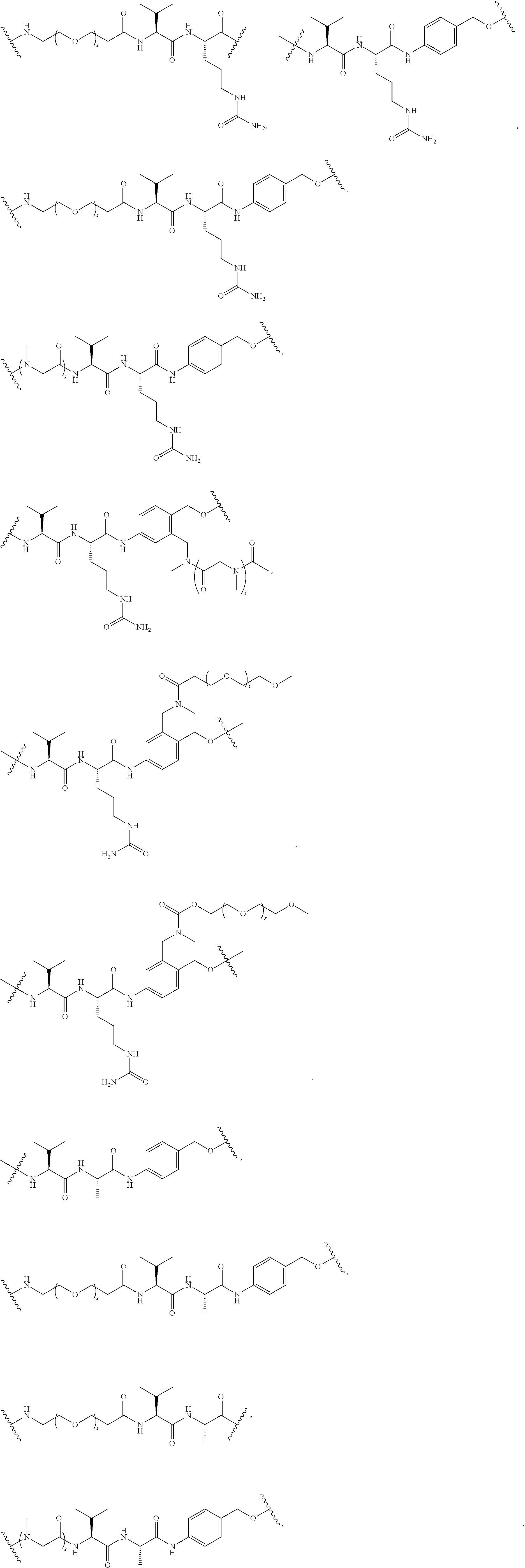

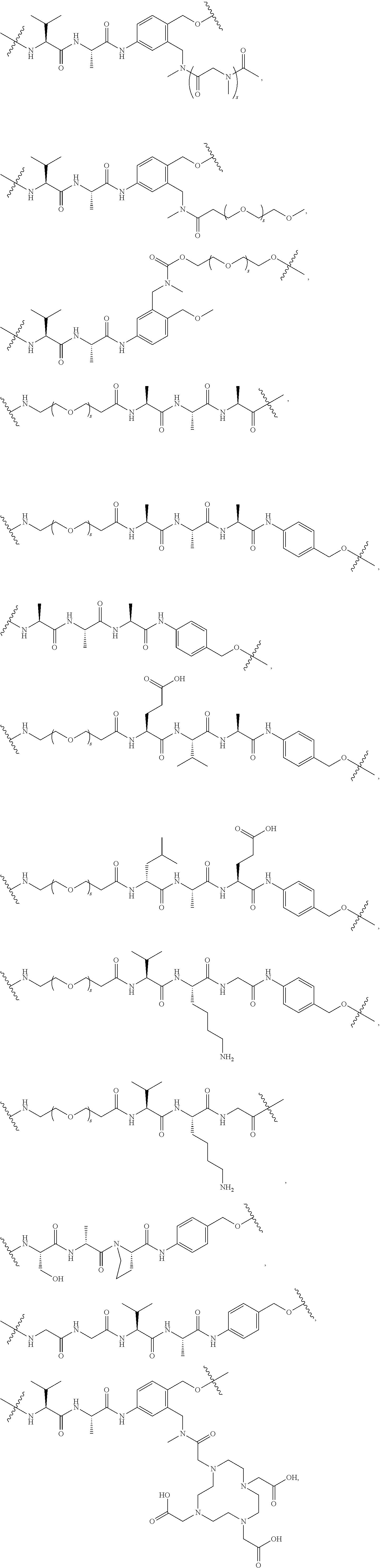

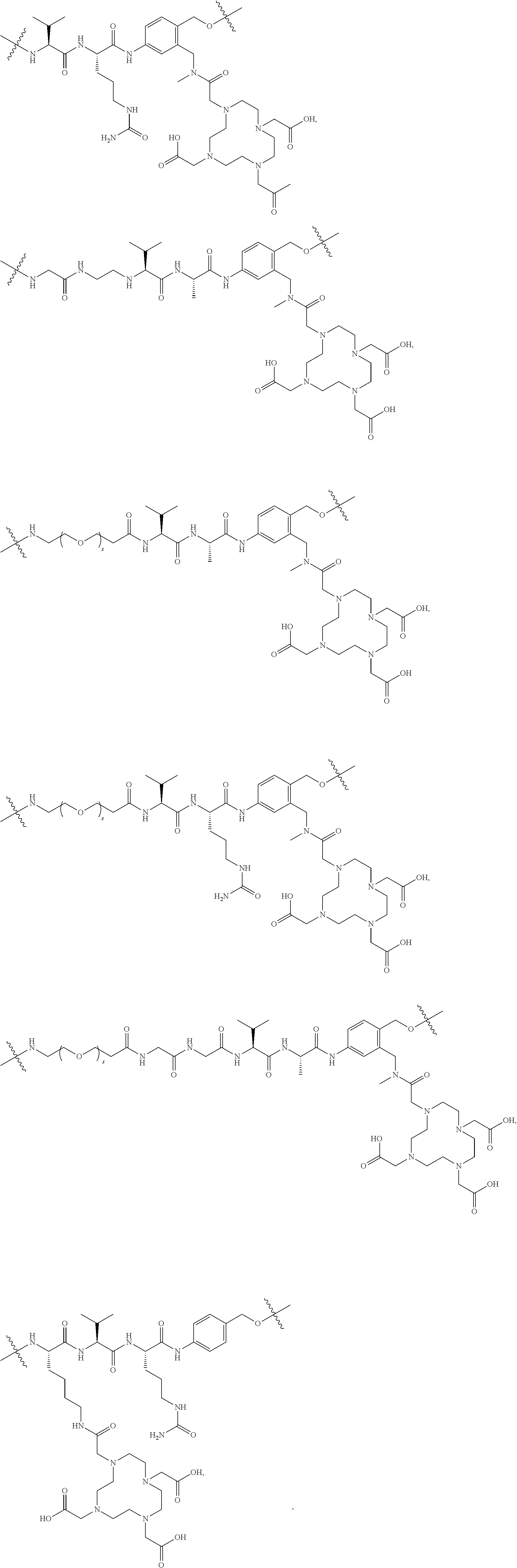

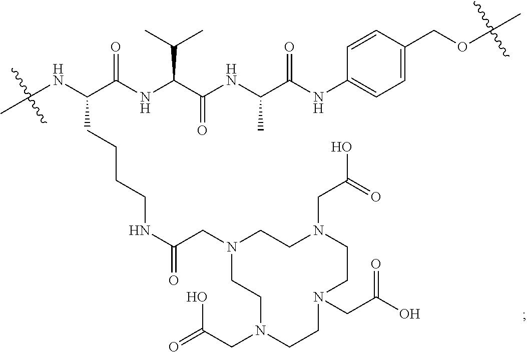 [SMILES: *N[C@@H](CCCCNC(=O)CN1CCN(CC(=O)O)CCN(CC(=O)O)CCN(CC(=O)O)CC1)C(=O)N[C@H](C(=O)N[C@@H](C)C(=O)Nc1ccc(CO*)cc1)C(C)C]

s is an integer selected from 1 to 20.

In any of the above embodiments, s is selected from the group consisting of 1, 2, 3, 4, 5, 6, 7, 8, 9, 10, 11, 12, 13, 14, 15, 16, 17, 18, 19 and 20.

In any of the above embodiments, s is an integer selected from 5 to 15, such as 5, 6, 7, 8, 9, 10, 11, 12, 13, 14 or 15.

In some embodiments, L is selected from the group consisting of:

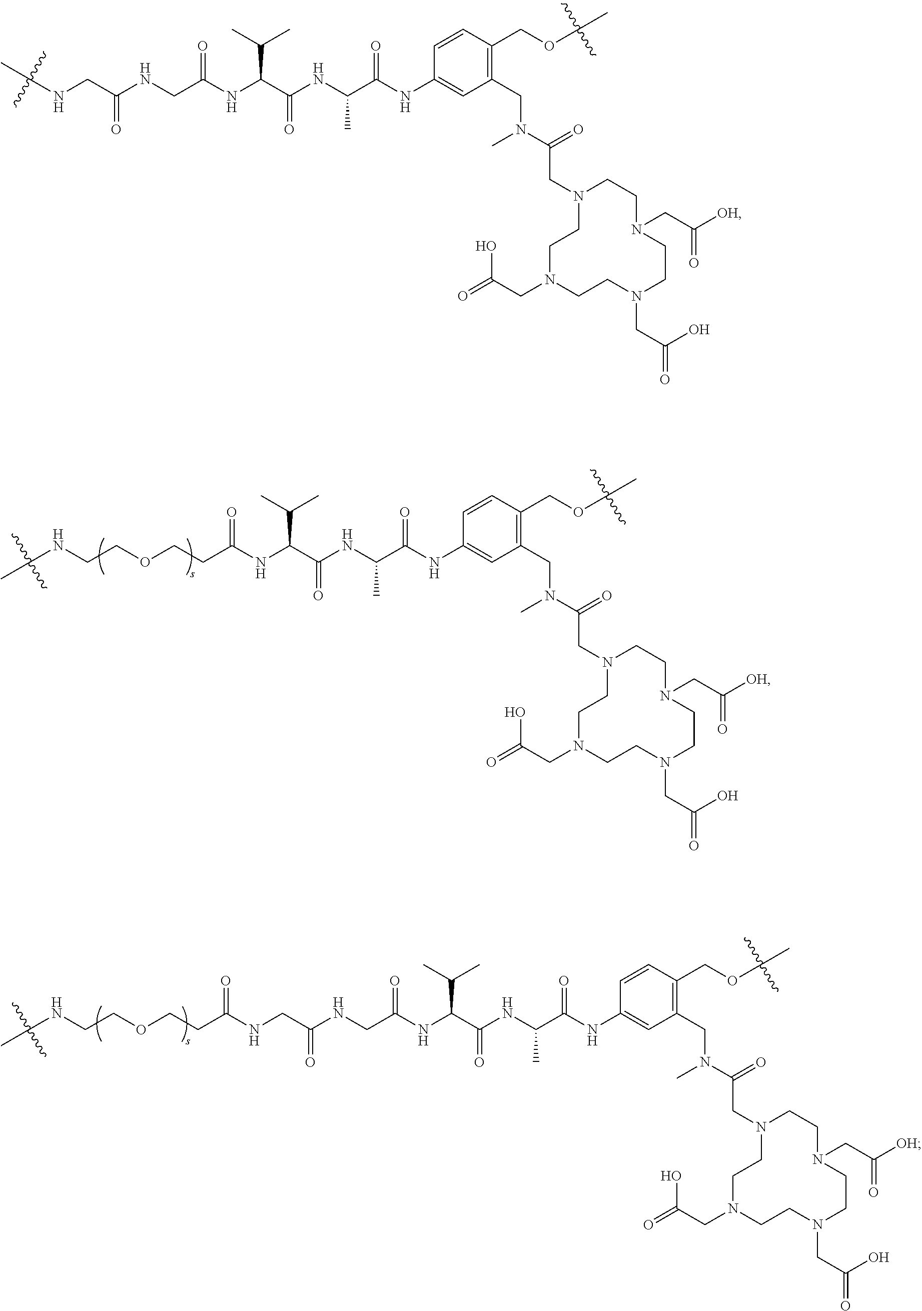

s is an integer selected from 5 to 15, such as 5, 6, 7, 8, 9, 10, 11, 12, 13, 14 or 15.

In some embodiments, E is a single bond, —NH—CH₂—, or is selected from the group consisting of the following structural fragments:

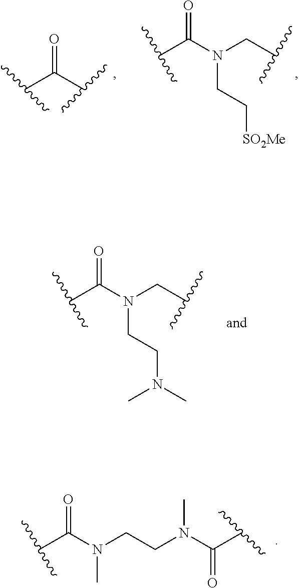

In some embodiments, E is a single bond, —NH—CH₂— or

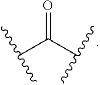 [SMILES: *C(*)=O]

In some embodiments, E is —NH—CH₂— or

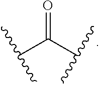 [SMILES: *C(*)=O]

In some embodiments, E is

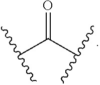 [SMILES: *C(*)=O]

In some embodiments,

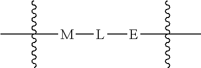

is selected from the group consisting of the following structures:

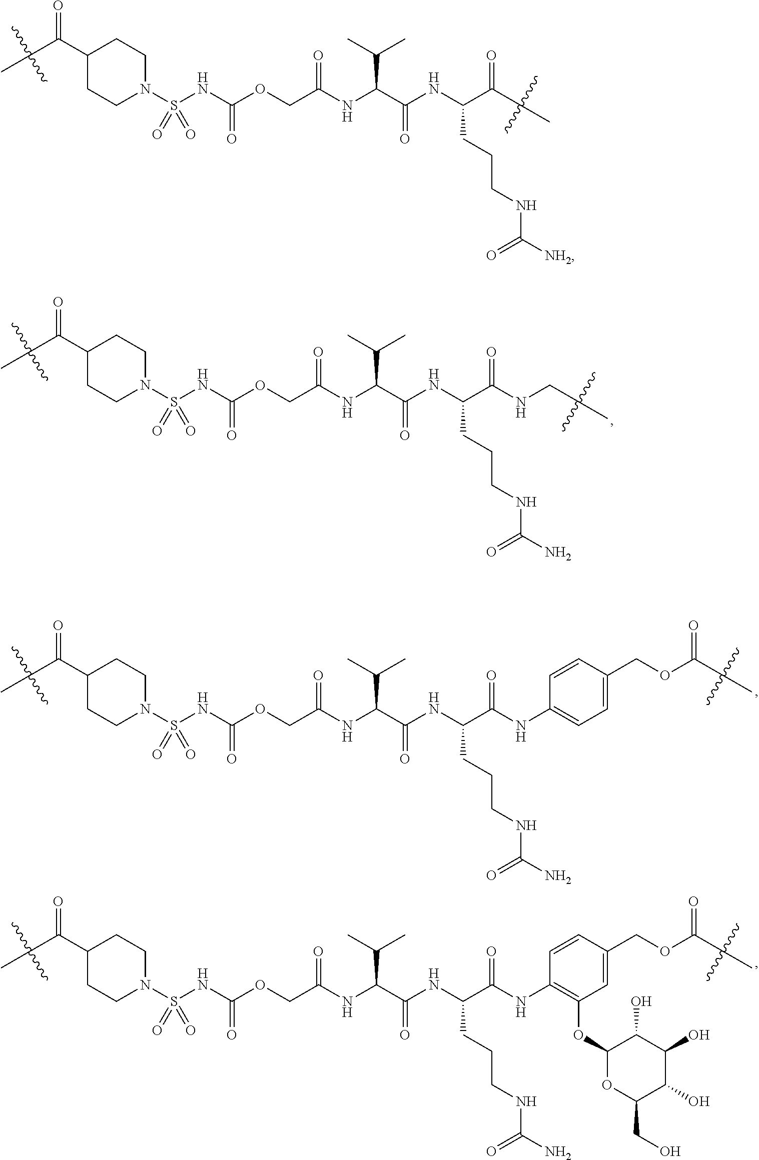 [SMILES: *C(=O)C1CCN(S(=O)(=O)NC(=O)OCC(=O)N[C@H](C(=O)N[C@@H](CCCNC(N)=O)C(*)=O)C(C)C)CC1 ; *CNC(=O)[C@H](CCCNC(N)=O)NC(=O)[C@@H](NC(=O)COC(=O)NS(=O)(=O)N1CCC(C(*)=O)CC1)C(C)C ; *C(=O)OCc1ccc(NC(=O)[C@H](CCCNC(N)=O)NC(=O)[C@@H](NC(=O)COC(=O)NS(=O)(=O)N2CCC(C(*)=O)CC2)C(C)C)cc1 ; *C(=O)OCc1ccc(NC(=O)[C@H](CCCNC(N)=O)NC(=O)[C@@H](NC(=O)COC(=O)NS(=O)(=O)N2CCC(C(*)=O)CC2)C(C)C)c(O[C@@H]2O[C@H](CO)[C@@H](O)[C@H](O)[C@H]2O)c1]

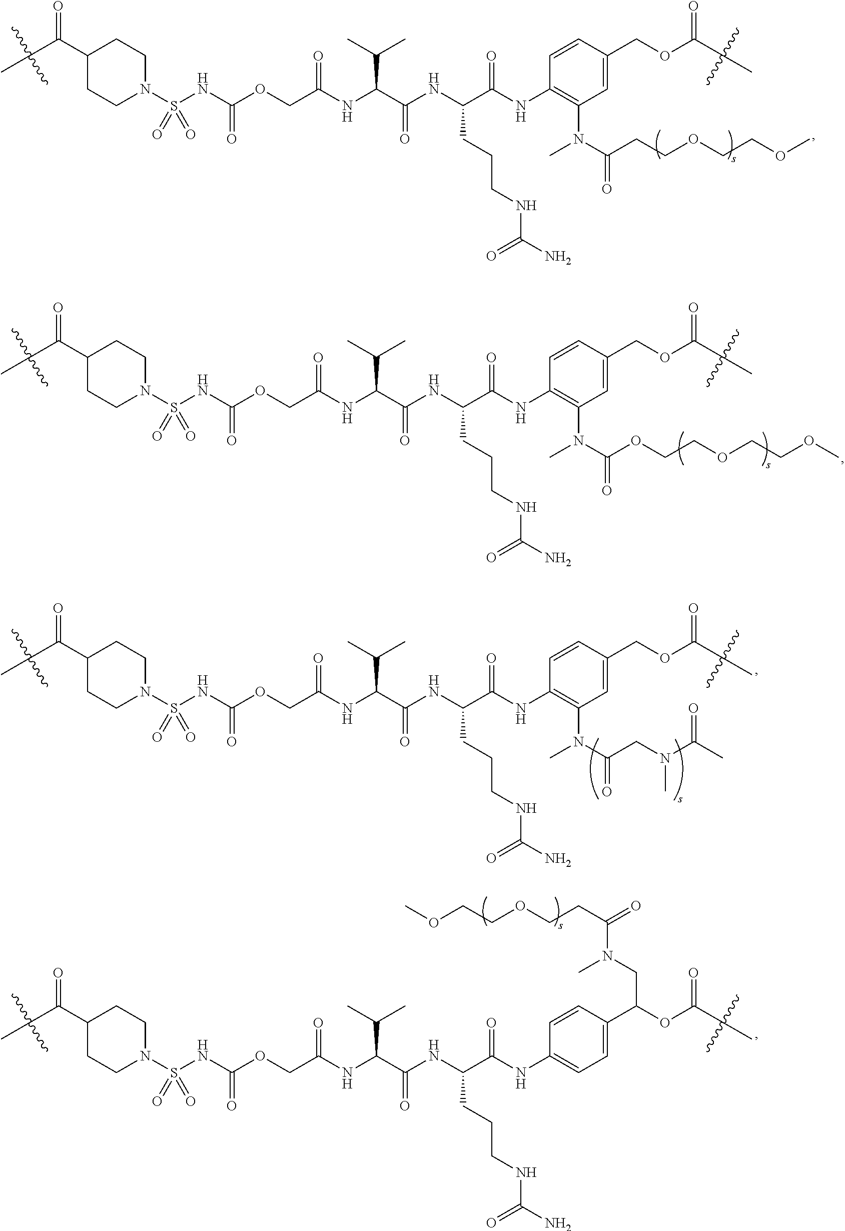

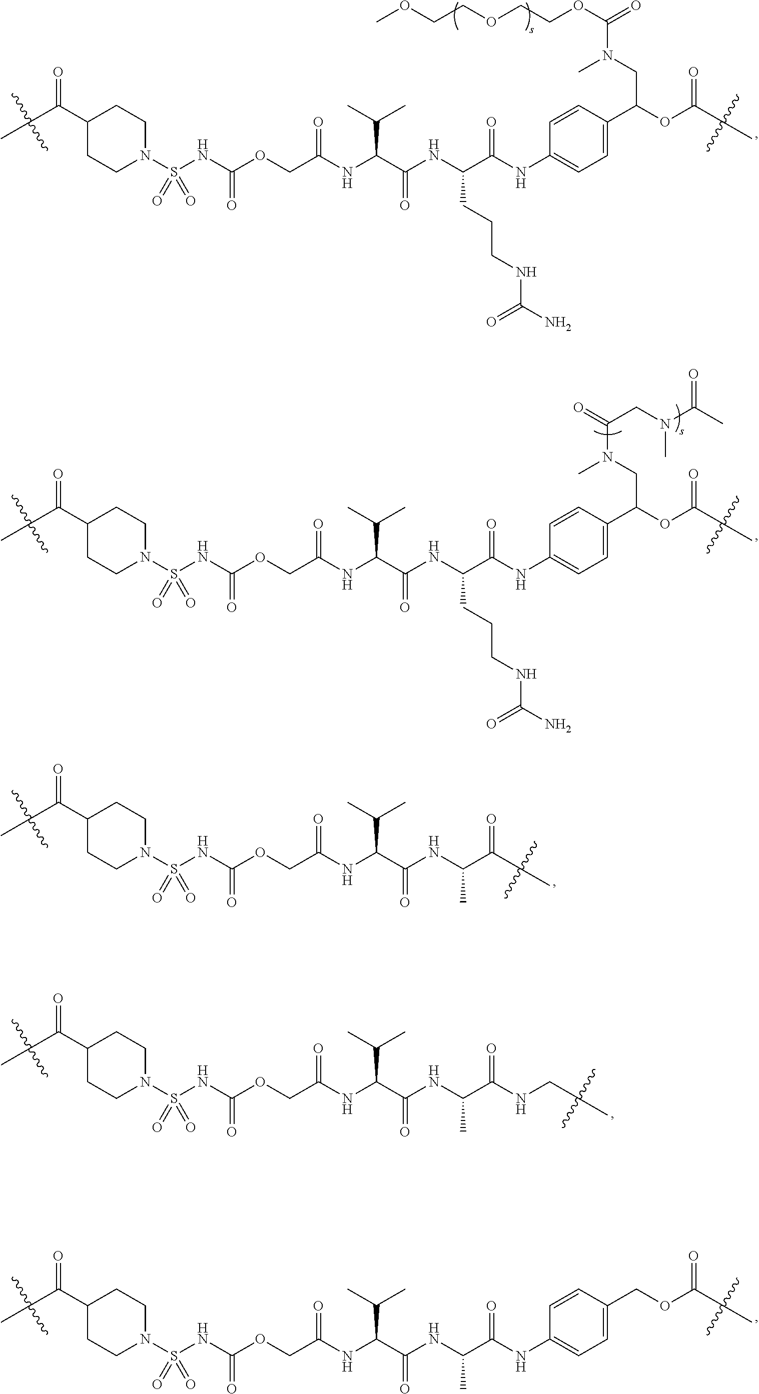

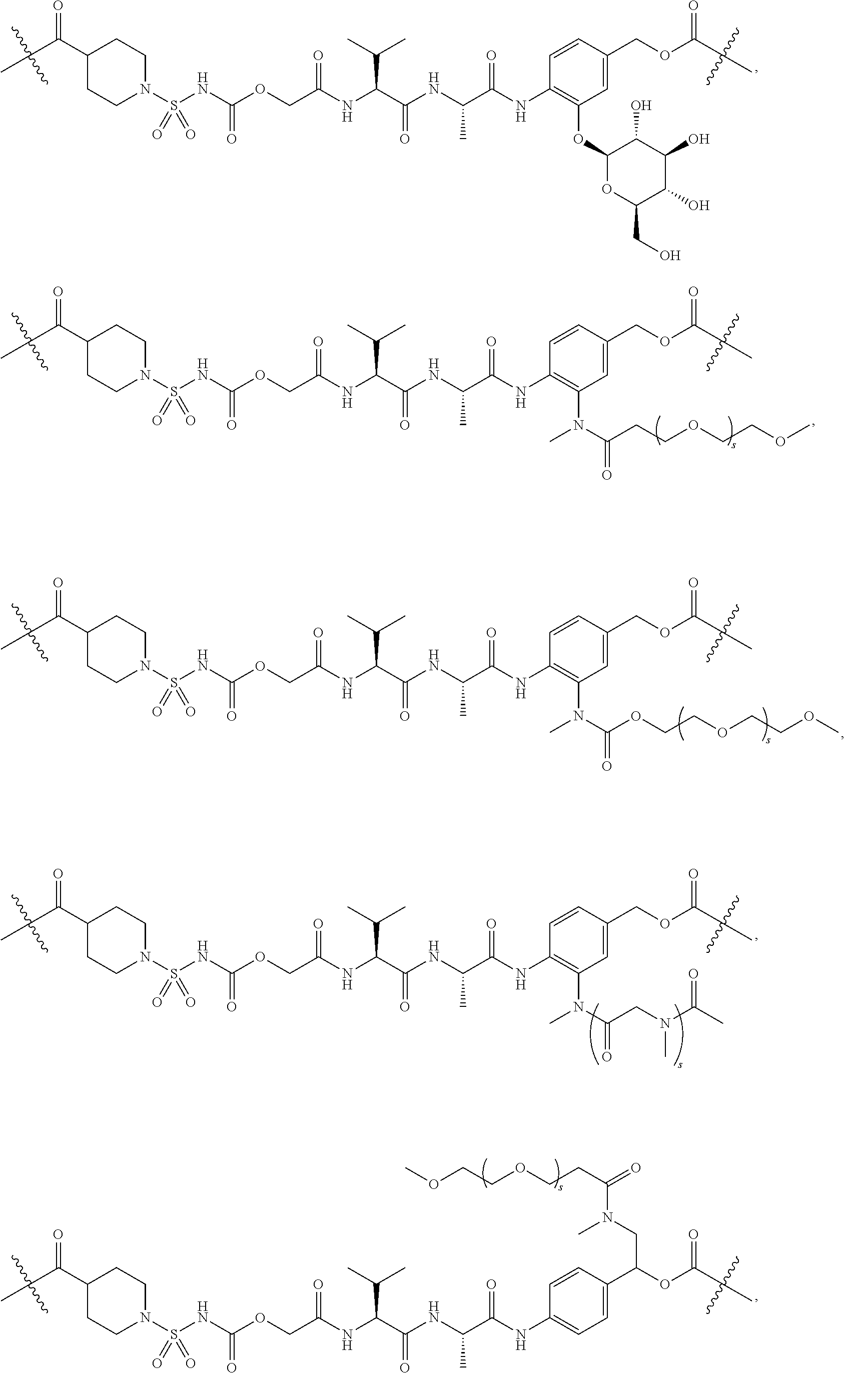

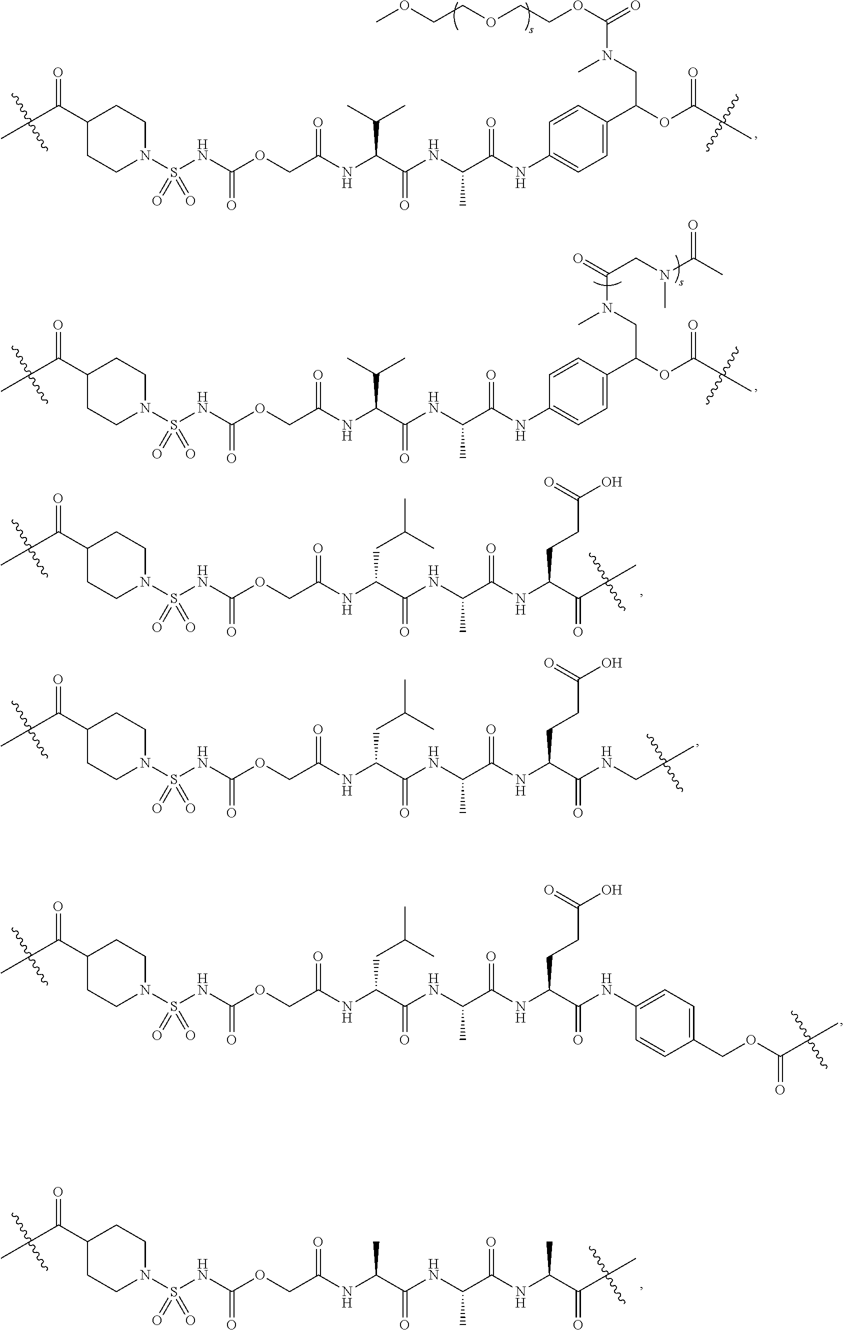

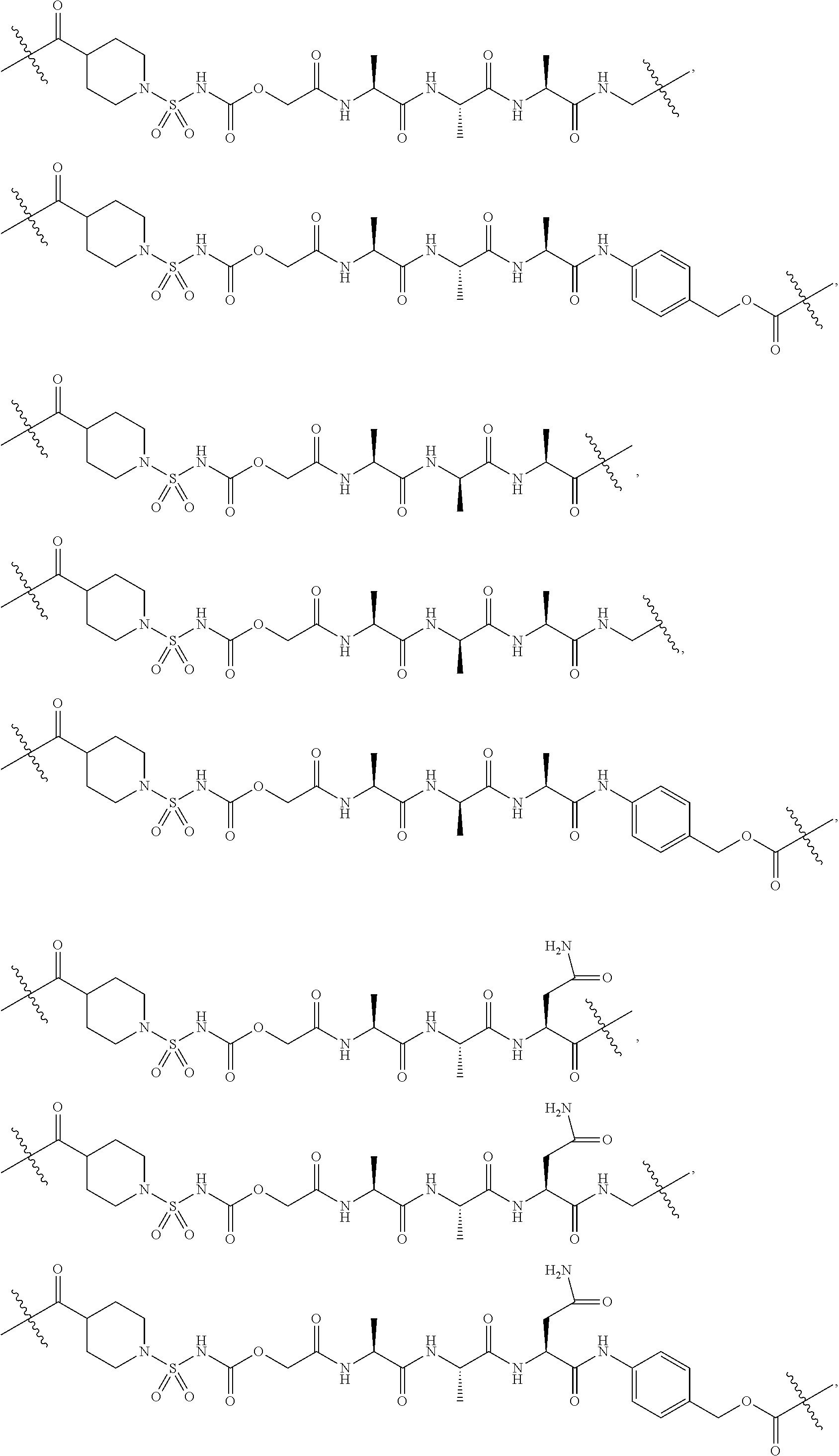

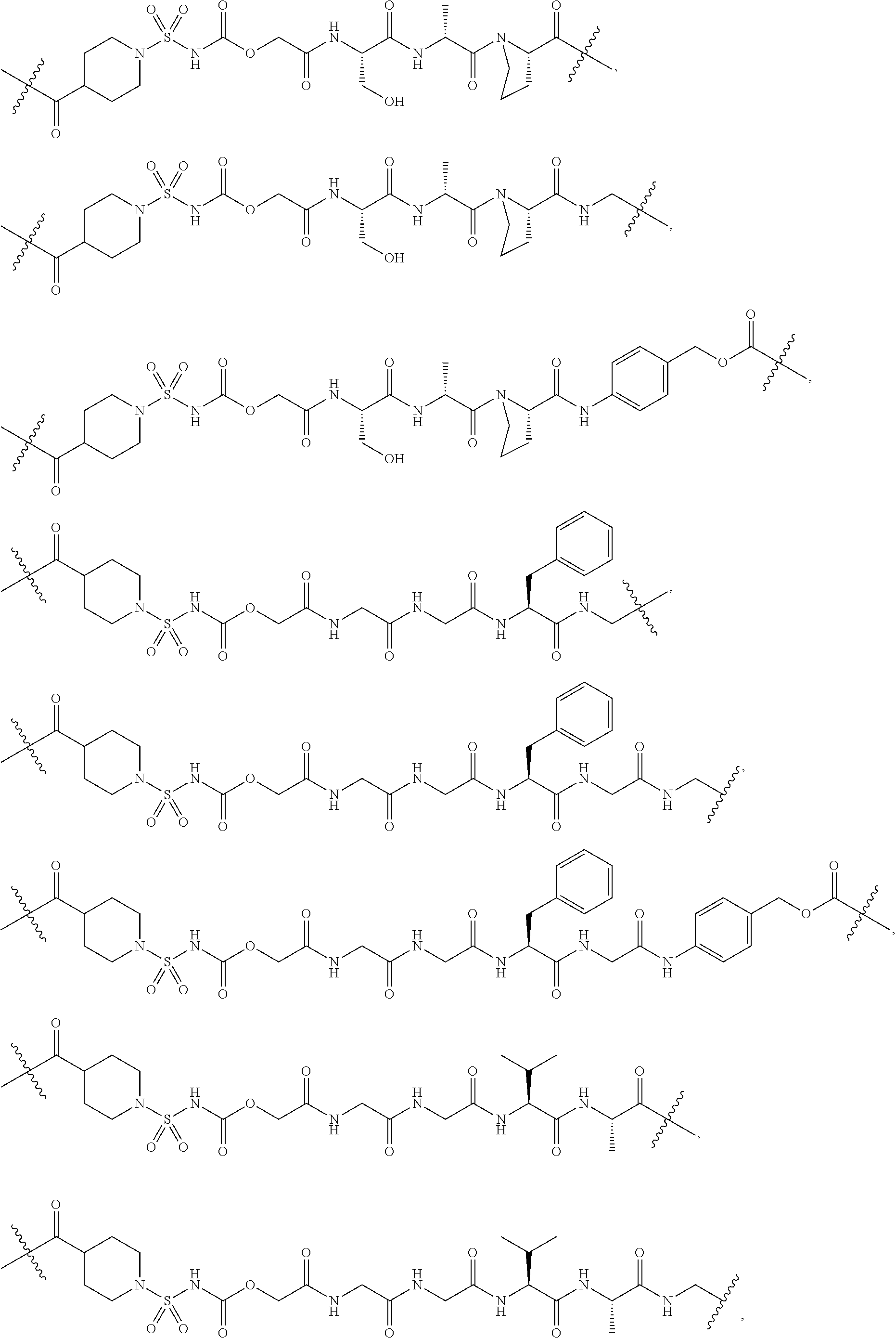

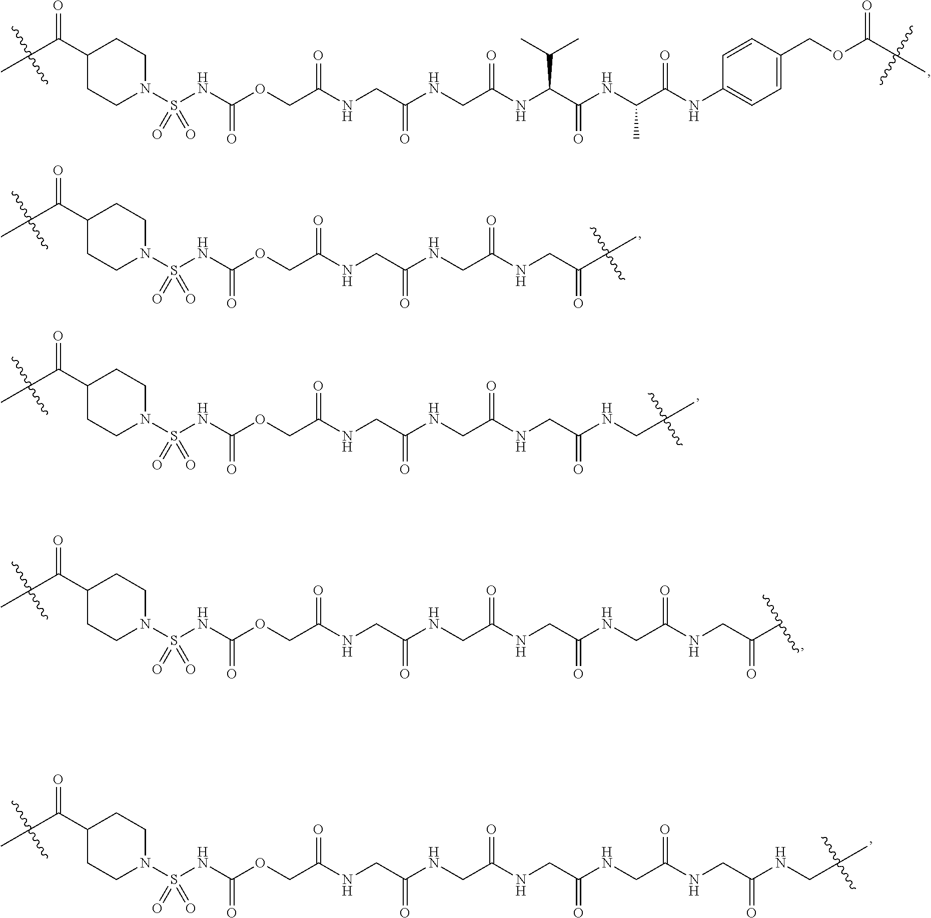 [SMILES: *C(=O)OCc1ccc(NC(=O)[C@H](C)NC(=O)[C@@H](NC(=O)CNC(=O)CNC(=O)COC(=O)NS(=O)(=O)N2CCC(C(*)=O)CC2)C(C)C)cc1 ; *C(=O)CNC(=O)CNC(=O)CNC(=O)COC(=O)NS(=O)(=O)N1CCC(C(*)=O)CC1 ; *CNC(=O)CNC(=O)CNC(=O)CNC(=O)COC(=O)NS(=O)(=O)N1CCC(C(*)=O)CC1 ; *C(=O)CNC(=O)CNC(=O)CNC(=O)CNC(=O)CNC(=O)COC(=O)NS(=O)(=O)N1CCC(C(*)=O)CC1 ; *CNC(=O)CNC(=O)CNC(=O)CNC(=O)CNC(=O)CNC(=O)COC(=O)NS(=O)(=O)N1CCC(C(*)=O)CC1]

s is an integer selected from 1 to 20; preferably, s is an integer selected from 5 to 15, such as 5, 6, 7, 8, 9, 10, 11, 12, 13, 14 or 15.

In some embodiments,

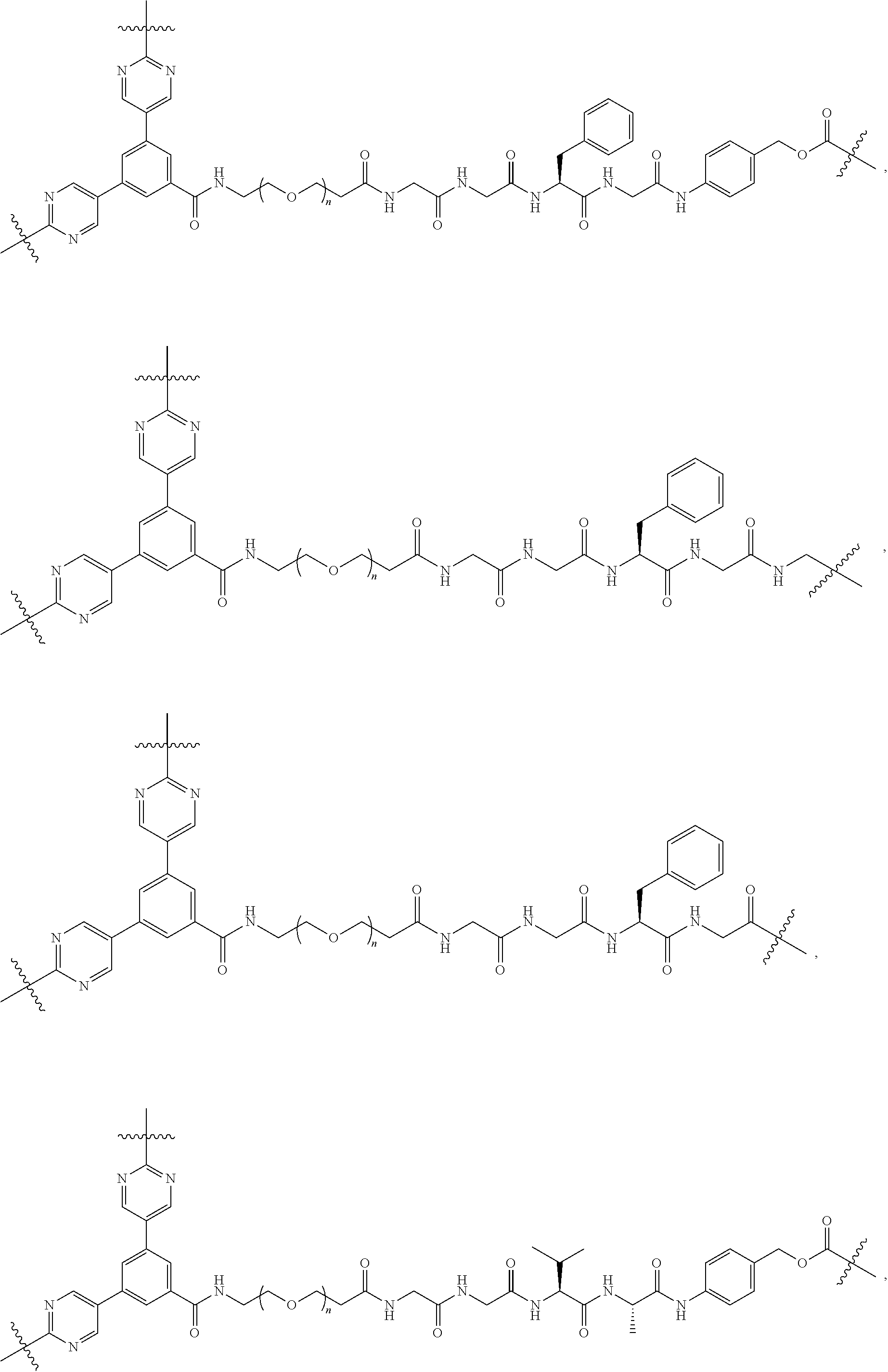

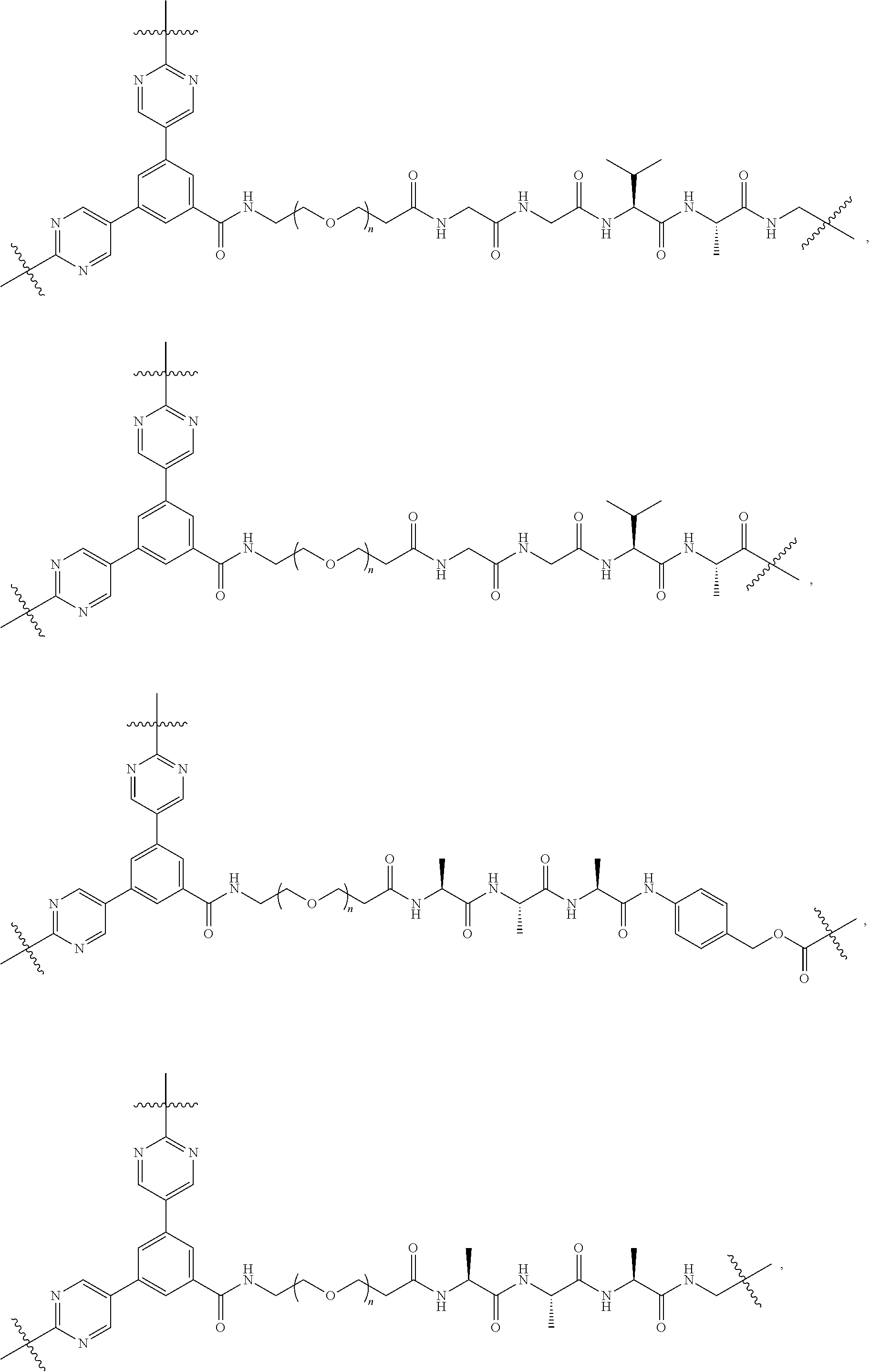

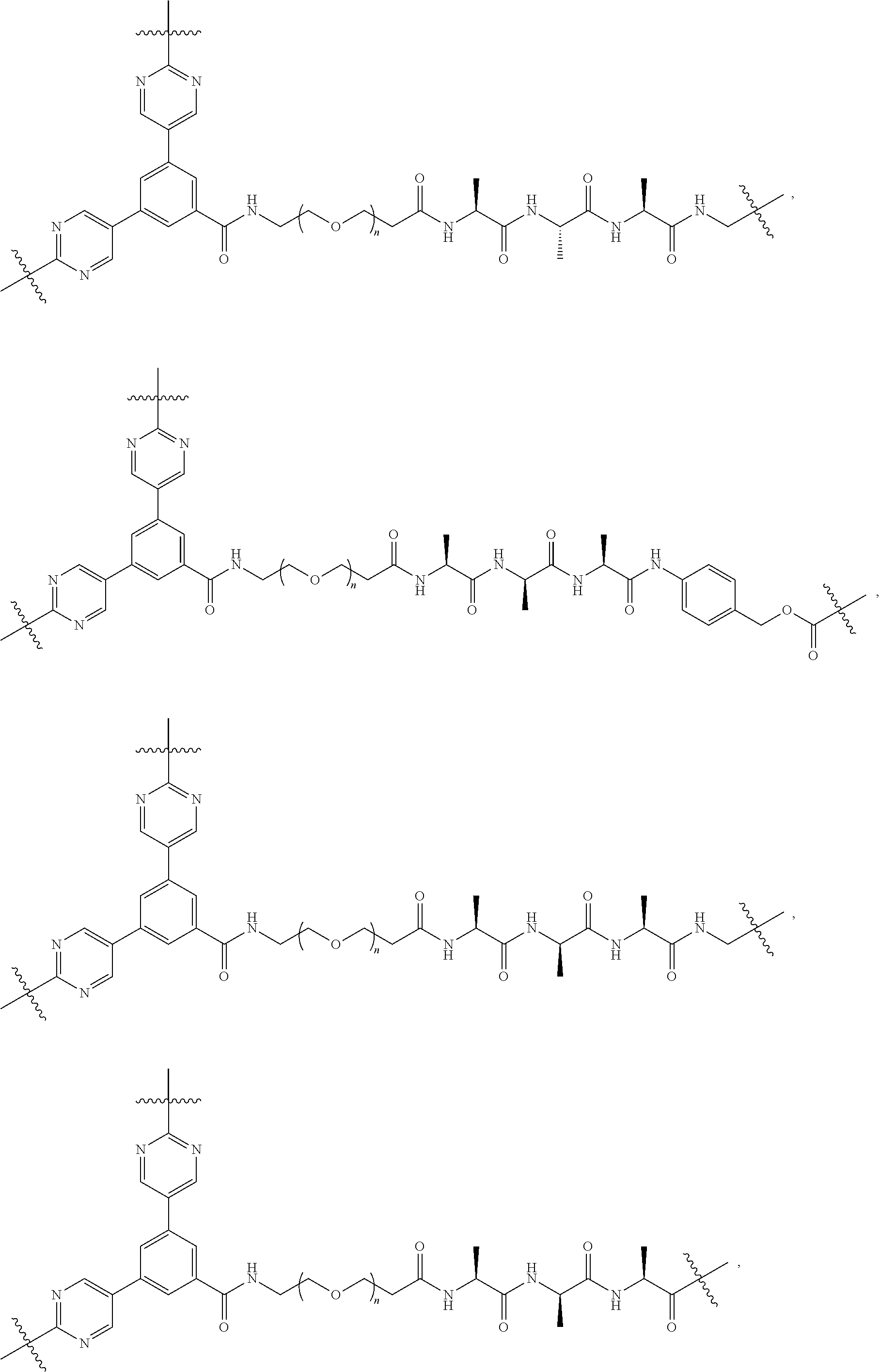

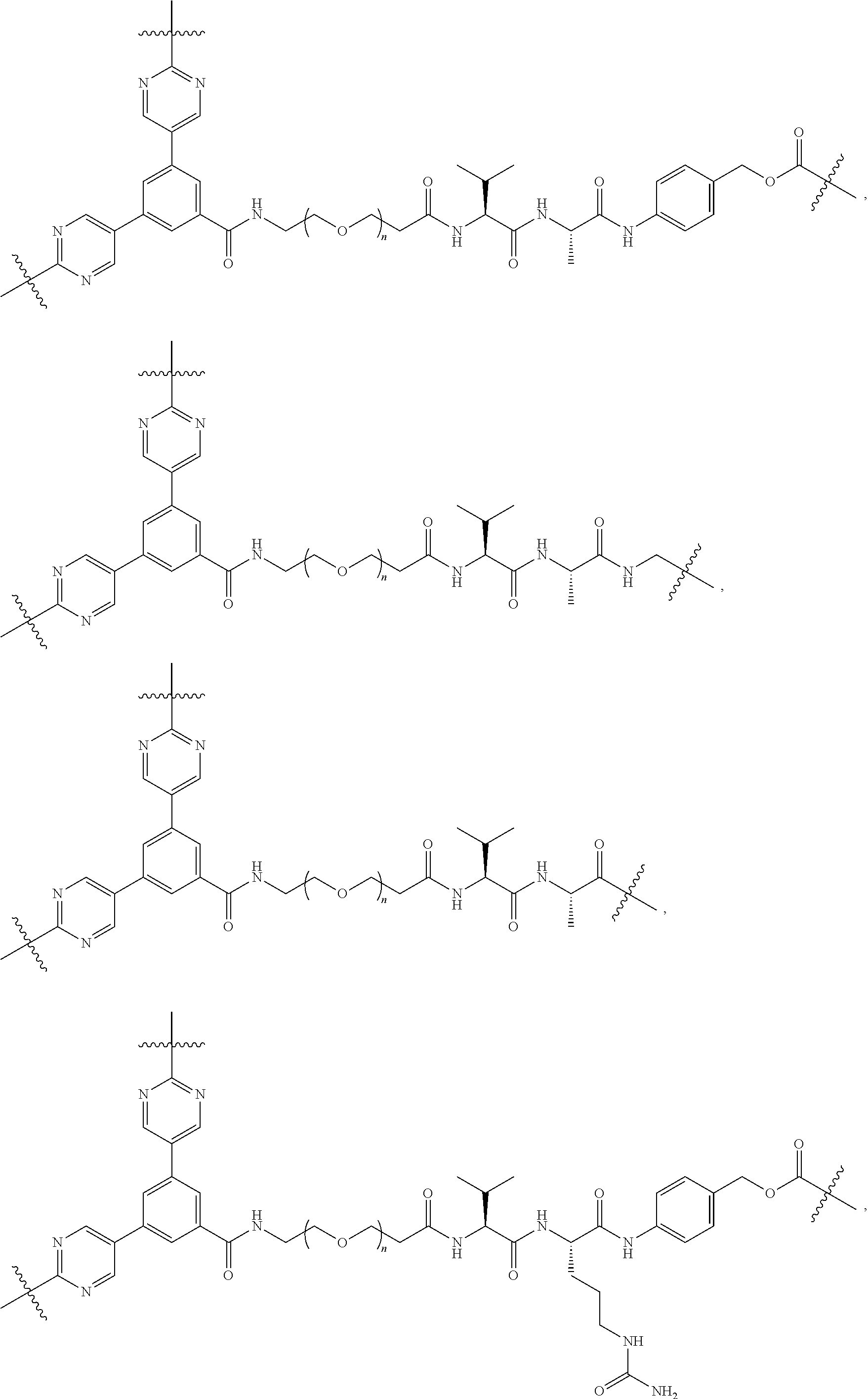

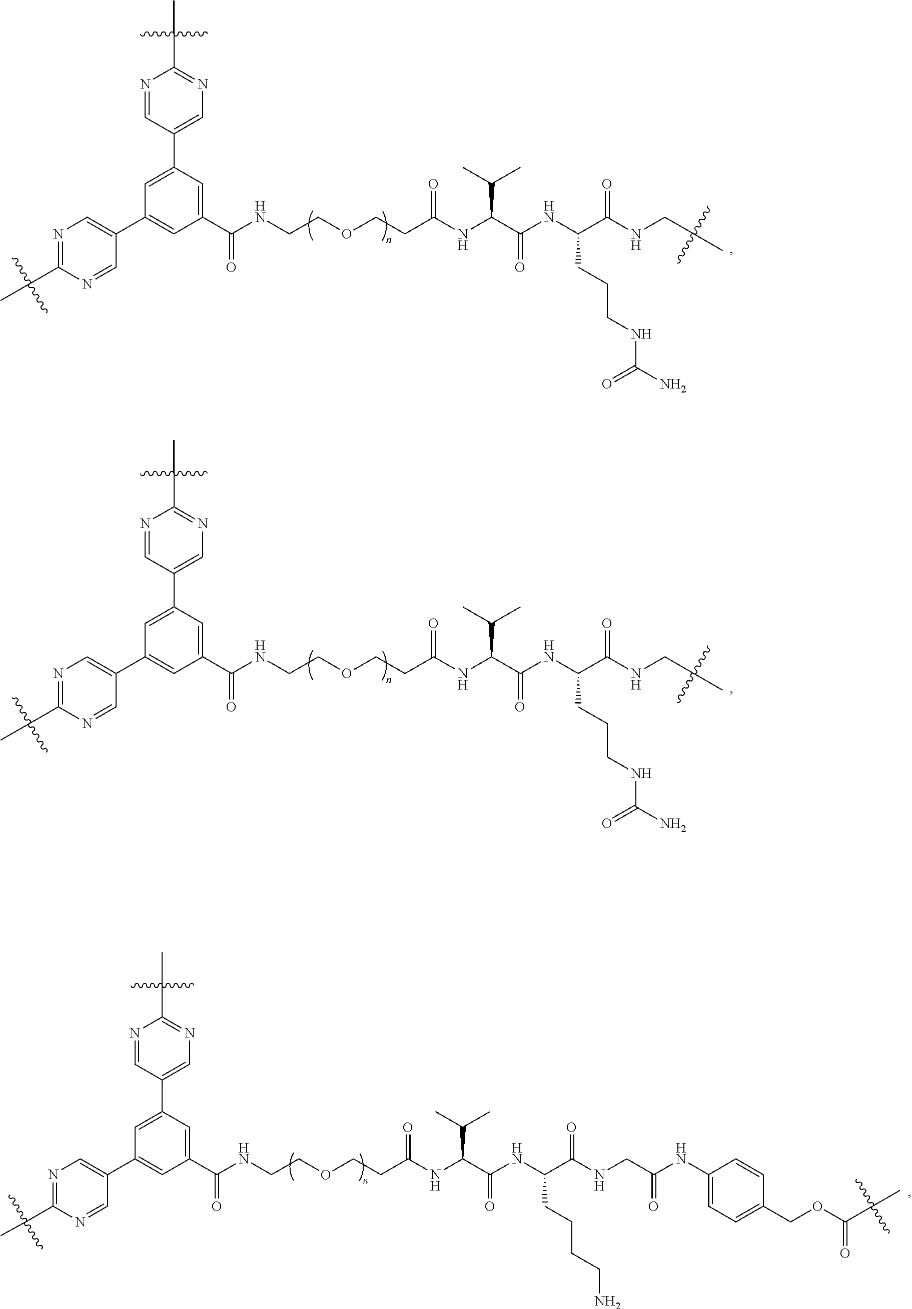

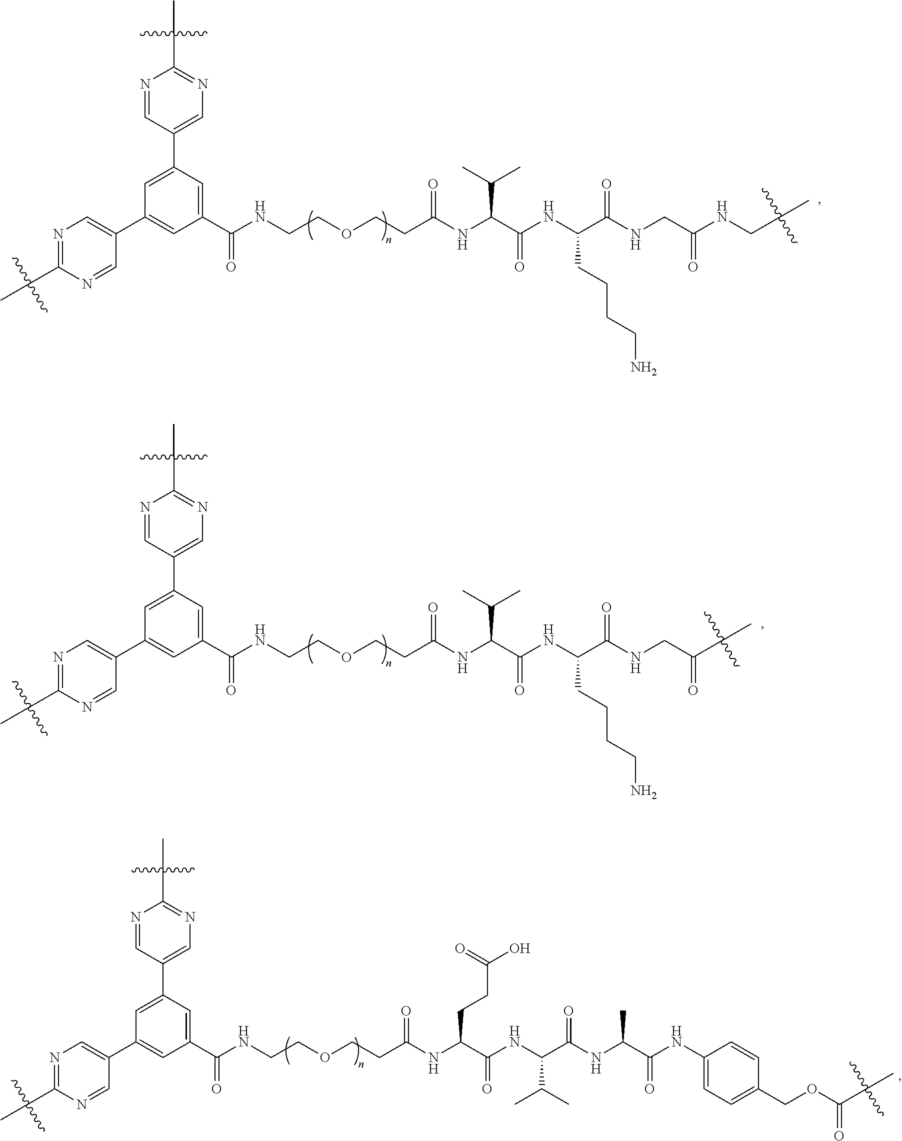

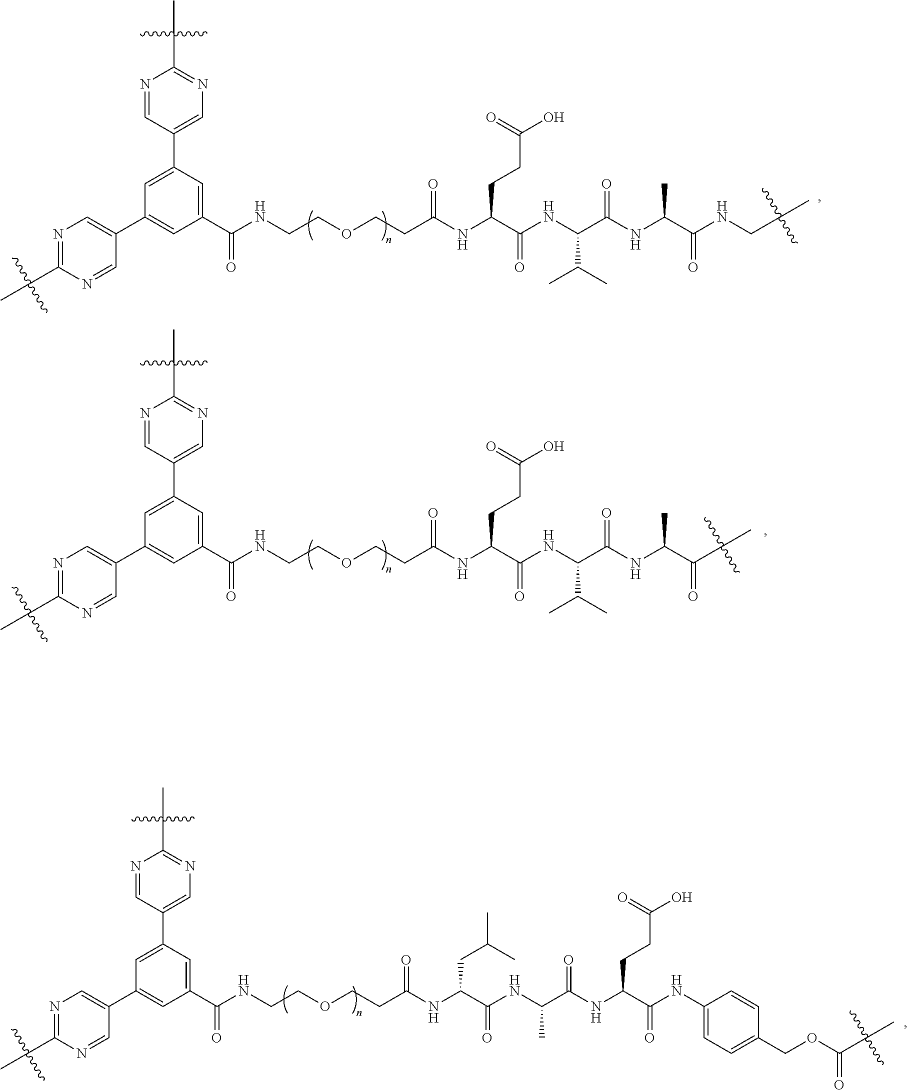

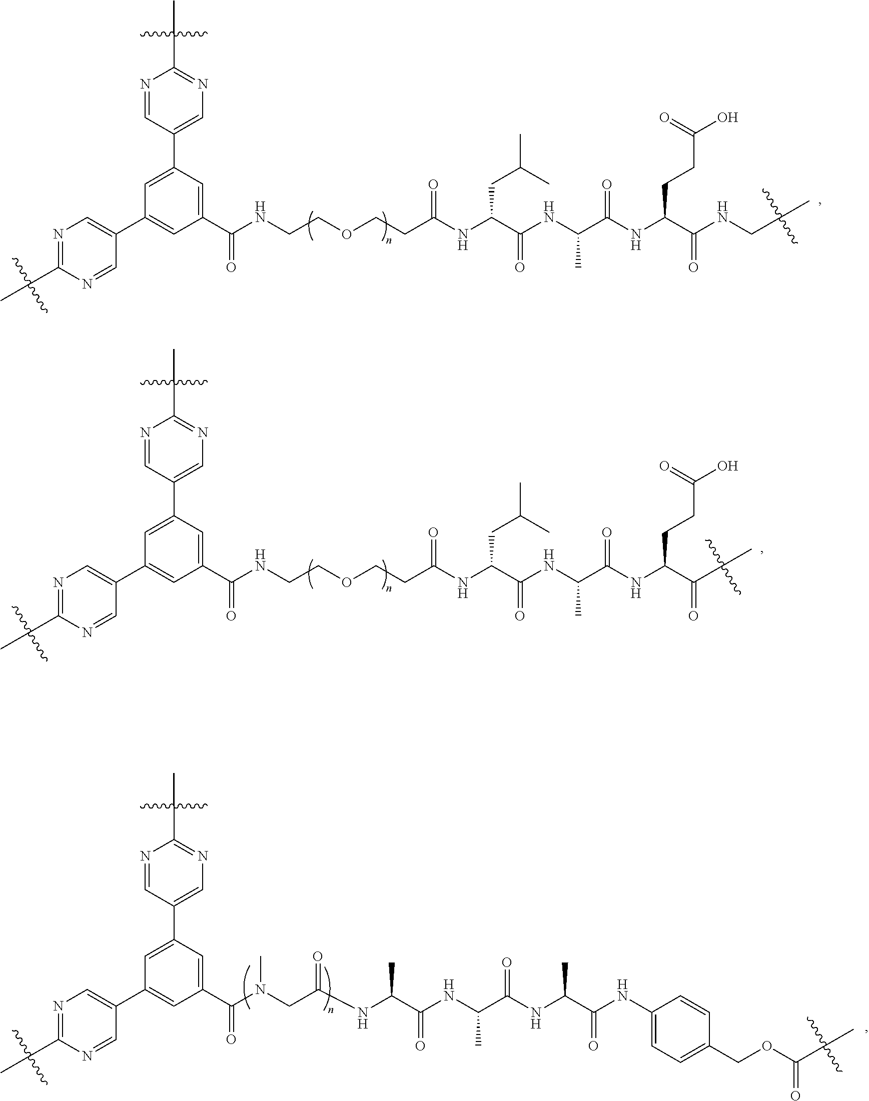

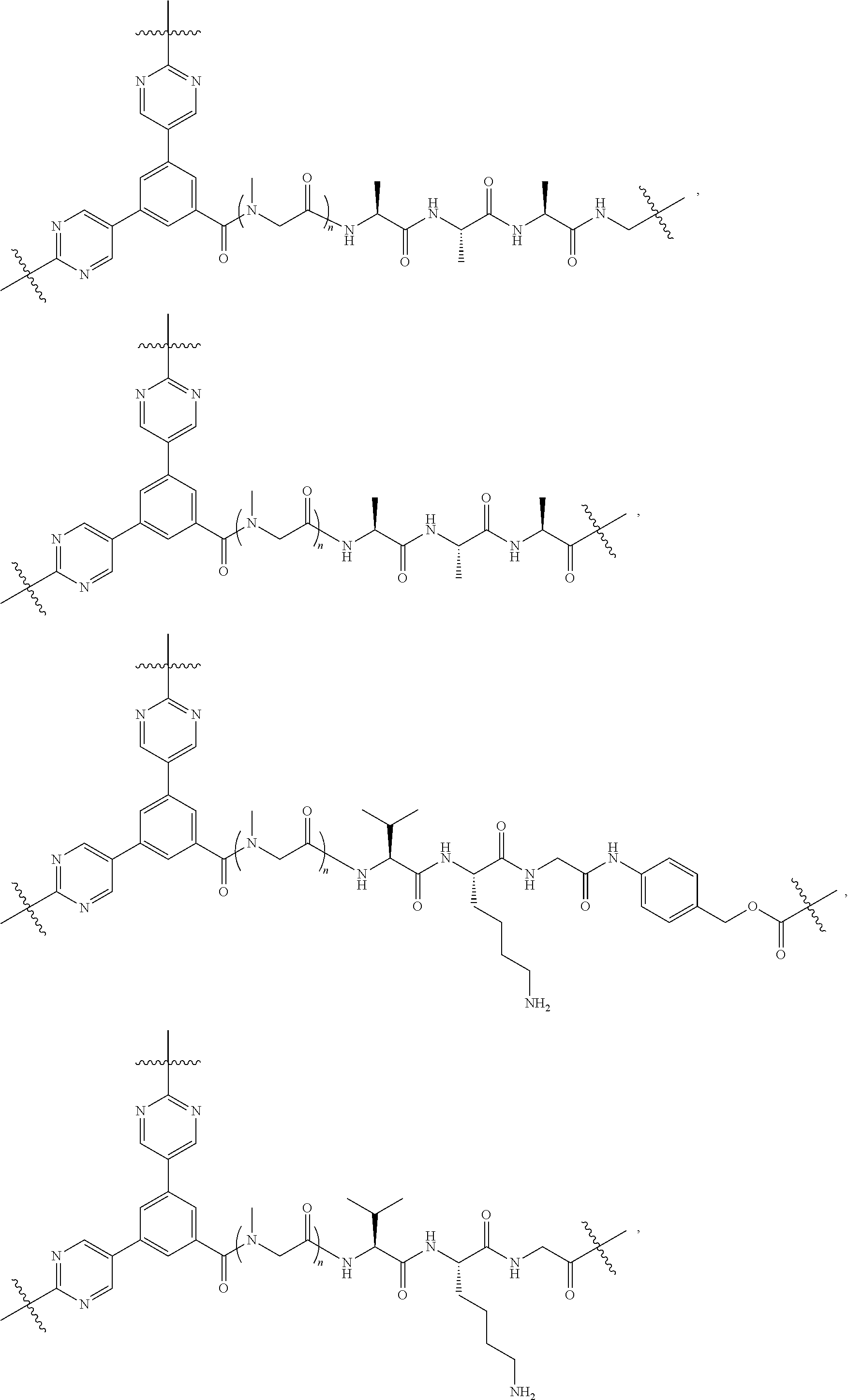

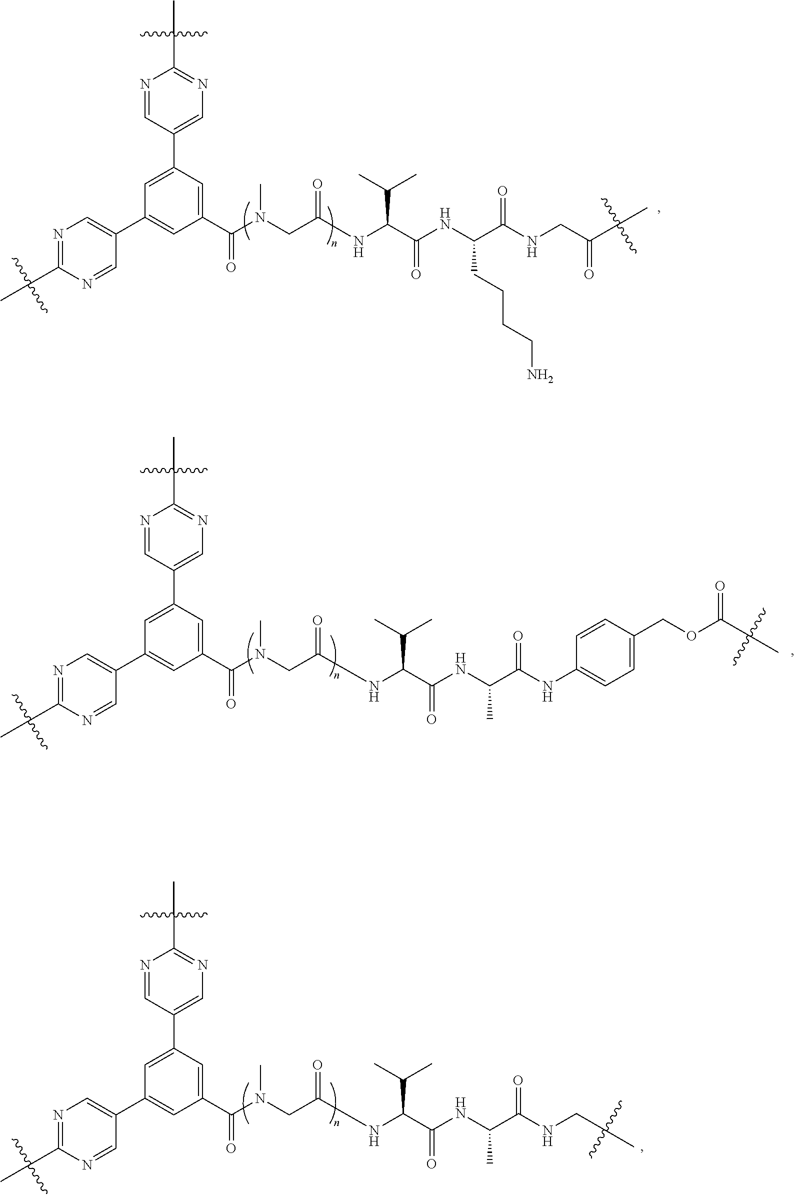

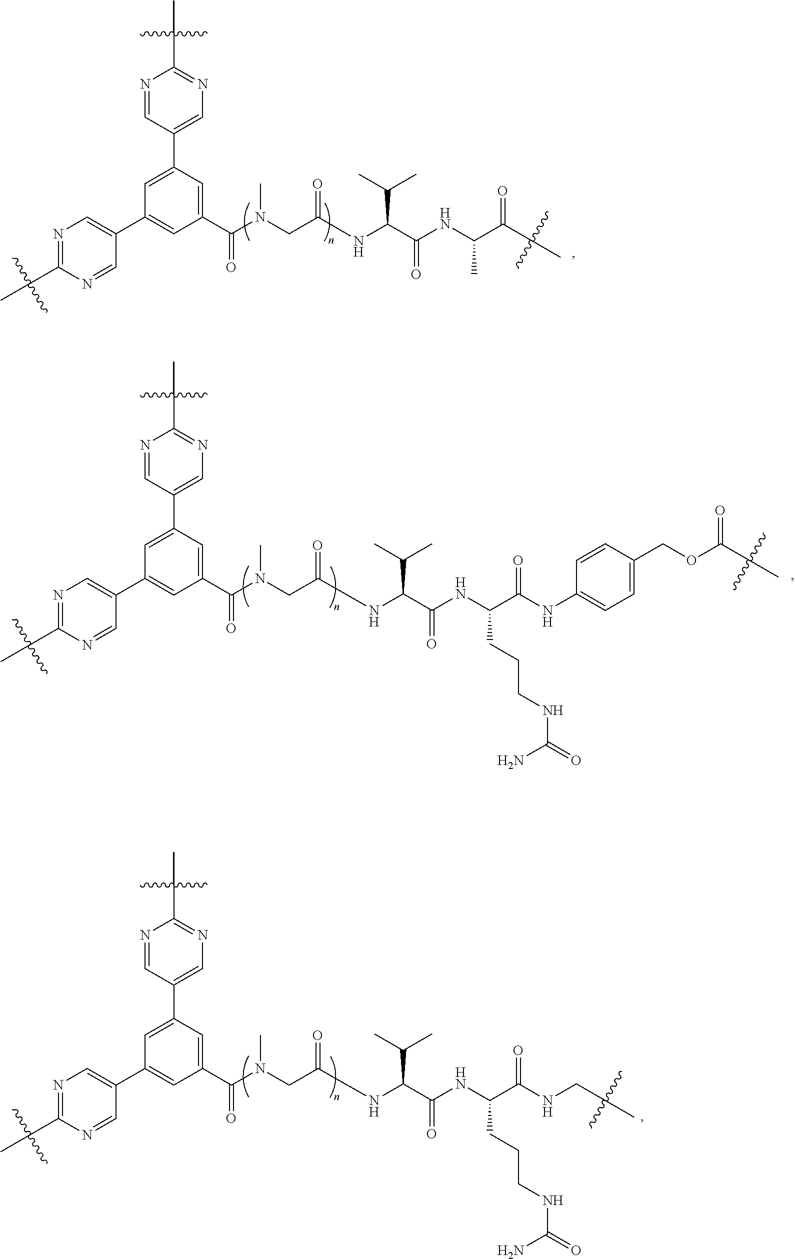

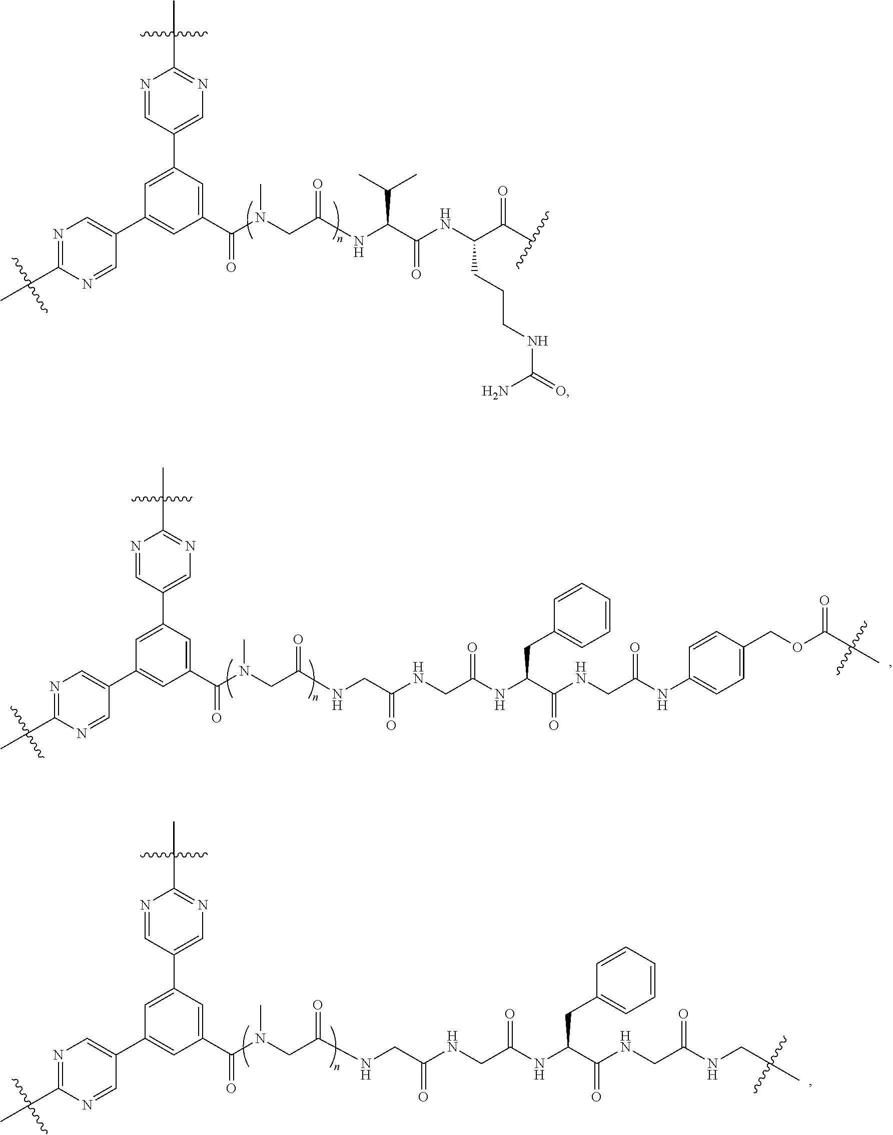

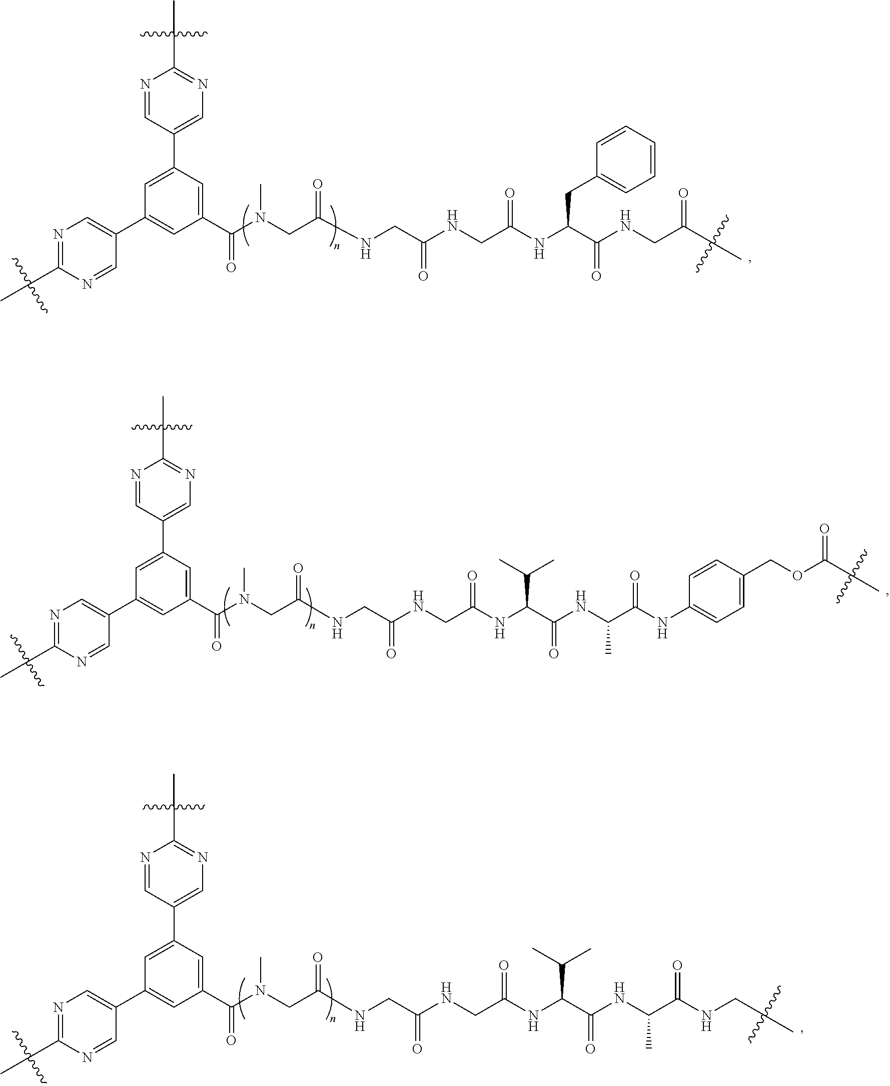

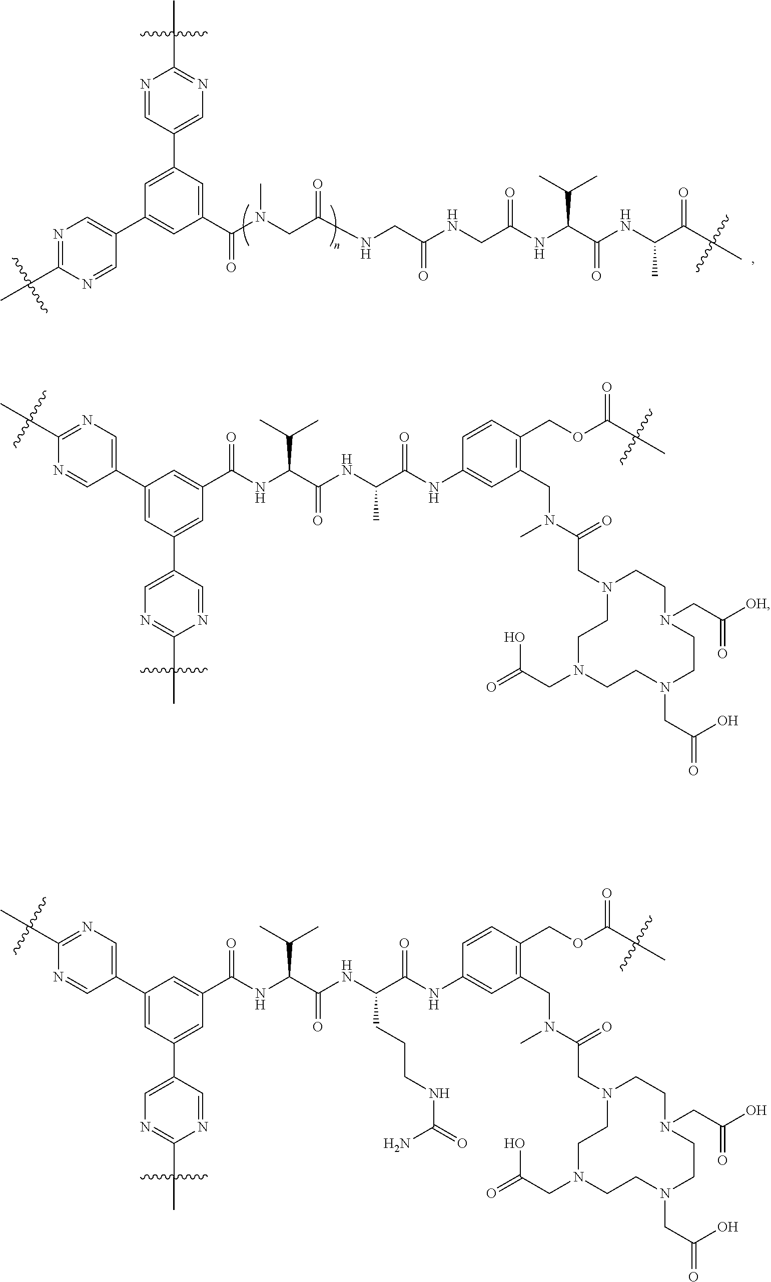

is selected from the group consisting of the following substituted or unsubstituted structures:

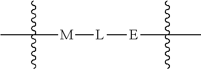

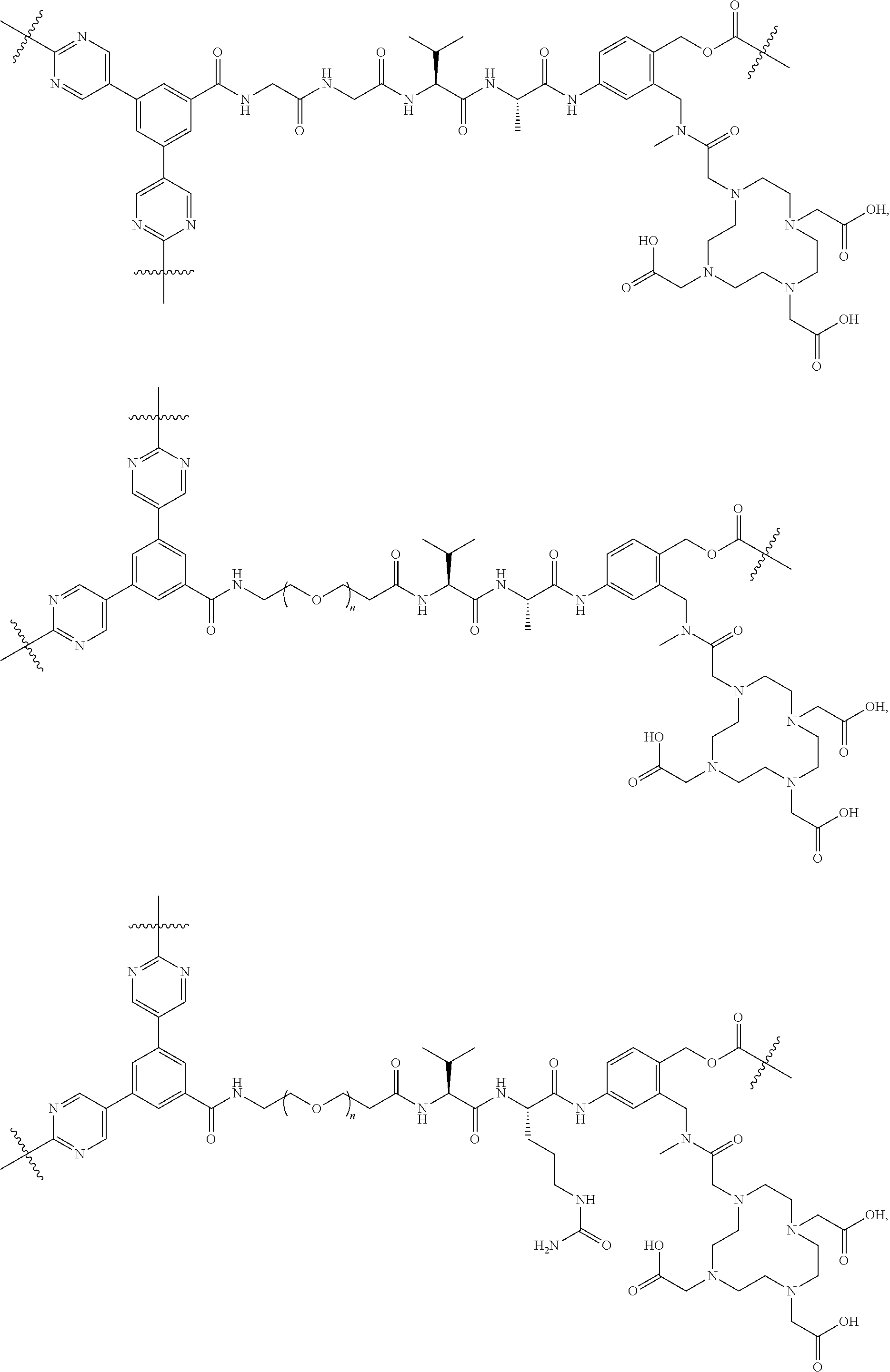

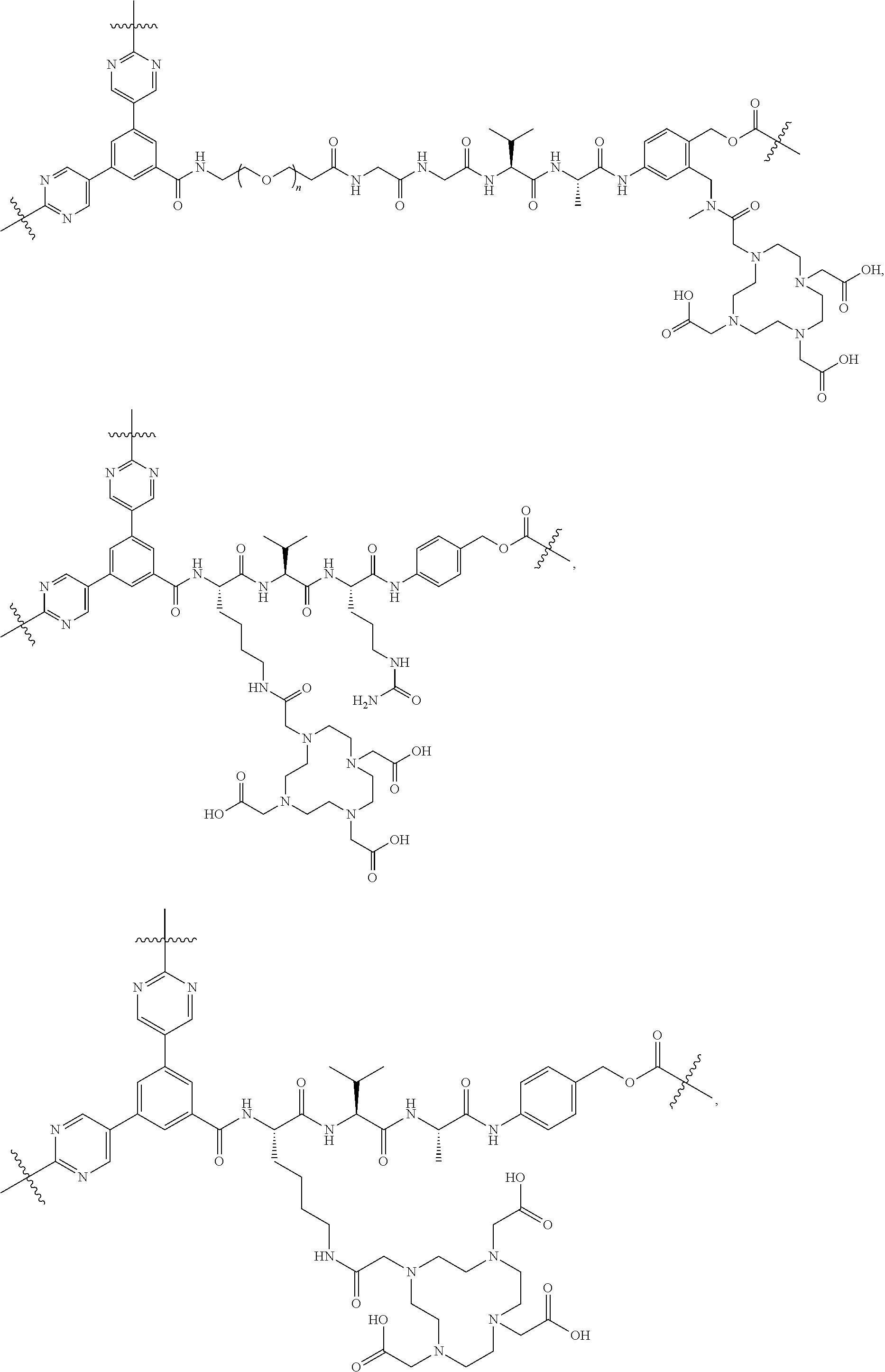

n is an integer selected from 1 to 20; preferably, n is an integer selected from 5 to 15, such as 5, 6, 7, 8, 9, 10, 11, 12, 13, 14 or 15.

In some embodiments, -{-M-L-E-}- is selected from the group consisting of the following substituted or unsubstituted structures:

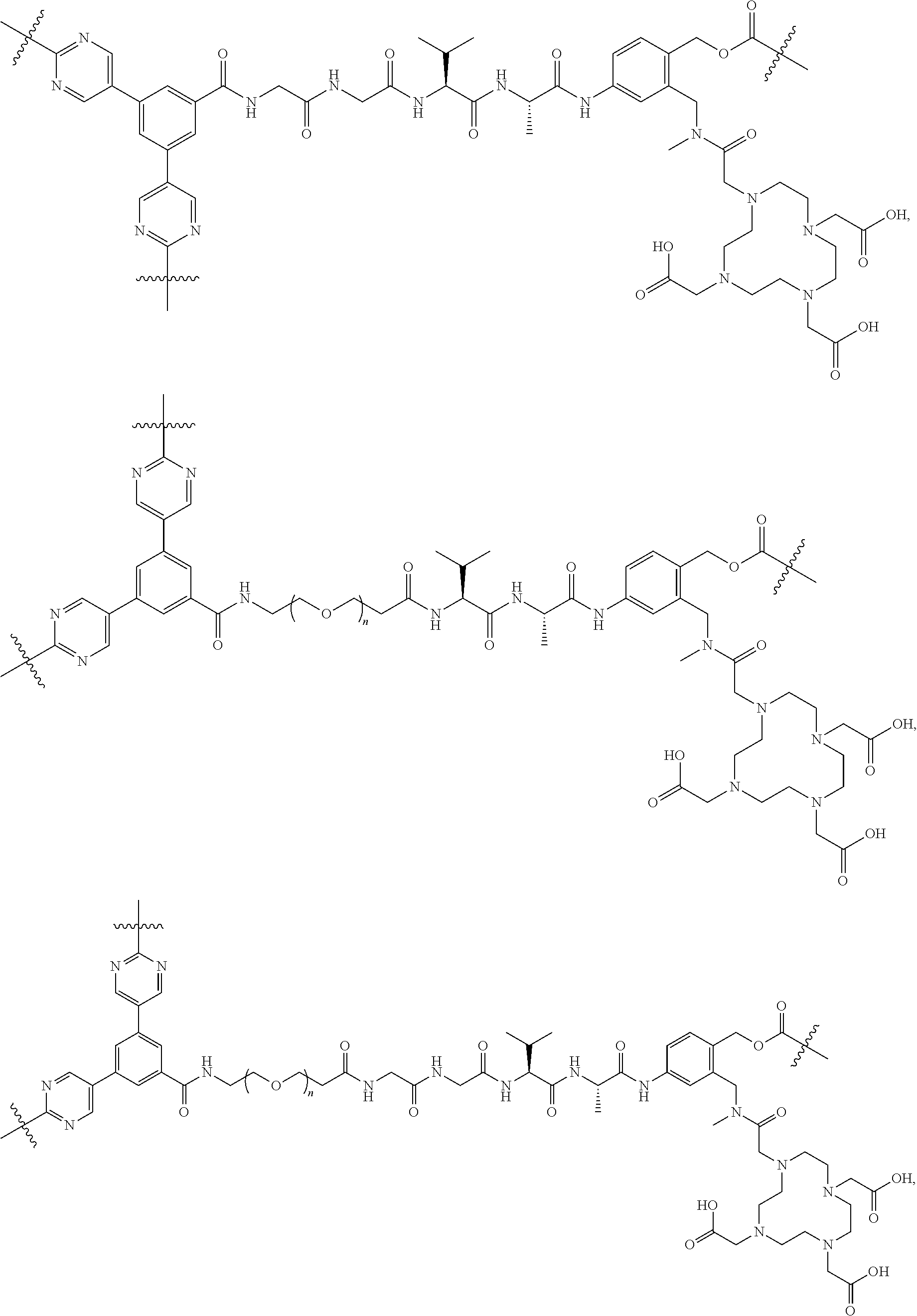

n is an integer selected from 5 to 15, such as 5, 6, 7, 8, 9, 10, 11, 12, 13, 14 or 15.

In some embodiments, the cytotoxic drug is selected from anti-tubulin agents. In some embodiments, the tubulin inhibitor is an auristatin compound.

In some embodiments, the cytotoxic drug is selected from the group consisting of the following compounds or isotopically labeled compounds thereof:

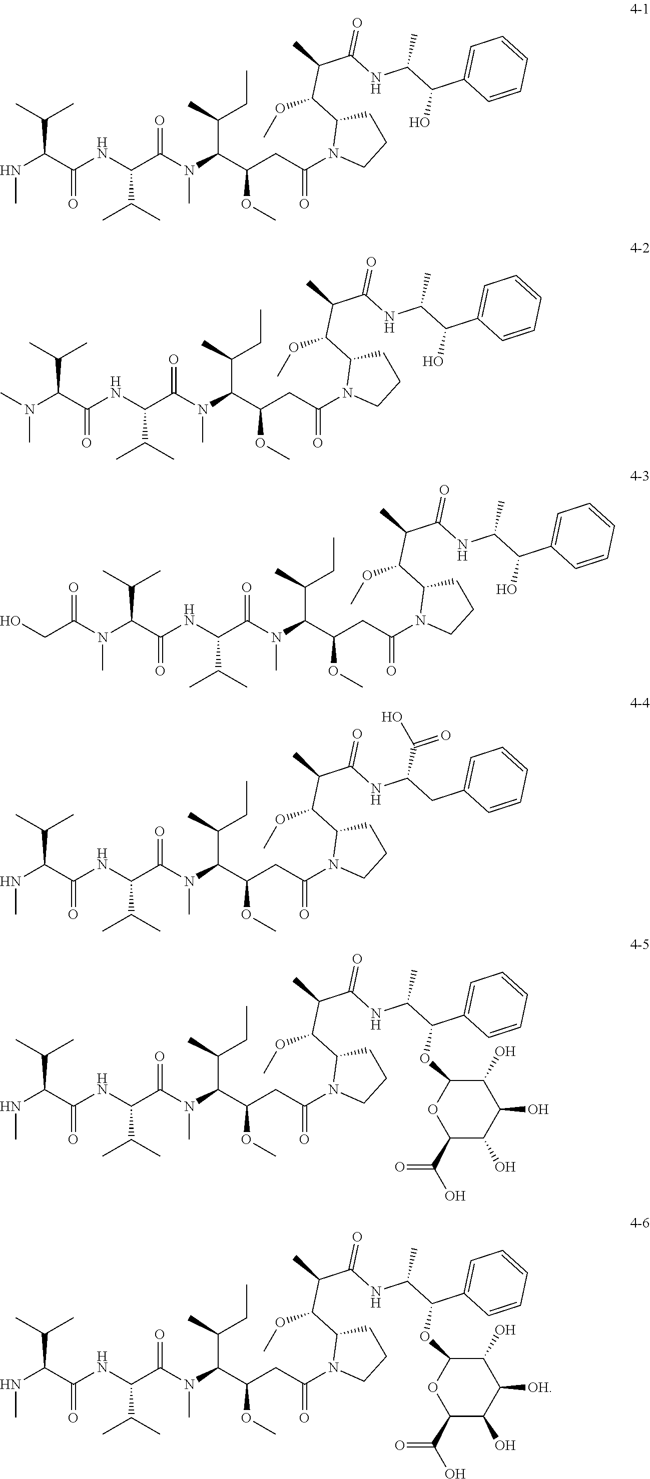

The cytotoxic drugs disclosed in the present application typically contain a variety of functional groups, such as hydroxyl (—OH), carboxyl (—COOH), primary amino (—NH₂), secondary amino (—NR₁H), tertiary amino (—NR₂R₃), where R₁, R₂, and R₃ here merely represent non-hydrogen substituents on N, or sulfhydryl (—SH), and these functional groups are capable of reacting with appropriate functional groups in the rest of the conjugate to form covalent attachments.

In some embodiments, the cytotoxic drug is attached to E in the antibody-drug conjugate through-OH, primary amino, secondary amine or tertiary amine, or —SH thereon. In some embodiments, D is a monovalent structure obtained by losing one H from —OH, —NH₂ or secondary amine on the cytotoxic drug.

In some embodiments, the cytotoxic drug fragment D is selected from the group consisting of:

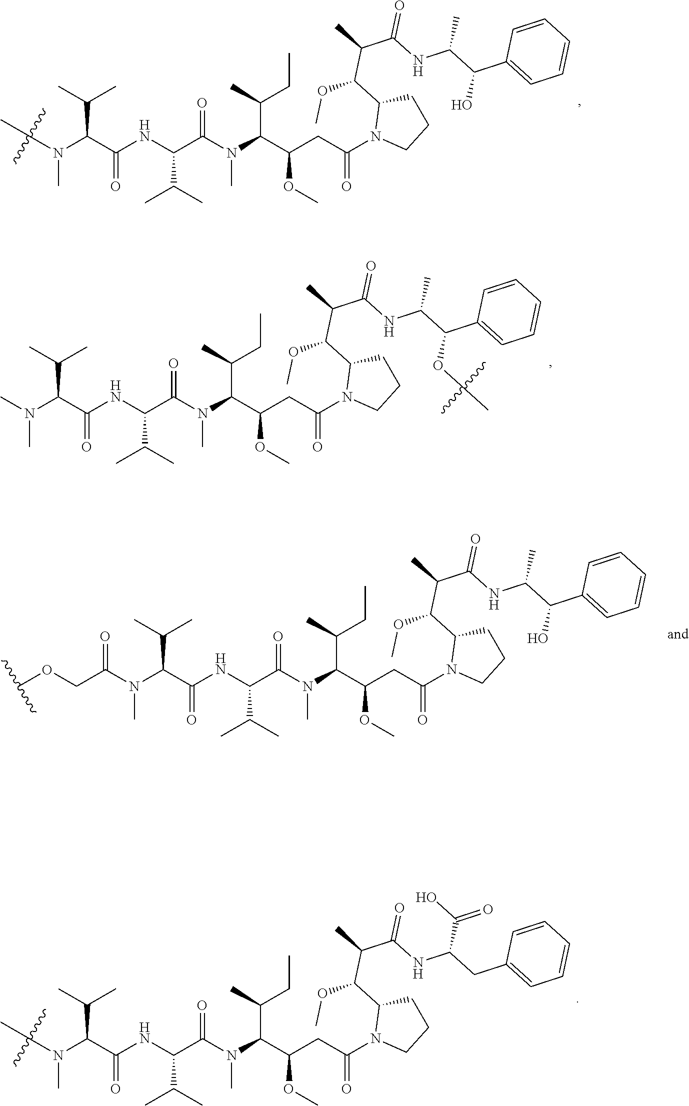

Those skilled in the art should understand that the antibody-drug conjugate described in the present application can be prepared modularly. For example, a compound of free form of “drug-linker complex” (which can be understood as M′-L-E-D, where M′ represents the structural form of M before covalently linking to an antibody or antigen-binding fragment thereof) is first obtained, and then covalently linked to the antibody or antigen-binding fragment thereof to obtain the antibody-drug conjugate described in the present application. Accordingly, M′ in the free form of the “drug-linker complex” is attached to one or more sulfhydryl groups (—SH) or amino groups (—NH₂) on the antibody or antigen-binding fragment thereof by a substitution reaction (e.g., removing structures such as —SO₂Me or pentafluorophenol thereon) or by an addition reaction.

In one aspect, the present application provides a compound or pharmaceutically acceptable salt thereof, which has a structure represented by the formula D-E-L-M′, where:
M′ represents -M-Lg, where Lg is a leaving group for a nucleophilic substitution reaction, and M is a structural fragment capable of binding to a targeting moiety (e.g., an antibody or antigen-binding fragment thereof);
L represents a structural fragment connecting M and E;
E represents a structural fragment connecting L and D;
D represents a cytotoxic drug fragment.

In some embodiments, Lg is selected from the group consisting of a halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), sulfhydryl (—SH), amino (—NH₂), nitro, azido, cyano, alkenyl, alkynyl, and alkynyl-containing structural fragment, where the halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, alkenyl, alkynyl, and alkynyl-containing structural fragment are optionally substituted with one or more suitable substituents.

In some embodiments, Lg is selected from the group consisting of a halogen (e.g., F, Cl, Br, I), halogenated C₁₋₆ alkyl, C₁₋₆ alkylsulfonyl, halogenated C₁₋₆ alkylsulfonyl, halogenated sulfonyl, C₁₋₆ alkylsulfonate group, halogenated C₁₋₆ alkylsulfonate group, C₁₋₆ alkylsulfinate group, C₁₋₆ alkylsulfoxide group, halogenated phenoxy, hydroxyl (—OH), sulfhydryl (—SH), amino (—NH₂), nitro, azido, cyano, alkenyl, alkynyl, and alkynyl-containing structural fragment.

In some embodiments, Lg is selected from the group consisting of a halogen, substituted or unsubstituted C₁₋₆ alkylsulfonyl (C₁₋₆ alkyl-SO₂—), halogenated phenoxy, hydroxyl (—OH), sulfhydryl (—SH), and amino (—NH₂).

In some embodiments, Lg is selected from the group consisting of a halogen, substituted or unsubstituted methylsulfonyl, halogenated phenoxy, hydroxyl (—OH), sulfhydryl (—SH), and amino (—NH₂).

In some embodiments, Lg is selected from the group consisting of C₁₋₆ alkylsulfonyl and halogenated phenoxy.

In some embodiments, Lg is selected from the group consisting of methylsulfonyl and pentafluorophenoxy.

In some embodiments, M, L, E and D are as defined in any of the above aspects.

In some embodiments, the free form of the “drug-linker” is selected from the group consisting of F-1 to F-20, L-1 to L-11 as shown below:

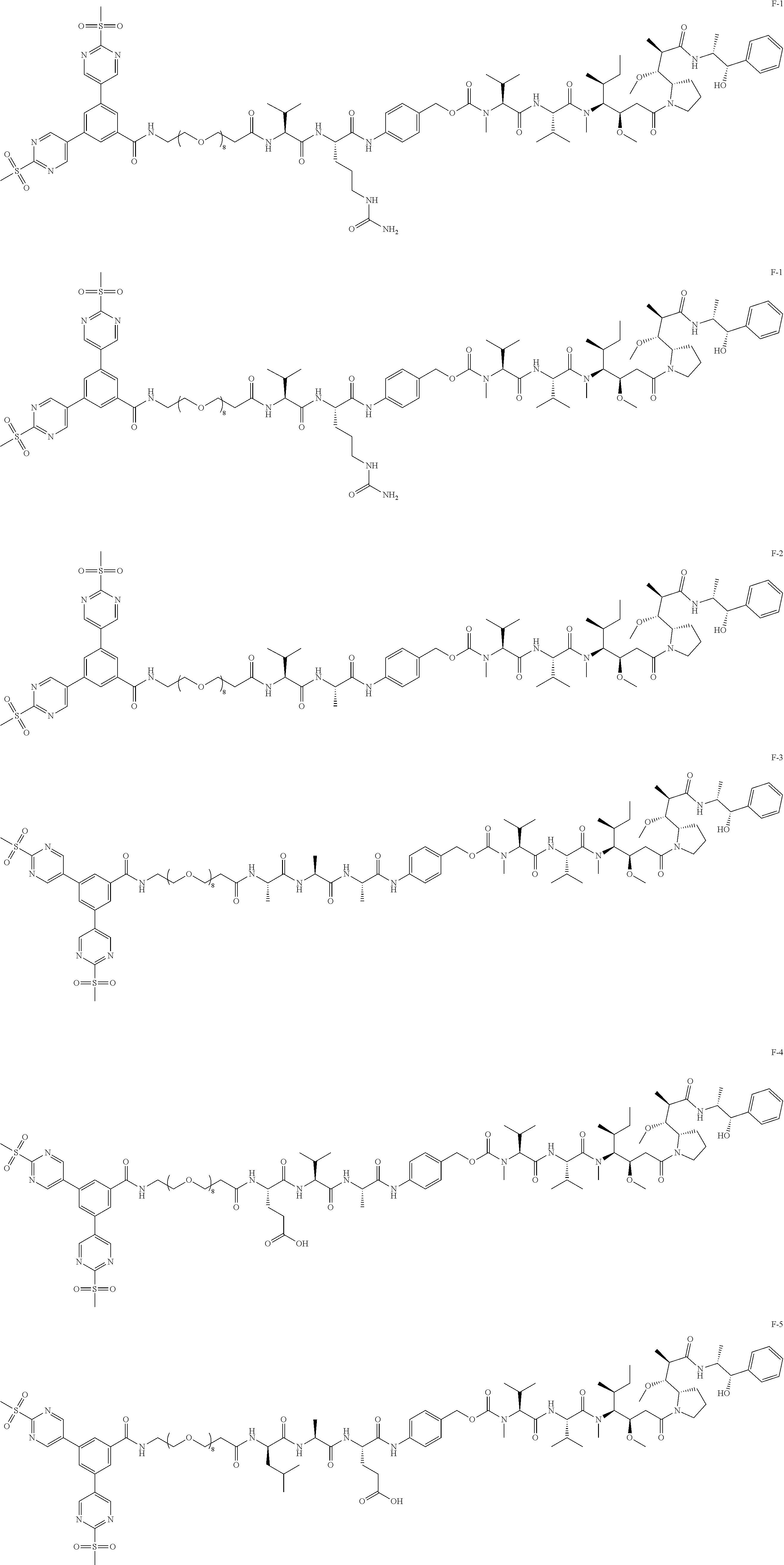

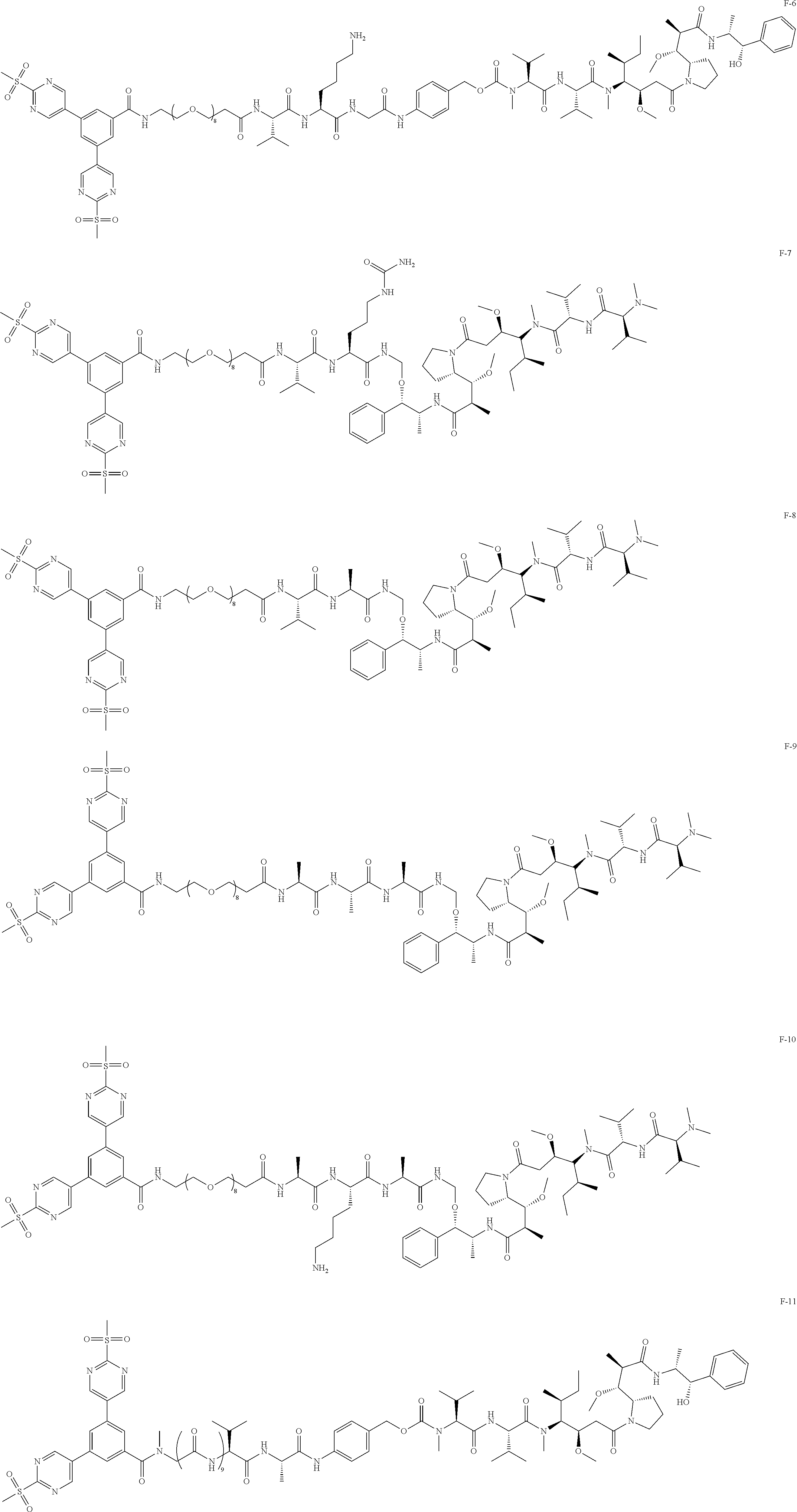

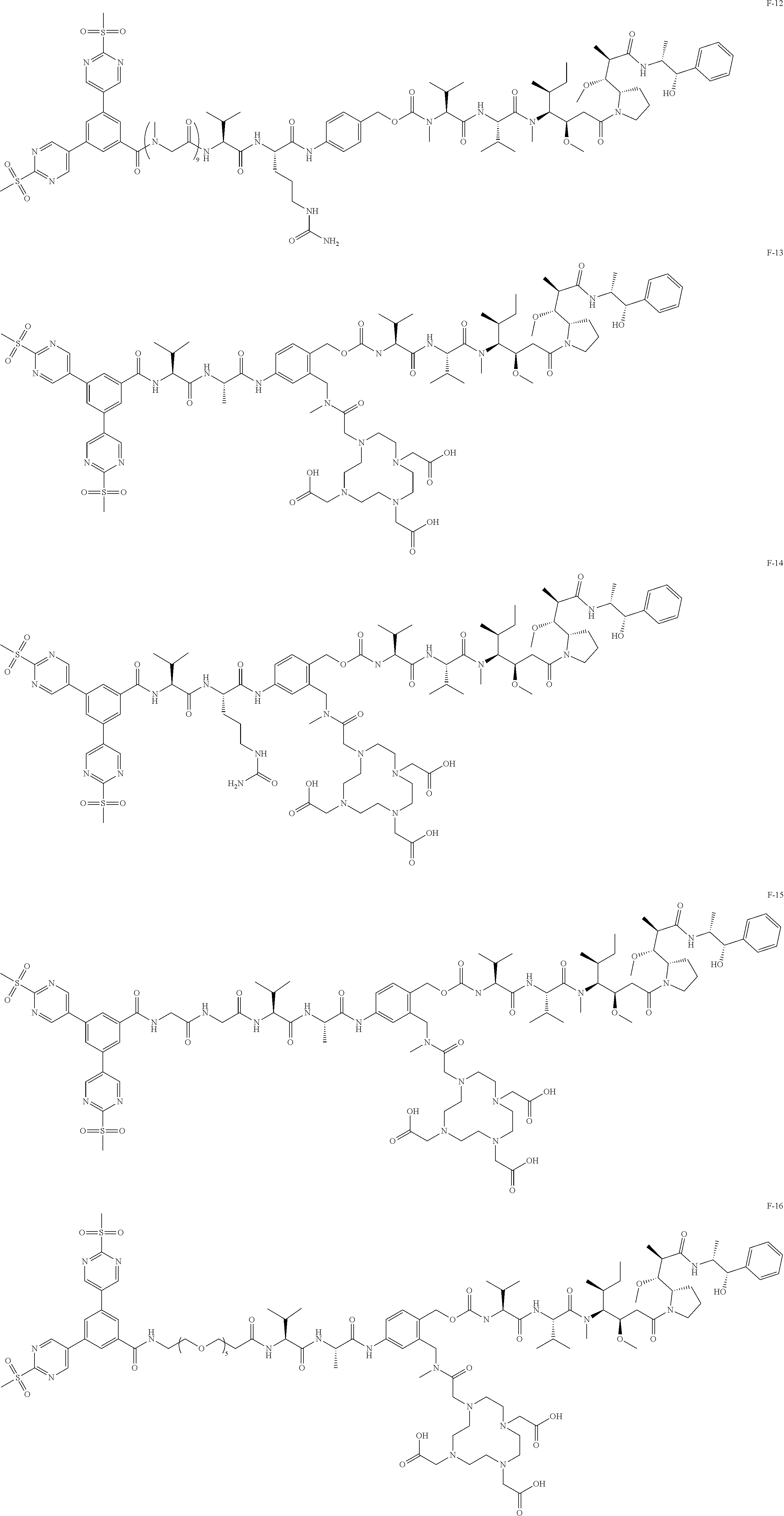

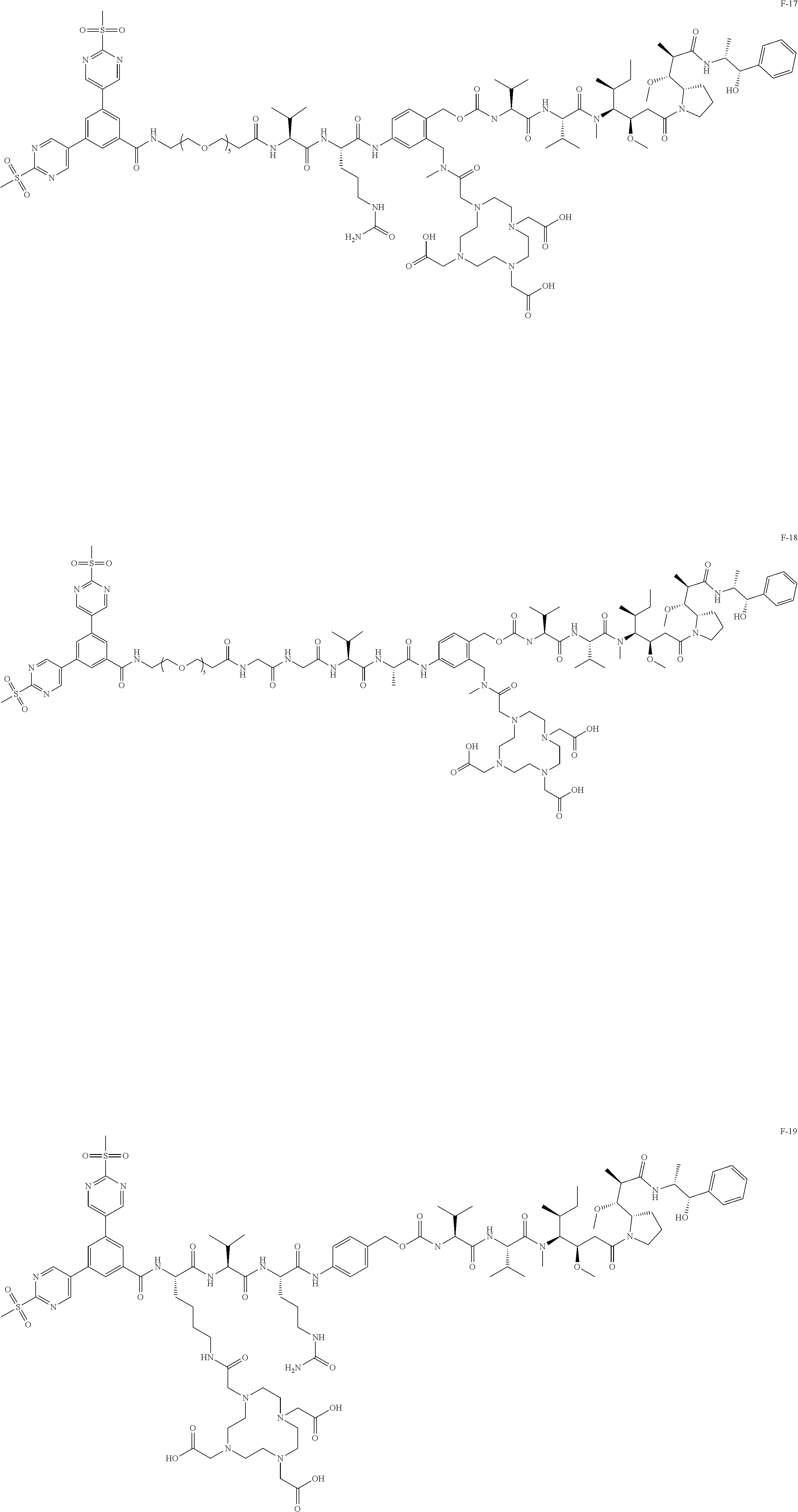

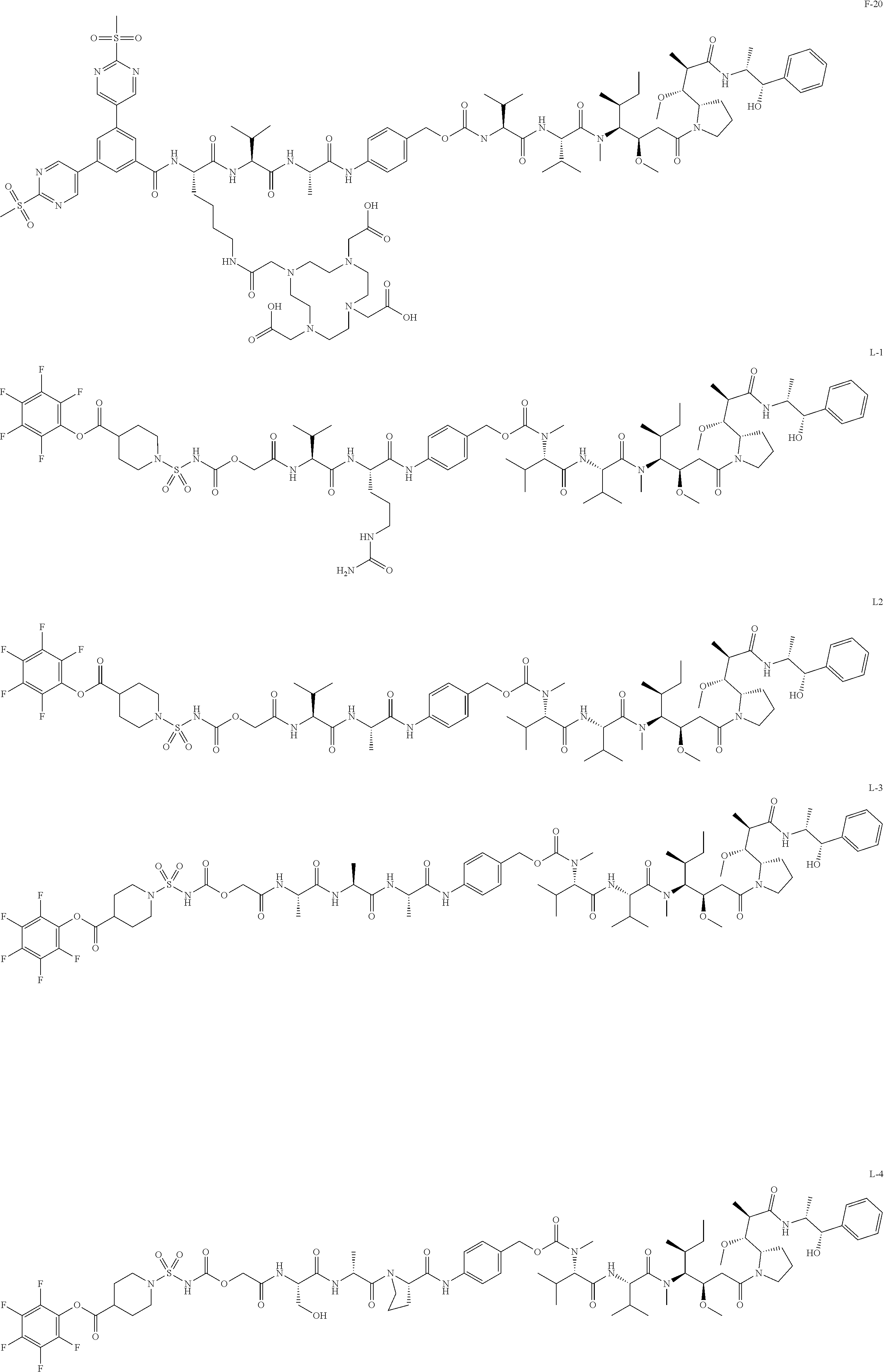

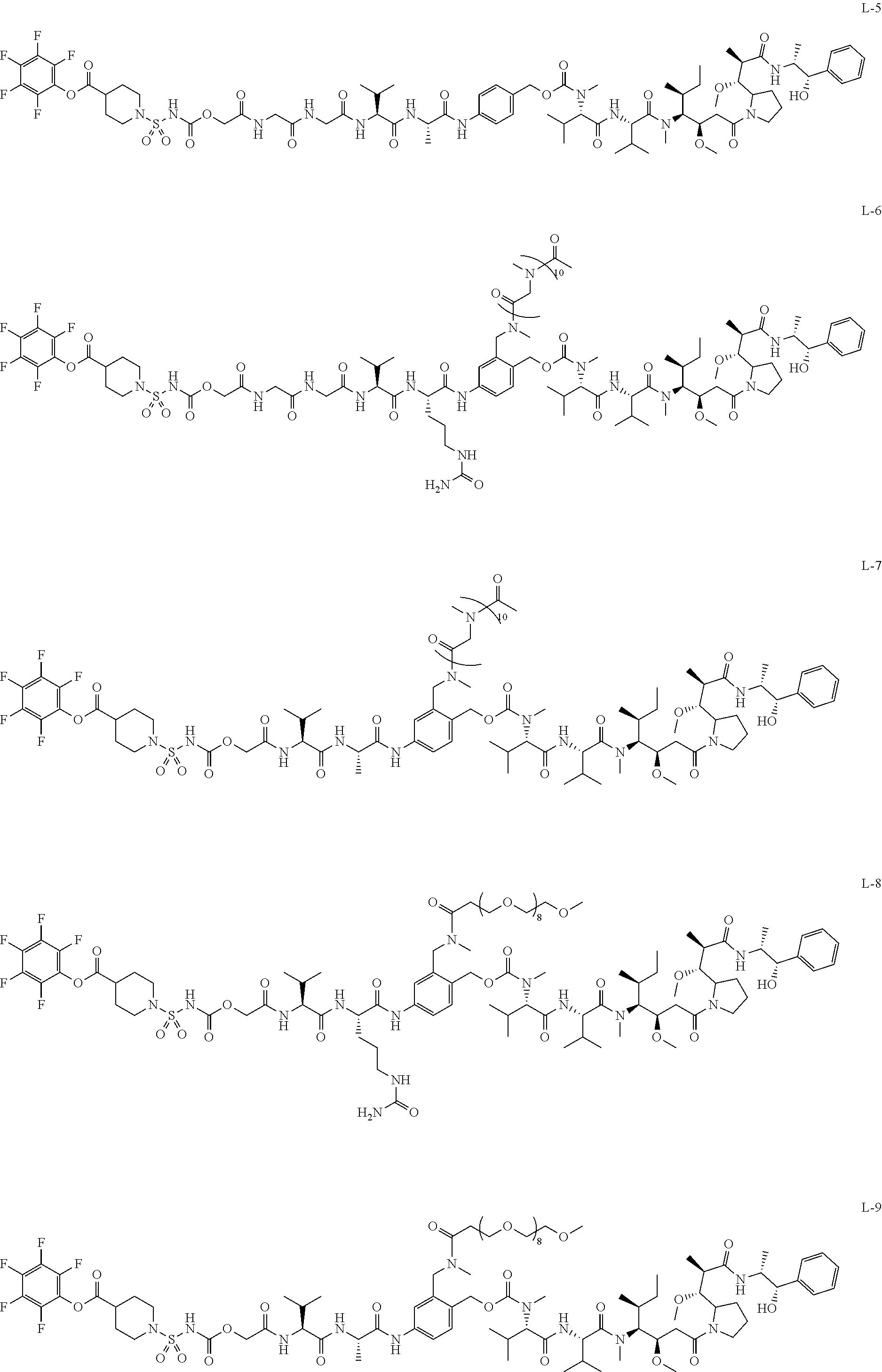

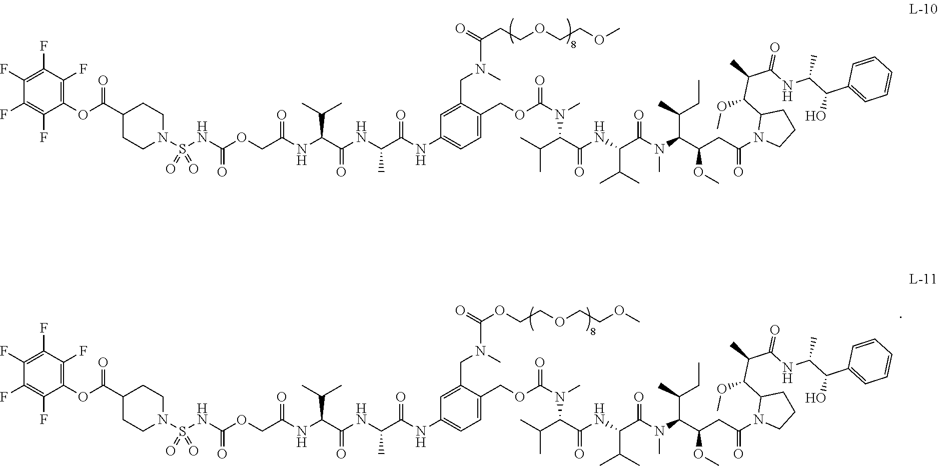

In some embodiments, the free form of the “drug-linker” is selected from the group consisting of F′-1 to F′-12, F′-16 to F′-18, L′-6 to L′-11 as shown below:

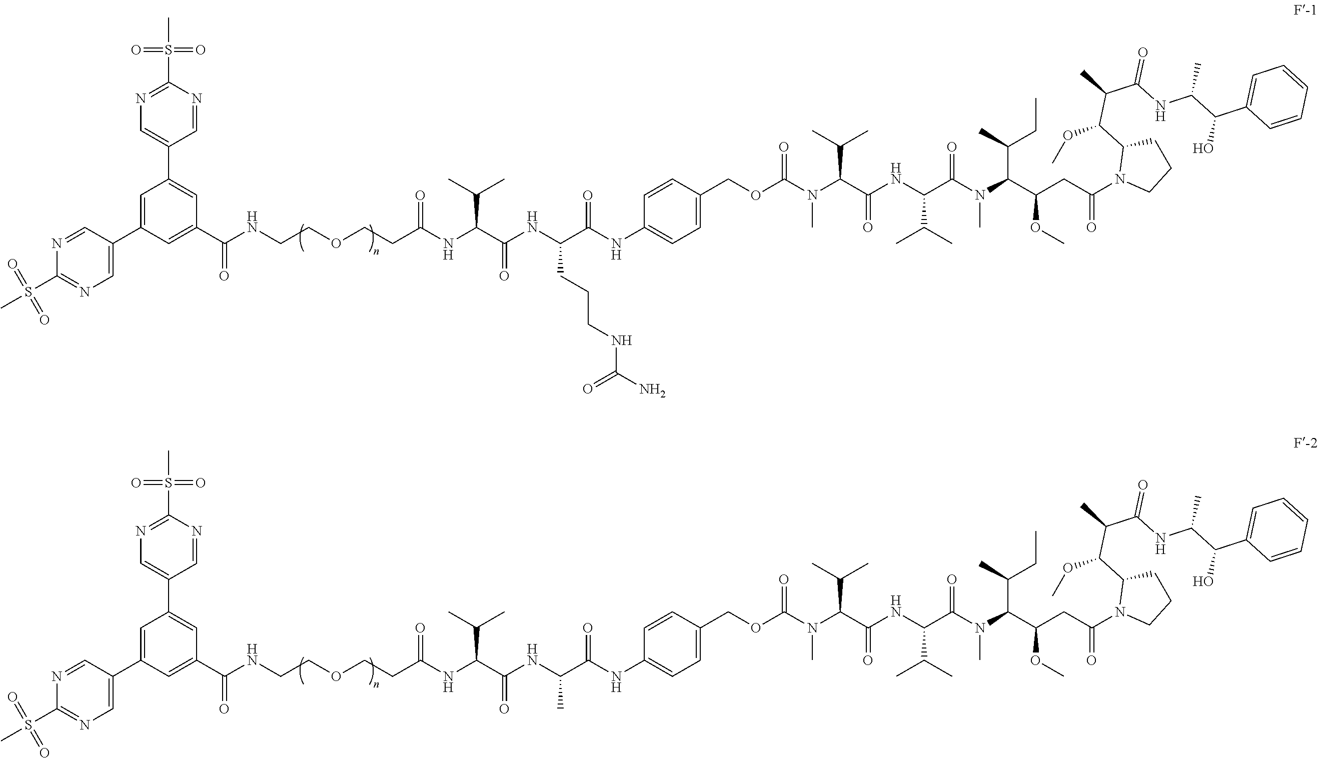

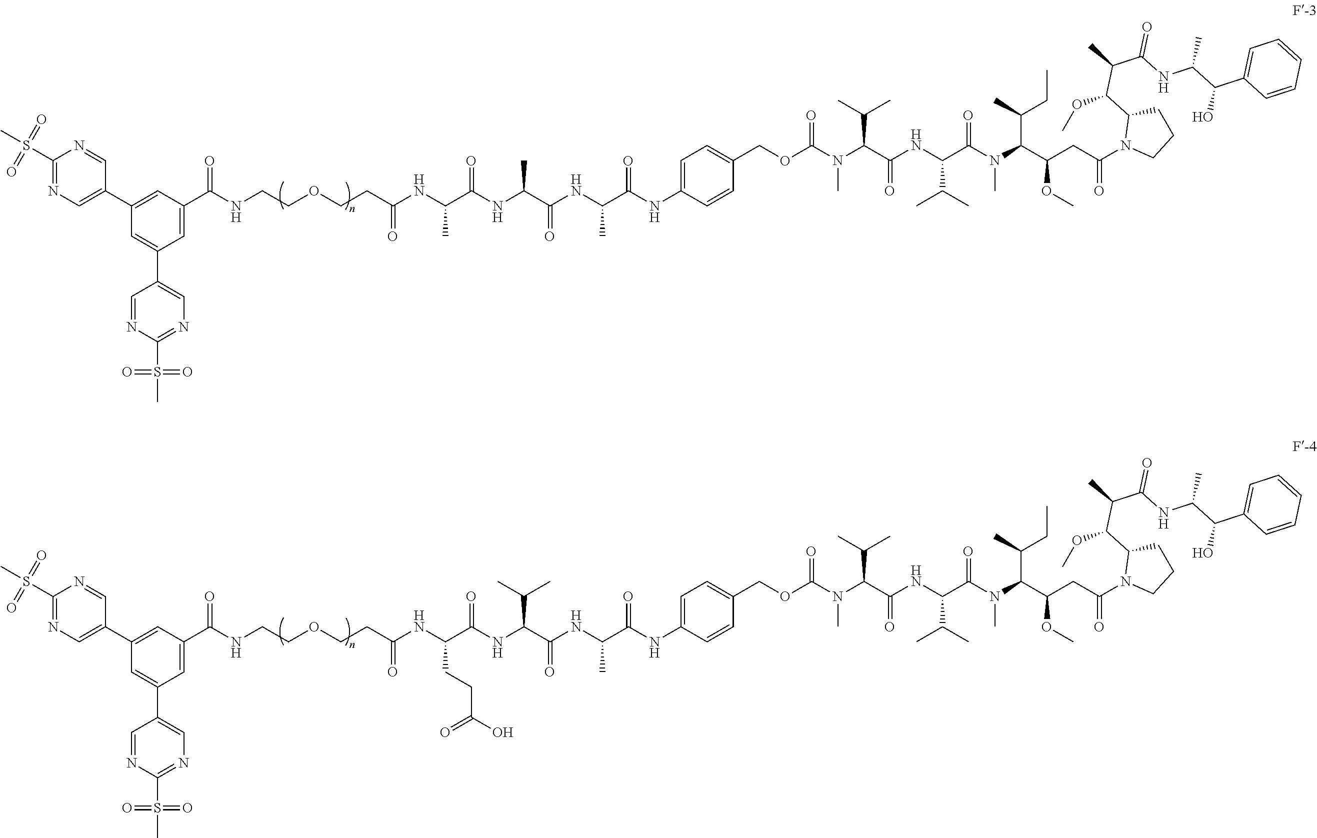

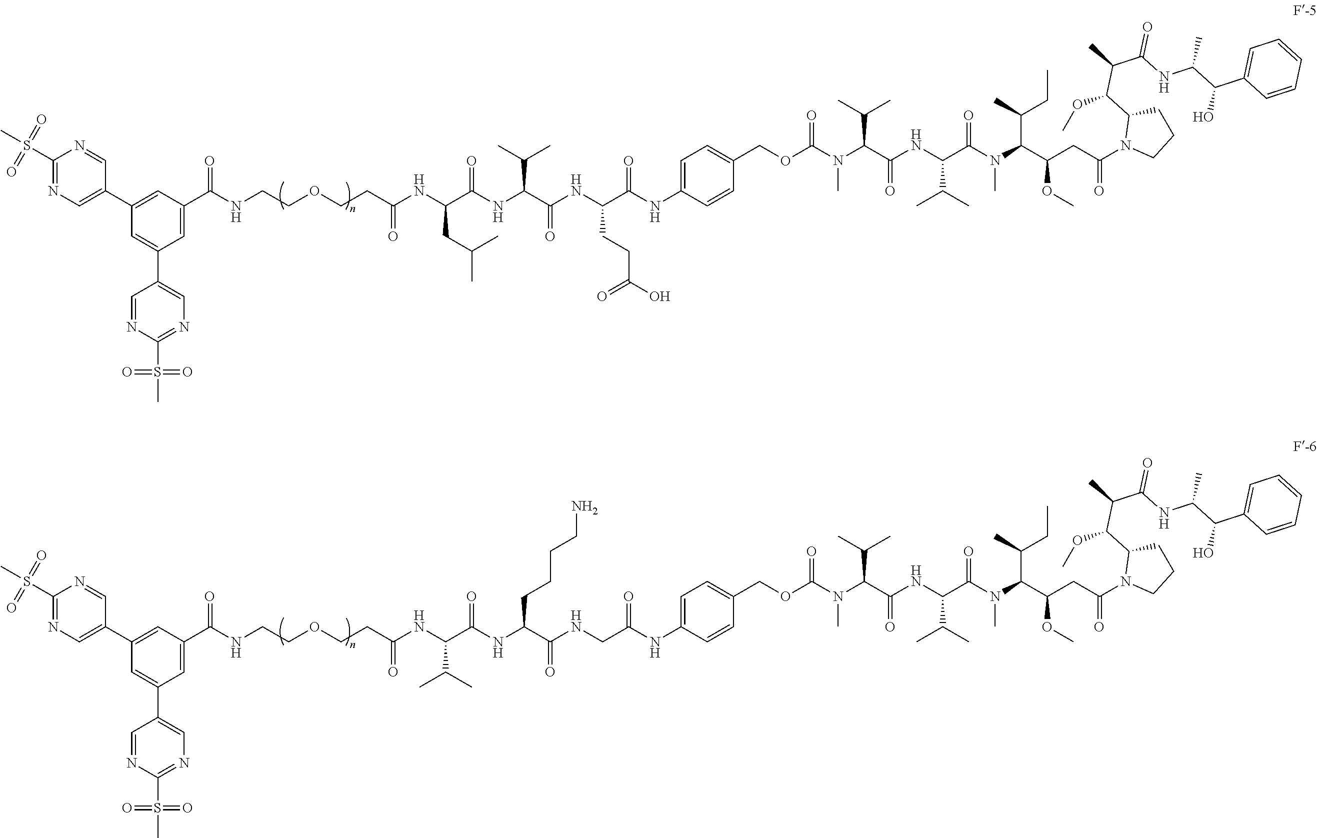

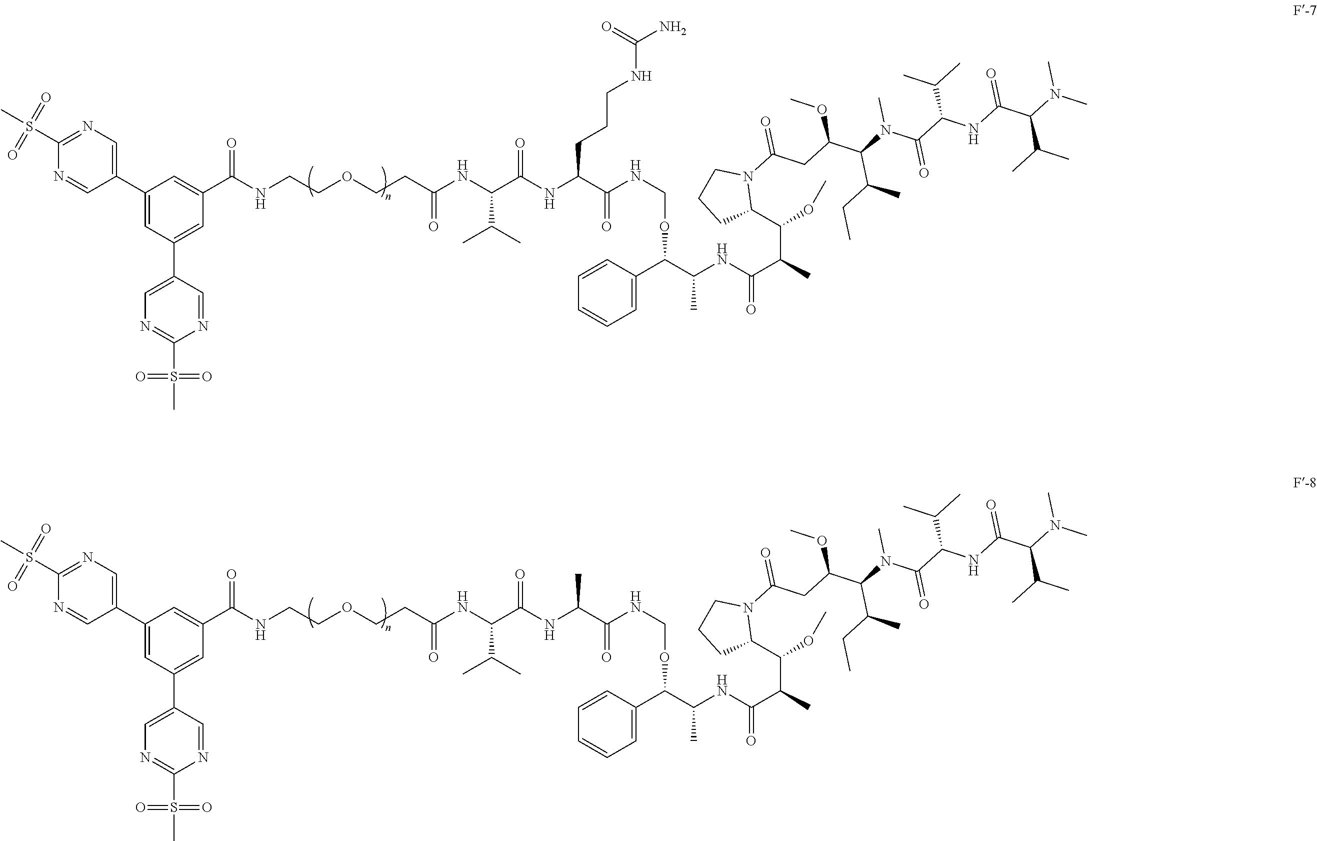

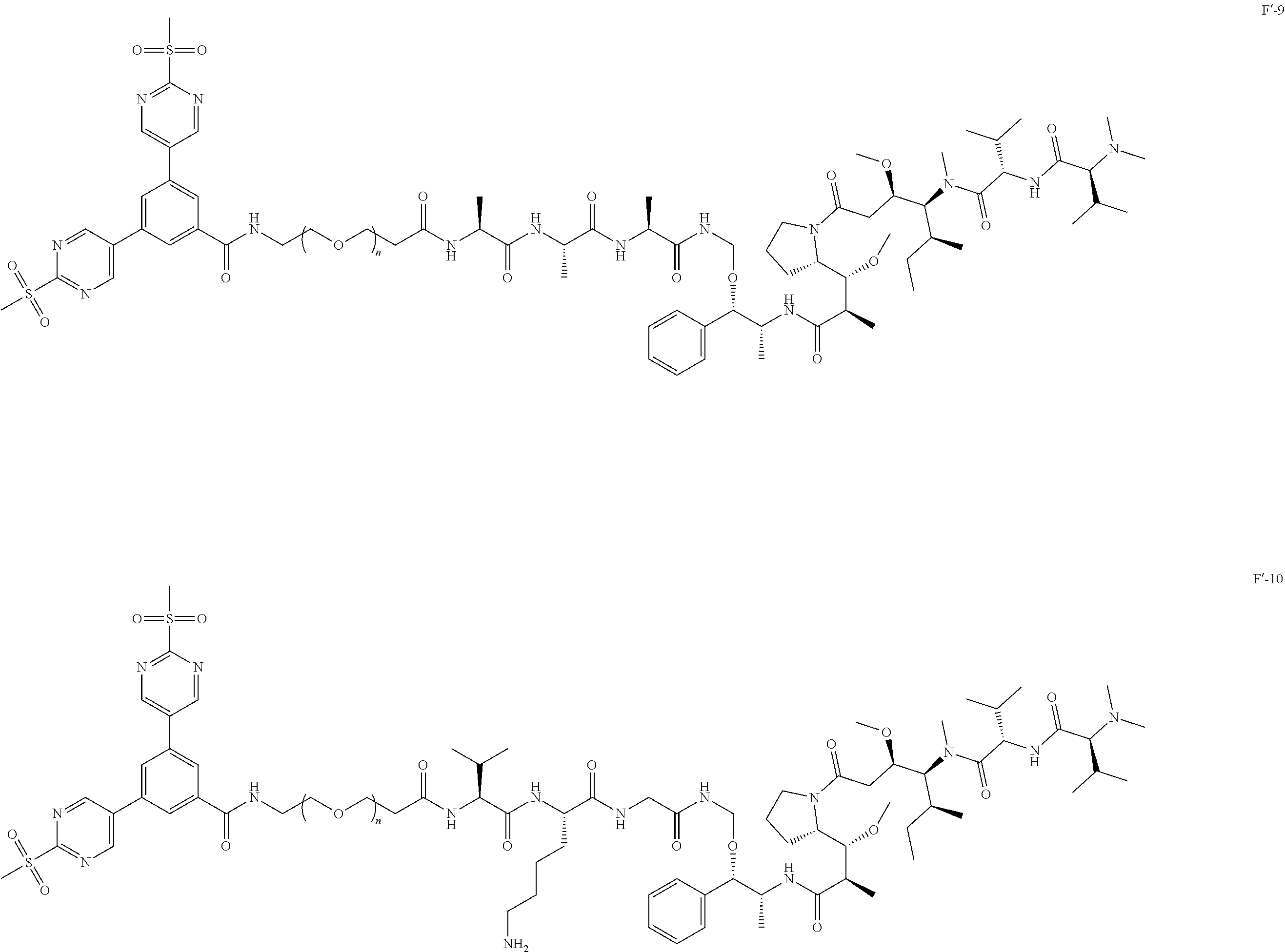

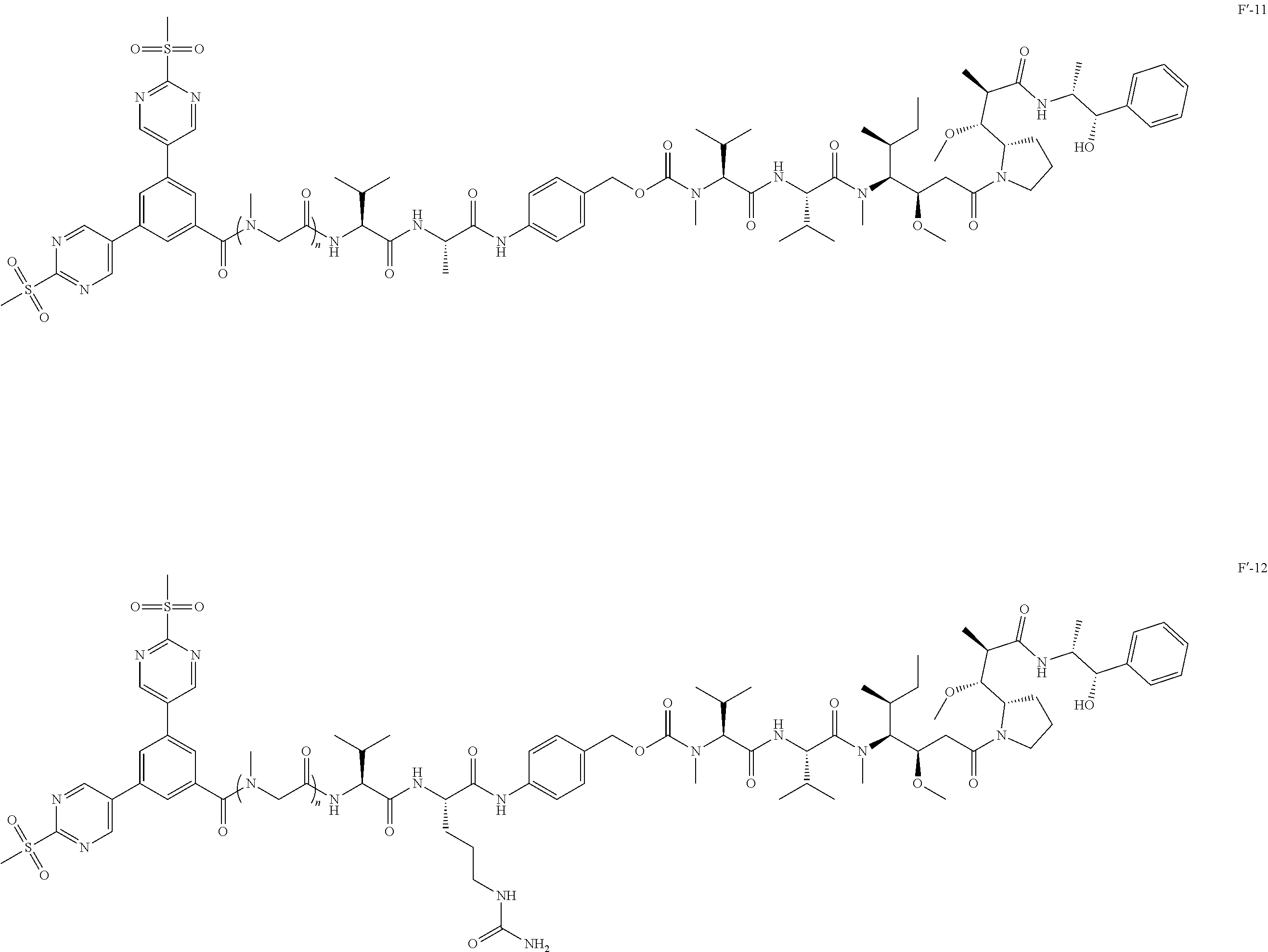

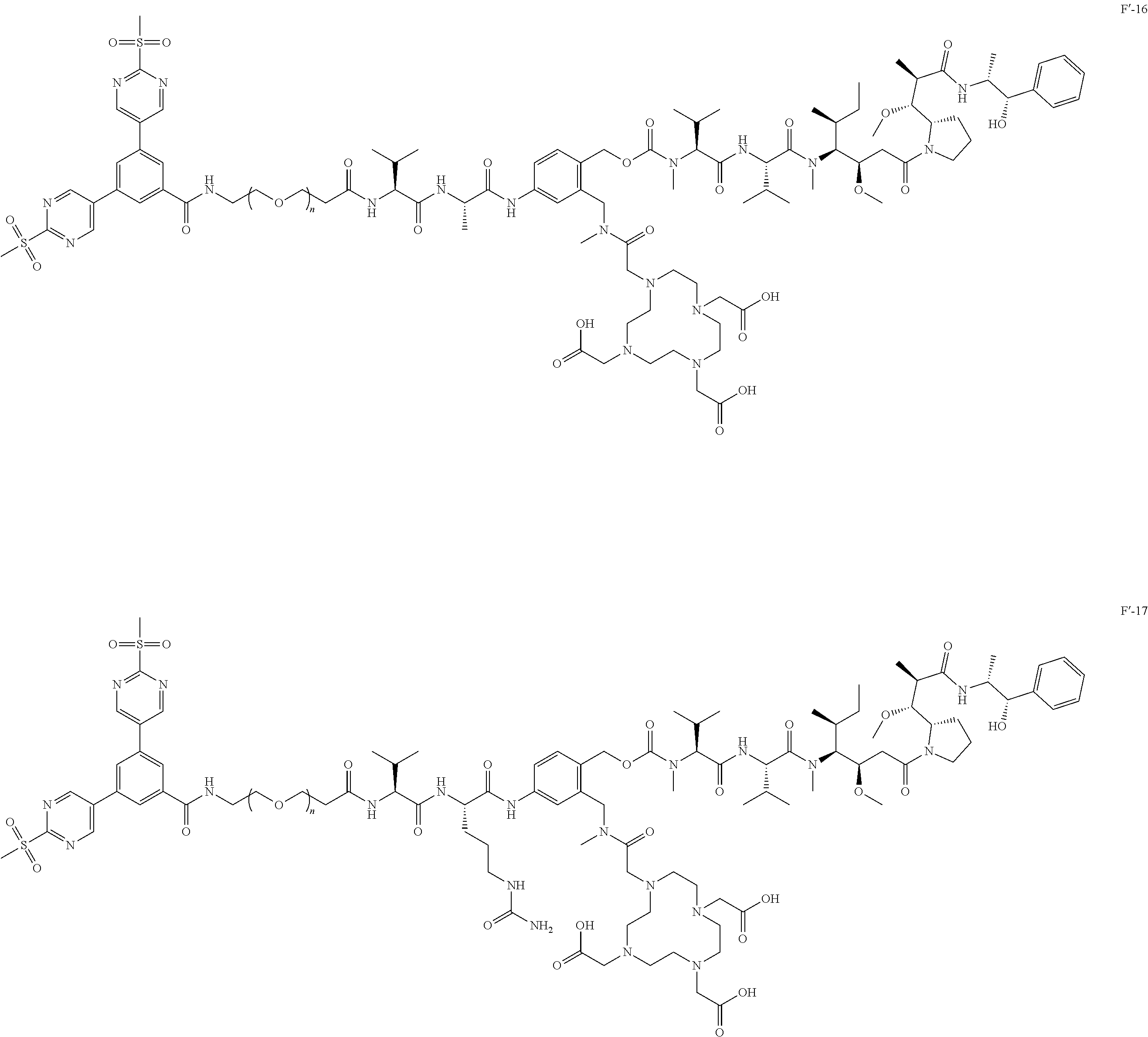

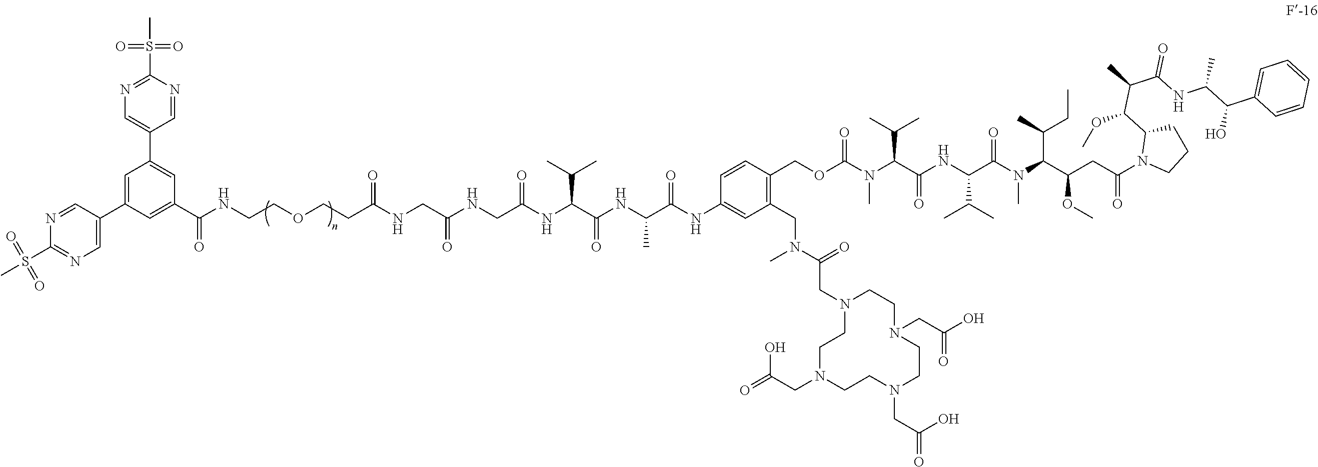

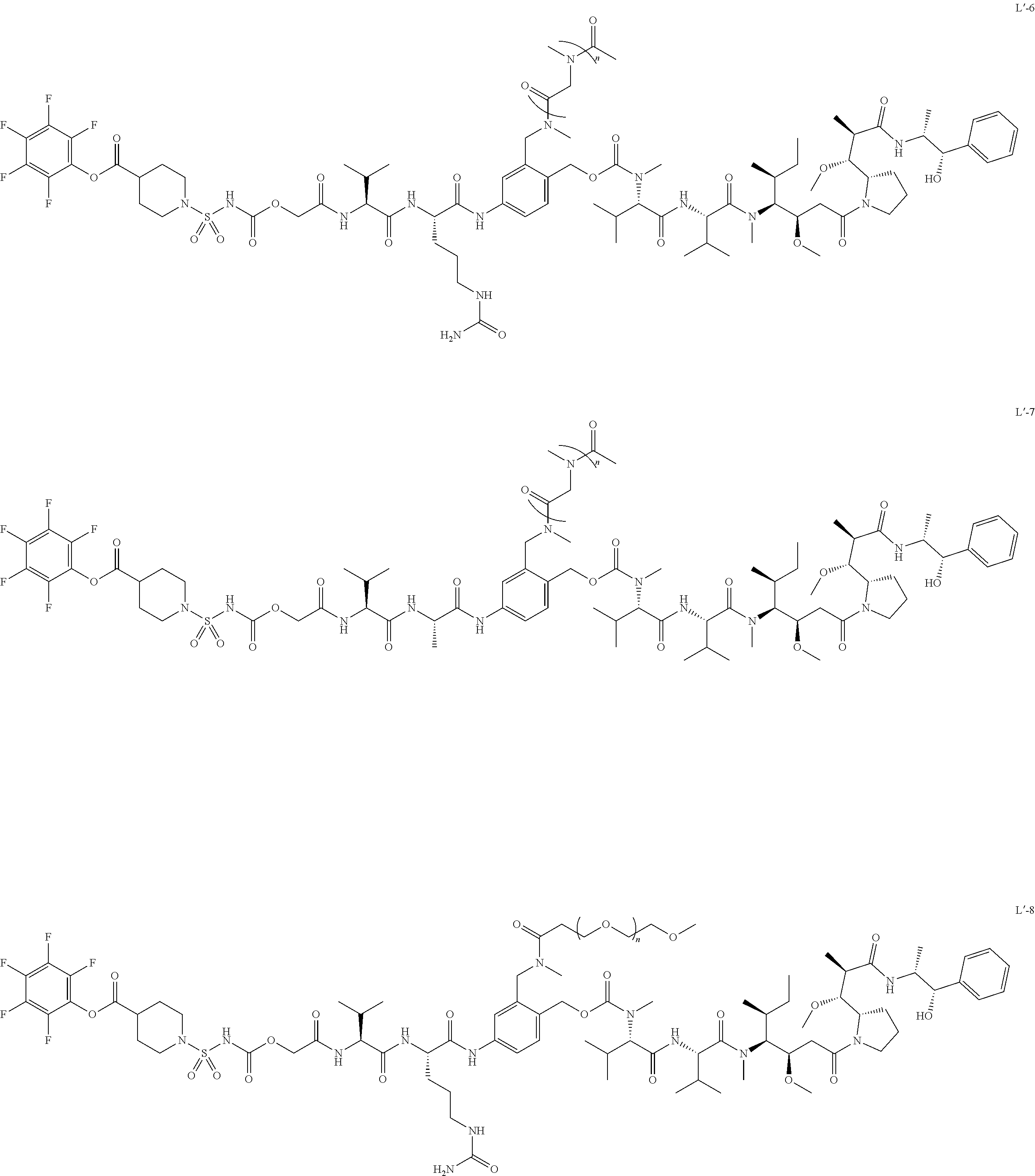

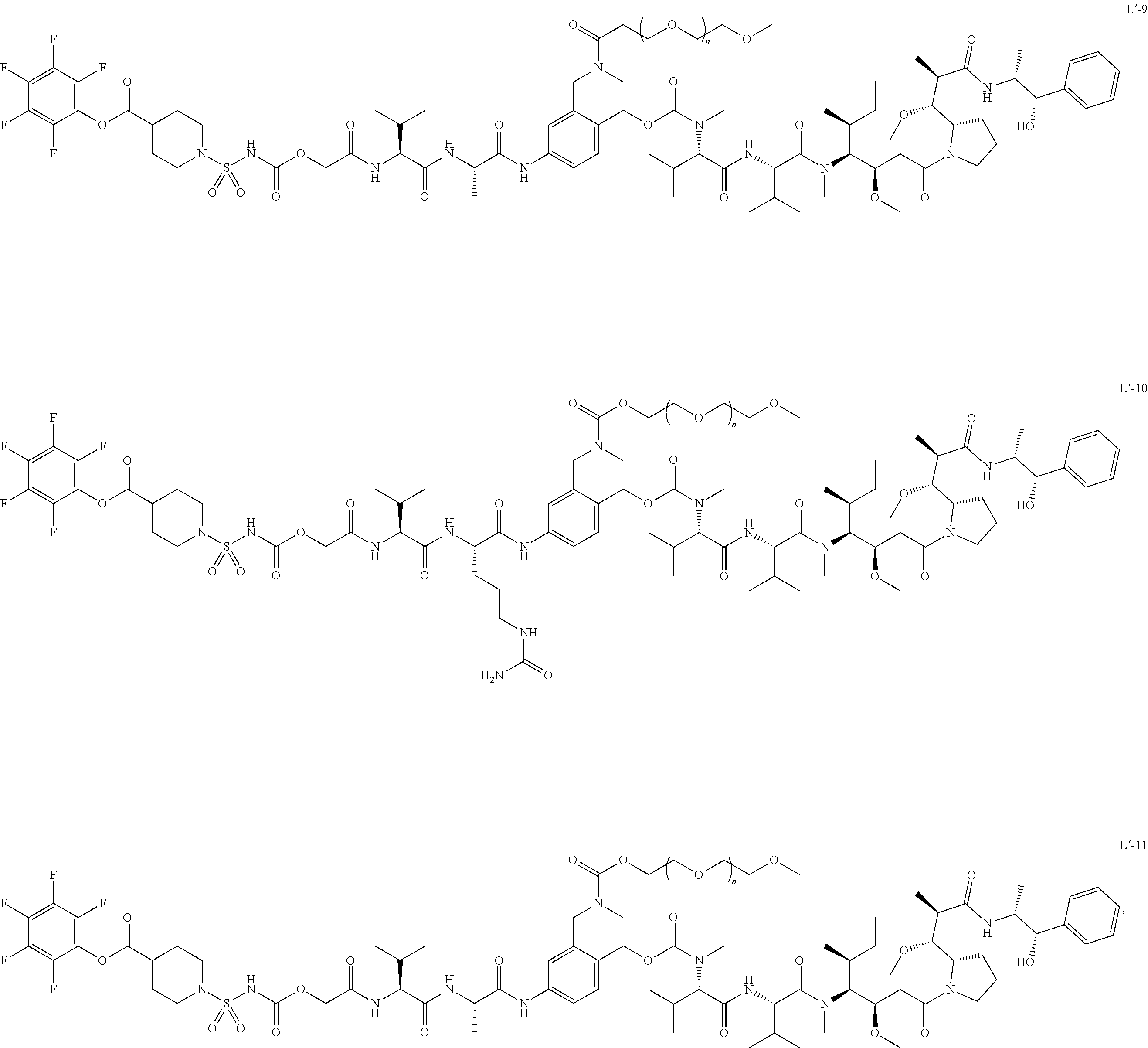

where n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 5, 8, 9 or 10.

In some embodiments, the drug-linker described above may be optionally substituted with one or more appropriate substituents.

In some embodiments, the antibody-drug conjugate of the present application has a structure represented by the formula Ab-[M-L-E-D]ₓ, where:
Ab represents an antibody or antigen-binding fragment thereof capable of specifically binding to epidermal growth factor receptor 2 (Her2), a member of the ErbB family of receptor tyrosine kinases;
x is 1 to 10, preferably, x is 1 to 4, and further preferably, x is 4;
-M-L-E-D is formed from the above-mentioned compounds F′-1 to F′-12, F′-16 to F′-18, L′-6 to L′-11, F-1 to F-20 or L-1 to L-11 (preferably F-1 to F-20 or L-1 to L-11).

In some embodiments, the antibody-drug conjugate of the present application has a structure represented by the formula Ab-[M-L-E-D]ₓ, where:
Ab represents an antibody or antigen-binding fragment thereof capable of specifically binding to epidermal growth factor receptor 2 (Her2), a member of the ErbB family of receptor tyrosine kinases;
x is 1 to 10, preferably, x is 1 to 4, and more preferably, x is 4;
-M-L-E-D is formed by removing a mesyl group from the compounds F-1 to F-20 as described above.

In some embodiments, the antibody-drug conjugate of the present application has a structure represented by the formula Ab-[M-L-E-D]ₓ, where:
Ab represents an antibody or antigen-binding fragment thereof capable of specifically binding to epidermal growth factor receptor 2 (Her2), a member of the ErbB family of receptor tyrosine kinases;
x is 1 to 10, preferably, x is 1 to 4, and more preferably, x is 2;
-M-L-E-D is formed by removing a pentafluorophenoxy group from the compounds L-1 to L-11 as described above.

In some embodiments, the antibody or antigen-binding fragment thereof comprises:
(1) the following heavy chain variable region (VH) and/or light chain variable region (VL):
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof; or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof, and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or, (1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof. L2, and a CDR-L3 having a sequence as set forth in SEQ ID NO: 10 or variant thereof; or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof; and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO. 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and/or light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and/or, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and/or, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
of,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or, (2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and/or light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or, (4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, the antibody or antigen-binding fragment thereof comprises:
(a) a VH as set forth in SEQ ID NO: 1 or variant thereof, and/or, a VL as set forth in SEQ ID NO: 2 or variant thereof; or
(b) a VH as set forth in SEQ ID NO: 3 or variant thereof, and/or, a VL as set forth in SEQ ID NO: 4 or variant thereof;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, the antibody or antigen-binding fragment thereof comprises:
(a) a VH as set forth in SEQ ID NO: 1, and/or a VL as set forth in SEQ ID NO: 2; or
(b) a VH as set forth in SEQ ID NO: 3, and/or a VL as set forth in SEQ ID NO: 4.

In some embodiments, the antibody or antigen-binding fragment thereof comprises:
(a) a VH as set forth in SEQ ID NO: 1 or a variant thereof, and a VL as set forth in SEQ ID NO: 2 or a variant thereof, or
(b) a VH as set forth in SEQ ID NO: 3 or a variant thereof, and a VL as set forth in SEQ ID NO: 4 or a variant thereof.

In some embodiments, the antibody or antigen-binding fragment thereof comprises:
(a) a VH as set forth in SEQ ID NO: 1, and a VL as set forth in SEQ ID NO: 2; or
(b) a VH as set forth in SEQ ID NO: 3, and a VL as set forth in SEQ ID NO: 4.

In some embodiments, the antibody or antigen-binding fragment thereof further comprises:
(a) a heavy chain constant region (CH) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids); and
(b) a light chain constant region (CL) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids).

In some embodiments, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region.

In some embodiments, the antibody or antigen-binding fragment thereof comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35.

In some embodiments, the antibody or antigen-binding fragment thereof comprises a heavy chain constant region (CH) as set forth in SEQ ID NO: 35.

In some embodiments, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36.

In some embodiments, the antibody or antigen-binding fragment thereof comprises a light chain constant region (CL) as set forth in SEQ ID NO: 36.

In some embodiments, the antibody or antigen-binding fragment thereof comprises a heavy chain constant region (CH) as set forth in SEQ ID NO: 35 and a light chain constant region (CL) as set forth in SEQ ID NO: 36.

In some embodiments, the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, the antibody or antigen-binding fragment thereof comprises:
(1) a heavy chain comprising the sequence as set forth in SEQ ID NO: 37, and, a light chain comprising the sequence as set forth in SEQ ID NO: 38; or
(2) a heavy chain comprising the sequence as set forth in SEQ ID NO: 39, and, a light chain comprising the sequence as set forth in SEQ ID NO: 40.

In some embodiments, the antibody or antigen-binding fragment thereof is selected from Trastuzumab or Pertuzumab. The query accession number (IMGT/mAb-DB ID) for the amino acid sequence of Trastuzumab in the IMGT database is: 97. The query accession number (IMGT/mAb-DB ID) for the amino acid sequence of Pertuzumab in the IMGT database is: 80.

In certain embodiments, the antibody or antigen-binding fragment disclosed herein has a heavy chain constant region that may comprise a C-terminal lysine, lack a C-terminal lysine, or lack a C-terminal glycine-lysine dipeptide. In some embodiments, the N-terminal amino acid of the antibody or antigen-binding fragment thereof may be cyclized to pyroglutamic acid.

As known to those skilled in the art, pyroglutamic acid is the conjugate acid of pyroglutamate and is in equilibrium with pyroglutamate in solution.

In certain embodiments, provided herein is a composition comprising the antibody or antigen-binding fragments disclosed herein, wherein each antibody or antigen-binding fragment thereof may independently comprise a C-terminal lysine, lack a C-terminal lysine, lack a C-terminal glycine-lysine and/or comprise a N-terminal glutamine or glutamic acid, or a N-terminal amino acid cyclized to pyroglutamic acid or pyroglutamate.

In certain embodiments, the antibody or antigen-binding fragment disclosed herein comprises those capable of specifically binding to an antigen and may comprise post-translational modifications thereof (e.g., cleavage of the C-terminal lysine in the heavy chain, conversion of the N-terminal glutamine or glutamic acid in the heavy or light chain to pyroglutamic acid or pyroglutamate), which may occur during recombinant expression in a host cell (e.g., CHO cells) or arise during purification/storage.

In certain embodiments, the N-terminal glutamine of the VH having the sequence as set forth in SEQ ID NO: 1 or 3 or variant thereof or the heavy chain having the sequence as set forth in SEQ ID NO: 37 or 39 or variant thereof undergoes cyclization to form pyroglutamic acid or pyroglutamate.

In certain embodiments, the heavy chain constant region (CH) as set forth in SEQ ID NO: 35 or variant thereof or the heavy chain having the sequence as set forth in SEQ ID NO: 37 or 39 or variant thereof lacks C-terminal lysine.

In some embodiments, the antibody-drug conjugate is selected from the group consisting of ADC F-1 to ADC F-20, ADC L-1 to ADC L-11 as shown below:

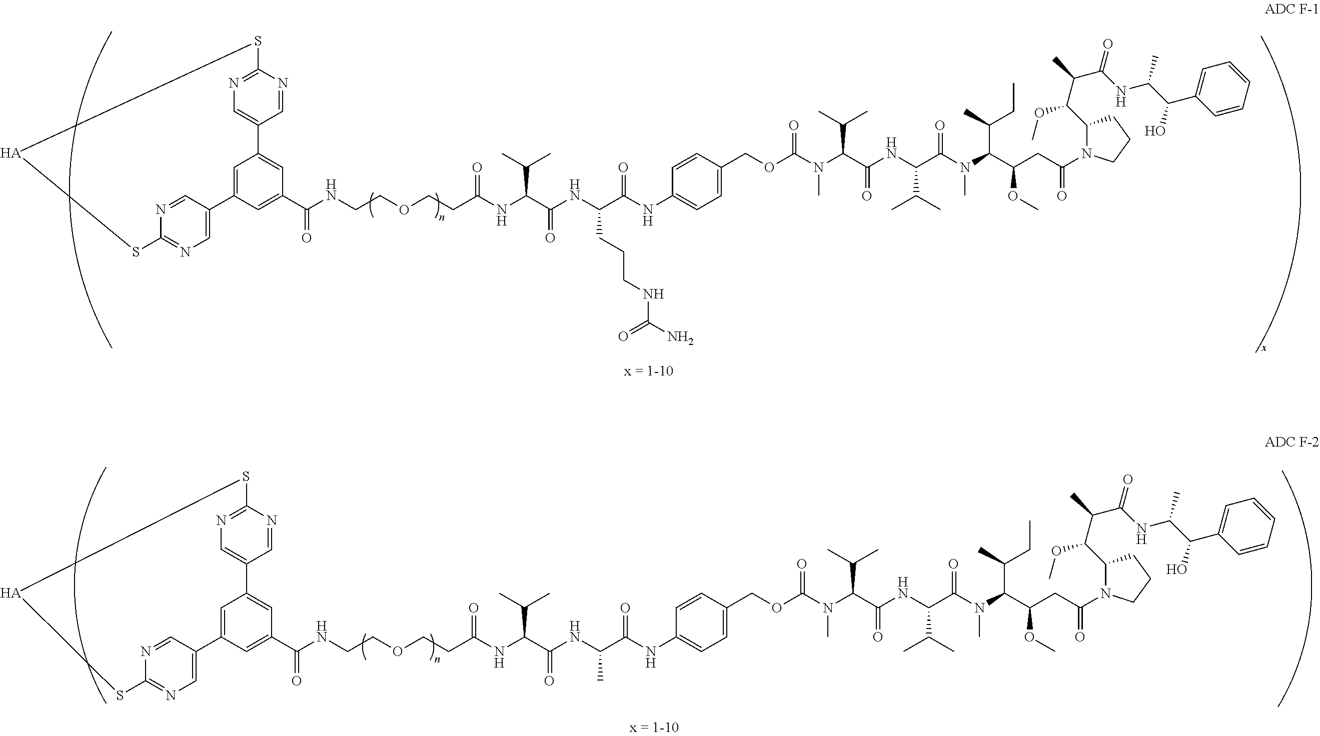

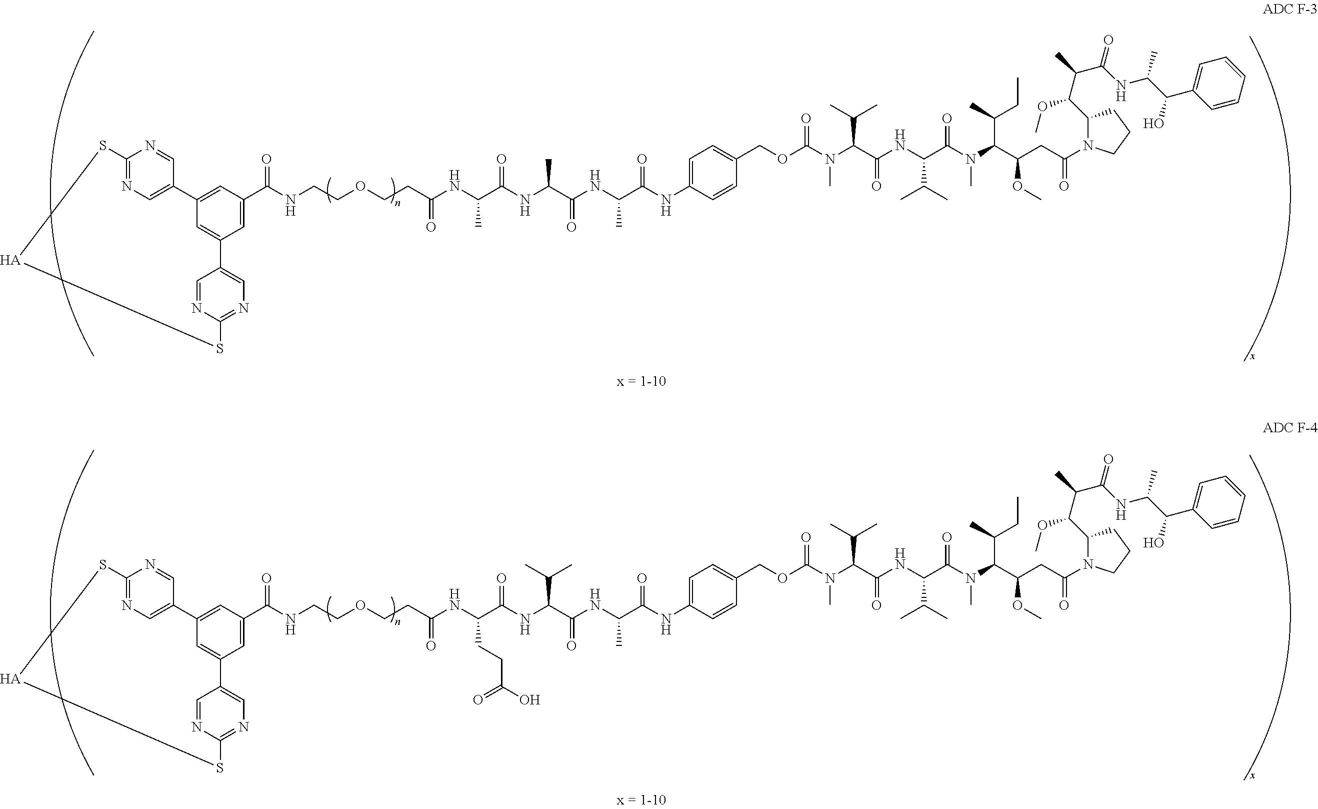

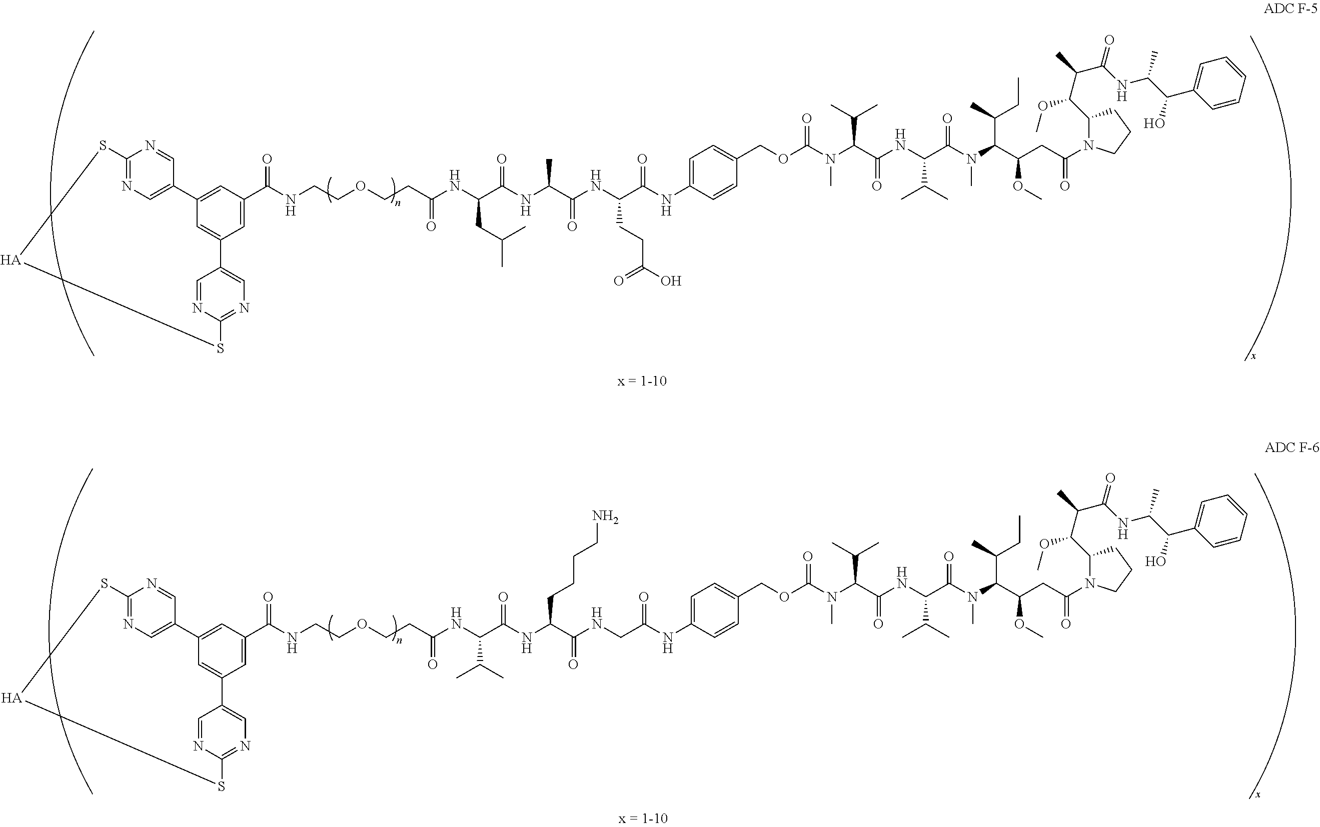

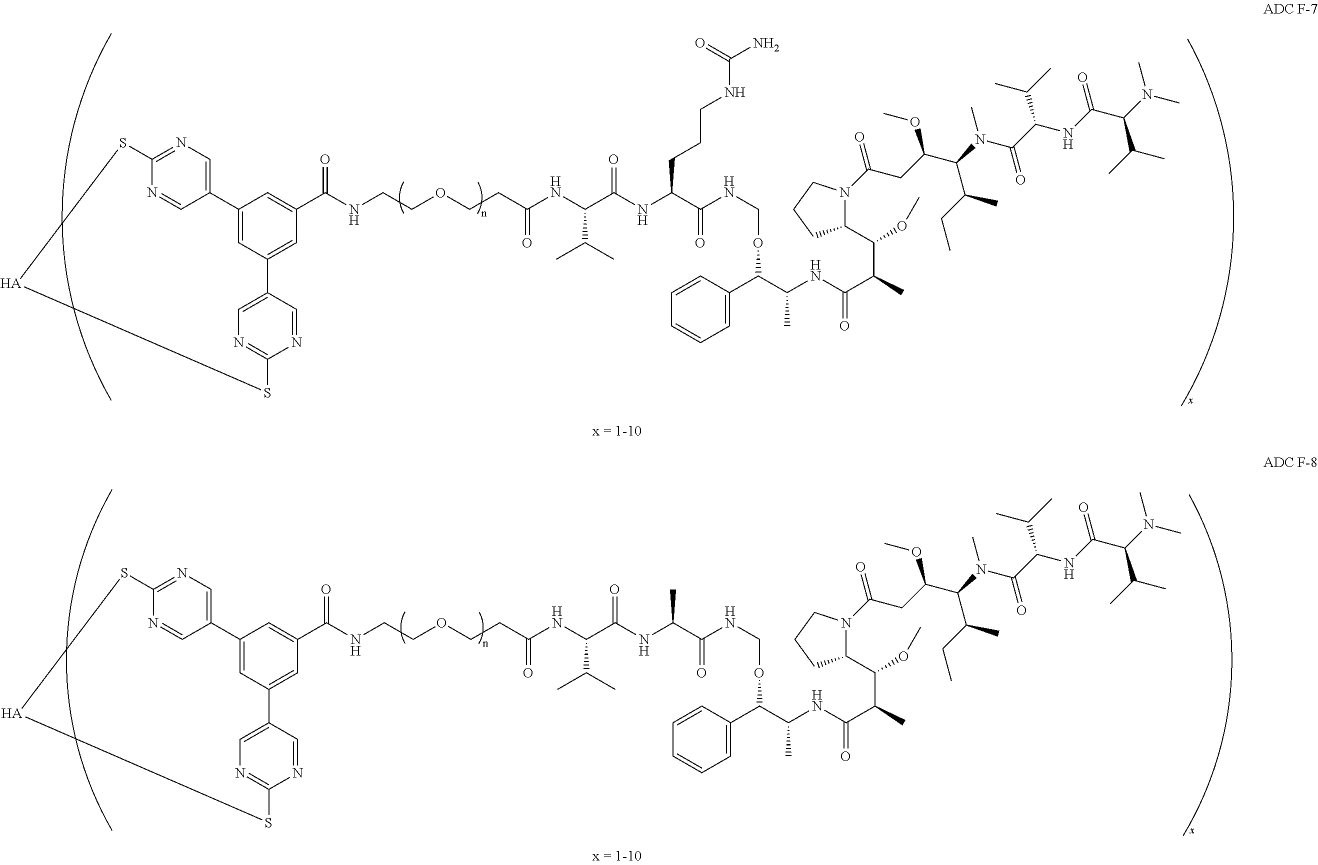

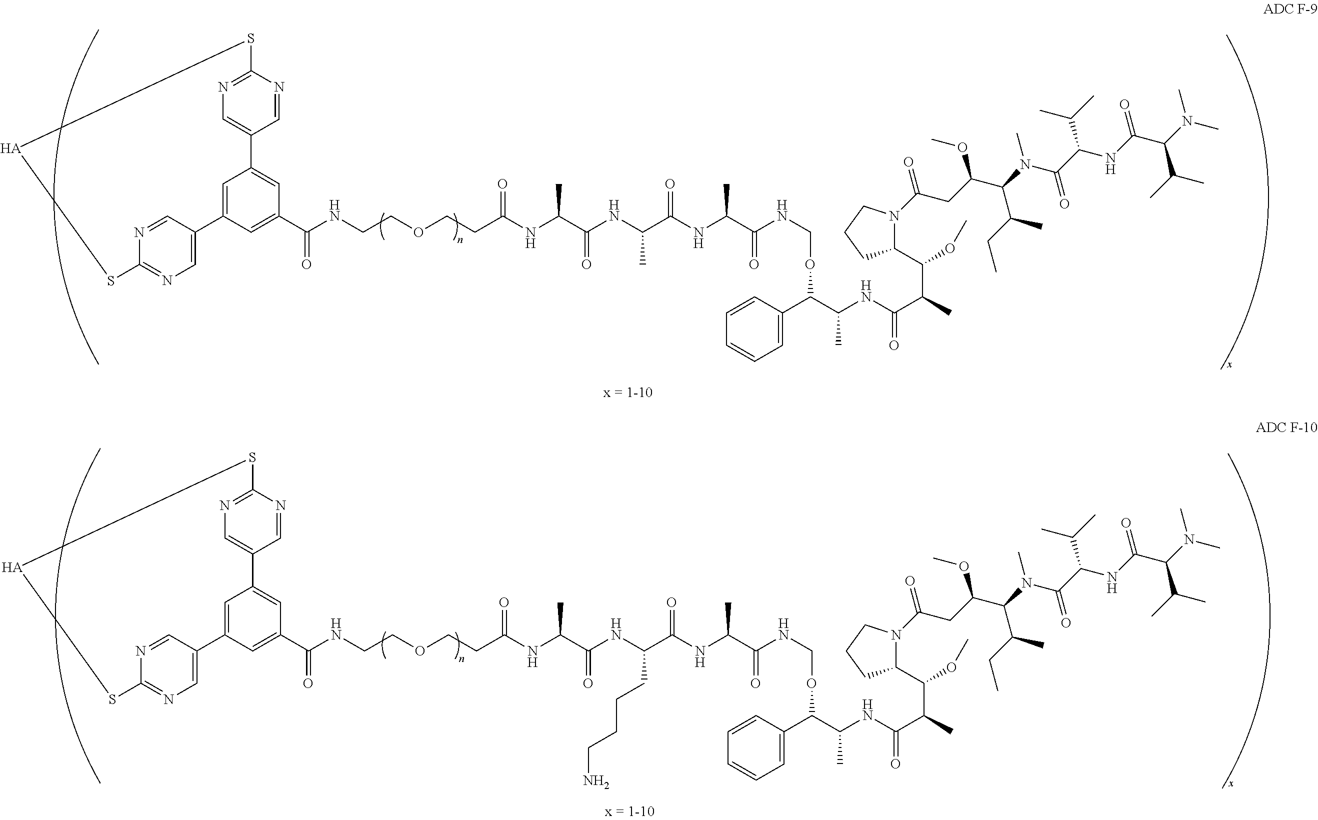

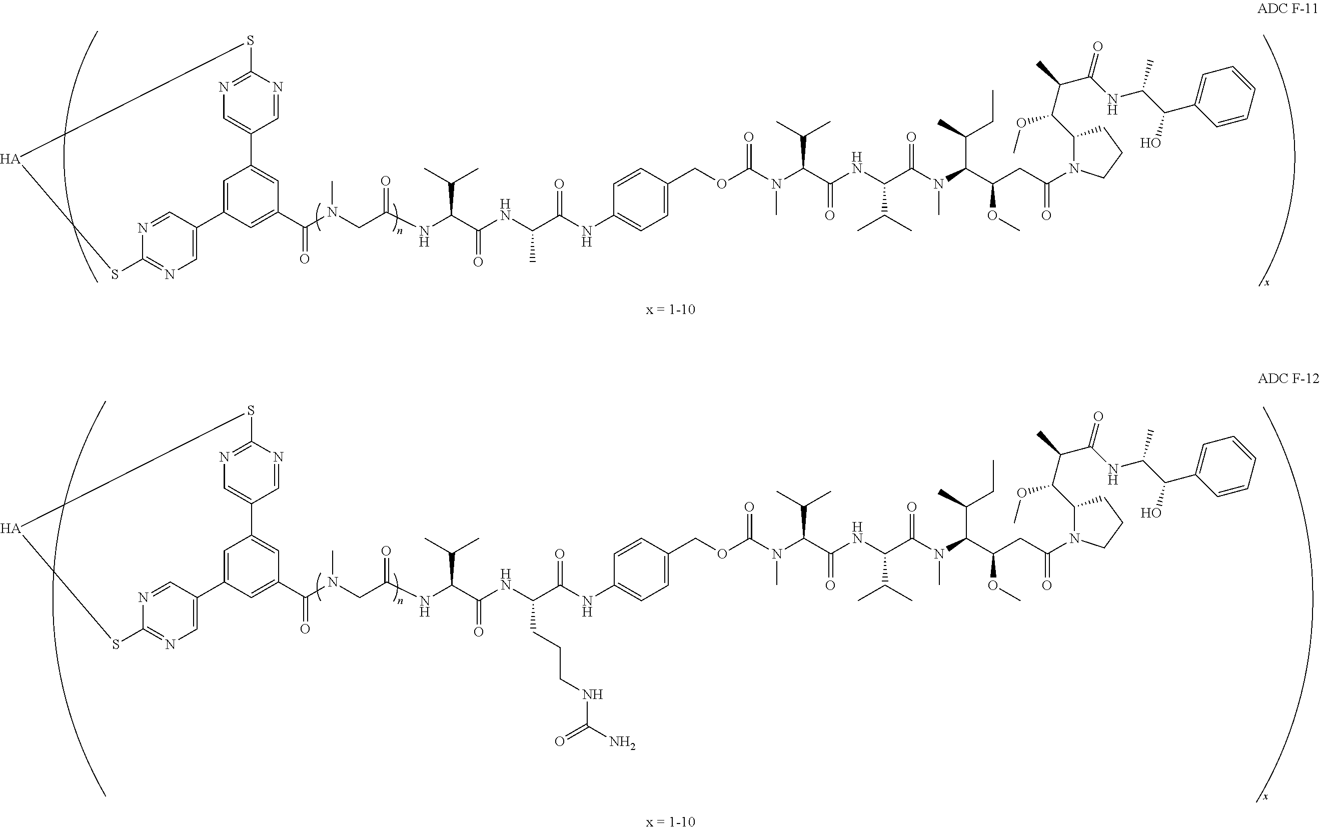

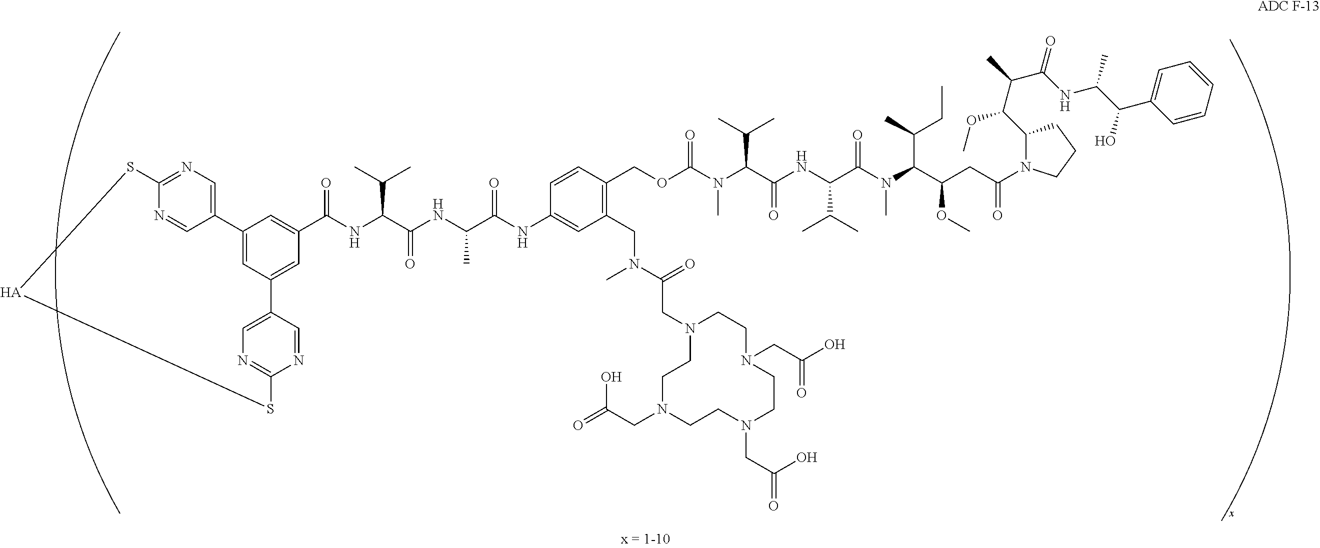

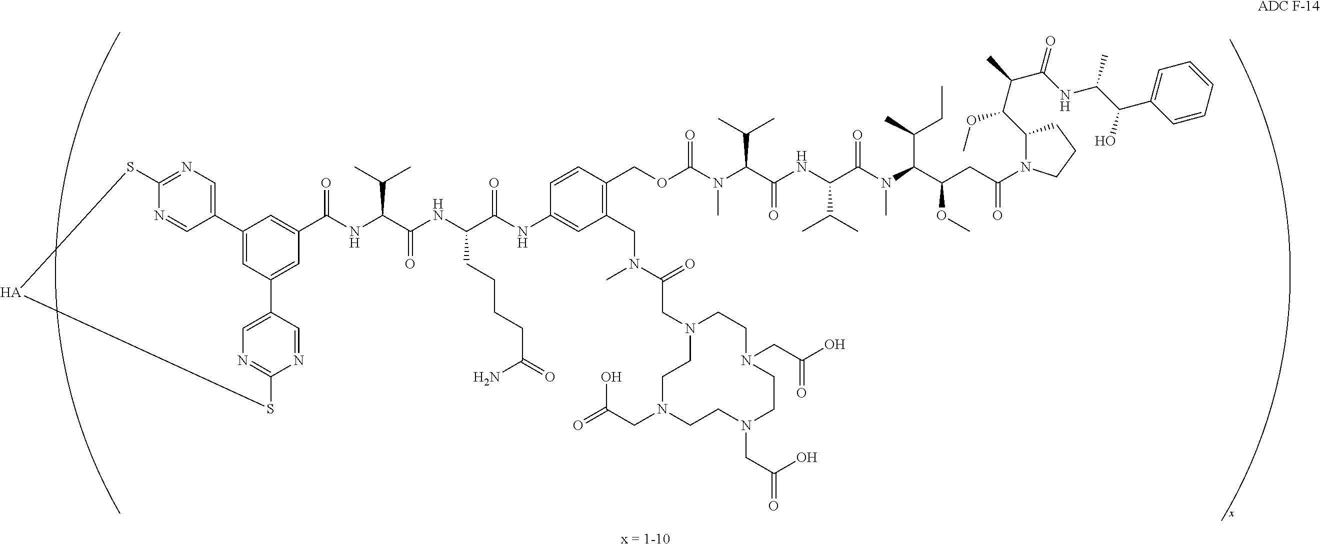

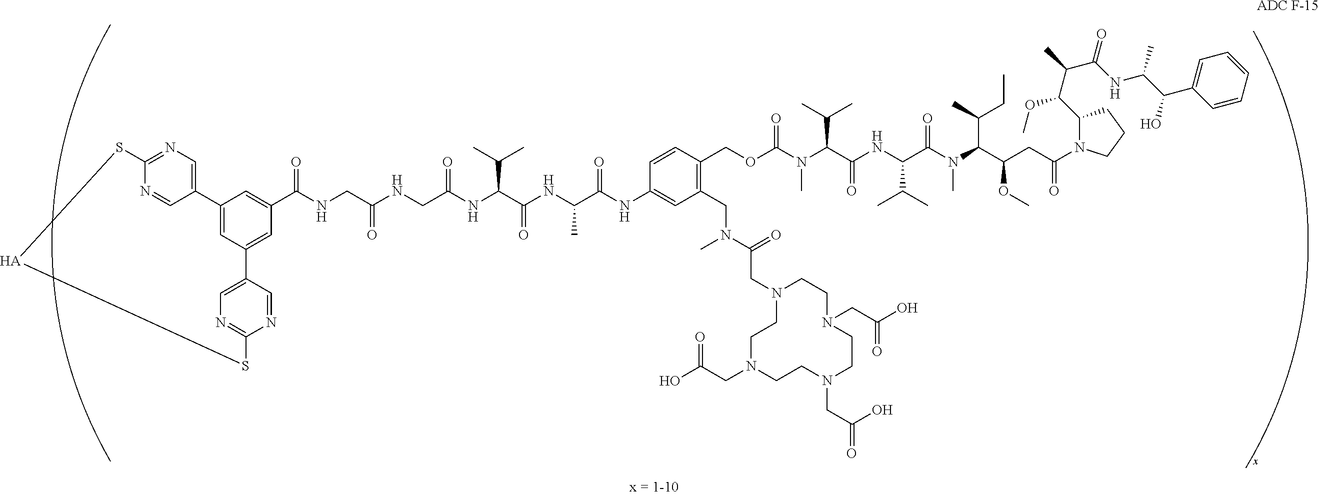

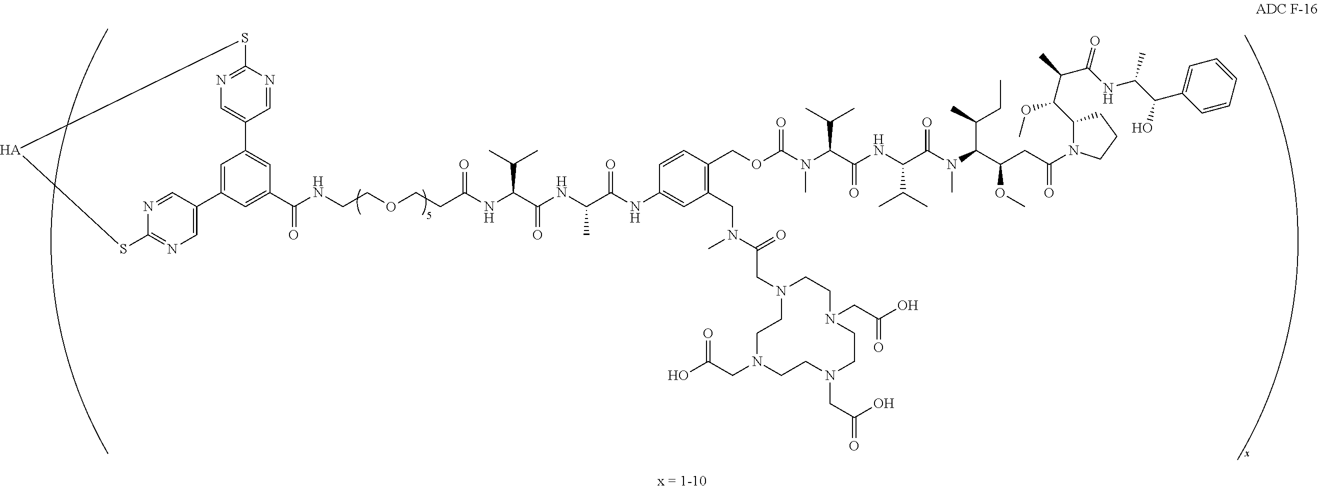

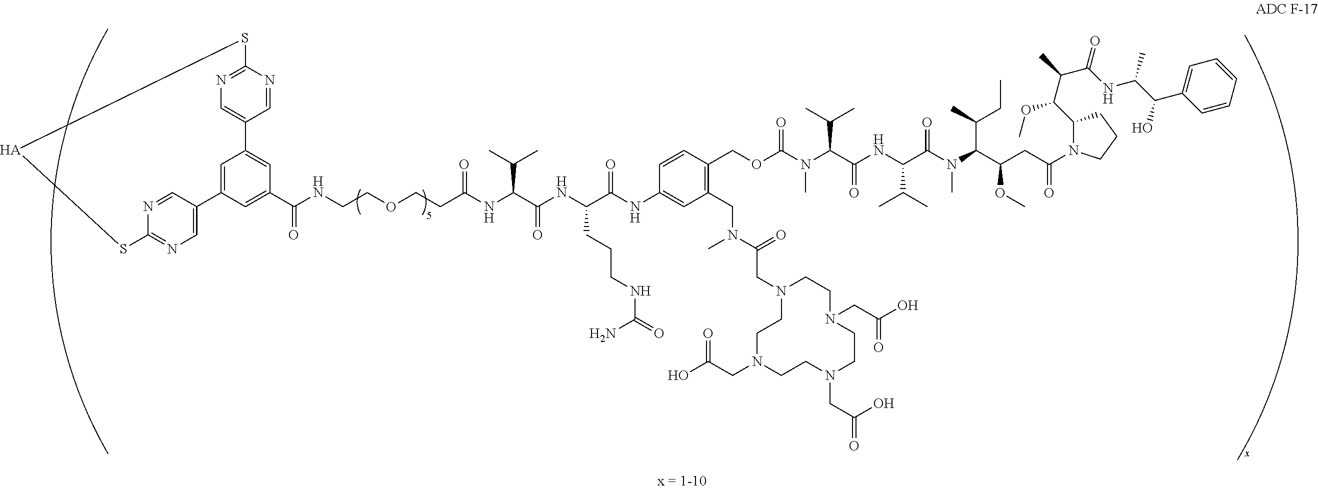

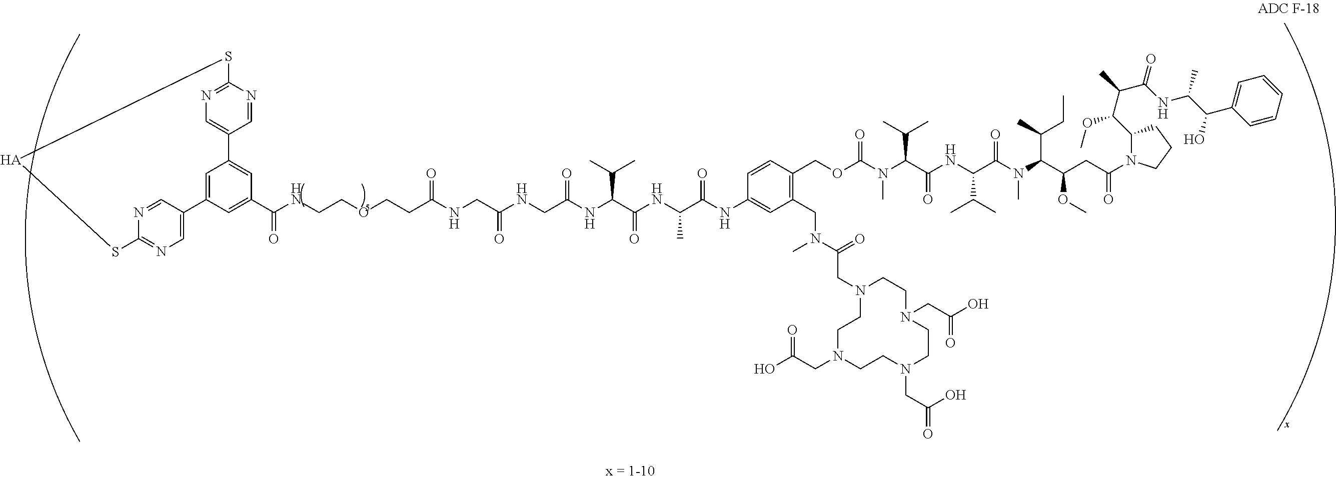

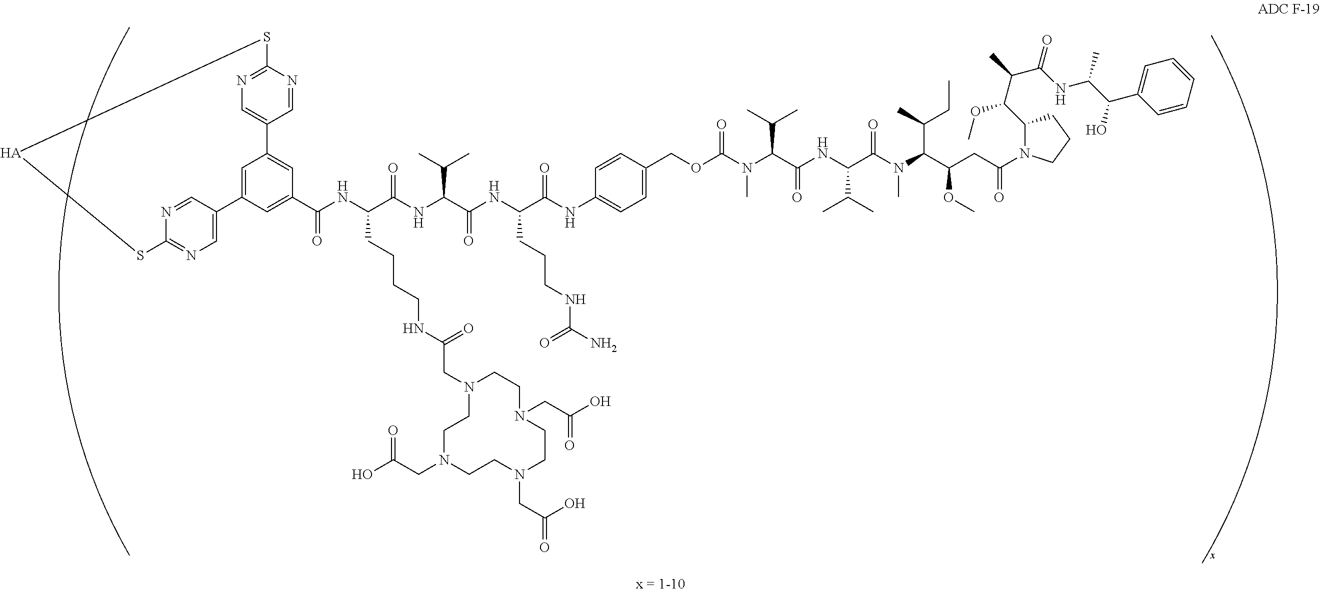

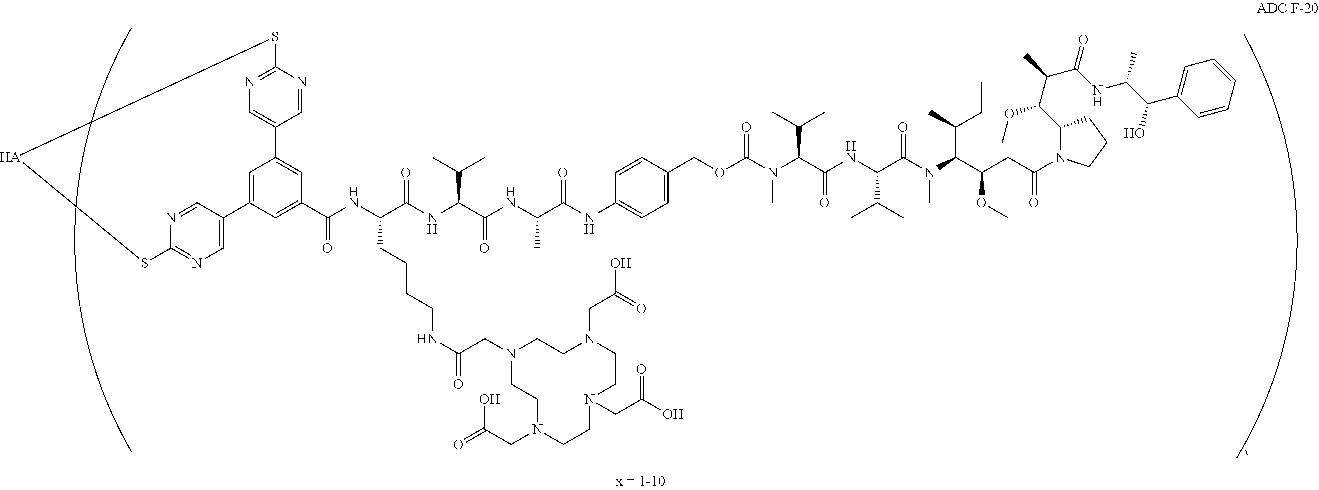

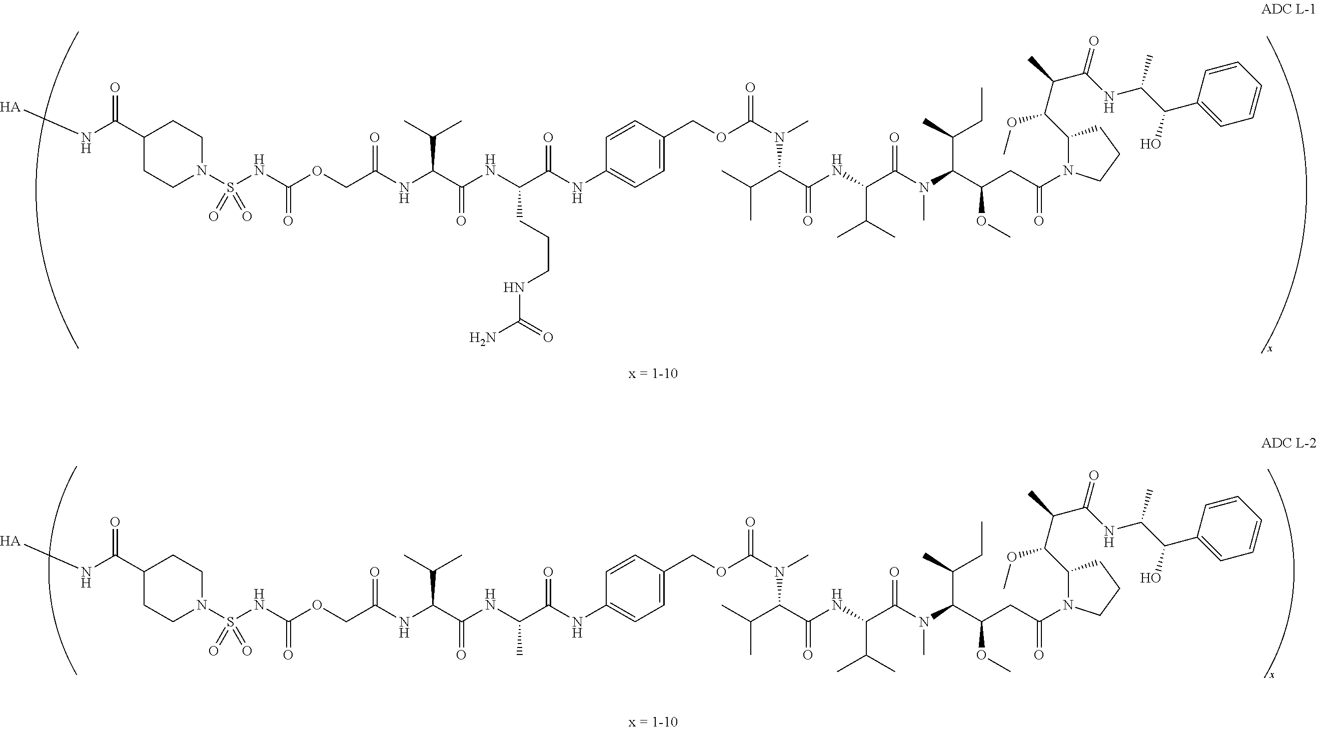

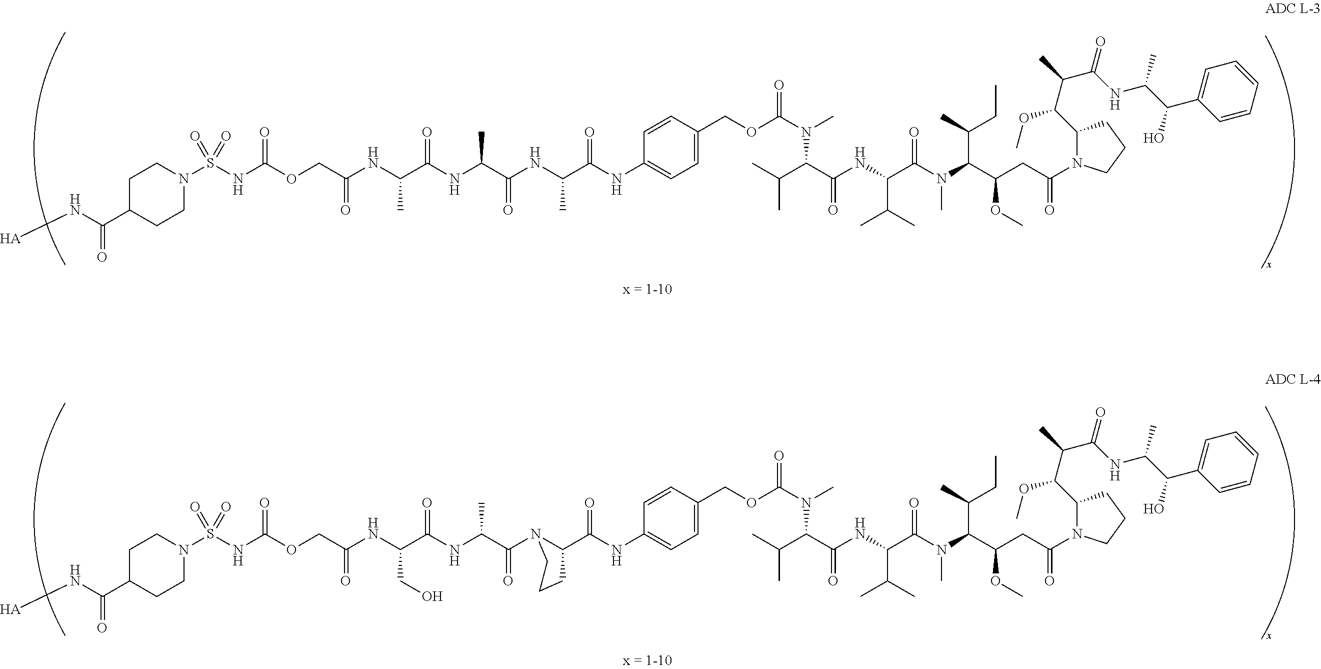

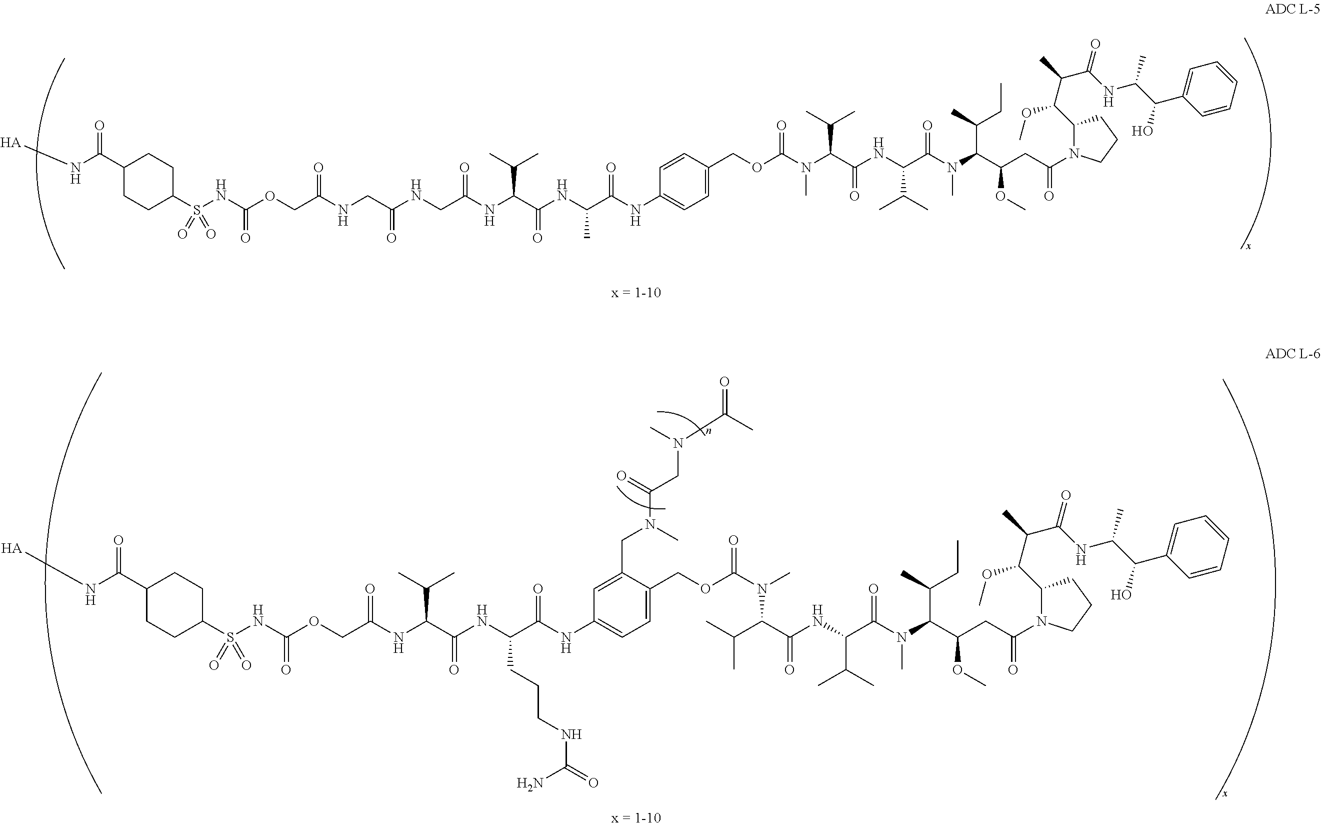

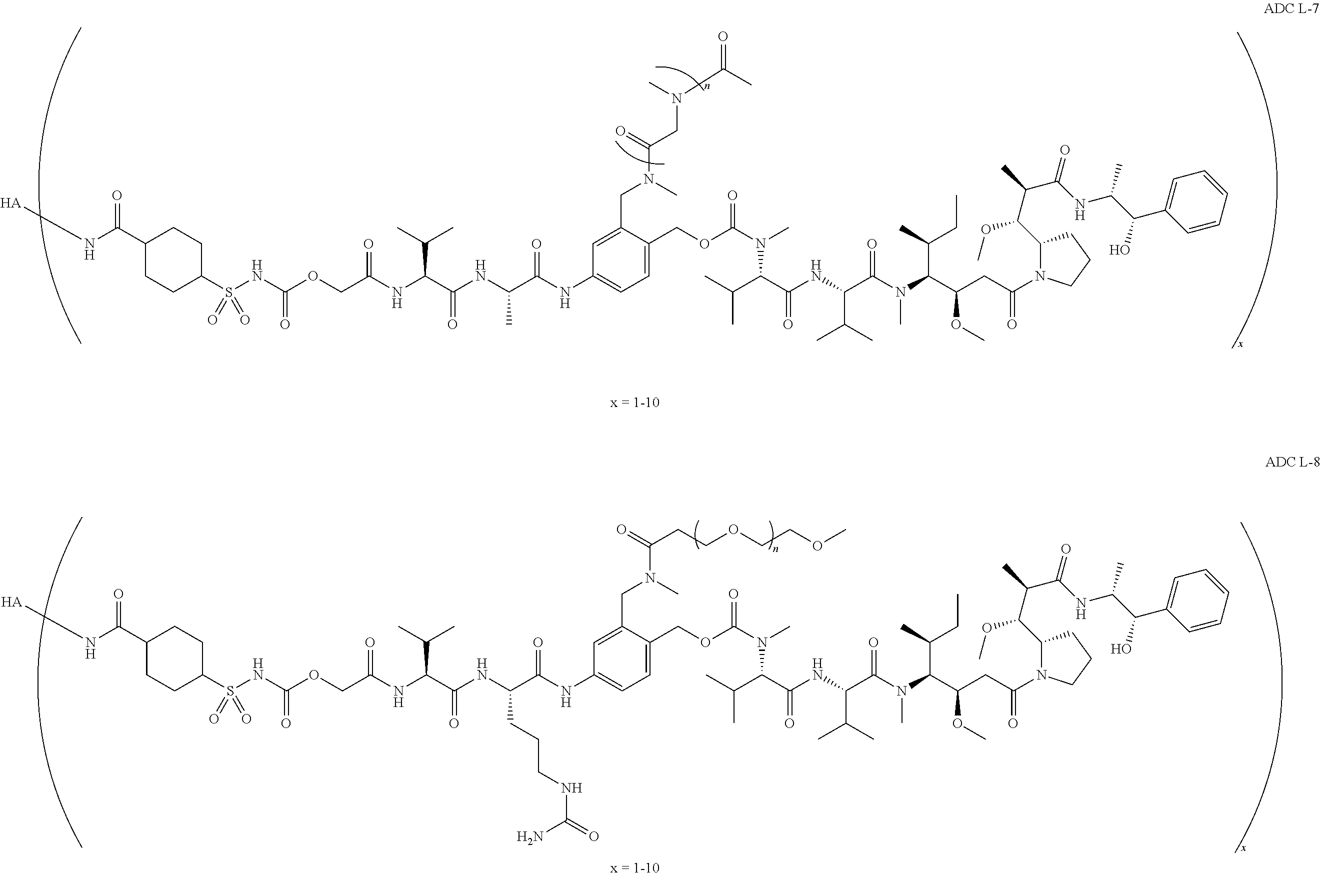

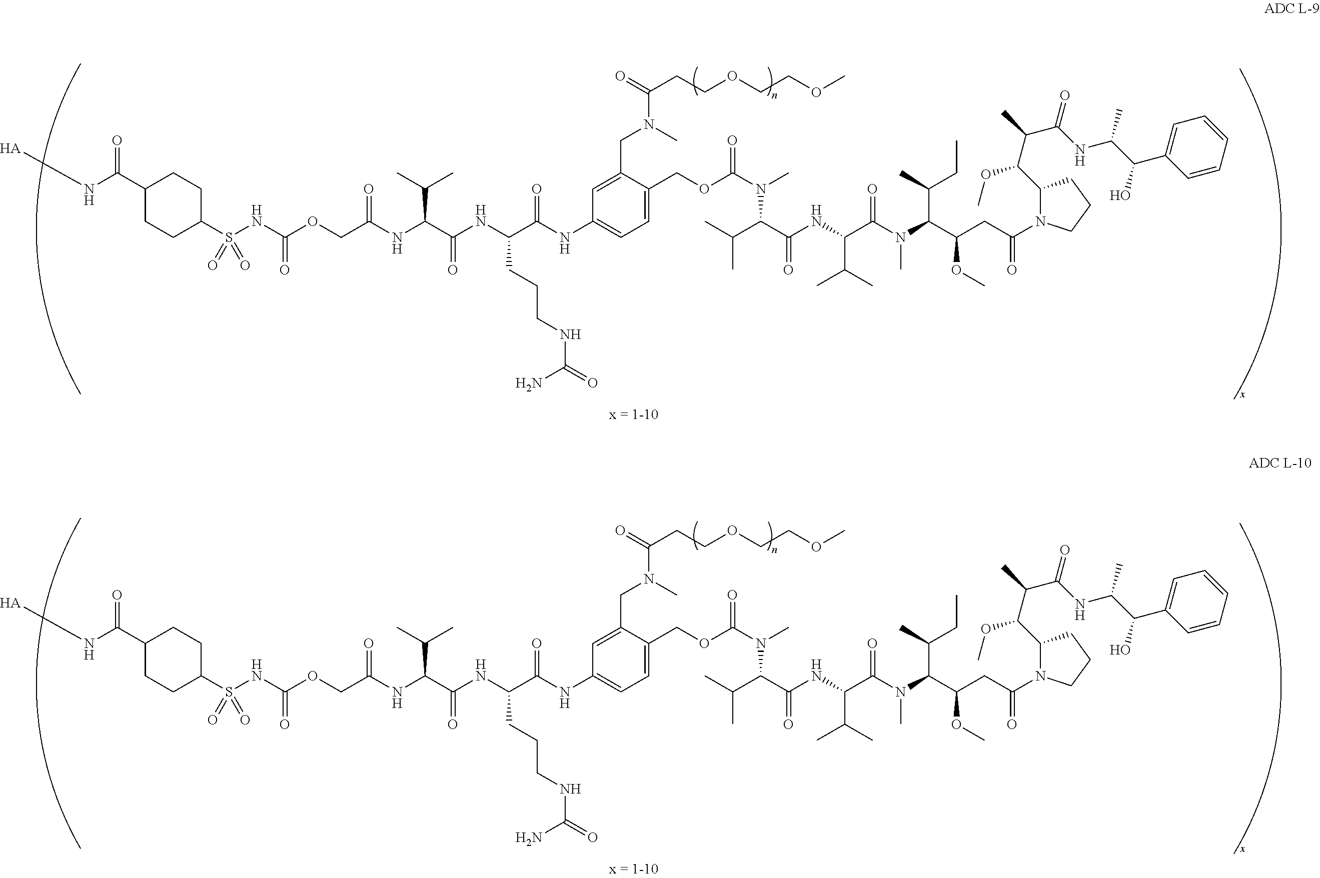

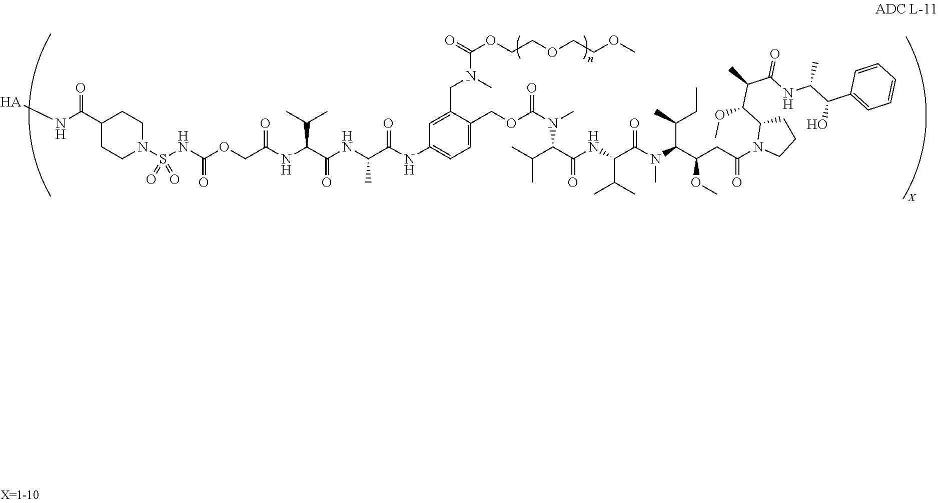

where, HA in each antibody-drug conjugate represents an antibody or antigen-binding fragment thereof capable of specifically binding to epidermal growth factor receptor 2 (Her2), a member of the ErbB family of receptor tyrosine kinases; n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 5, 8, 9 or 10;
where,

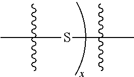 [SMILES: *S*]

represents a specific linkage between a sulfhydryl group in the antibody or antigen-binding fragment thereof and the M fragment;

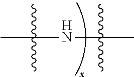 [SMILES: *N*]

represents a specific linkage between an amino group in the antibody or antigen-binding fragment thereof and the M fragment.

In some embodiments, the antibody-drug conjugate is selected from the group consisting of ADC F′-1 to ADC F′-12, ADC F′-16 to ADC F′-18, ADC L′-6 to ADC L′-11 as shown below:

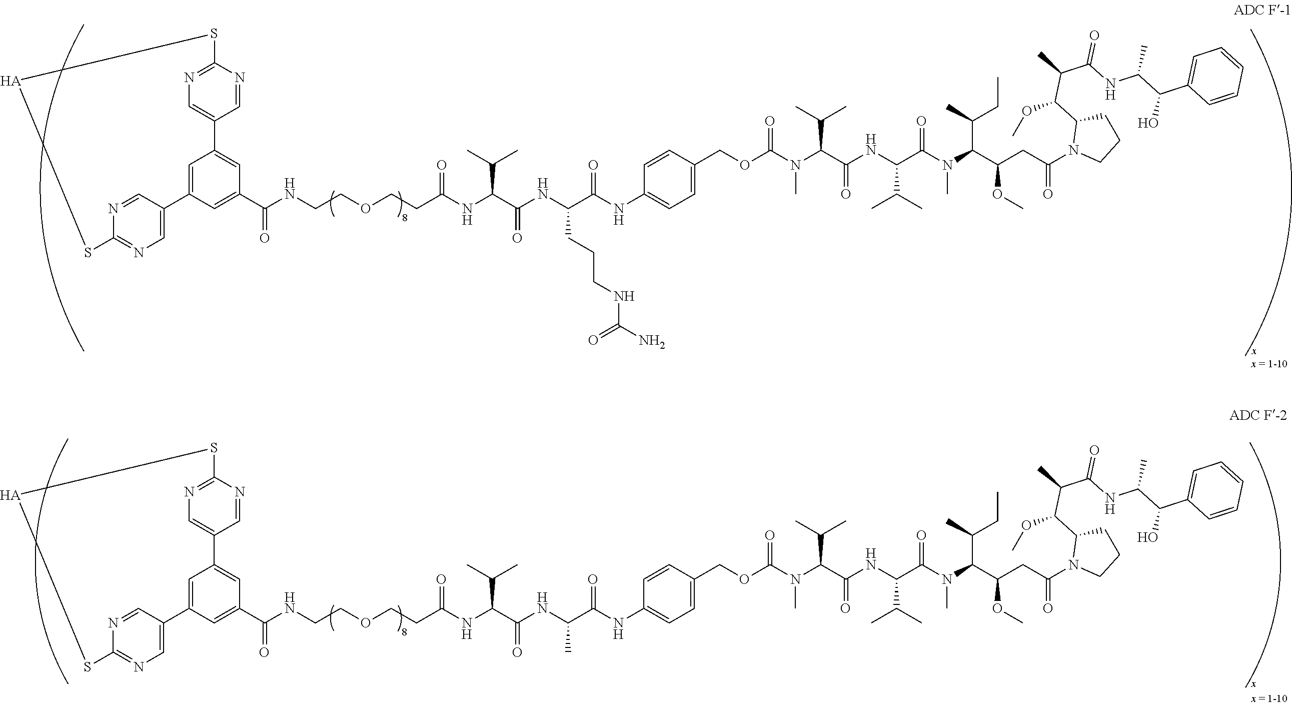

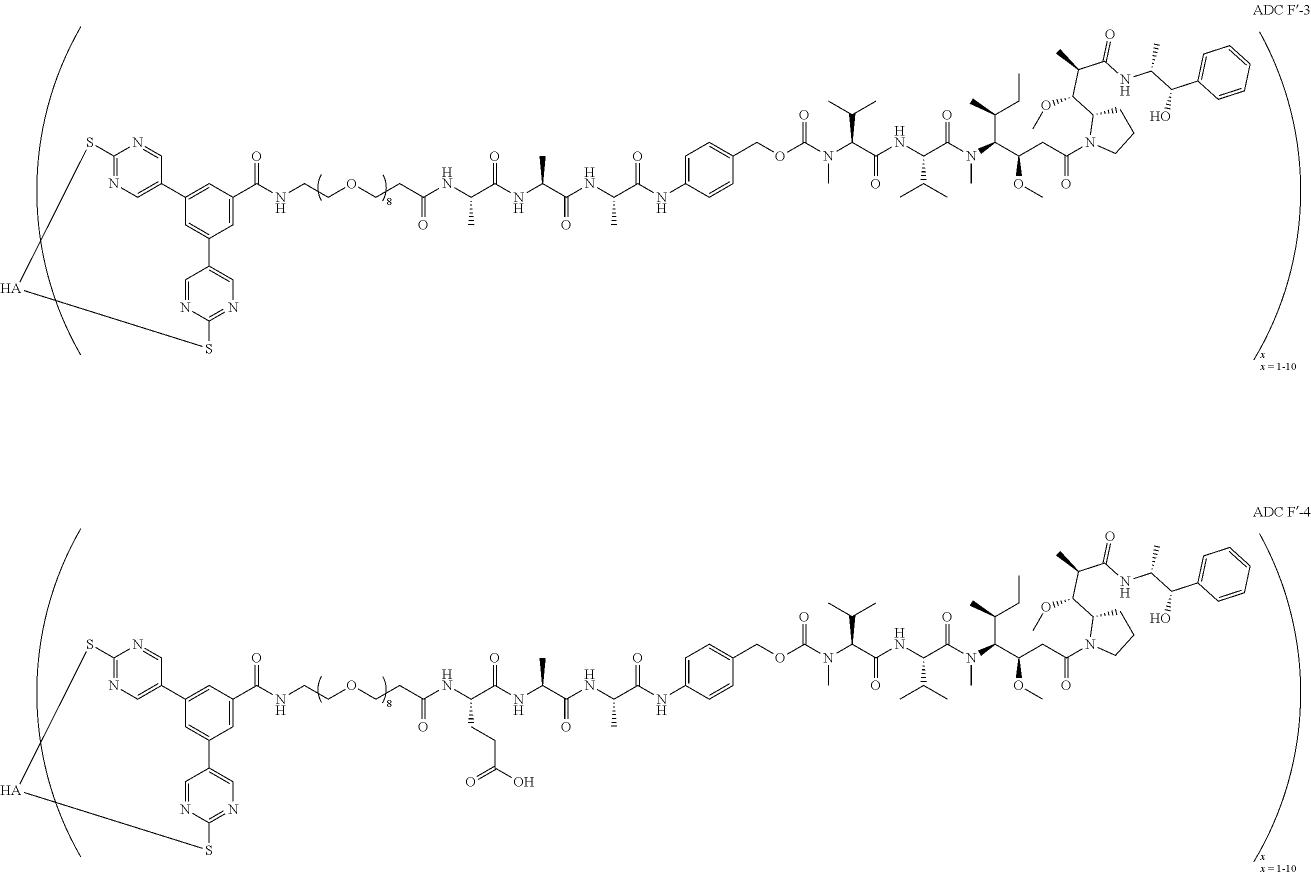

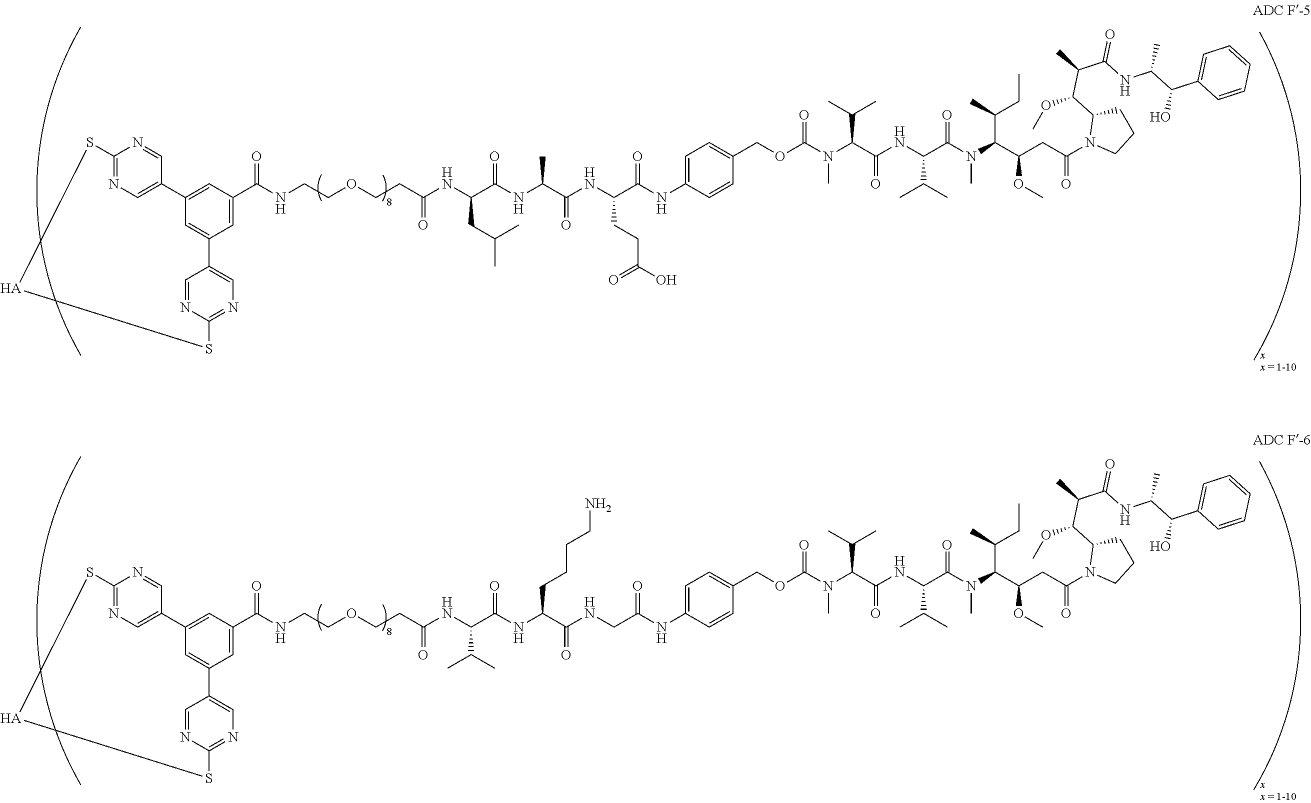

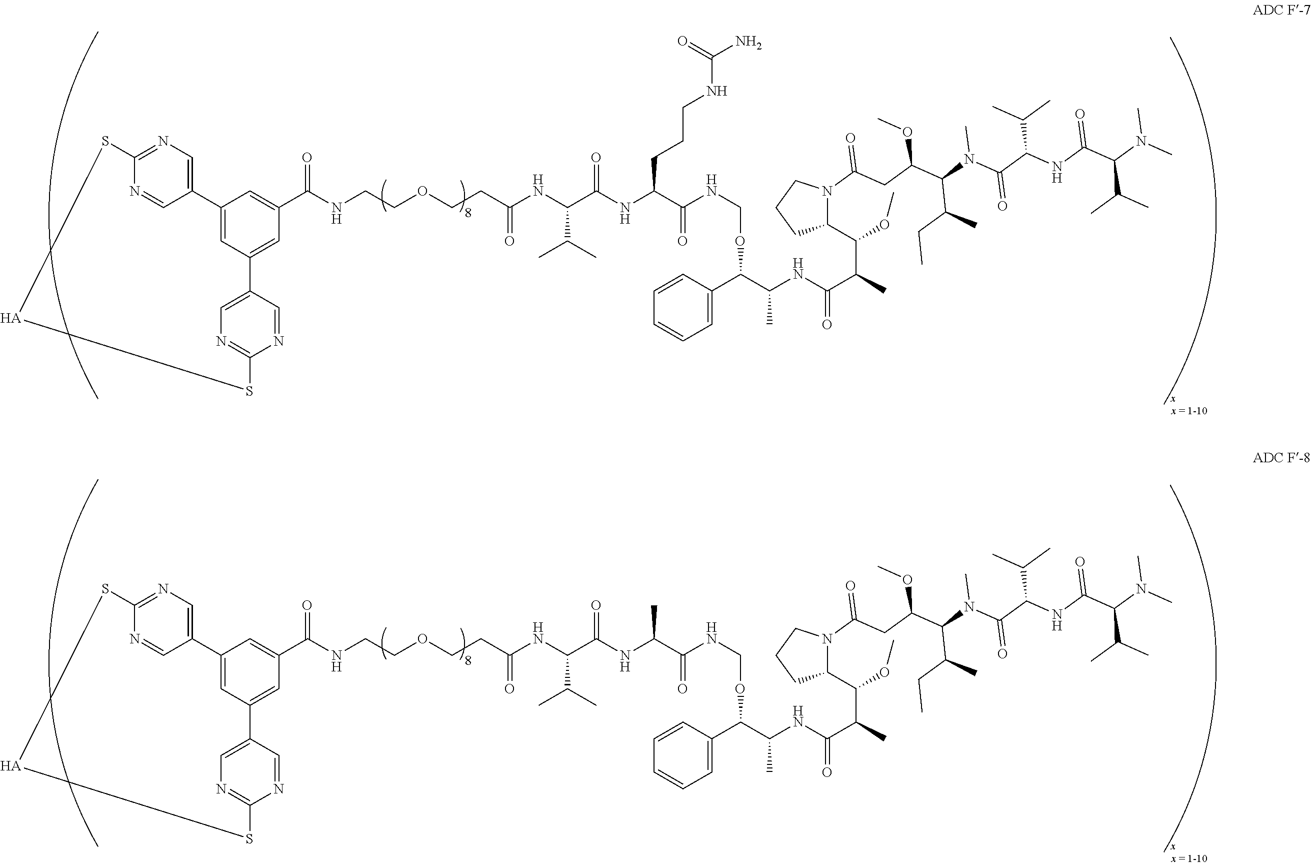

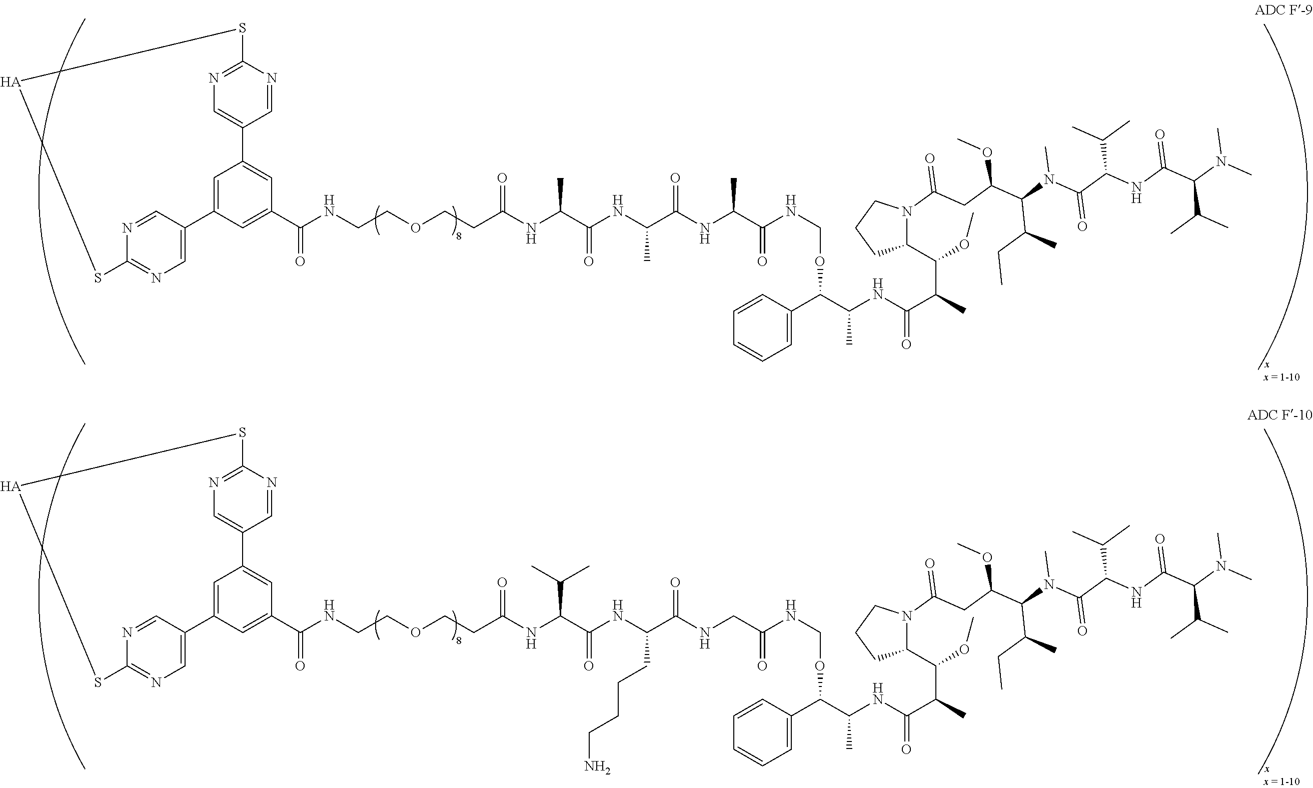

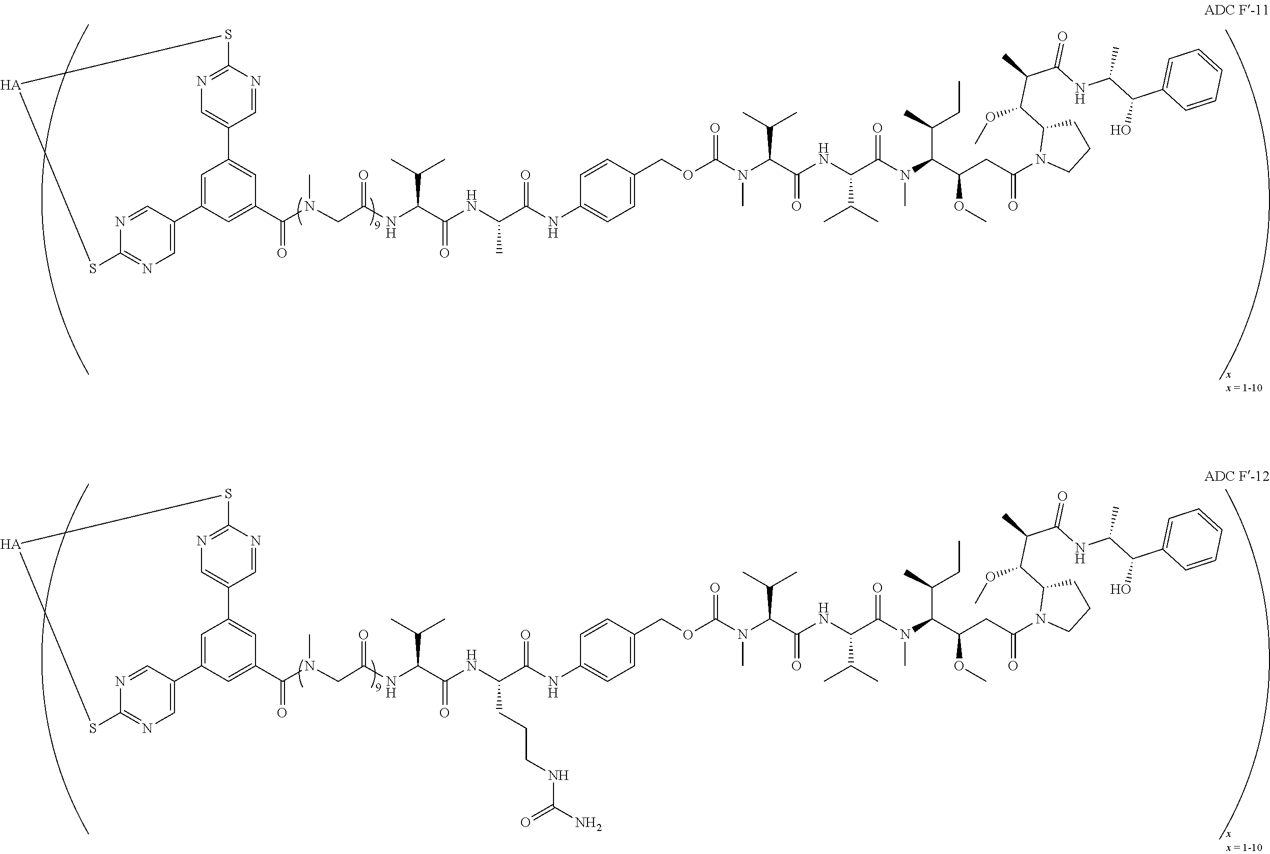

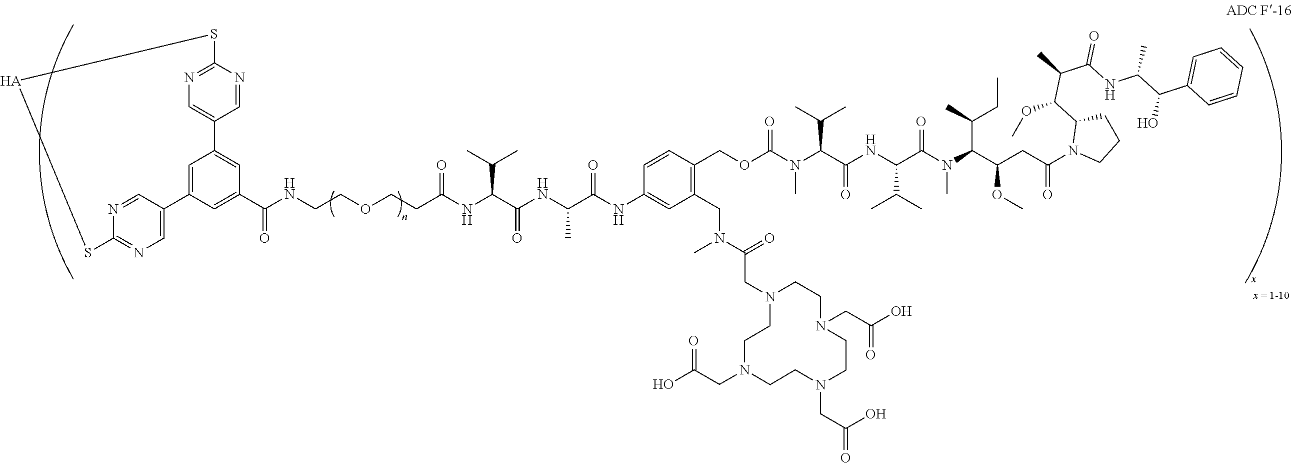

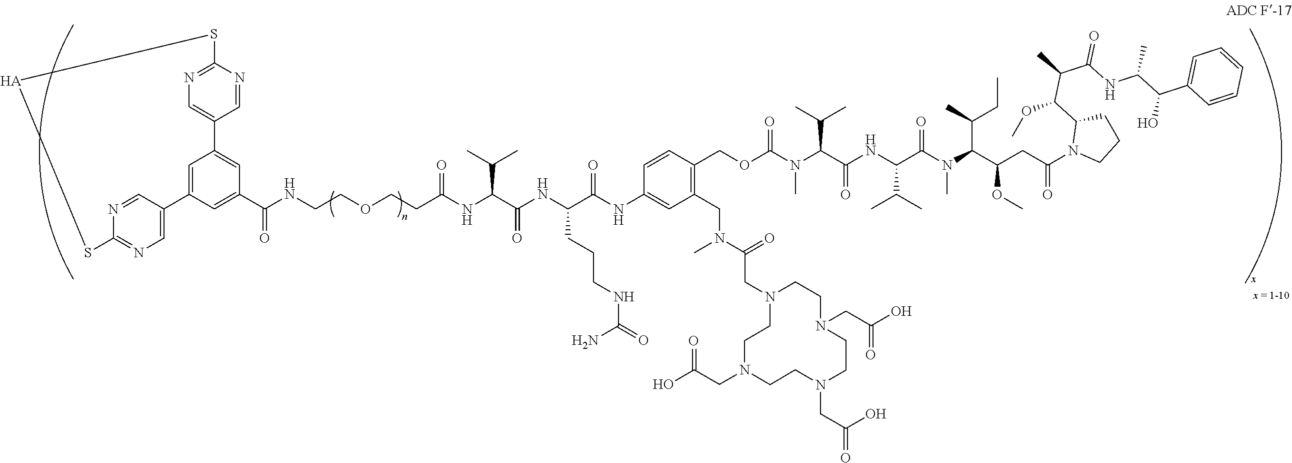

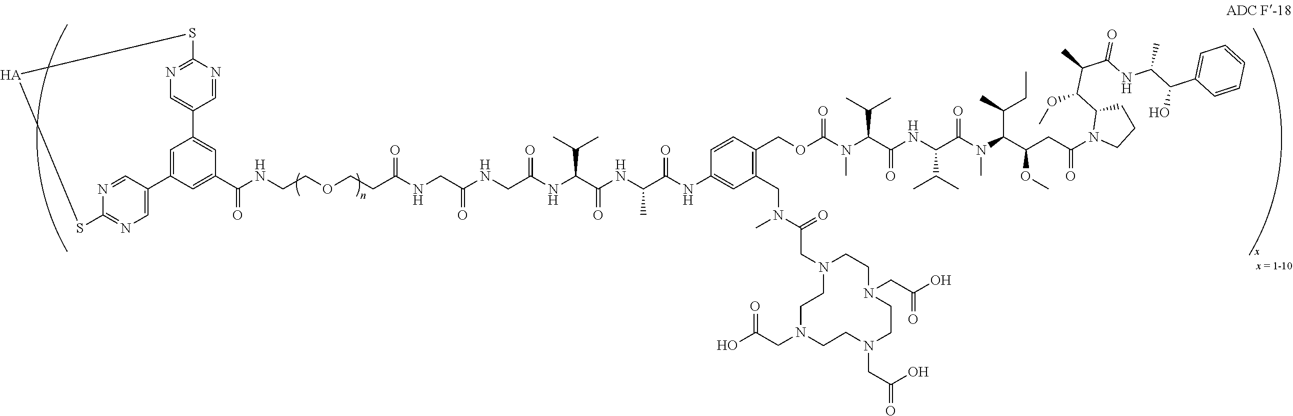

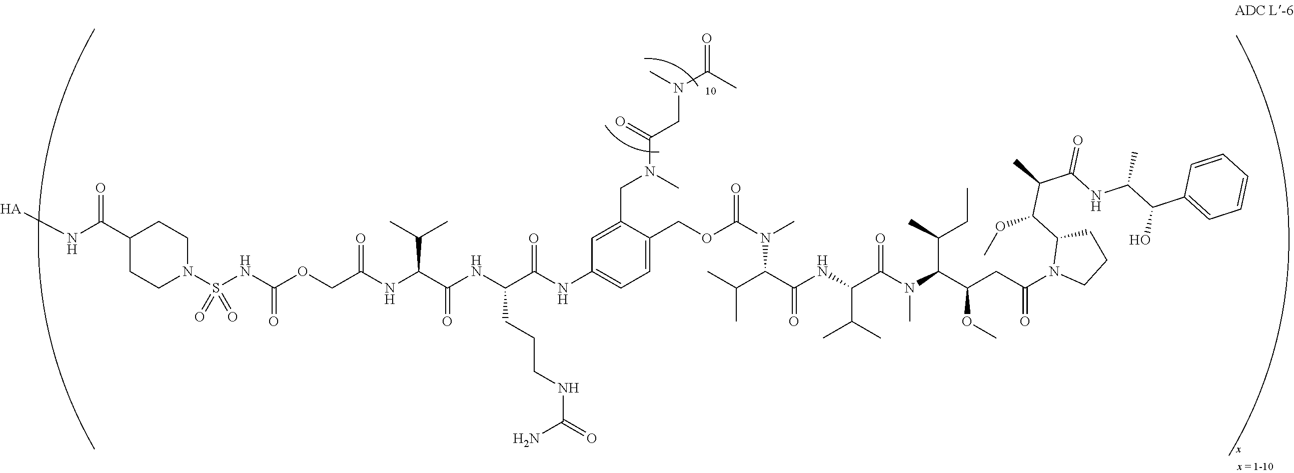

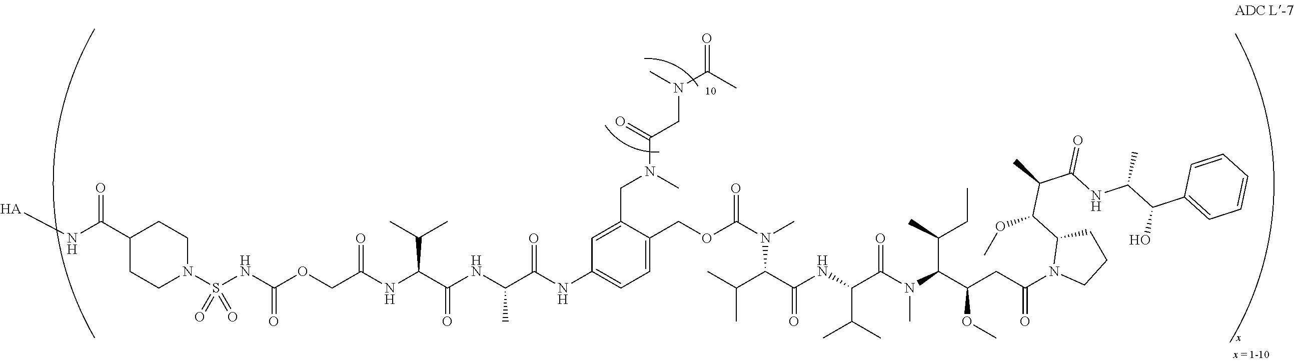

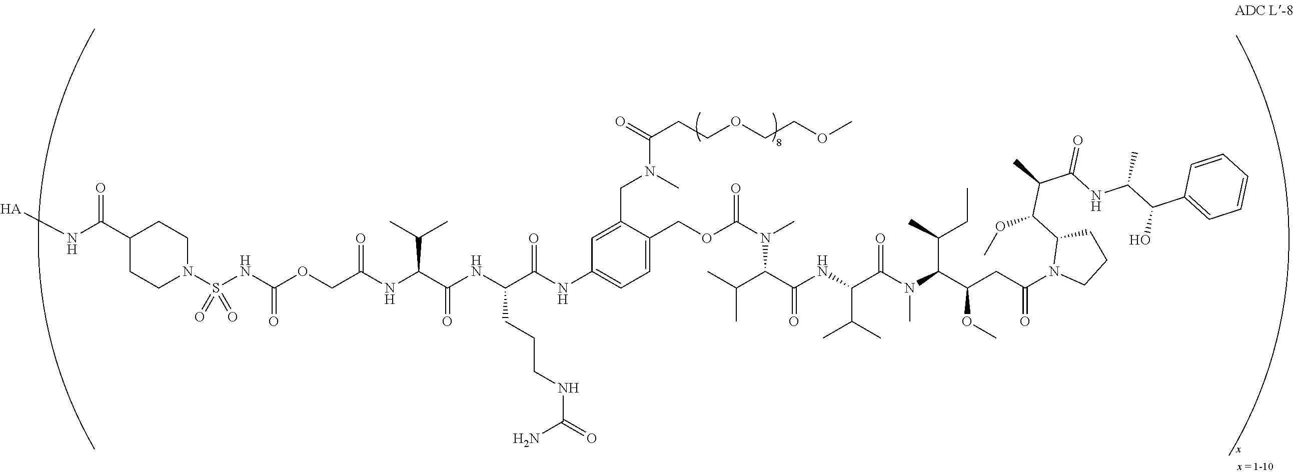

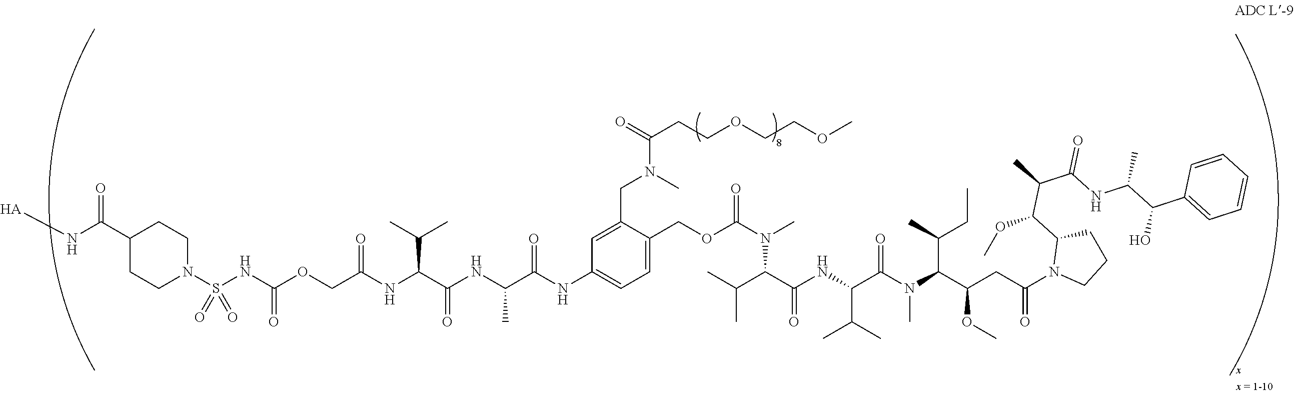

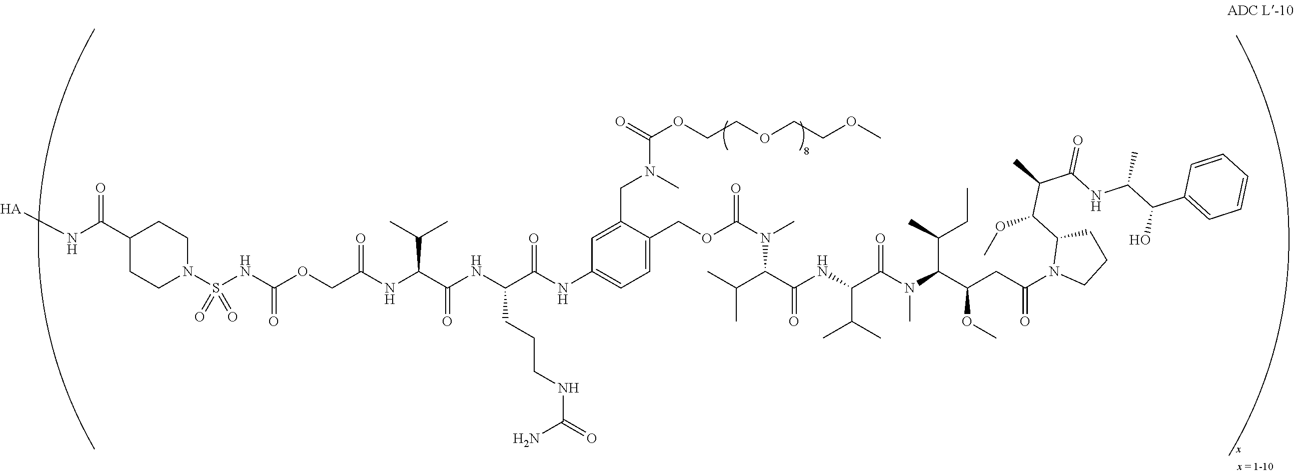

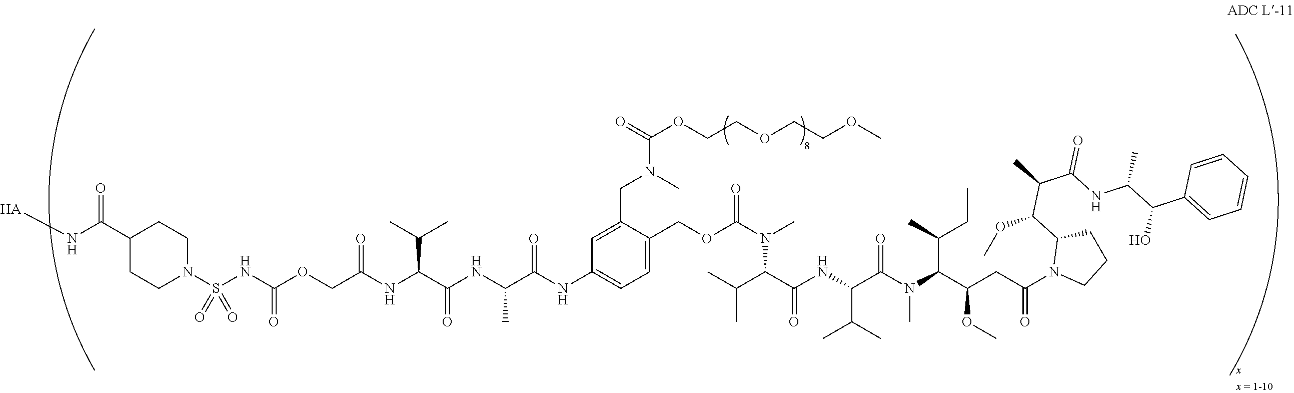

where, HA in each antibody-drug conjugate represents an antibody or antigen-binding fragment thereof; n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 5, 8, 9 or 10;
where,

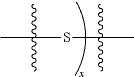 [SMILES: *S*]

represents a specific linkage between a sulfhydryl group in the antibody or antigen-binding fragment thereof and the M fragment;

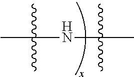 [SMILES: *N*]

represents a specific linkage between an amino group in the antibody or antigen-binding fragment thereof and the M fragment.

In some embodiments, HA of each antibody-drug conjugate comprises:
(1) the following heavy chain variable region (VH) and/or light chain variable region (VL):
(1a) having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof; or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof; and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

In some preferred embodiments, HA of each antibody-drug conjugate comprises:
(1) the following heavy chain variable region (VH) and/or light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof, or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a) and (3b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable regions (VH) and/or light chain variable regions (VL), wherein the CDRs are defined according to the IMGT numbering system:
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following three CDRs: a CDR-L1 having a sequence as set forth in SEQ ID NO: 16 or variant thereof, and a CDR-L2 having a sequence as set forth in SEQ ID NO: 17 or variant thereof. L2, and a CDR-L3 having a sequence as set forth in SEQ ID NO: 10 or variant thereof; 01,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, a CDR-H3 having a sequence as set forth in SEQ ID NO: 30 or variant thereof; and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared to the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution,
In some embodiments, HA of each antibody-drug conjugate comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
of,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and/or, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and/or, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and/or, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and/or, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, HA of each antibody-drug conjugate comprises:
(1) the following heavy chain variable region (VH) and light chain variable region (VL), wherein the CDRs are defined according to the Chothia numbering system:
(1a) a heavy chain variable region (VH) comprising the following three CDRs: a CDR-H1 having a sequence as set forth in SEQ ID NO: 5, a CDR-H2 having a sequence as set forth in SEQ ID NO: 6, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(1b) a heavy chain variable region (VH) comprising the following three CDRs: a CDR-H1 having a sequence as set forth in SEQ ID NO: 20, a CDR-H2 having a sequence as set forth in SEQ ID NO: 21, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(2) the following heavy chain variable region (VH) and light chain variable region (VL), wherein the CDRs are defined according to the AbM numbering system:
(2a) a heavy chain variable region (VH) comprising the following three CDRs: a CDR-H1 having a sequence as set forth in SEQ ID NO: 18, a CDR-H2 having a sequence as set forth in SEQ ID NO: 19, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(2b) a heavy chain variable region (VH) comprising the following three CDRs: a CDR-H1 having a sequence as set forth in SEQ ID NO: 33, a CDR-H2 having a sequence as set forth in SEQ ID NO: 34, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(3) the following heavy chain variable region (VH) and light chain variable region (VL), wherein the CDRs are defined according to the Kabat numbering system:
(3a) a heavy chain variable region (VH) comprising the following three CDRs: a CDR-H1 having a sequence as set forth in SEQ ID NO: 11, a CDR-H2 having a sequence as set forth in SEQ ID NO: 12, and a CDR-H3 having a sequence as set forth in SEQ ID NO: 7; and, a light chain variable region (VL) comprising the following three CDRs: a CDR-L1 having a sequence as set forth in SEQ ID NO: 8, a CDR-L2 having a sequence as set forth in SEQ ID NO: 9, and a CDR-L3 having a sequence as set forth in SEQ ID NO: 10; or,
(3b) a heavy chain variable region (VH) comprising the following three CDRs: a CDR-H1 having a sequence as set forth in SEQ ID NO: 26, a CDR-H2 having a sequence as set forth in SEQ ID NO: 27, and a CDR-H3 having a sequence as set forth in SEQ ID NO: 22; and, a light chain variable region (VL) comprising the following three CDRs: a CDR-L1 having a sequence as set forth in SEQ ID NO: 23, a CDR-L2 having a sequence as set forth in SEQ ID NO: 24, and a CDR-L3 having a sequence as set forth in SEQ ID NO: 25;
or,
(4) the following heavy chain variable region (VH) and light chain variable region (VL), wherein the CDRs are defined according to the IMGT numbering system:
(4a) a heavy chain variable region (VH) comprising the following three CDRs: a CDR-H1 having a sequence as set forth in SEQ ID NO: 13, a CDR-H2 having a sequence as set forth in SEQ ID NO: 14, and a CDR-H3 having a sequence as set forth in SEQ ID NO: 15; and, a light chain variable region (VL) comprising the following three CDRs: a CDR-L1 having a sequence as set forth in SEQ ID NO: 16, a CDR-L2 having a sequence as set forth in SEQ ID NO: 17, and a CDR-L3 having a sequence as set forth in SEQ ID NO: 10; or,
(4b) a heavy chain variable region (VH) comprising the following three CDRs: a CDR-H1 having a sequence as set forth in SEQ ID NO: 28, a CDR-H2 having a sequence as set forth in SEQ ID NO: 29, and a CDR-H3 having a sequence as set forth in SEQ ID NO: 30; and, a light chain variable region (VL) comprising the following three CDRs: a CDR-L1 having a sequence as set forth in SEQ ID NO: 31, a CDR-L2 having a sequence as set forth in SEQ ID NO: 32, and a CDR-L3 having a sequence as set forth in SEQ ID NO: 25.

In some embodiments, HA of each antibody-drug conjugate comprises:
(a) a VH as set forth in SEQ ID NO: 1 or variant thereof, and/or, a VL as set forth in SEQ ID NO: 2 or variant thereof, or
(b) a VH as set forth in SEQ ID NO: 3 or variant thereof, and/or, a VL as set forth in SEQ ID NO: 4 or variant thereof; or
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

In some embodiments, HA of each antibody-drug conjugate comprises:
(a) a VH as set forth in SEQ ID NO: 1, and/or a VL as set forth in SEQ ID NO: 2; or
(b) a VH as set forth in SEQ ID NO: 3, and/or a VL as set forth in SEQ ID NO: 4.

In some embodiments, HA of each antibody-drug conjugate comprises:
(a) a VH as set forth in SEQ ID NO: 1 or a variant thereof, and a VL as set forth in SEQ ID NO: 2 or a variant thereof; or
(b) a VH as set forth in SEQ ID NO: 3 or a variant thereof, and a VL as set forth in SEQ ID NO: 4 or a variant thereof.

In some embodiments, HA of each antibody-drug conjugate comprises:
(a) a VH as set forth in SEQ ID NO: 1, and a VL as set forth in SEQ ID NO: 2; or
(b) a VH as set forth in SEQ ID NO: 3, and a VL as set forth in SEQ ID NO: 4.

In some embodiments, HA of each antibody-drug conjugate further comprises:
(a) a heavy chain constant region (CH) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids); and
(b) a light chain constant region (CL) of a human immunoglobulin or a variant thereof, wherein the variant has a substitution, deletion or addition of one or more amino acids as compared with the wild-type sequence from which it is derived (e.g., a substitution, deletion or addition of up to 20, up to 15, up to 10, or up to 5 amino acids; e.g., a substitution, deletion or addition of 1, 2, 3, 4 or 5 amino acids).

In some embodiments, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region.

In some embodiments, HA of each antibody-drug conjugate comprises a heavy chain constant region (CH) as set forth in SEQ ID NO: 35 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 35.

In some embodiments, HA of each antibody-drug conjugate comprises a heavy chain constant region (CH) as set forth in SEQ ID NO: 35.

In some embodiments, HA of each antibody-drug conjugate comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36.

In some embodiments, HA of each antibody-drug conjugate comprises a light chain constant region (CL) as set forth in SEQ ID NO: 36.

In some embodiments, HA of each antibody-drug conjugate comprises a heavy chain constant region (CH) as set forth in SEQ ID NO: 35 and a light chain constant region (CL) as set forth in SEQ ID NO: 36.

In some embodiments, HA of each antibody-drug conjugate comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

In some embodiments, HA of each antibody-drug conjugate comprises:
(1) a heavy chain comprising the sequence as set forth in SEQ ID NO: 37, and, a light chain comprising the sequence as set forth in SEQ ID NO: 38; or
(2) a heavy chain comprising the sequence as set forth in SEQ ID NO: 39, and, a light chain comprising the sequence as set forth in SEQ ID NO: 40.

In some embodiments, HA of each antibody-drug conjugate is selected from Trastuzumab or Pertuzumab.

In some embodiments, the query accession number (IMGT/mAb-DB ID) for the amino acid sequence of Trastuzumab in the IMGT database is: 97, the query accession number (IMGT/mAb-DB ID) for the amino acid sequence of Pertuzumab in the IMGT database is: 80.

In certain embodiments, HA of each antibody-drug conjugate disclosed herein may independently comprise a C-terminal lysine, lack a C-terminal lysine, lack a C-terminal glycine-lysine dipeptide, and/or comprise N-terminal glutamine or glutamic acid, or N-terminal amino acid may be cyclized to pyroglutamic acid or N-terminal amino acid may be cyclized to pyroglutamate.

In certain embodiments, HA of each antibody-drug conjugate may comprise post-translational modifications thereof (e.g., cleavage of the C-terminal lysine in the heavy chain, conversion of the N-terminal glutamine or glutamic acid in the heavy or light chain to pyroglutamic acid or pyroglutamate), which may occur during recombinant expression in a host cell (e.g., CHO cell) or arise during purification/storage.

In certain embodiments, the N-terminal glutamine of the VH having the sequence as set forth in SEQ ID NO: 1 or 3 or variant thereof or the heavy chain having the sequence as set forth in SEQ ID NO: 37 or 39 or variant thereof undergoes cyclization to form pyroglutamic acid or pyroglutamate.

In certain embodiments, the heavy chain constant region (CH) as set forth in SEQ ID NO: 35 or variant thereof or the heavy chain having the sequence as set forth in SEQ ID NO: 37 or 39 or variant thereof lacks C-terminal lysine.

In certain embodiments, the antibody in the antibody-drug conjugate is conjugated to 1 to 4 of the following structures:

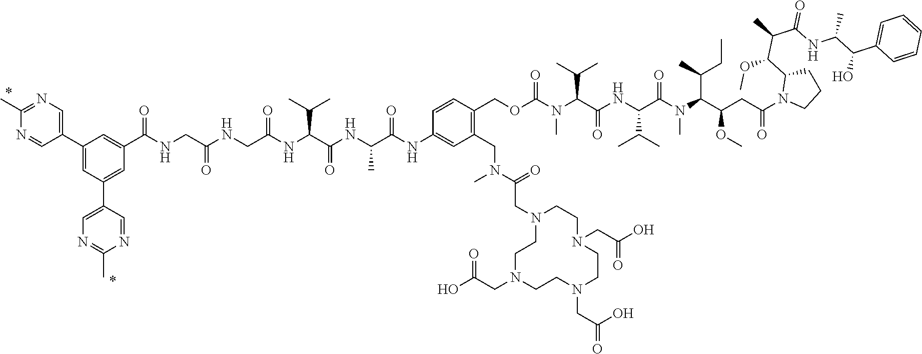 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CNC(=O)CNC(=O)c2cc(-c3cnc(C)nc3)cc(-c3cnc(C)nc3)c2)C(C)C)cc1CN(C)C(=O)CN1CCN(CC(=O)O)CCN(CC(=O)O)CCN(CC(=O)O)CC1)C(C)C]

where * represents the site linking to the sulfhydryl group of the cysteine of the antibody.

In certain embodiments, the antibody in the antibody-drug conjugate is conjugated to 1 to 4 of the following structures:

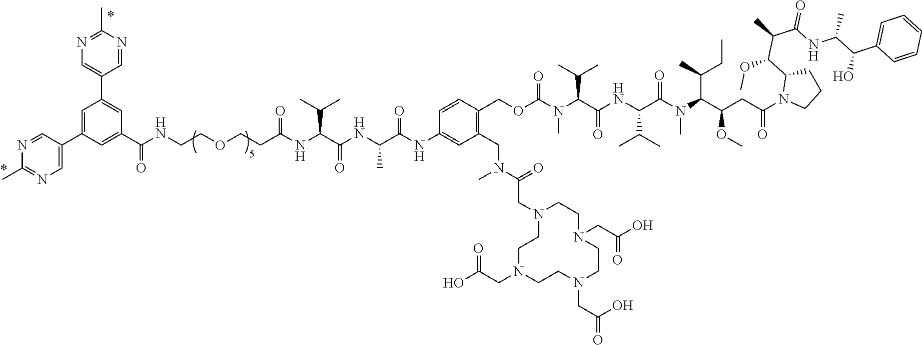

where * represents the site linking to the sulfhydryl group of the cysteine of the antibody.

In certain embodiments, the antibody in the antibody-drug conjugate is conjugated to 1 to 4 of the following structures:

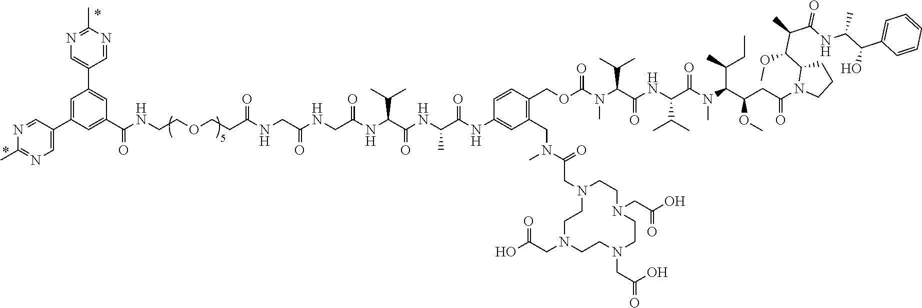

where * represents site linking to the sulfhydryl group of the cysteine of the antibody.

In some embodiments, x in the antibody-drug conjugate represented by Ab-[M-L-E-D], is 1 to 10, such as 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10 or 9 to 10.

In some embodiments, x in the antibody-drug conjugate represented by Ab-[M-L-E-D]ₓ is 1, 2, 3, 4, 5, 6, 7, 8, 9 or 10.

The antibody-drug conjugate of the present invention is optionally substituted by one or more suitable substituents.

The present application further provides an antibody-drug conjugate as represented by the formula Ab-[L′-D]ₓ, where:
Ab represents an antibody or antigen-binding fragment thereof;
D represents a cytotoxic drug fragment;
L′ represents a linker connecting Ab and D;
x is an integer selected from 1 to 10;

The antibody or antigen-binding fragment thereof is conjugated to a drug-linker selected from the followings to form the antibody-drug conjugate:

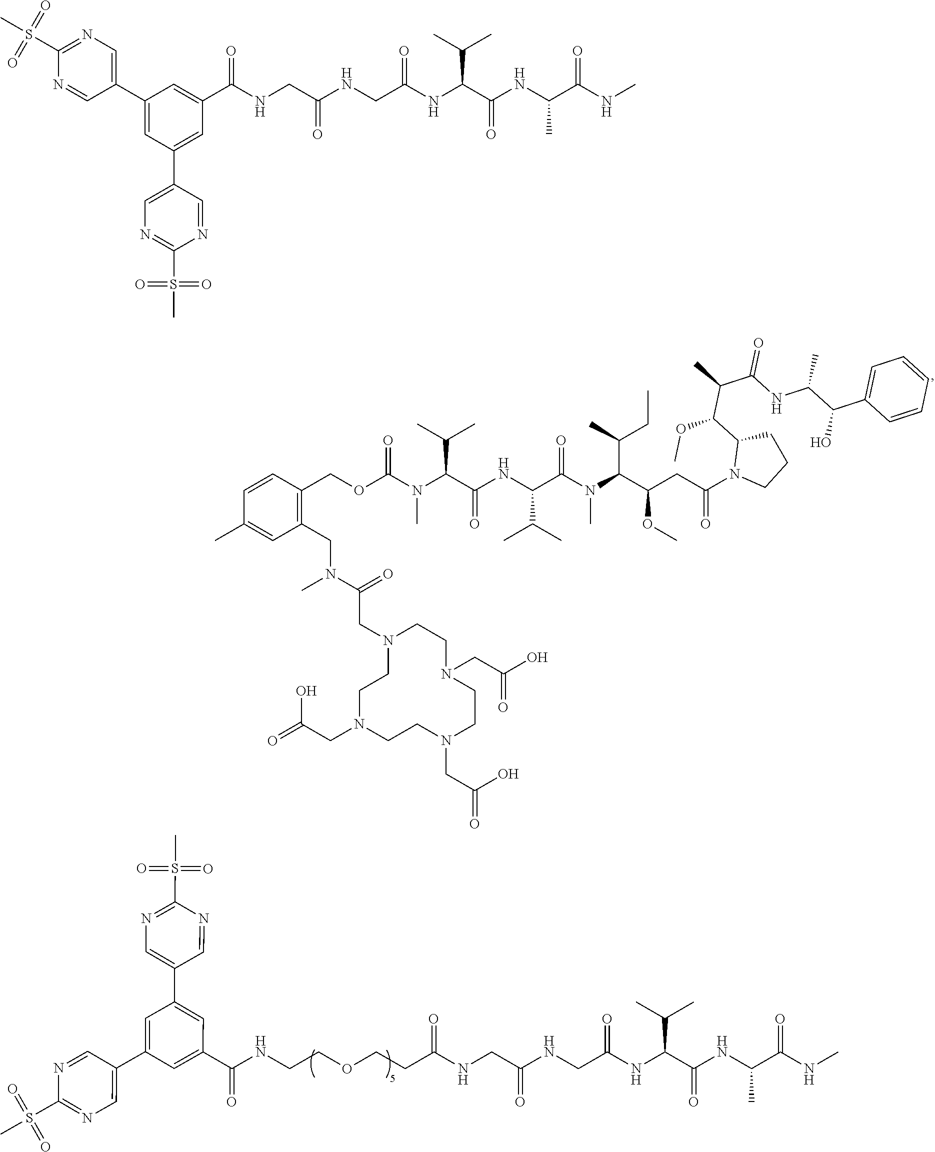

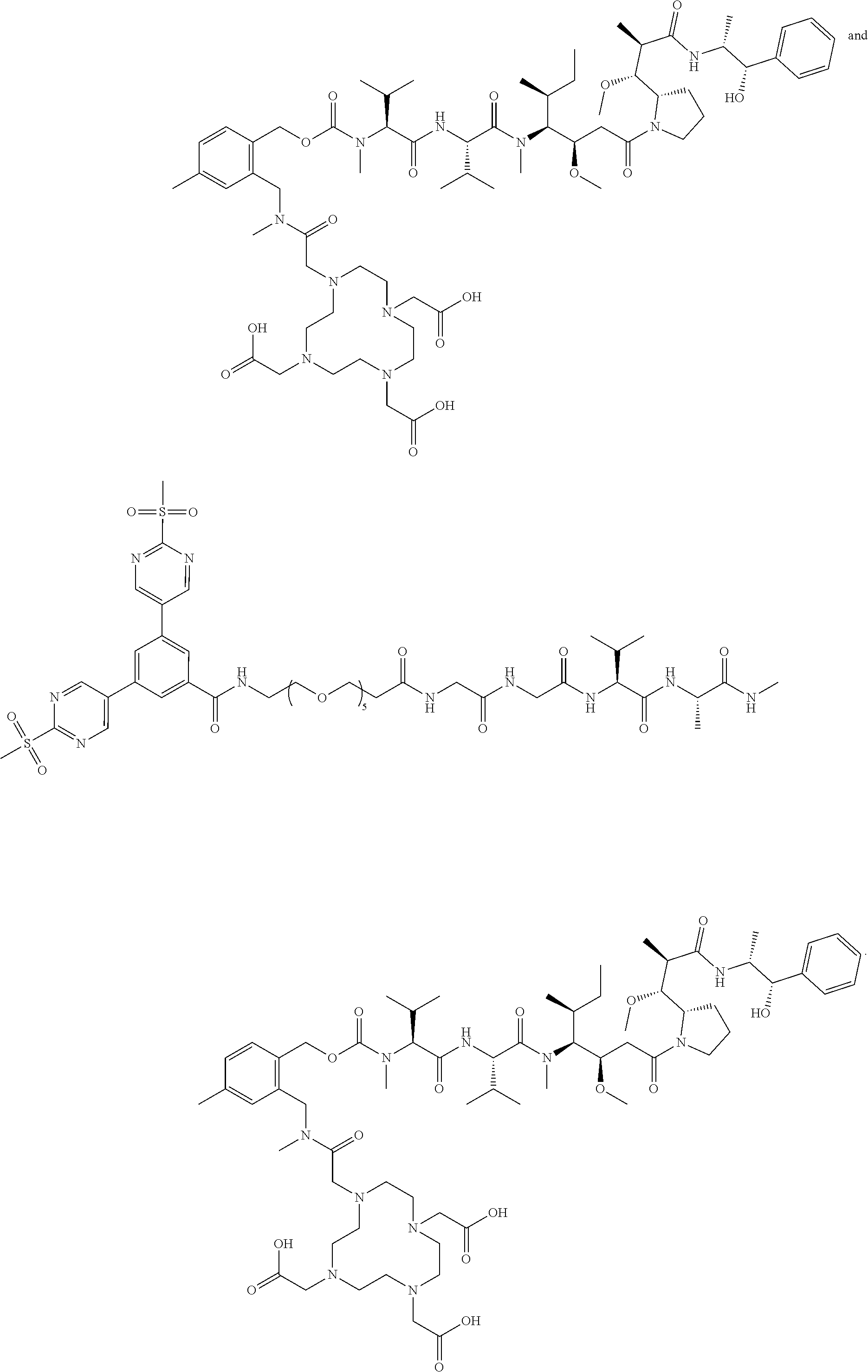

In certain embodiments, the antibody comprises a heavy chain amino acid sequence as set forth in SEQ ID NO: 37, and, a light chain amino acid sequence as set forth in SEQ ID NO: 38.

In certain embodiments, the antibody is Trastuzumab.

In certain embodiments, the antibody in the antibody-drug conjugate is conjugated to 1 to 4 (e.g., 1, 2, 3, or 4) of the following structures:

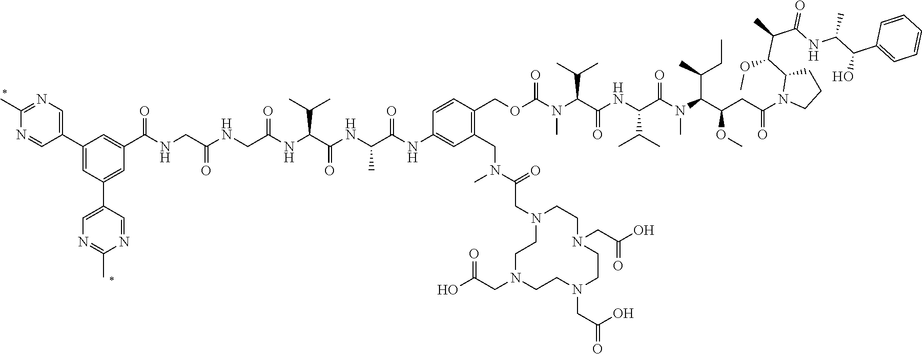 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CNC(=O)CNC(=O)c2cc(-c3cnc(C)nc3)cc(-c3cnc(C)nc3)c2)C(C)C)cc1CN(C)C(=O)CN1CCN(CC(=O)O)CCN(CC(=O)O)CCN(CC(=O)O)CC1)C(C)C]

where * represents the site linking to the sulfhydryl group of the cysteine of the conjugate antibody.

In certain embodiments, the antibody in the antibody-drug conjugate is conjugated to 1 to 4 (e.g., 1, 2, 3, or 4) of the following structures:

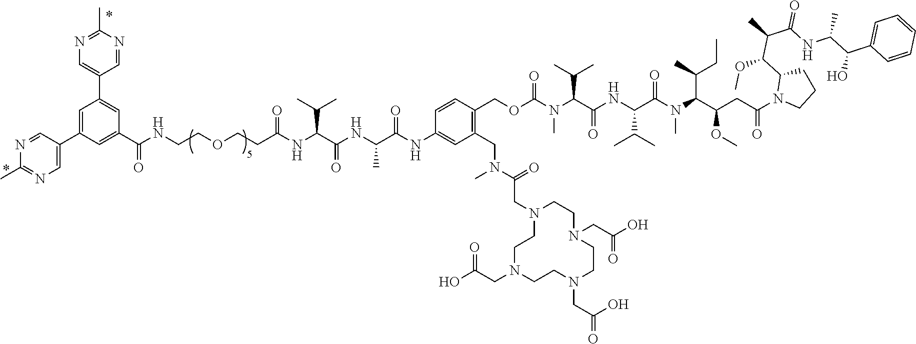

where * represents the site linking to the sulfhydryl group of the cysteine of the conjugate antibody.

In certain embodiments, the antibody in the antibody-drug conjugate is conjugated to 1 to 4 (e.g., 1, 2, 3, or 4) of the following structures:

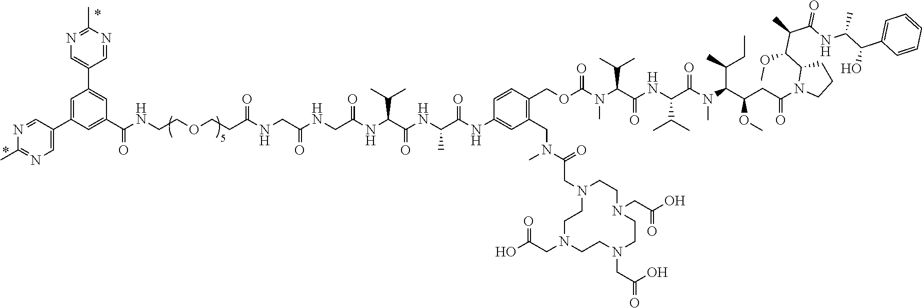

where * represents the site linking to the sulfhydryl group of the cysteine of the conjugate antibody.

### Composition

In another aspect, the present application provides a composition of the antibody-drug conjugate (ADC) as described herein. Such a composition may comprise a plurality of ADCs as described herein, where each ADC comprises a drug-linker as described herein, where x is independently 1, 2, 3, 4, 5, 6, 7, 8, 9 or 10. In other words, each antibody molecule in the composition can be conjugated to 1, 2, 3, 4, 5, 6, 7, 8, 9 or 10 drug-linkers. Therefore, the composition is characterized by a “drug-to-antibody” ratio (DAR) in the range of about 1 to about 10. Methods for determining DAR are well known to technicians in the art, including methods using reverse phase chromatography or HPLC-MS.

For example, in any embodiment, the ADC composition as described herein has a DAR of about 1 to about 10, or any subrange therebetween, e.g., about 1, about 2, about 3, about 4, about 5, about 6, about 7, about 8, about 9, about 10, about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, about 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, the DAR of the ADC compositions described herein is about 3 to 9, for example, about 3.0 to 3.5, about 3.0 to 4.0, about 3.0 to 4.5, about 3.0 to 5.0, about 3.0 to 6.0, about 3.5 to 4.0, about 3.5 to 4.5, about 3.5 to 5.0, about 3.5 to 5.5, about 3.5 to 6.0, about 3.5 to 6.5, about 4.0 to 4.5, about 4.0 to 5.0, about 4.0 to 5.5, about 4.0 to 6.0, about 4.0 to 6.5, about 4.0 to 7.0, about 4.0 to 8.0, about 4.5 to 5.0, about 4.5 to 5.5, about 4.5 to 6.0, about 4.5 to 6.5, about 4.5 to 7.0, about 4.5 to 7.5, about 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 7.5, about 6.5 to 8.5, about 7.0 to 7.5.

In some embodiments, the DAR of the ADC compositions described herein is about 1.0 to 3.0, or about 3.0 to 5.0; for example, about 1.0, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, about 1.2, about 1.21, about 1.22, about 1.23, about 1.24, about 1.25, about 1.26, about 1.27, about 1.28, about 1.29, about 1.3, about 1.31, about 1.32, about 1.33, about 1.34, about 1.35, about 1.36, about 1.37, about 1.38, about 1.39, about 1.4, about 1.41, about 1.42, about 1.43. 43, about 1.44, about 1.45, about 1.46, about 1.47, about 1.48, about 1.49, about 1.5, about 1.51, about 1.52, about 1.53, about 1.54, about 1.55, about 1.56, about 1.57, about 1.58, about 1.59, about 1.6, about 1.61, about 1.62, about 1.63, about 1.64, about 1.65, about 1.66, about 1.67, about 1.68, about 1.69, about 1.7, about 1.71, about 1.72, about 1.73, about 1.74, about 1.75, about 1.76, about 1.77, about 1.78, about 1.79, about 1.8, about 1.81, about 1.82, about 1.83, about 1.84, about 1.85, about 1.86, about 1.87, about 1.88, about 1.89, about 1.9, about 1.91, about 1.92, about 1.93, about 1.94, about 1.96 94, about 1.95, about 1.96, about 1.97, about 1.98, about 1.99, about 2.0, about 2.01, about 2.02, about 2.03, about 2.04, about 2.05, about 2.06, about 2.07, about 2.08, about 2.09, about 2.1, about 2.11, about 2.12, about 2.13, about 2.14, about 2.15, about 2.16, about 2.17, about 2.18, about 2.19, about 2.2, about 2.21, about 2.22, about 2.23, about 2.24, about 2.25, about 2.26, about 2.27, about 2.28, about 2.29, about 2.3, about 2.31, about 2.32, about 2.33, about 2.34, about 2.35, about 2.36, about 2.37, about 2.38, about 2.39, about 2.4, about 2.41, about 2.42, about 2.43, about 2.44, about 2.45, about 2.46, about 2.47, about 2.48, about 2.49, about 2.5, about 2.51, about 2.52, about 2.53, about 2.54, about 2.55, about 2.56, about 2.57, about 2.58, about 2.59, about 2.6, about 2.61, about 2.62, about 2.63, about 2.64, about 2.65, about 2.66, about 2.67, about 2.68, about 2.69, about 2.7, about 2.71, about 2.72, about 2.73, about 2.74, about 2.75, about 2.76, about 2.77, about 2.78, about 2.79, about 2.8, about 2.81, about 2.82, about 2.83, about 2.84, about 2.85, about 2.86, about 2.87, about 2.88, about 2.89, about 2.9, about 2.91, about 2.92, about 2.93, about 2.94, about 2.95, about 2.96, about 2.97, about 2.98, about 2.99, about 3.0, about 3.01, about 3.02, about 3.03, about 3.04, about 3.05, about 3.06, about 3.07, about 3.08, about 3.09, about 3.1, about 3.11, about 3.12, about 3.13, about 3.14, about 3.15, about 3.16, about 3.17, about 3.18, about 3.19, about 3.2, about 3.21, about 3.22, about 3.23, about 3.24, about 3.25, about 3.26, about 3.27, about 3.28, about 3.29, about 3.3, about 3.31, about 3.32, about 3.33, about 3.34, about 3.35, about 3.36, about 3.37, about 3.38, about 3.39, about 3.4, about 3.41, about 3.42, about 3.43, about 3.44, about 3.45, about 3.46, about 3.47, about 3.48, about 3.49, about 3.5, about 3.51, about 3.52, about 3.53, about 3.54, about 3.55, about 3.56, about 3.57, about 3.58, about 3.59, about 3.6, about 3.61, about 3.62, about 3.63, about 3.64, about 3.65, about 3.66, about 3.67, about 3.68, about 3.69, about 3.7, about 3.71, about 3.72, about 3.73, about 3.74, about 3.75, about 3.76, about 3.77, about 3.78, about 3.79, about 3.8, about 3.81, about 3.82, about 3.83, about 3.84, about 3.85, about 3.86, about 3.87, about 3.88, about 3.89, about 3.9, about 3.91, about 3.92, about 3.93, about 3.94, about 3.95, about 3.96, about 3.97, about 3.98 8, about 3.99, about 4.0, about 4.01, about 4.02, about 4.03, about 4.04, about 4.05, about 4.06, about 4.07, about 4.08, about 4.09, about 4.1, about 4.11, about 4.12, about 4.13, about 4.14, about 4.15, about 4.16, about 4.17, about 4.18, about 4.19, about 4.2, about 4.21, about 4.22, about 4.23, about 4.24, about 4.25, about 4.26, about 4.27, about 4.28, about 4.29, about 4.3, about 4.31, about 4.32, about 4.33, about 4.34, about 4.35, about 4.36, about 4.37, about 4.38, about 4.39, about 4.4, about 4.41, about 4.42, about 4.43, about 4.44, about 4.45, about 4.46, about 4.47, about 4.48, about 4.49, about 4.5, about 4.51, about 4.52, about 4.53, about 4.54, about 4.55, about 4.56, about 4.57, about 4.58, about 4.59, about 4.6, about 4.61, about 4.62, about 4.63, about 4.64, about 4.65, about 4.66, about 4.67, about 4.68, about 4.69, about 4.7, about 4.71, about 4.72, about 4.73, about 4.74, about 4.75, about 4.76, about 4.77, about 4.78, about 4.79, about 4.8, about 4.81, about 4.82, about 4.83, about 4.84, about 4.85, about 4.86, about 4.87, about 4.88, about 4.89, about 4.9, about 4.91, about 4.92, about 4.93, about 4.94, about 4.95, about 4.96, about 4.97, about 4.98, about 4.99, about 5.0.

In certain embodiments, the DAR of the ADC compositions described herein is about 2.28, about 2.35, about 4.12, about 3.6, or about 4.08.

In certain embodiments, the DAR of the ADC compositions described herein is 2.28, 2.35, 4.12, 3.6, or 4.08.

In certain embodiments, the composition is composed of the antibody-drug conjugate represented by Ab-[L′-D]ₓ. In certain embodiments, the DAR of the composition is 2 to 6, such as 3 to 5, such as about 4. In certain embodiments, the DAR of the composition is 4.12. In certain embodiments, the DAR of the composition is 3.6. In certain embodiments, the DAR of the composition is 4.08. In certain embodiments, the DAR of the composition is 4.12, and the antibody-drug conjugates with x of 4 account for 80% to 90% (e.g., 85% to 90%, for another example, 88.43%). In certain embodiments, the DAR of the composition is 3.6, and the antibody-drug conjugates with x of 4 account for 70% to 80% (e.g., 70% to 75%, for another example, 72.01%). In certain embodiments, the DAR of the composition is 4.08, and the antibody-drug conjugates with x of 4 account for 85% to 95% (e.g., 90% to 95%, for another example, 92.32%).

### Pharmaceutical Composition

In another aspect, the present application provides a pharmaceutical composition, which comprises the antibody-drug conjugate described in any of the above aspects or the compound described in any of the above aspects, and one or more pharmaceutical excipients.

The antibody-drug conjugate, compound or drug-linker as described herein is typically formulated together with a pharmaceutically acceptable parenteral vehicle for parenteral use into a unit injectable form such as bolus injection, intravenous injection, intratumoral injection, etc. Optionally, the antibody-drug conjugate in the form of a lyophilized agent or solution with the desired purity is mixed with a pharmaceutically acceptable diluent, carrier, excipient or stabilizer (Remington's Pharmaceutical Sciences (1980) 16th edition, Osol, A. Ed.). The antibody-drug conjugate described herein or the pharmaceutical composition containing the antibody-drug conjugate can be administered via any route suitable for the individual to be treated.

### Use

The antibody-drug conjugate, composition or pharmaceutical composition as described herein can be used for the treatment of a variety of diseases or conditions, such as Her2-expressing cancer, including solid tumors or hematological malignancies, such as urothelial carcinoma, gastric cancer, breast cancer, lung cancer (e.g., non-small cell lung cancer, specifically lung adenocarcinoma), or lymphoma.

Therefore, the present application provides a use of the antibody-drug conjugate, composition or pharmaceutical composition as described in any of the above aspects in the manufacture of a medicament for treating Her2-expressing cancer.

At the same time, the present application provides the antibody-drug conjugate, composition or pharmaceutical composition as described in any of the above aspects, for use in treating Her2-expressing cancer.

At the same time, the present application also provides a method for treating Her2-expressing cancer, which comprises a step of administering to a subject in need thereof an effective amount of the antibody-drug conjugate, composition or pharmaceutical composition described in any of the above aspects.

In some embodiments, the antibody-drug conjugate, composition or pharmaceutical composition is sufficient (e.g., in a subject) to:
(1) inhibit the proliferation of cells (e.g., tumor cells);
(2) inhibit tumor growth;
(3) induce and/or increase antibody-dependent cellular cytotoxicity activity;
(4) inhibit HER2-mediated signal transduction;
(5) prevent and/or treat HER2-mediated diseases/disorders; or
(6) any combination of the above (1) to (5).

In some embodiments, the cancer disease is selected from the group consisting of solid tumors or hematological malignancies; for example, selected from the group consisting of gastric cancer, breast cancer, lung cancer (e.g., non-small cell lung cancer, specifically lung adenocarcinoma) and urothelial carcinoma.

### Intermediates

In some embodiments, the present invention provides the following synthetic intermediate compounds or salts, stereoisomers, tautomers or isotopically labeled compounds thereof:

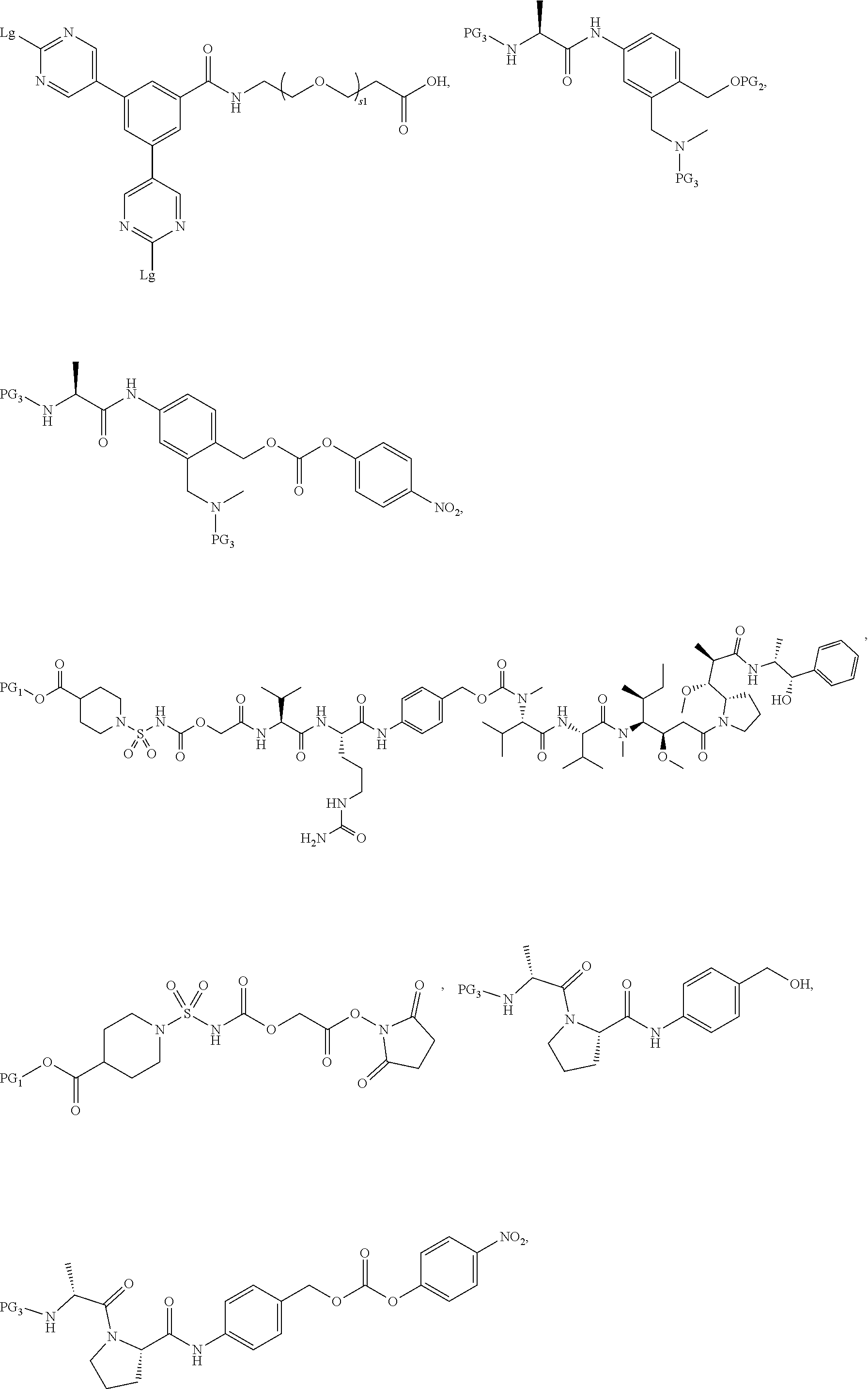

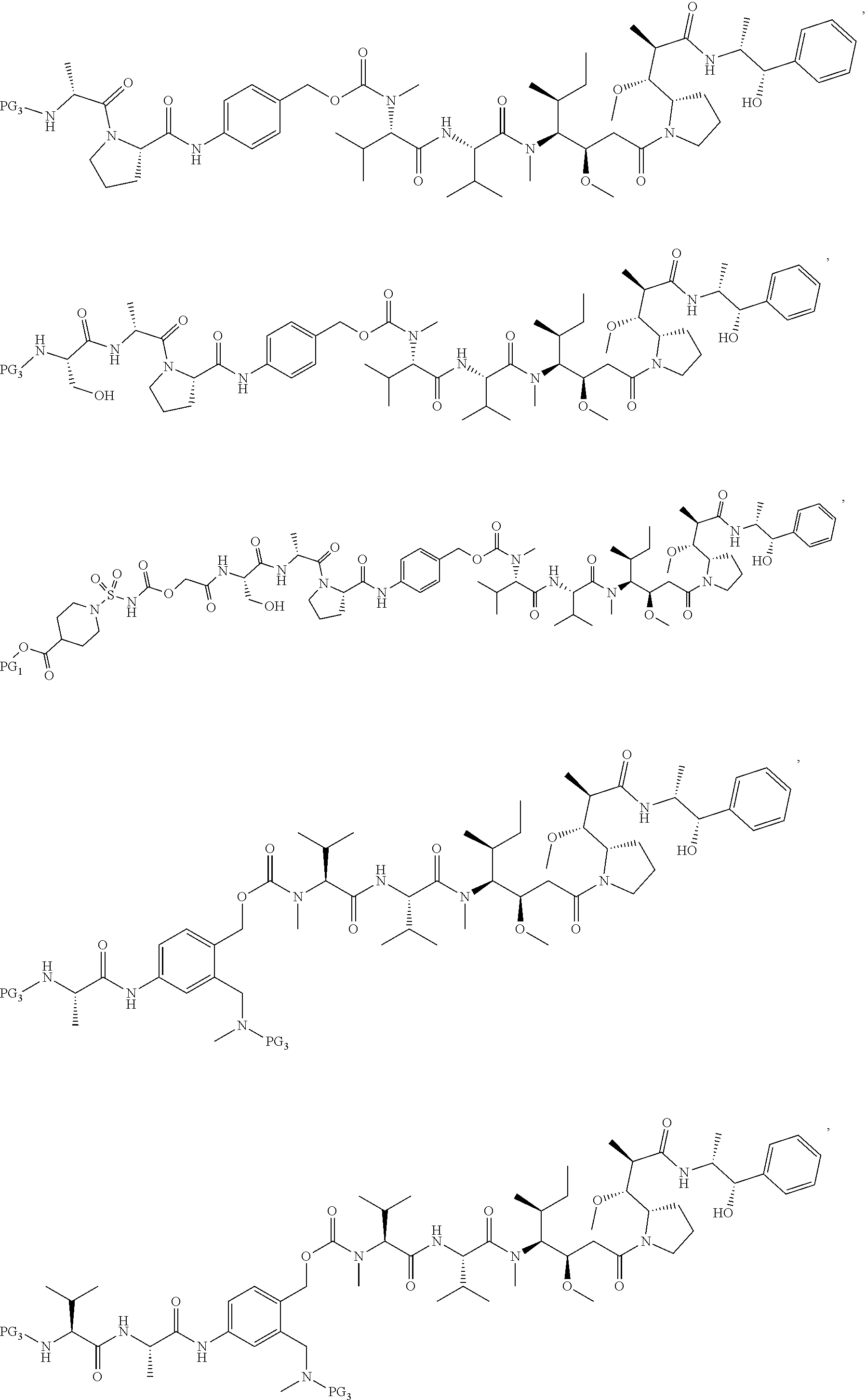

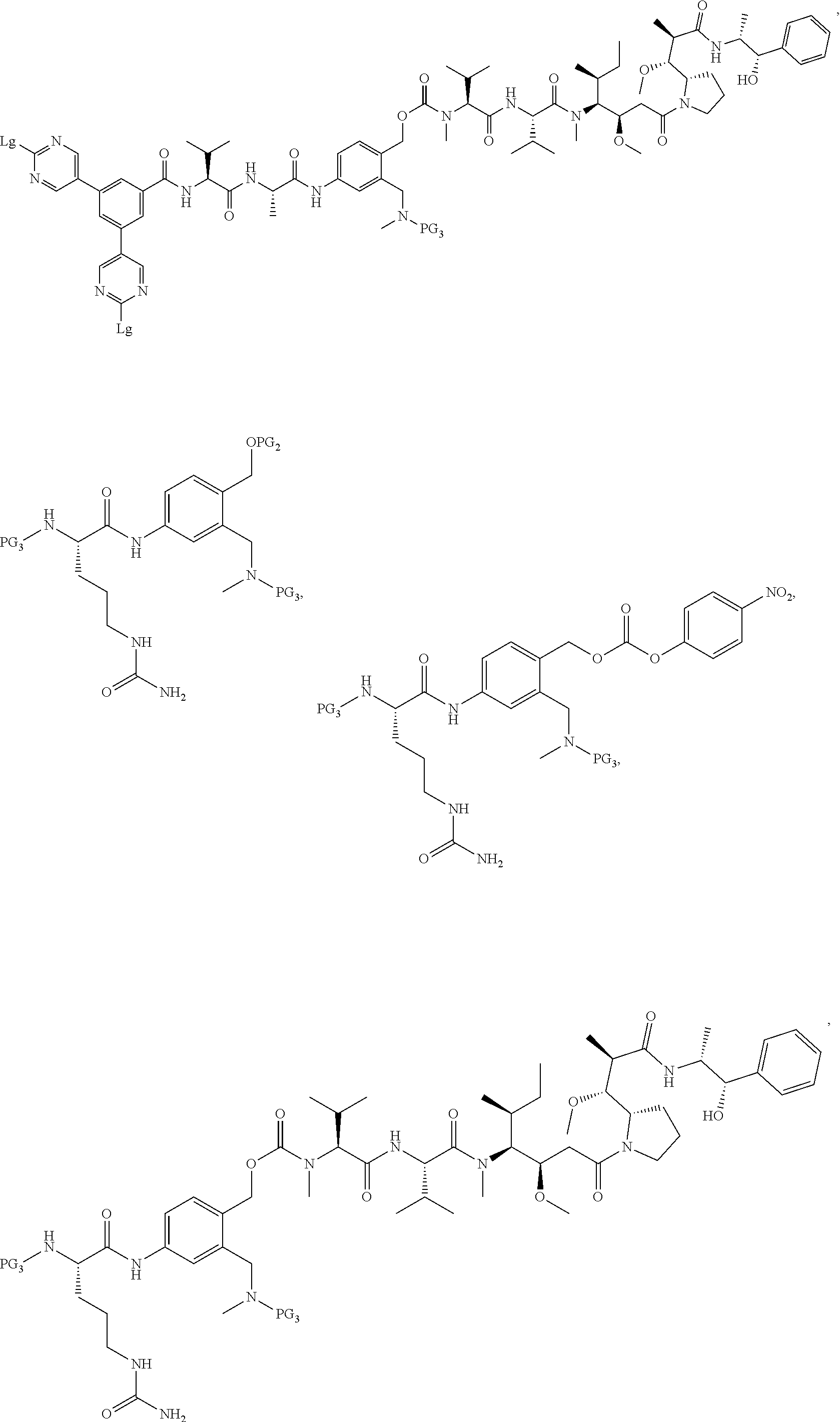

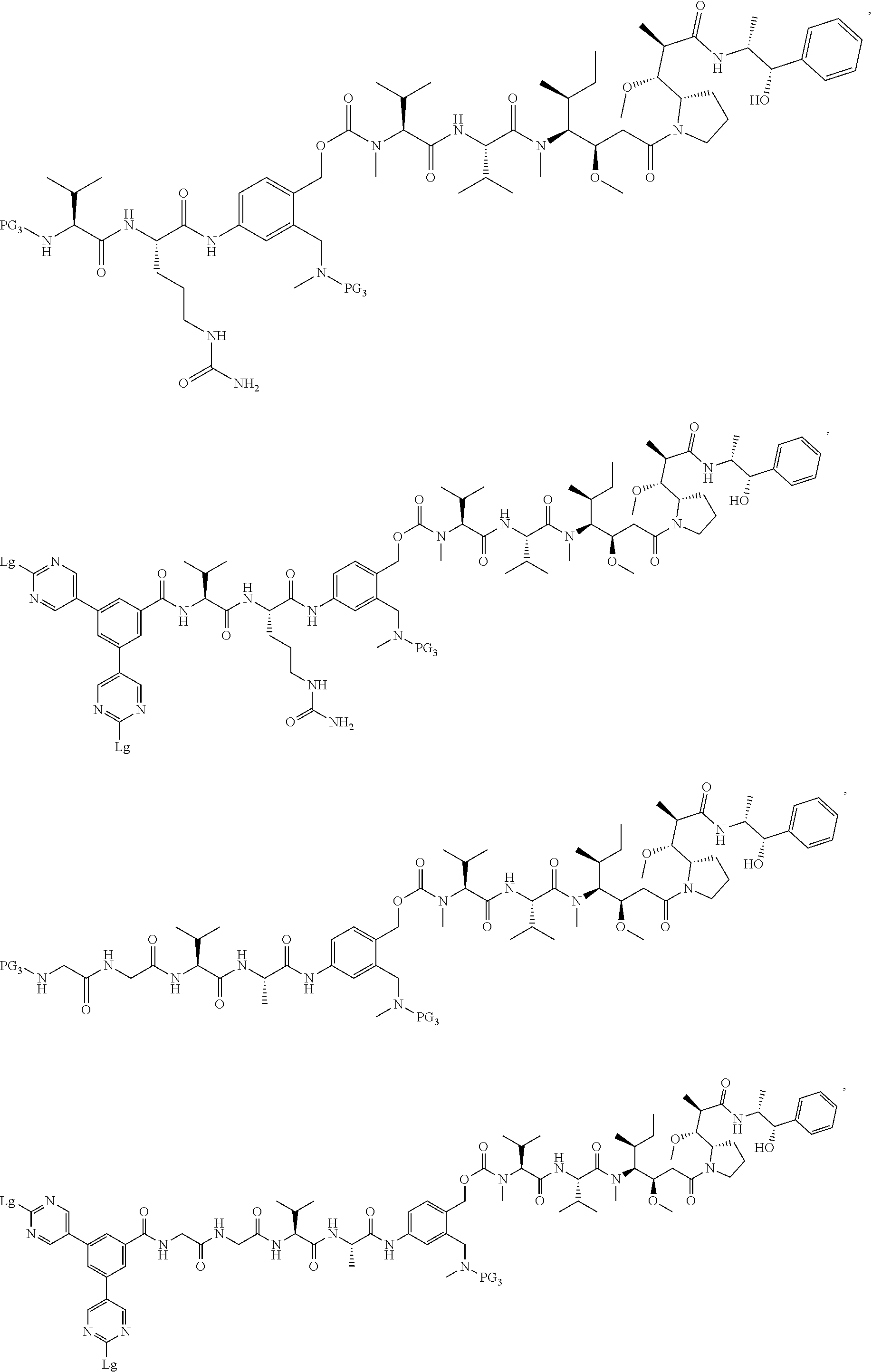

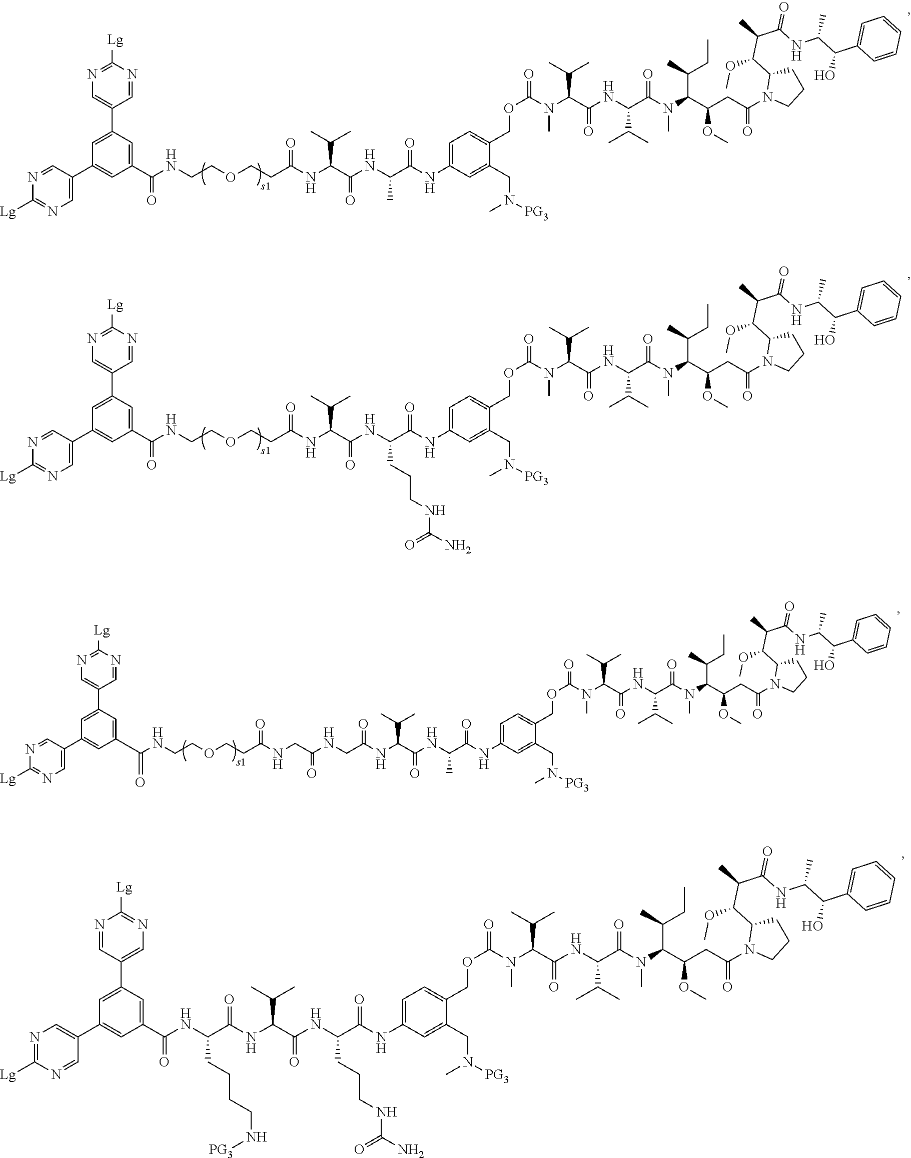

where:
PG₁ is each independently H or a carboxyl protecting group, for example, the carboxyl protecting group is C₁₋₆ alkyl, allyl, benzyl, 2,4-dimethoxybenzyl, p-methoxybenzyl, methoxyethoxymethyl, pentafluorophenyl, or 4-p-methylbenzyloxybenzyl;
PG₂ is each independently H or a hydroxyl protecting group, for example, the hydroxyl protecting group is trimethylsilyl (TMS), triethylsilyl (TES), triisopropylsilyl (TIPS), tert-butyldimethylsilyl (TBS), tert-butyldiphenylsilyl (TBDPS), methyl, tert-butyl, allyl, benzyl, methoxymethyl (MOM), ethoxyethyl, 2-tetrahydropyranyl (THP), formyl, acetyl, benzoyl, or p-nitrobenzoyl;
PG₃ is each independently H or an amino protecting group, for example, the amino protecting group is an alkoxycarbonyl-based amino protecting group, such as benzyloxycarbonyl (Cbz), tert-butyloxycarbonyl (Boc), fluorenylmethyloxycarbonyl (Fmoc), allyloxycarbonyl (Alloc), trimethylsilylethoxycarbonyl (Teoc), methyl (or ethyl)oxycarbonyl; an acyl-based amino protecting group, such as phthalyl (Pht), p-toluenesulfonyl (Tos), trifluoroacetyl (Tfa), 0-(p-) nitrobenzenesulfonyl (Ns), pivaloyl, benzoyl, tert-butyloxycarbonyl, 9-fluorenylmethoxycarbonyl, allyloxycarbonyl, trichloroethoxycarbonyl, trimethylsilylethoxycarbonyl, benzyloxycarbonyl, p-toluenesulfonyl, p-nitrobenzenesulfonyl, trifluoroacetyl, methoxycarbonyl, or ethoxycarbonyl; an alkyl-based amino protecting group, such as trityl (Trt), 2,4-dimethoxybenzyl (Dmb), 4-methoxybenzyl (PMB), benzyl (Bn);
Lg is a leaving group, as defined above;
sl is an integer selected from 1 to 20, preferably an integer of 1 to 15, such as an integer of 1 to 12, 3 to 12, such as 1, 2, 3, 4, 5, 6, 7, 8, 9, 10, 11, 12, 13, 14 or 15, preferably 5, 8, 10.

In some embodiments, the present invention provides the following synthetic intermediate compounds or salts, stereoisomers, tautomers or isotopically labeled compounds thereof:

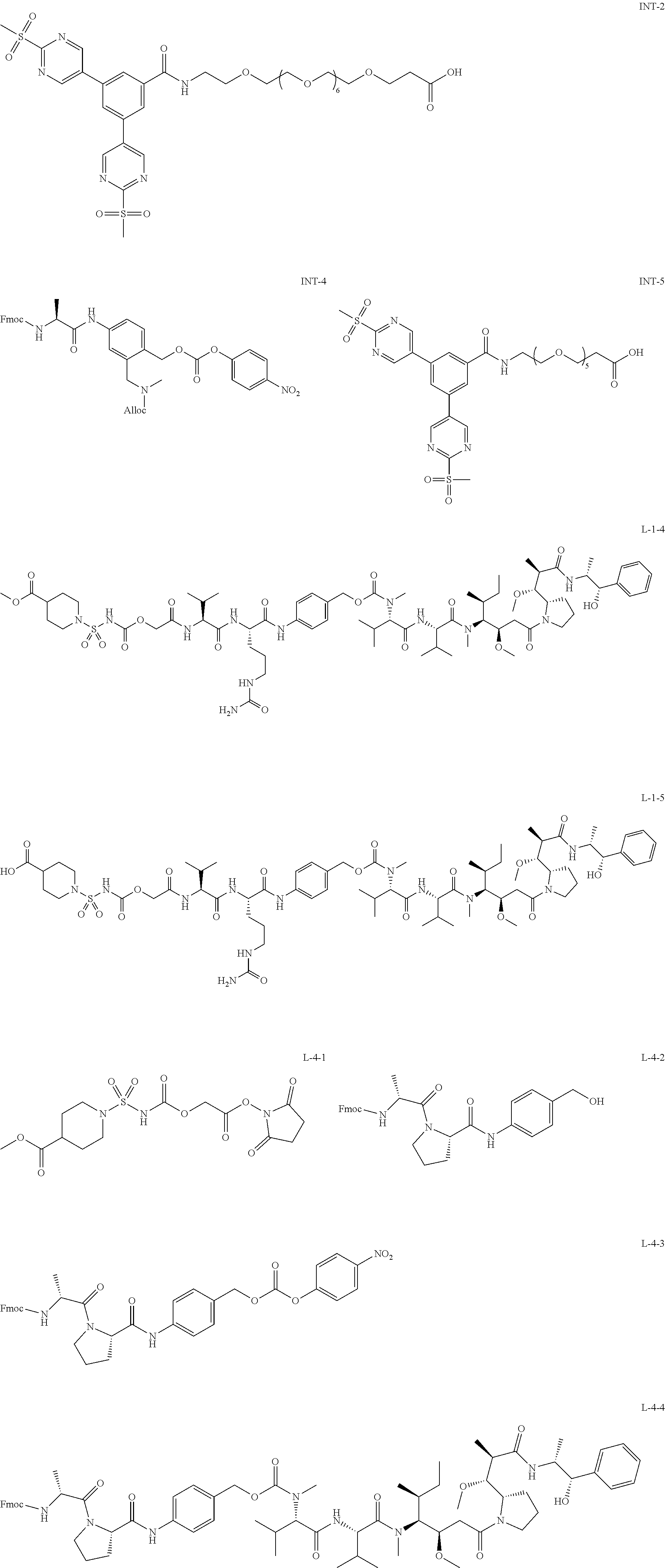

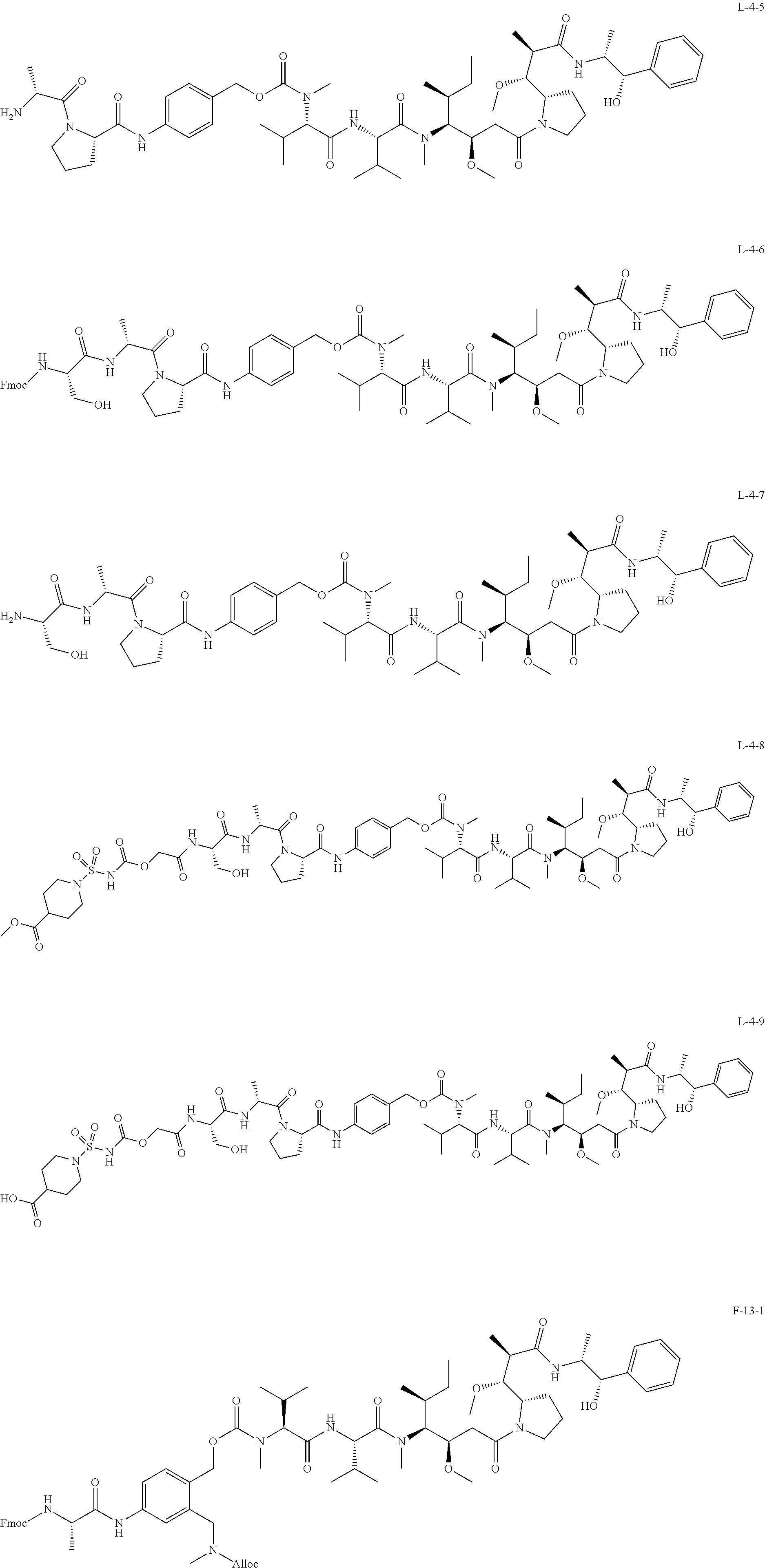

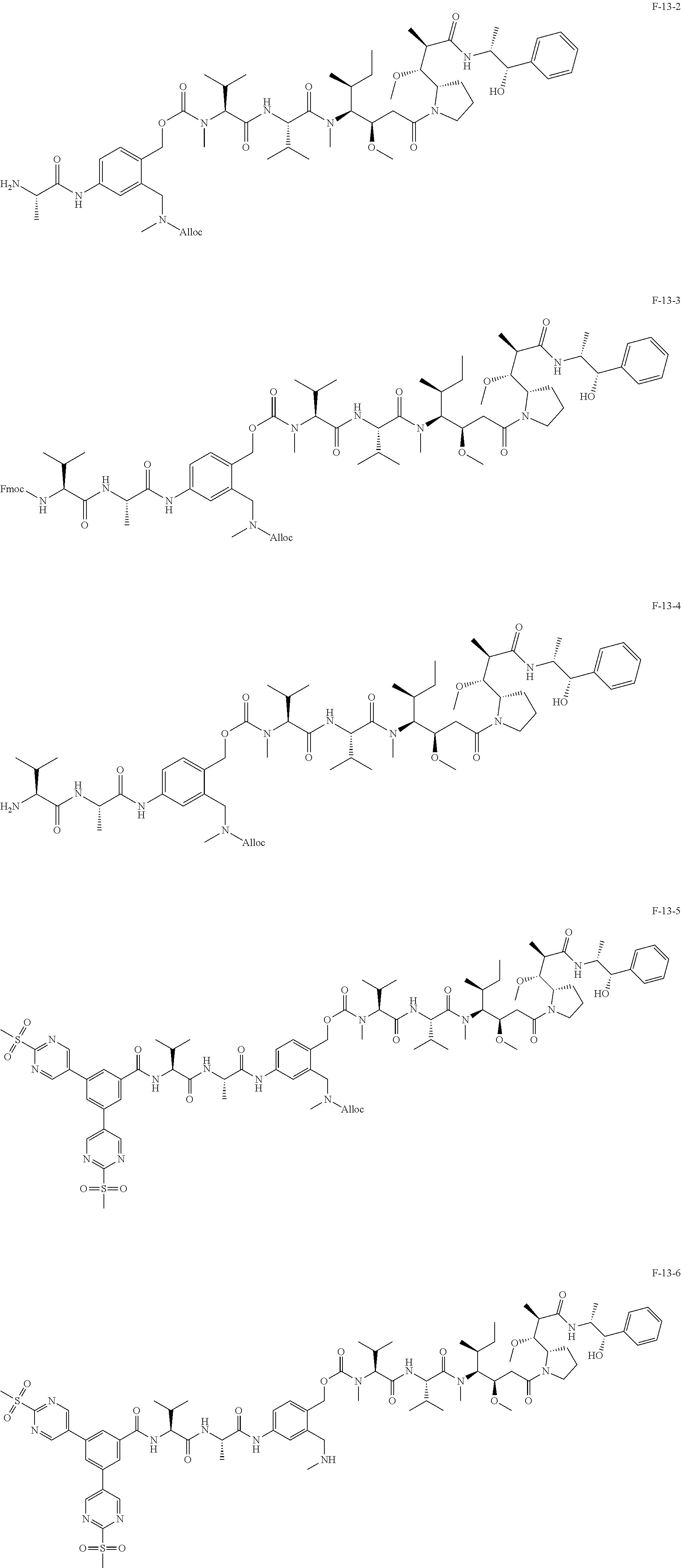

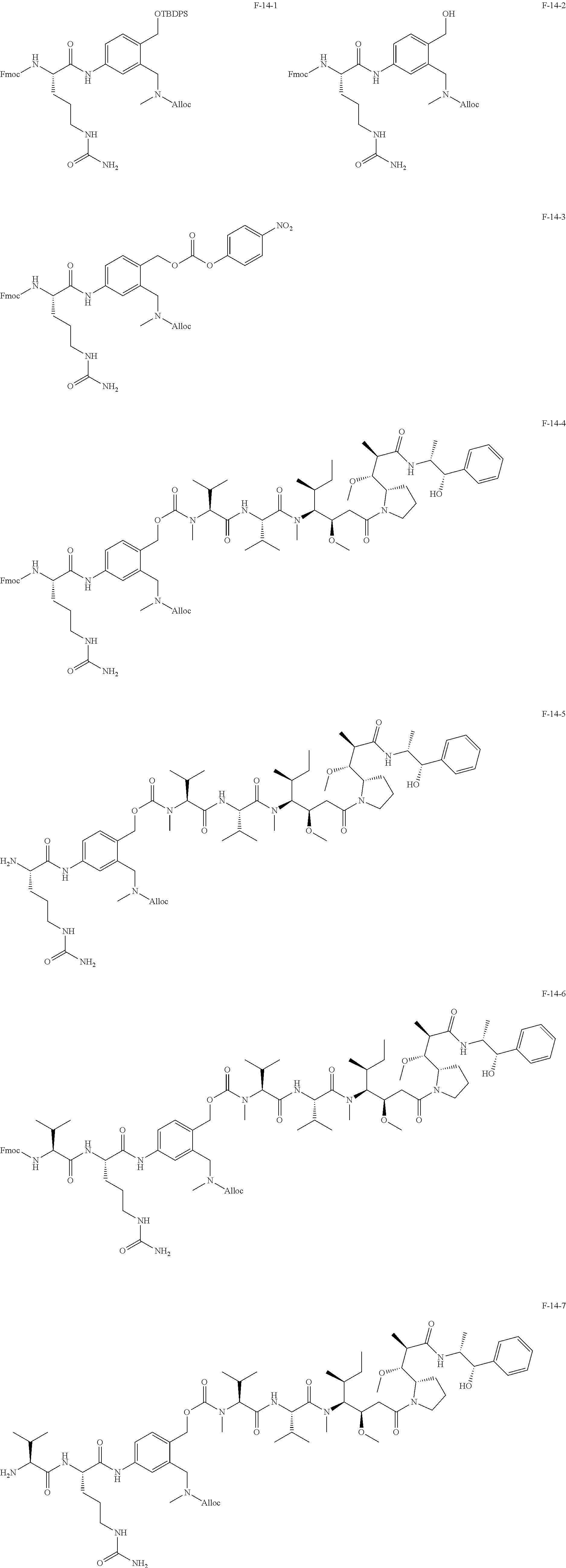

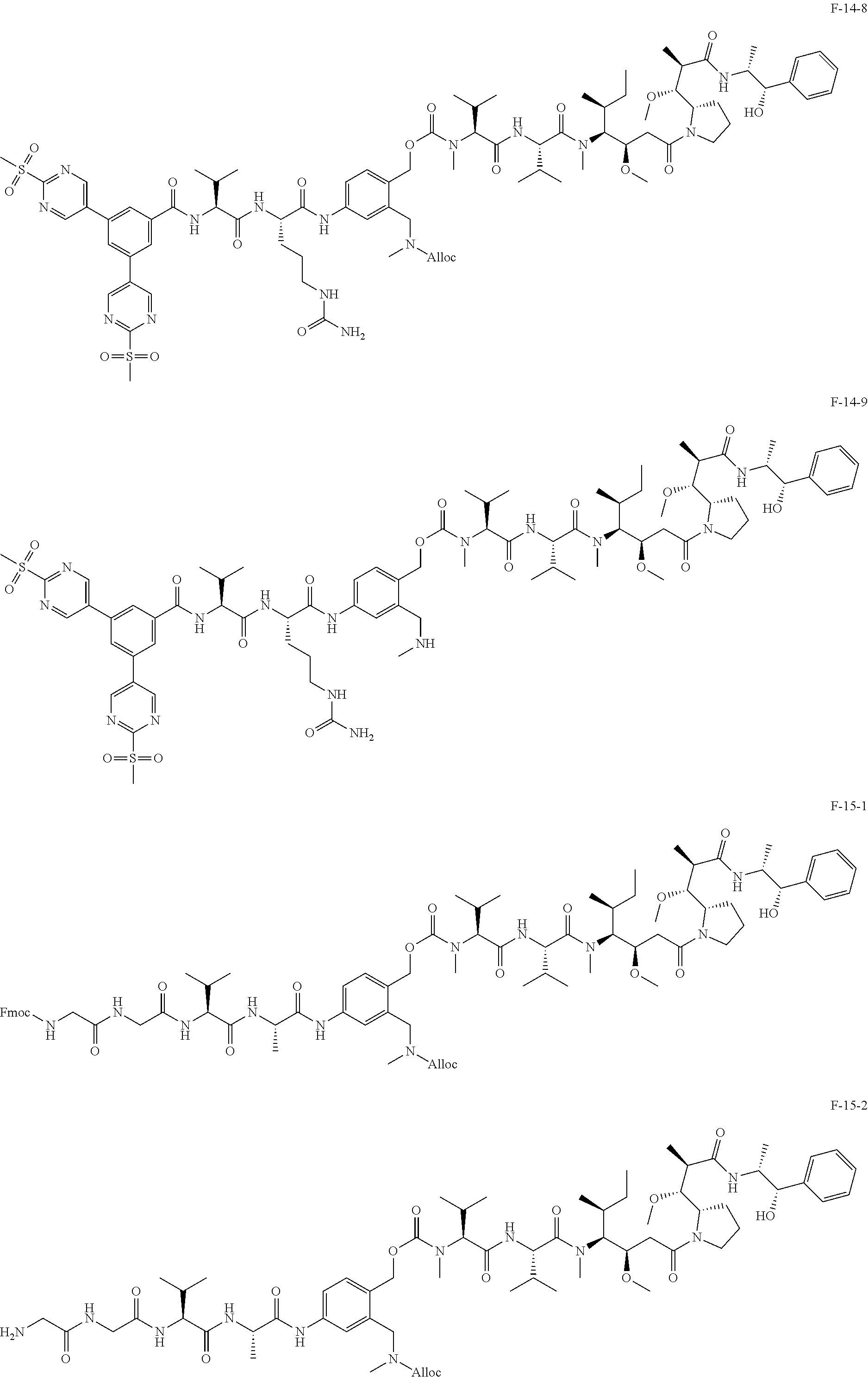

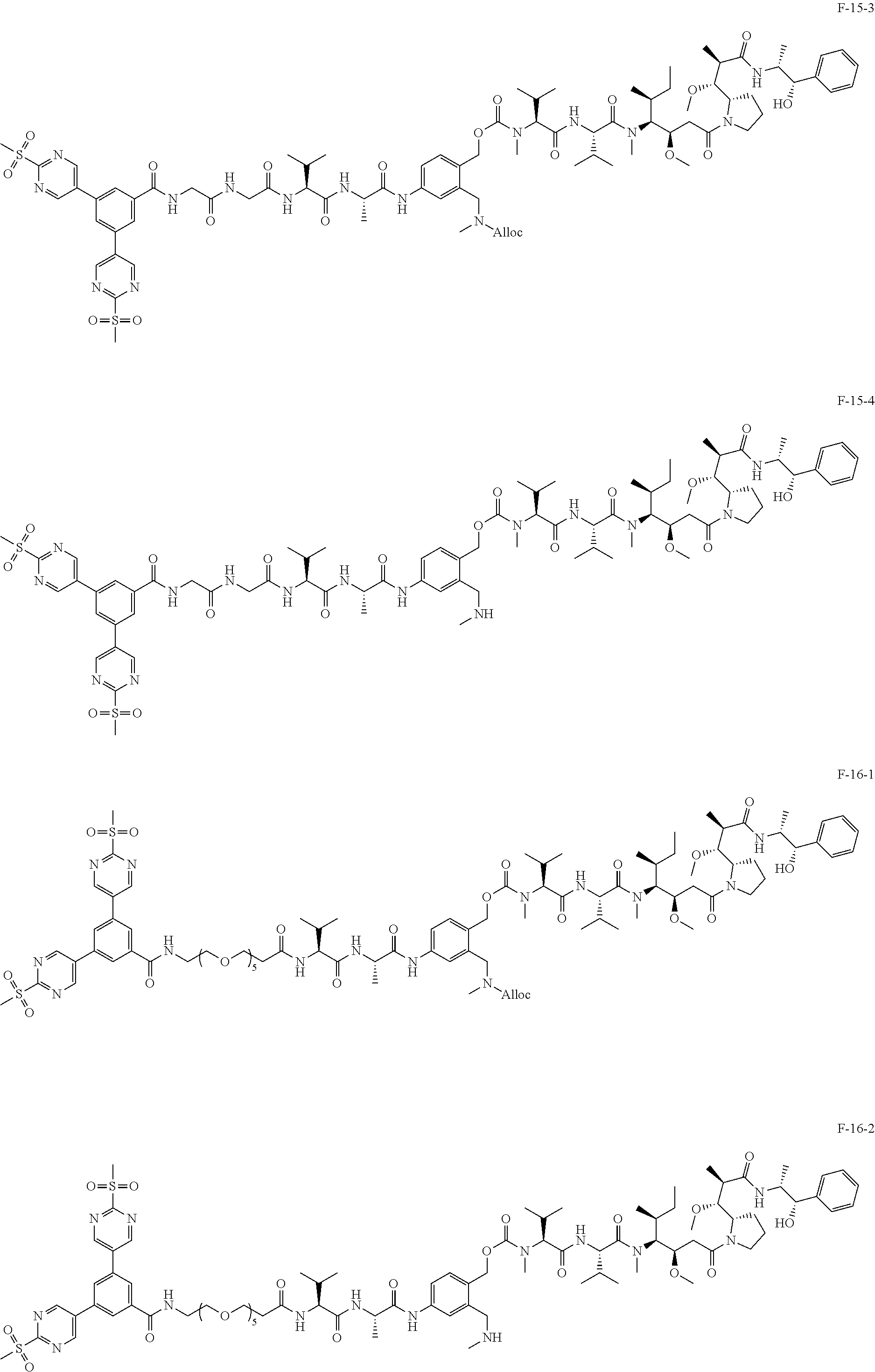

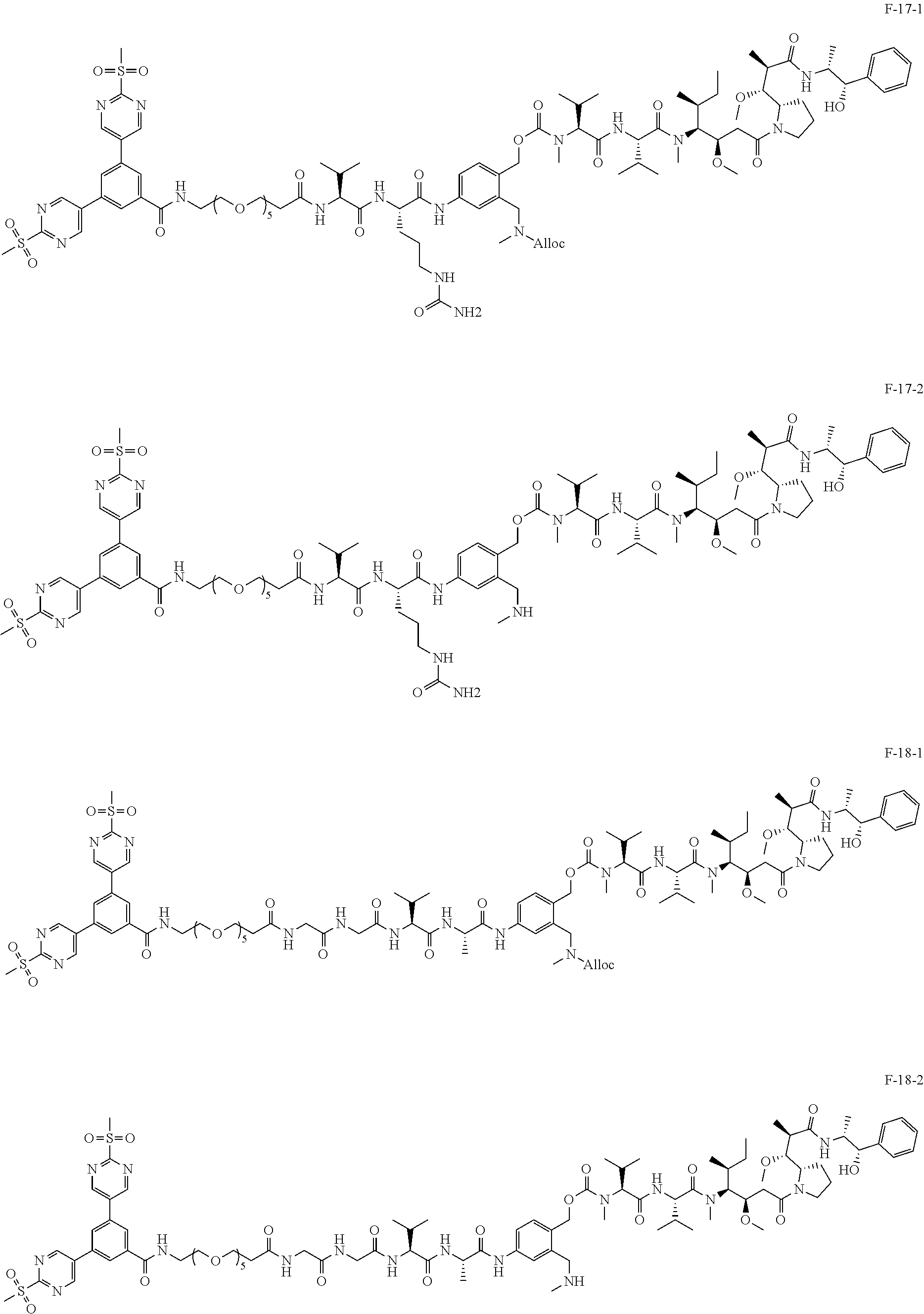

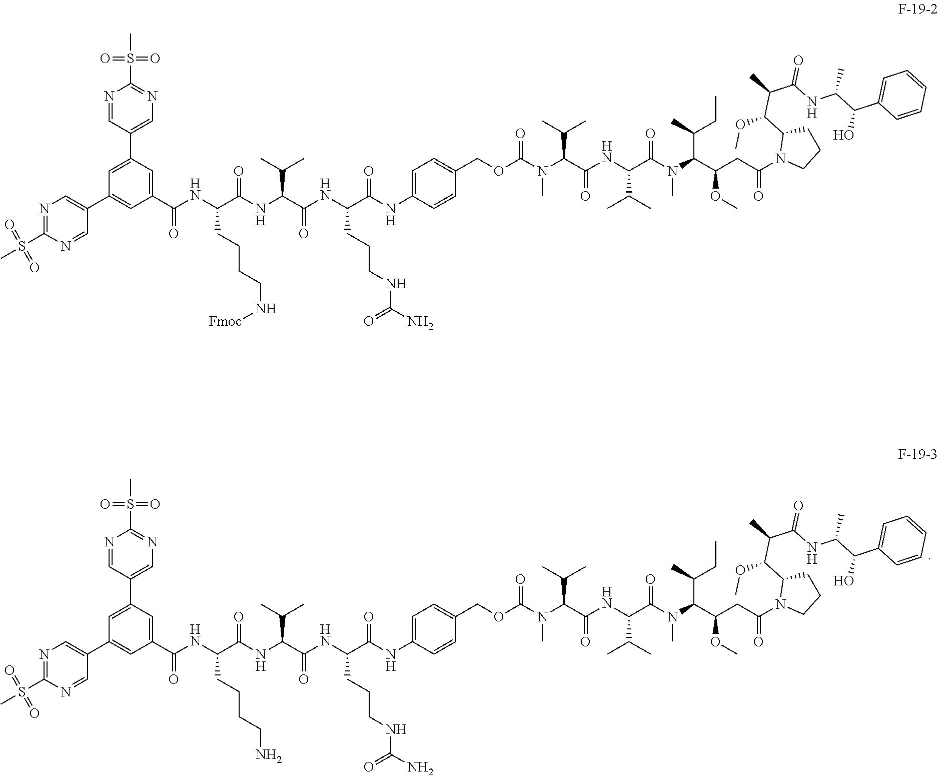

### Synthesis Methods

In some embodiments, the present application provides Method 1 for synthesizing the following synthetic intermediate compound:

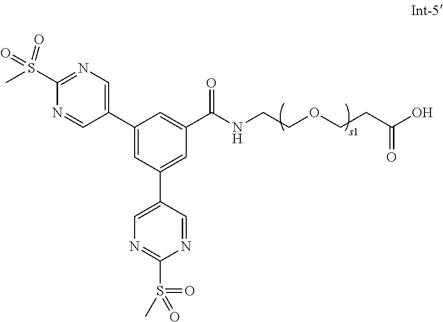

the method comprises the following steps:

### Step 1: Subjecting Compound Int-2-3 to a Coupling Reaction to Synthesize Compound Int-5-1

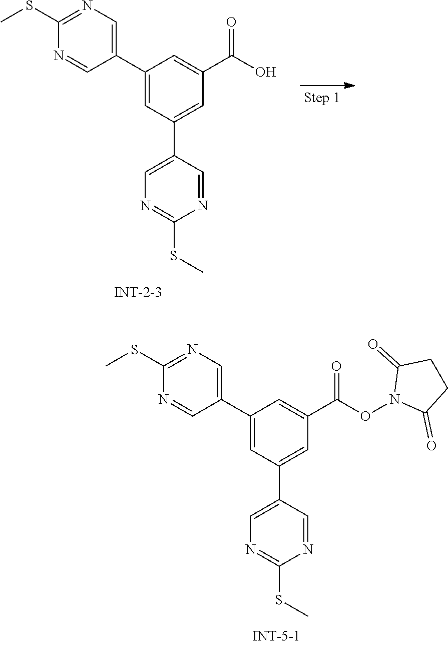 [SMILES: CSc1ncc(-c2cc(C(=O)O)cc(-c3cnc(SC)nc3)c2)cn1 ; CSc1ncc(-c2cc(C(=O)ON3C(=O)CCC3=O)cc(-c3cnc(SC)nc3)c2)cn1]

### Step 2: Subjecting Compound Int-5-1 to a Substitution Reaction to Synthesize Compound Int-5-2′

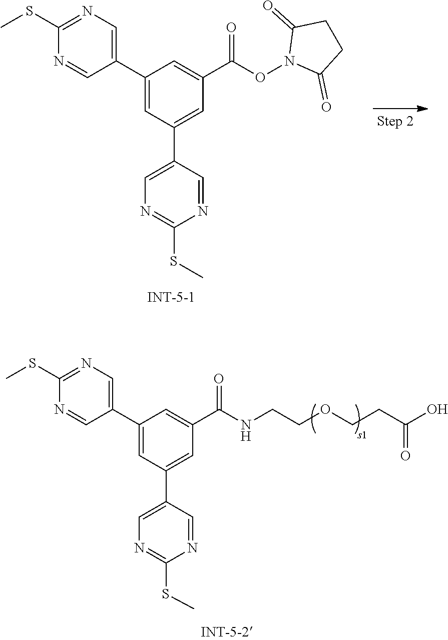

### Step 3: Subjecting Compound Int-5-2′ to an Oxidation Reaction to Synthesize Compound Int-5′

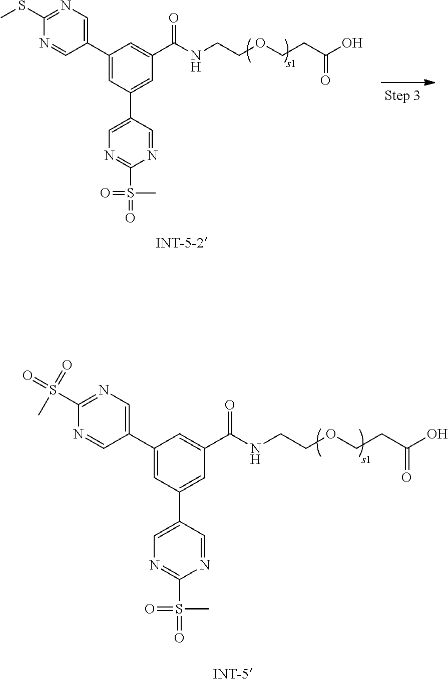

In some embodiments, step 1 is carried out in the presence of N-hydroxysuccinimide; In some embodiments, step 1 is carried out in the presence of dicyclohexylcarbodiimide.

In some embodiments, Step 2 is carried out in the presence of 1-amino-3,6,9,12,15-pentaoxaoctadecan-18-oic acid.

In some embodiments, step 3 is carried out in the presence of an oxidant.

In some embodiments, the oxidant is sodium periodate.

In some embodiments, step 3 is carried out in the presence of ruthenium trichloride monohydrate.

In some embodiments, the present application provides Method 1 for synthesizing the following synthetic intermediate compound:

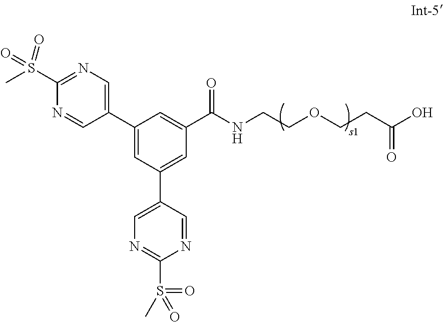

the method comprises the following steps:

### Step 1: Subjecting Compound Int-2-3 to a Coupling Reaction to Synthesize Compound Int-2-4′

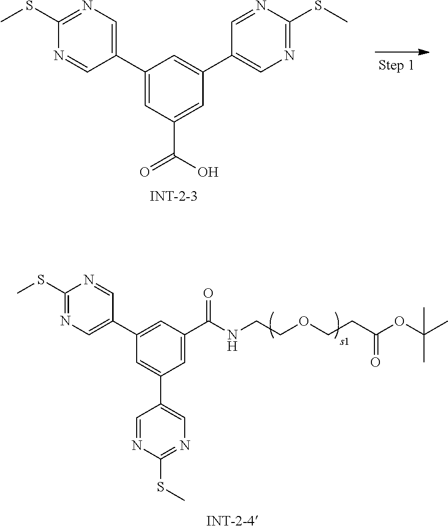

### Step 2: Deprotecting Compound Int-2-4′ to Form Compound Int-2-5′

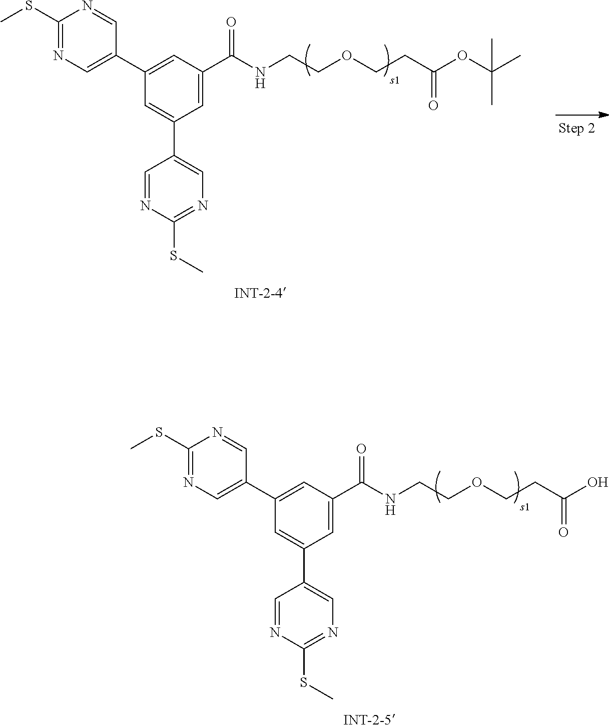

### Step 3: Subjecting Compound Int-2-5′ to a Substitution Reaction to Form Compound Int-5′

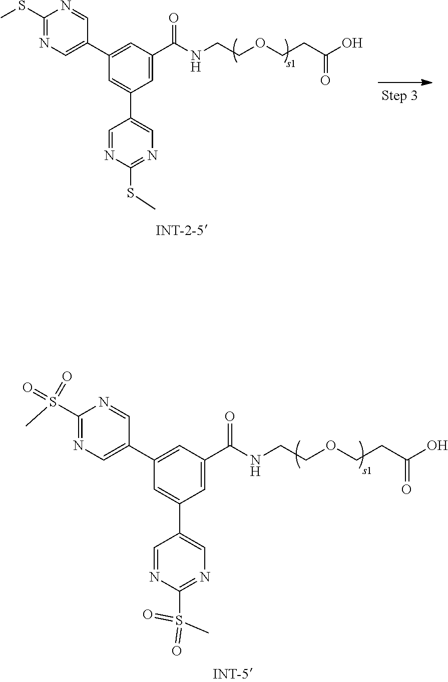

In some embodiments, step 1 is carried out in the presence of the following compounds:

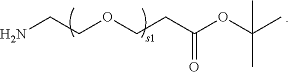

In some embodiments, step 1 is carried out in the presence of HOBt, EDCl and/or DIPEA.

In some embodiments, step 2 is carried out in the presence of an organic acid.

In some embodiments, the organic acid is TFA.

In some embodiments, step 3 is carried out in the presence of an oxidant.

In some embodiments, the oxidant is sodium periodate.

In some embodiments, step 3 is carried out in the presence of ruthenium trichloride monohydrate.

In some embodiments, the present application provides a method for synthesizing the following synthetic intermediate compounds:

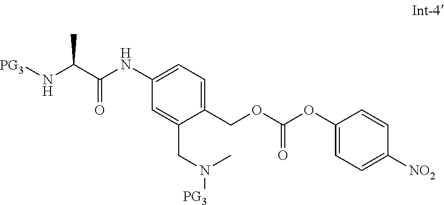

the method comprises the following steps:

### Step 1: Subjecting Compound Int-3′ to a Coupling Reaction to Synthesize Compound Int-4-1′

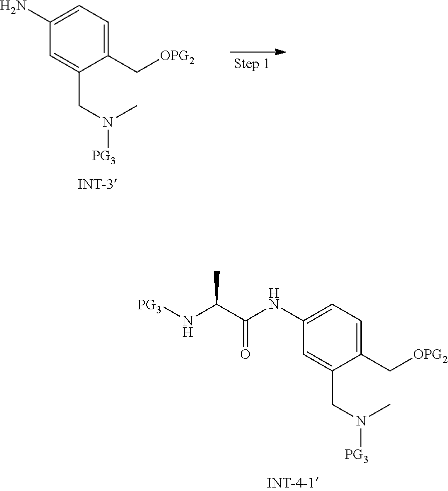

### Step 2: Deprotecting Compound Int-4-1′ to Remove the Protecting Group to Form Compound Int-4-2′

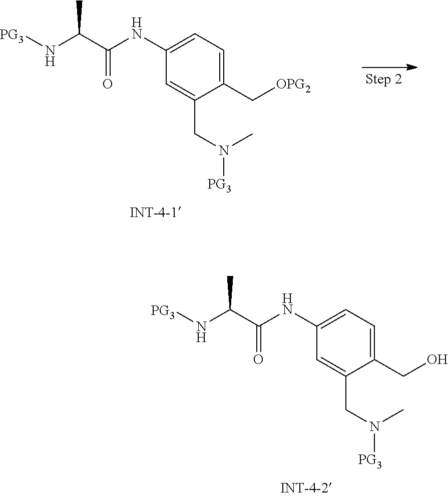

### Step 3: Obtaining Compound Int-4′ from Compound Int-4-2′

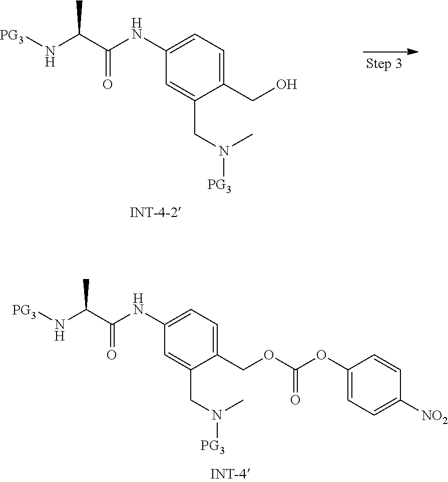

In some embodiments, step 1 is carried out in the presence of 2-ethoxy-1-ethoxycarbonyl-1,2-dihydroquinoline.

In some embodiments, step 2 is carried out in the presence of pyridine hydrofluoride.

In some embodiments, step 3 is carried out in the presence of p-nitrophenyl chloroformate.

In some embodiments, step 3 is carried out in the presence of DIPEA.

For all technical features disclosed in this specification, such as the definition of groups, etc., except for mutually exclusive technical features, all embodiments thereof can be combined in any way to obtain different general formula ranges or specific embodiments. These ranges and embodiments are within the scope of the present invention.

### Definitions

Unless otherwise defined below, the meanings of all technical and scientific terms used herein are intended to be the same as those generally understood by a skilled person in the art. References to the technology used herein shall mean the technology generally recognized in the art, including technological modifications or equivalent substitutions apparent to those skilled in the art. In addition, the laboratory procedures in genomics, nucleic acid chemistry, molecular biology, etc. used herein are all routine steps widely adopted in the respective fields. Although it is believed that the following terms are well understood by those skilled in the art, the following definitions are still provided herein to better explain the present invention.

The term “antibody” refers to an immunoglobulin molecule that is typically composed of two pairs of polypeptide chains, each pair having one light chain (LC) and one heavy chain (HC) Antibody light chains can be categorized into κ (kappa) and 2. (lambda) types. Heavy chains may be categorized into μ, δ, γ, α or ε, with corresponding antibody isotypes defined as IgM, IgD, IgG, IgA and IgE, respectively. In the light chain and heavy chain, the variable region and the constant region are connected by a “J” region of about 12 or more amino acids, and the heavy chain also contains a “D” region of about 3 or more amino acids. Each heavy chain consists of a heavy chain variable region (V_{H}) and a heavy chain constant region (CH). The heavy chain constant region consists of 3 domains (CHI, CH₂ and CH₃). Each light chain consists of a light chain variable region (V_{L}) and a light chain constant region (CL). The light chain constant region consists of one domain CL. The constant domain does not directly participate in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The V_{H} and V_{L} regions may also be subdivided into highly variable regions (called complementarity determining regions (CDRs)), interspersed with more conserved regions called framework regions (FRs). Each of V_{H} and V_{L} consists of three CDRs and four FRs arranged from the amino terminus to the carboxyl terminus in the following order: FR1, CDR1, FR2, CDR2, FR3, CDR3, FR4. The variable regions (V_{H} and V_{L}) of each heavy chain/light chain pair form antigen binding sites, respectively. Amino acid numbering in each region or domain may follow various numbering systems known in the art. The term “antibody” further includes variants where the heavy chain constant region has a C-terminal lysine, lacks a C-terminal lysine, or lacks a C-terminal glycine-lysine dipeptide. It also includes variants where the N-terminal amino acid of the variable region is cyclized to pyroglutamate. Thus, in a composition comprising the disclosed antibodies, each antibody may independently have a C-terminal lysine, lack a C-terminal lysine or C-terminal glycine-lysine, and/or have a N-terminal glutamine or glutamic acid, or pyroglutamate resulting from cyclization of the N-terminal amino acid.

The term “complementarity determining region” or “CDR” refers to the amino acid residues in the variable region of an antibody that are responsible for antigen binding. The variable regions of the heavy chain and light chain each contains three CDRs, designated CDR1, CDR2, and CDR3. The precise boundaries of these CDRs may be defined according to various numbering systems known in the art, for example according to the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), or the AbM numbering system (Martin A C R, Cheetham J C, Rees A R (1989) Modelling antibody hypervariable loops: A combined algorithm. Proc Natl Acad Sci USA 86:9268-9272). For a given antibody, a person skilled in the art will readily identify the CDRs defined by each numbering system. Furthermore, the correspondence between different numbering systems is well known to a person skilled in the art (e.g., see Lefranc et al., Dev. Comparat. Immunol. 27:55-77, 2003).

In the present invention, the CDRs comprised in an antibody or antigen-binding fragment thereof may be determined according to various numbering systems known in the art, such as by Kabat, Chothia, IMGT or AbM numbering systems. In certain embodiments, the CDRs comprised in an antibody or antigen-binding fragment thereof are determined by the Chothia numbering system.

The following general rules disclosed in www.bioinf.org.uk: Professor Andrew C. R. Martin's research group and produce below may be used to define CDRs in an antibody sequence that includes those amino acids that specifically interact with the amino acids comprising the epitope in the antigen to which the antibody binds. There are rare examples where these generally constant features do not occur; however, the Cys residues are the most conserved features.

| Loop | Kabat | AbM | Chothia 1 | Contact 2 | IMGT |
|---|---|---|---|---|---|
| L1 | L24--L34 | L24--L34 | L24--L34 | L30--L36 | L27--L32 |
| L2 | L50--LS6 | L50--L56 | L50--L56 | L46--L55 | L50--L52 |
| L3 | L89--L97 | L89--L97 | L89--L97 | L89--L96 | L89--L97 |
| H1 | H31-H35B | H26--H35B | H26--H32 . . . 34 | H30--H35B | H26--H35B |
| | (Kabat numbering | | | | |
| | system)3 | | | | |
| H1 | H31--H35 | H26--H35 | H26--H32 | H30--H35 | H26--H33 |
| | (Chothia numbering | | | | |
| | system) | | | | |
| H2 | H50--H65 | H50--H58 | H52--H56 | H47--H58 | H51--H56 |
| H3 | H95--H102 | H95--H102 | H95--H102 | H93--H101 | H93--H102 |

1. Some of these numbering systems (particularly for Chothia numbering system) vary depending on the individual publication examined.
2. Any of the numbering system can be used for these CDR definitions, except the Contact numbering scheme uses the Chothia or Martin (Enhanced Chothia) definition.
3. The end of the Chothia CDR-H1 loop when numbered using the Kabat numbering convention varies between H32 and H34 depending on the length of the loop. (This is because the Kabat numbering system places insertions at H35A and H35B).
If neither H35A nor H35B is present, the loop ends at H32;
If only H35A is present, the loop ends at H33;
If both H35A and H35B are present, the loop ends at H34.

The entire amino acid sequence of VH is commonly numbered according to Kabat, while the three CDRs within the variable region may be defined according to any of the aforementioned numbering systems. In particular embodiments, the numbering of the amino acid positions in VH may be sequential beginning with amino acid position 1 continuing sequentially to the end of the sequence or according to Kabat. Unless specified otherwise, the amino acid positions in Vu and VL described herein are defined according to sequential numbering.

The numbering of amino acid positions in the heavy chain constant region may be sequential beginning with amino acid position 1 and continuing sequentially to the end of the sequence or according to Eu numbering. The IgG1 heavy chain constant region amino acid sequence has 330 amino acids sequentially numbered 1 to 330. The corresponding sequence numbered according to Eu begin with position number 118 and ends with position number 447. Unless specified otherwise, the amino acid positions in the heavy and light chains described herein are defined according to sequential numbering.

The term “framework region” or “FR” residues refers to those amino acid residues in the antibody variable region other than the CDR residues as defined above.

The term “antibody” is not limited to any particular method of producing the antibody. This includes, for example, recombinant antibodies, monoclonal antibodies, and polyclonal antibodies. The antibodies may be of different isotypes, for example, IgG (e.g., IgG1, IgG2, IgG3 or IgG4 subtypes), IgA1, IgA2, IgD, IgE or IgM antibodies.

The term “antigen-binding fragment” of an antibody refers to a polypeptide of a fragment of an antibody, such as a polypeptide of a fragment of a full-length antibody, which retains the ability to specifically bind to the same antigen bound by the full-length antibody and/or competes with the full-length antibody for specific binding to the antigen, which is also referred to as an “antigen-binding portion”. See generally, Fundamental Immunology, Ch. 7 (Paul, W., ed., 2nd ed., Raven Press, N.Y. (1989), which is incorporated herein by reference in its entirety for all purposes. An antigen-binding fragment of an antibody may be produced by a recombinant DNA technique or by an enzymatic or chemical cleavage of the intact antibody. Non-limiting examples of antigen-binding fragment include Fab fragment, Fab′ fragment, F(ab)′2 fragment, F(ab)′₃ fragment, Fd, Fv, scFv, di-scFv, (scFv)₂, disulfide-stabilized Fv protein (“dsFv”), single domain antibody (sdAb, nanobody), and a polypeptide that comprises at least a portion of an antibody sufficient to confer specific antigen binding ability to the polypeptide. Engineered antibody variants are reviewed in Holliger et al., 2005; Nat Biotechnol, 23:1126-1136.

The term “Fd” refers to an antibody fragment consisting of the VH and CH₁ domains; the term “dAb fragment” refers to an antibody fragment consisting of the Va domain (Ward et al., Nature 341:544 546 (1989)); the term “Fab fragment” refers to an antibody fragment consisting of the VL, VH, CL and CH₁ domains; the term “F(ab′)₂ fragment” refer to an antibody fragment comprising two Fab fragments connected by a disulfide bridge on the hinge region; the term “Fab′ fragment” refers to a fragment obtained after reducing the disulfide bonds connecting the two heavy chain fragments in the F(ab′)₂ fragment, which consists of a complete light chain and the Fd fragment of the heavy chain (consisting of the VH and CH₁ domains).

The term “Fv” refers to an antibody fragment consisting of the VL and Vu domains of a single arm of an antibody. The Fv fragment is generally considered to be the smallest antibody fragment that may form a complete antigen binding site. It is generally believed, six CDRs confer antigen-binding specificity to an antibody. However, even a single variable region (e.g., an Fd fragment, which contains only three CDRs specific for an antigen) is able to recognize and bind to an antigen, although its affinity may be lower than that of the complete binding site.

The term “Fc” refers to an antibody fragment formed by disulfide bonding of the second and third constant regions of the first heavy chain of an antibody to the second and third constant regions of the second heavy chain. The Fc fragment of an antibody has a variety of different functions but is not involved in antigen binding.

The term “scFv” refers to a single polypeptide chain containing VL and Vu domains connected by a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have a general structure: NH₂-VL-linker-VH—COOH or NH₂—VH-linker-VL-COOH. A suitable prior art peptide linker consists of the repeated GGGGS (SEQ ID NO: 46) amino acid sequence or its variants. For example, a linker having the amino acid sequence (GGGGS)₄ (SEQ ID NO: 47) may be used, but variants thereof may also be used (Holliger et al. (1993), Proc. Natl. Acad. Sci. USA 90:6444-6448). Other linkers that may be used in the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the Vu and Vi of the scFv. In certain embodiments, the VH and VL domains may be positioned relative to each other in any suitable arrangement. For example, a scFv may comprise NH₂—VH—VH—COOH, NH₂—VL-VL-COOH.

The term “single-domain antibody (sdAb)” has the meaning commonly understood by those skilled in the art, which refers to an antibody fragment composed of a single monomeric variable antibody domain (e.g., a single heavy chain variable region) that retains the ability to specifically bind to the same antigen bound by a full-length antibody (Holt, L. et al., Trends in Biotechnology, 21(11):484-490, 2003). Single-domain antibody is also called nanobody.

The above-mentioned antibody fragments all retain the ability to specifically bind to the same antigen bound by a full-length antibody, and/or compete with the full-length antibody for specific binding to the antigen.

As used therein, unless the context clearly indicates otherwise, when referring to the term “antibody”, it includes not only an intact antibody but also an antigen-binding fragment of the antibody.

An antigen-binding fragment of antibody (e.g., the above-mentioned antibody fragment) may be obtained from a given antibody (e.g., the antibody provided by the present invention) using a conventional technique known to a skilled person in the art (e.g., recombinant DNA technology or enzymatic or chemical cleavage method), and the antigen-binding fragment of antibody may be screened for specificity in the same manner as for the intact antibody.

The term “murine antibody” refers to an antibody obtained by the following method: fusing B cells of immunized mice with myeloma cells, screening for murine hybrid fusion cells that may both proliferate indefinitely and secrete antibodies, followed by screening, antibody preparation and antibody purification; or refers to an antibody secreted by plasma cells formed by differentiation and proliferation of B cells after an antigen invades mice.

The term “humanized antibody” refers to a non-human antibody modified by genetic engineering, of which amino acid sequence is modified to increase the homology with the sequence of a human antibody. Generally speaking, all or part of the CDR region of a humanized antibody comes from a non-human antibody (donor antibody), and all or part of the non-CDR region (e.g., variable region FR and/or constant region) comes from a human immunoglobulin (recipient antibody). the humanized antibody generally retains the expected properties of the donor antibody, including, but not limited to, antigen specificity, affinity, reactivity, ability to increase immune cell activity, ability to enhance immune response, etc. The donor antibody may be a mouse, rat, rabbit or non-human primate (e.g., cynomolgus monkey) antibody with the expected properties (e.g., antigen specificity, affinity, reactivity, ability to increase immune cell activity and/or ability to enhance immune response).

The term “identity” is used to refer to the a match of sequences between two polypeptides or between two nucleic acids. When a position in both sequences being compared is occupied by the same base or amino acid monomer subunit (e.g., a position in each of two DNA molecules is occupied by adenine, or a position in each of two polypeptides is occupied by lysine), then the molecules are identical at that position. “Percent identity” between two sequences is a function of the number of matching positions common to the two sequences divided by the number of positions compared×100. For example, if 6 out of 10 positions of two sequences match, then the two sequences are 60% identical. For example, the DNA sequences CTGACT and CAGGTT share 50% identity (3 positions match out of 6 total positions). Typically, comparisons are made when two sequences are aligned to yield maximum identity. Such alignments can be achieved using, for example, the method of Needleman et al. (1970) J. Mol Biol. 48:443-453, which can be conveniently performed by computer programs such as the Align program (DNAstar, Inc.). The algorithm of E. Meyers and W. Miller (Comput. Appl Biosci., 4:11-17 (1988)) which has been incorporated into the ALIGN program (version 2.0), can be used to determine the percent identity between two amino acid sequences by using the PAM120 weight residue table, a gap length penalty of 12, and a gap penalty of 4. Alternatively, the algorithm of Needleman and Wunsch (J Mol Biol. 48:444-453 (1970)) which has been integrated into the GAP program of the GCG software package (available at www.gcg.com) and can be used to determine the percent identity between two amino acid sequences by using the Blossum 62 matrix or PAM250 matrix with a gap weight of 16, 14, 12, 10, 8, 6, or 4, and a length weight of 1, 2, 3, 4, 5, or 6.

The term “conservative substitution” refers to an amino acid substitution that does not adversely affect or change the expected properties of the protein/polypeptide comprising the amino acid sequence. For example, a conservative substitution may be introduced by a standard technique known in the art such as site-directed mutagenesis and PCR-mediated mutagenesis. Conservative amino acid substitutions include substitutions in which amino acid residues are substituted with amino acid residues having similar side chains, such as substitutions with residues that are physically or functionally similar to the corresponding amino acid residues (e.g., having similar size, shape, charge, chemical properties, including the ability to form covalent bonds or hydrogen bonds, etc.). Families of amino acid residues with similar side chains have been defined in the art. These families include amino acids with basic side chains (e.g., lysine, arginine, and histidine), acidic side chains (e.g., aspartic acid, glutamic acid), uncharged polar side chains (e.g., glycine, asparagine, glutamine, serine, threonine, tyrosine, cysteine, tryptophan), non-polar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, it is preferred to replace a corresponding amino acid residue with another amino acid residue from the same side chain family. Methods for identifying conservative substitutions of amino acids are well known in the art (see, for example, Brummell et al., Biochem. 32:1180-1187 (1993); Kobayashi et al. Protein Eng. 12 (10): 879-884 (1999); and Burks et al. Proc. Natl Acad. Set USA 94:412-417 (1997), which are incorporated herein by reference).

The twenty conventional amino acids used herein are written in accordance with conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine may be represented by A or Ala.

The term “linker” refers to a structural fragment that connects a cytotoxic drug to an antibody or antigen-binding fragment thereof. For example, it refers to the -M-L-E- structural fragment in the formula Ab-[M-L-E-D]ₓ.

The term “drug-linker” refers to a structure of the cytotoxic drug and linker described in the present invention before being connected to an antibody or antigen-binding fragment thereof. For example, “drug-linker” refers to M′-L-E-D, wherein M′ is the structural form of M before covalently connecting to an antibody or antigen-binding fragment thereof. “Drug-linker” is covalently connected to an antibody or antigen-binding fragment thereof to obtain the antibody-drug conjugate described in the present application.

The “drug-linker” also includes all pharmaceutically acceptable isotopically labeled compounds thereof, which are the same as the “drug-linker” compound of the present invention, except that one or more atoms are replaced by atoms having the same atomic number but an atomic mass or mass number different from the atomic mass or mass number that predominates in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

The terms “include,” “comprises,” “have,” “contain,” or “involve,” and other variations thereof as used herein, are inclusive or open-ended and do not exclude other unlisted elements or method steps.

The term “alkyl” refers to a group, such as “C₁₋₂₀ alkyl”, “C₁₋₁₀ alkyl”, “C₁₋₆ alkyl”, “C₁₋₄ alkyl”, “C₁₋₃ alkyl”, etc., which is obtained by removing one hydrogen atom from a straight or branched hydrocarbonyl. Its specific examples include, but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “isotopically labeled compound” means that the compound is identical in structure to the compound of the present invention except that one or more atoms are replaced by atoms having the same atomic number but an atomic mass or mass number different from the atomic mass or mass number that prevails in nature. Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

As used herein, the term “suitable substituent” refers to a modification that a person skilled in the art can make to a compound according to the need of the compound substituent. “Suitable substituent” includes oxo (═O), halogen, cyano, NR⁸R⁹, carboxyl, mercapto, hydroxyl, ester (e.g., —C₁₋₆ alkyl-C(═O)—OC₁₋₆ alkyl), C₁₋₆ alkyl, C₂₋₆ alkenyl, C₂₋₆ alkynyl, C₁₋₆ alkyl-O—C₁₋₆ alkyl, C₁₋₆ haloalkyl, C₁₋₆ alkoxy, C₃₋₆ cycloalkyl, 3- to 10-membered heterocyclyl, 5- to 10-membered heteroaryl, C₆₋₁₀ aryl, benzyl, hydroxy-substituted benzyl, indolylmethylene and C₁₋₆ haloalkoxy, wherein R⁸, R⁹ are each independently selected from the group consisting of H, C₁₋₆ alkyl, C₃₋₆ cycloalkyl, 3- to 10-membered heterocyclyl, 5- to 10-membered heteroaryl, C₆₋₁₀ aryl, C₁₋₆ alkoxy, C₁₋₆ haloalkyl, C₁₋₆ haloalkoxy, halogen, hydroxyl, carboxyl and ester (e.g. —C₁₋₆ alkyl-C(═O)—OC₁₋₆ alkyl).

The term “substituted” means that one or more (e.g. 1, 2, 3, 4 or 5) hydrogen atoms on the specified compound or structural fragment are replaced by a substituent, provided that the normal valence of the specified atom in the current situation is not exceeded and the substitution forms a stable compound. Combinations of substituents and/or variables are permissible only if they yield stable compounds. In some embodiments, the substituent is each independently composed of one or more of the following structures: —O—, —S—, —NR′—, halogen, —CN, —OH, —NH₂, —NO₂, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 8-membered heterocyclyl(ene), C₆-C₁₀ aryl(ene) and 5- to 10-membered heteroaryl(ene), etc. In some embodiments, the substituent is each independently composed of one or more of the following structures: NR⁸R⁹, —O—, —S—, —NR′—, halogen, —CN, —OH, —SH, —NH₂, —NO₂, —C(O)—, —CN, ═O, C₁-C₆ alkyl(ene), C₁-C₆ haloalkyl(ene), C₁-C₆ alkoxy, C₂-C₆ alkenyl(ene), C₂-C₆ alkynyl(ene), C₃-C₈ cycloalkyl(ene), 3- to 10-membered heterocyclyl(ene), C₆-C₁₀ aryl(ene), and 5- to 10-membered heteroaryl(ene), etc., wherein R⁸, R⁹ and R′ are as defined above. For example, the substituents may be suitable substituents as described above.

If a functional group or structural fragment is described as “substituted or unsubstituted”, the functional group or structural fragment may be (1) unsubstituted or (2) substituted.

As used herein, the term “one or more” means 1 or more than 1, such as 2, 3, 4, 5 or 10, under reasonable conditions.

Unless otherwise specified, as used herein, the site for attachment of a substituent may be from any suitable position of the substituent.

Whether or not explicitly indicated, all numerical values in the present application are modified by the term “about”. The term “about” refers to a range of ±20% of the stated value, ±10% of the stated value, preferably ±5% of the stated value, and more preferably ±2% of the stated value.

### BRIEF DESCRIPTION OF THE DRAWINGS

FIG. 1 shows the efficacy results of anti-human HER2 antibody-drug conjugates (ADCs) in NCI-N87 cell subcutaneous tumor-bearing mouse model.

FIG. 2 shows the efficacy results of anti-human HER2 antibody-drug conjugates (ADCs) in NCI-N87 cell subcutaneous tumor-bearing mouse model.

FIG. 3 shows the efficacy results of anti-human HER2 antibody-drug conjugates (ADCs) on NCI-N87 cell subcutaneous tumor-bearing mouse model.

FIG. 4 shows the changes in the body weight of each group of mice in the human gastric cancer cell NCI-N87 CDX model.

### SPECIFIC MODELS FOR CARRYING OUT THE PRESENT INVENTION

The present invention is further illustrated through the description of the specific models as follows, but this is not a limitation of the present invention. Based on the teachings of the present invention, those skilled in the art can make various modifications or improvements without departing from the basic concept and scope of the present invention.

The information of the sequences involved in the present invention is described in the following table:

| SEQ ID | | |
|---|---|---|
| NO: | Sequence Name | Sequence Information |
| 1 | Trastuzumab VH amino acid | EVQLVESGGGLVQPGGSLRLSCAASGFNIKDT |
| | sequence | YIHWVRQAPGKGLEWVARIYPTNGYTRYADS |
| | | VKGRFTISADTSKNTAYLQMNSLRAEDTAVY |
| | | YCSRWGGDGFYAMDYWGQGTLVTVSS |
| 2 | Trastuzumab VL amino acid | DIQMTQSPSSLSASVGDRVTITCRASQDVNTA |
| | sequence | VAWYQQKPGKAPKLLIYSASFLYSGVPSRFSG |
| | | SRSGTDFTLTISSLQPEDFATYYCQQHYTTPPT |
| | | FGQGTKVEIK |
| 3 | Pertuzumab VH amino acid | EVQLVESGGGLVQPGGSLRLSCAASGFTFTDY |
| | sequence | TMDWVRQAPGKGLEWVADVNPNSGGSIYNQ |
| | | RFKGRFTLSVDRSKNTLYLQMNSLRAEDTAV |
| | | YYCARNLGPSFYFDYWGQGTLVTVSS |
| 4 | Pertuzumab VL amino acid | DIQMTQSPSSLSASVGDRVTITCKASQDVSIGV |
| | sequence | AWYQQKPGKAPKLLIYSASYRYTGVPSRFSGS |
| | | GSGTDFTLTISSLQPEDFATYYCQQYYTYPYTF |
| | | GQGTKVEIK |
| 5 | Trastuzumab Chothia CDR H1 | GFNIKDT |
| 6 | Trastuzumab Chothia CDR H2 | YPTNGY |
| 7 | Trastuzumab | WGGDGFYAMDY |
| | Chothia/kabat/AbM CDR H3 | |
| 8 | Trastuzumab | RASQDVNTAVA |
| | Chothia/Kabat/AbM CDR L1 | |
| 9 | Trastuzumab | SASFLYS |
| | Chothia/Kabat/AbM CDR L2 | |
| 10 | Trastuzumab | QQHYTTPPT |
| | Chothia/Kabat/IMGT/AbM | |
| | CDR L3 | |
| 11 | Trastuzumab Kabat CDR H1 | DTYIH |
| 12 | Trastuzumab Kabat CDR H2 | RIYPTNGYTRYADSVKG |
| 13 | Trastuzumab IMGT CDR H1 | GFNIKDTY |
| 14 | Trastuzumab IMGT CDR H2 | TYPINGYT |
| 15 | Trastuzumab IMGT CDR H3 | SRWGGDGFYAMDY |
| 16 | Trastuzumab IMGT CDR L1 | QDVNTA |
| 17 | Trastuzumab IMGT CDR L2 | SAS |
| 18 | Trastuzumab AbM CDR H1 | GFNIKDTYIH |
| 19 | Trastuzumab AbM CDR H2 | RIYPTNGYTR |
| 20 | Pertuzumab Chothia CDR H1 | GFTFTDY |
| 21 | Pertuzumab Chothia CDR H2 | NPNSGG |
| 22 | Pertuzumab | NLGPSFYFDY |
| | Chothia/Kabat/AbM CDR H3 | |
| 23 | Pertuzumab | KASQDVSIGVA |
| | Chothia/Kabat/AbM CDR L1 | |
| 24 | Pertuzumab | SASYRYT |
| | Chothia/Kabat/AbM CDR L2 | |
| 25 | Pertuzumab | QQYYIYPYT |
| | Chothia/Kabat/IMGT/AbM | |
| | CDR L3 | |
| 26 | Pertuzumab Kabat CDR H1 | DYTMD |
| 27 | Pertuzumab Kabat CDR H2 | DVNPNSGGSIYNQRFKG |
| 28 | Pertuzumab IMGT CDR H1 | GFTFTDYT |
| 29 | Pertuzumab IMGT CDR H2 | VNPNSGGS |
| 30 | Pertuzumab IMGT CDR H3 | ARNLGPSFYFDY |
| 31 | Pertuzumab IMGT CDR L1 | QDVSIG |
| 32 | Pertuzumab IMGT CDR L2 | SAS |
| 33 | Pertuzumab AbM CDR H1 | GFTFTDYTMD |
| 34 | Pertuzumab AbM CDR H2 | DVNPNSGGSI |
| 35 | Amino acid sequence of heavy | ASTKGPSVFPLAPSSKSTSGGTAALGCLVKDY |
| | chain constant region of | FPEPVTVSWNSGALTSGVHTFPAVLQSSGLYS |
| | Trastuzumab or Pertuzumab | LSSVVTVPSSSLGTQTYICNVNHKPSNTKVDK |
| | | KVEPKSCDKTHTCPPCPAPELLGGPSVFLFPPK |
| | | PKDTLMISRTPEVTCVVVDVSHEDPEVKFNW |
| | | YVDGVEVHNAKTKPREEQYNSTYRVVSVLTV |
| | | LHQDWLNGKEYKCKVSNKALPAPIEKTISKAK |
| | | GQPREPQVYTLPPSREEMTKNQVSLTCLVKGF |
| | | YPSDIAVEWESNGQPENNYKTTPPVLDSDGSF |
| | | FLYSKLTVDKSRWQQGNVFSCSVMHEALHNH |
| | | YTQKSLSLSPGK |
| 36 | Amino acid sequence of light | RTVAAPSVFIFPPSDEQLKSGTASVVCLLNNFY |
| | chain constant region of | PREAKVQWKVDNALQSGNSQESVTEQDSKDS |
| | Trastuzumab or Pertuzumab | TYSLSSTLTLSKADYEKHKVYACEVTHQGLSS |
| | | PVTKSFNRGEC |
| 37 | Amino acid sequence of heavy | EVQLVESGGGLVQPGGSLRLSCAASGFNIKDT |
| | chain of Trastuzumab | YIHWVRQAPGKGLEWVARIYPTNGYTRYADS |
| | | VKGRFTISADTSKNTAYLQMNSLRAEDTAVY |
| | | YCSRWGGDGFYAMDYWGQGTLVTVSSASTK |
| | | GPSVFPLAPSSKSTSGGTAALGCLVKDYFPEPV |
| | | TVSWNSGALTSGVHTFPAVLQSSGLYSLSSVV |
| | | TVPSSSLGTQTYICNVNHKPSNTKVDKKVEPK |
| | | SCDKTHTCPPCPAPELLGGPSVFLFPPKPKDTL |
| | | MISRTPEVTCVVVDVSHEDPEVKFNWYVDGV |
| | | EVHNAKTKPREEQYNSTYRVVSVLTVLHQDW |
| | | LNGKEYKCKVSNKALPAPIEKTISKAKGQPRE |
| | | PQVYTLPPSREEMTKNQVSLTCLVKGFYPSDI |
| | | AVEWESNGQPENNYKTTPPVLDSDGSFFLYSK |
| | | LTVDKSRWQQGNVFSCSVMHEALHNHYTQK |
| | | SLSLSPGK |
| 38 | Amino acid sequence of light | DIQMTQSPSSLSASVGDRVTITCRASQDVNTA |
| | chain of Trastuzumab | VAWYQQKPGKAPKLLIYSASFLYSGVPSRFSG |
| | | SRSGTDFTLTISSLQPEDFATYYCQQHYTTPPT |
| | | FGQGTKVEIKRTVAAPSVFIFPPSDEQLKSGTA |
| | | SVVCLLNNFYPREAKVQWKVDNALQSGNSQE |
| | | SVTEQDSKDSTYSLSSTLTLSKADYEKHKVYA |
| | | CEVTHQGLSSPVTKSFNRGEC |
| 39 | Amino acid sequence of heavy | EVQLVESGGGLVQPGGSLRLSCAASGFTFTDY |
| | chain of Pertuzumab | TMDWVRQAPGKGLEWVADVNPNSGGSIYNQ |
| | | RFKGRFTLSVDRSKNTLYLQMNSLRAEDTAV |
| | | YYCARNLGPSFYFDYWGQGTLVTVSSASTKG |
| | | PSVFPLAPSSKSTSGGTAALGCLVKDYFPEPVT |
| | | VSWNSGALTSGVHTFPAVLQSSGLYSLSSVVT |
| | | VPSSSLGTQTYICNVNHKPSNTKVDKKVEPKS |
| | | CDKTHTCPPCPAPELLGGPSVFLFPPKPKDTLM |
| | | ISRTPEVTCVVVDVSHEDPEVKFNWYVDGVE |
| | | VHNAKTKPREEQYNSTYRVVSVLTVLHQDWL |
| | | NGKEYKCKVSNKALPAPIEKTISKAKGQPREP |
| | | QVYTLPPSREEMTKNQVSLTCLVKGFYPSDIA |
| | | VEWESNGQPENNYKTTPPVLDSDGSFFLYSKL |
| | | TVDKSRWQQGNVFSCSVMHEALHNHYTQKS |
| | | LSLSPGK |
| 40 | Amino acid sequence of light | DIQMTQSPSSLSASVGDRVTITCKASQDVSIGV |
| | chain of Pertuzumab | AWYQQKPGKAPKLLIYSASYRYTGVPSRFSGS |
| | | GSGTDFTLTISSLQPEDFATYYCQQYYTYPYTF |
| | | GQGTKVEIKRTVAAPSVFIFPPSDEQLKSGTAS |
| | | VVCLLNNFYPREAKVQWKVDNALQSGNSQES |
| | | VTEQDSKDSTYSLSSTLTLSKADYEKHKVYAC |
| | | EVTHQGLSSPVTKSFNRGEC |
| 41 | Short peptide | GGFG |
| 42 | Short peptide | GGVA |
| 43 | Short peptide | GFLG |
| 44 | Short peptide | EAAA |
| 45 | Short peptide | GGGGG |
| 46 | Linker subunit | GGGGS |
| 47 | (GGGGS)₄ | GGGGSGGGGSGGGGSGGGGS |

The abbreviations used herein have the following meanings:

| Abbreviation | Meaning | Abbreviation | Meaning |
|---|---|---|---|
| HATU | N,N,N′,N′-Tetramethyl-O-(7-azabenzotriazol-1-yl)uronium | EDCI | 1-(3-Dimethylaminopropyl)-3-ethylcarbodiimide |
| | hexafluorophosphate | | hydrochloride |
| EEDQ | 2-Ethoxy-1-ethoxycarbonyl-1,2-dihydroquinoline | HOBt | 1-hydroxybenzotriazole |
| PyBOP | 1H-Benzotriazol-1-yloxytripyrrolidinyl | DMTMM | 4-(4,6-Dimethoxytriazin-2-yl)-4-methylmorpholine |
| | hexafluorophosphate | | hydrochloride |
| DIPEA | Diisopropylethylamine | DBU | 1,8-Diazobispiro[5.4.0]undec-7-ene |
| DMAP | 4-Dimethylaminopyridine | DCC | N,N′-dicyclohexylcarbodiimide |
| HOSu | N-hydroxysuccinimide | PPTS | Pyridinium p-toluenesulfonate |
| TsOH | p-Toluenesulfonic acid | TFA | Trifluoroacetic acid |
| AcOH | Acetic acid | LiOH•H₂O | Lithium hydroxide monohydrate |
| NaHCO₃ | Sodium bicarbonate | Pd(PPh₃)₄ | Tetratriphenylphosphine palladium |
| Pb(OAc)₄ | Lead tetraacetate | MMAE | Monomethyl auristatin E |
| AE | Auristatin E | Cit | Citrulline |
| Val | Valine | Boc | tert-Butyloxycarbonyl |
| PABC | p-Aminobenzyl alcohol | Pt/C | Platinum on carbon |
| Pd/C | Palladium on carbon | HPLC | High performance liquid |
| | | | chromatography |
| LCMS | Liquid chromatography-mass | Rf | Retardation factor |
| | spectrometry | | |
| Pre-TLC | Preparative thin layer | DMSO | Dimethyl sulfoxide |
| | chromatography | | |
| PE | Petroleum ether | EA | Ethyl acetate |
| THF | Tetrahydrofuran | DMF | N,N-dimethylformamide |
| MeOH | Methanol | DCM | Dichloromethane |
| IPA | Isopropanol | MeCN | Acetonitrile |
| MTBE | Methyl tert-butyl ether | H₂O | Water |

The structures of the compounds described in the following examples were determined by nuclear magnetic resonance (1H NMR) or mass spectrometry (MS).

Nuclear magnetic resonance (1H NMR) was measured using a Bruker 400 MHz nuclear magnetic resonance instrument; the deuterated reagent was hexadeuterated dimethylsulfoxide (DMSO-d6); and the internal standard substance was tetramethylsilane (TMS).

The abbreviations in the nuclear magnetic resonance (NMR) spectra used in the examples are shown below.
s: singlet, d: doublet, t: triplet, q: quartet, m: multiplet, br: broad, J: coupling constant, Hz: Hertz. DMSO-d6: deuterated dimethylsulfoxide. δ values are expressed in ppm.

The mass spectrometer (MS) was determined using an Agilent (ESI) mass spectrometer, model: Agilent 6120B.

### Intermediate Preparation Example 1: Preparation of 1-(3,5-bis(2-(methylsulfonyl) pyrimidin-5-yl)phenyl)-1-oxo-5,8,11,14,17,20,23,26-octaoxa-2-azanonacosan-29-oic acid (INT-2)

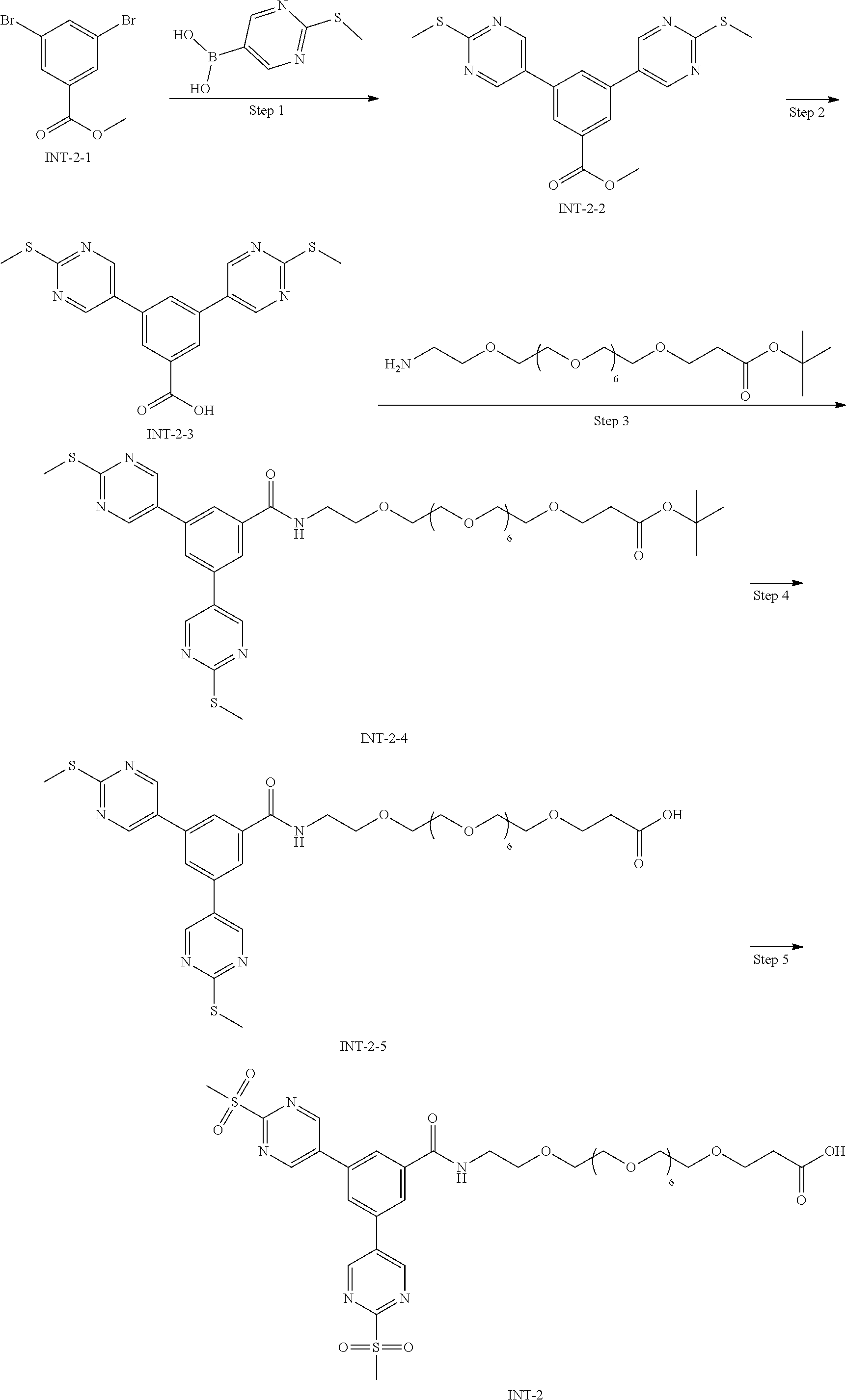

### Step 1: Preparation of methyl 3,5-bis(2-(methylthio)pyrimidin-5-yl)benzoate (INT-2-2)

The raw materials methyl 3,5-dibromobenzoate (720 mg, 2.45 mmol), 2-methylthiopyrimidine-5-boronic acid (874 mg, 5.14 mmol), XPhosPd G3 (207 mg, 245 μmol), and K₃PO₄ (1.56 g, 7.35 mmol) were added to dioxane (12 mL) and water (4 mL). The reaction system was stirred at 90° C. for 3 hours under nitrogen atmosphere. The reaction was monitored by LC-MS. The reaction solution was filtered with celite. Water and ethyl acetate were added to the filtrate for extraction, and concentrated to obtain a crude product, which was purified by column chromatography (EA/PE=0-25%) to obtain 710 mg of methyl 3,5-bis(2-(methylthio)pyrimidin-5-yl)benzoate.

Its structural characterization data are as follows:
ESI-MS (m/z): 385.1 [M+H]⁺.

### Step 2: Preparation of 3,5-bis(2-(methylthio)pyrimidin-5-yl)benzoic acid (INT-2-3)

Methyl 3,5-bis(2-(methylthio)pyrimidin-5-yl)benzoate (650 mg, 1.69 mol) and lithium hydroxide (121 mg, 5.07 mmol) were dissolved in THF (2 mL), MeOH (2 mL) and H₂O (2 mL). The reaction was carried out under stirring at 25° C. for 2 hours, and monitored by LC-MS. The reaction system was adjusted to a pH of about 2 with 1N HCl, precipitating a large amount of solid, and then filtered. The filter cake was collected and dried to obtain 560 mg of 3,5-bis(2-(methylthio)pyrimidin-5-yl)benzoic acid.

Its structural characterization data are as follows:
ESI-MS (m/z): 371.1 [M+H]⁺.

### Step 3: Preparation of tert-butyl 1-(3,5-bis(2-(methylthio)pyrimidin-5-yl)phenyl)-1-oxo-5,8, 11, 14, 17,20,23,26-octaoxa-2-azanonacosan-29-oate (INT-2-4)

3,5-Bis(2-(methylthio)pyrimidin-5-yl)benzoic acid (3.00 g, 8.10 mmol) and tert-butyl 1-amino-3,6,9,12,15,18,21,24-octaoxaheptan-27-oate (4.03 g, 8.10 mmol) were added to DMF (40 mL), then HOBt (3.28 g, 24.3 mmol), EDCI (4.66 g, 24.3 mmol) and DIPEA (4.19 g, 32.4 mmol, 5.64 mL) were added in sequence, and the reaction system was stirred at 60° C. for 2 hours. Water (100 mL) and ethyl acetate (60 mL×3) were added to the reaction solution for extraction. The organic phases were combined, dried over anhydrous sodium sulfate, filtered, and concentrated to obtain tert-butyl 1-(3,5-bis(2-(methylthio)pyrimidin-5-yl)phenyl)-1-oxo-5,8,11, 14, 17,20,23,26-octaoxa-2-azanonacosan-29-oate 4.20 g, 4.14 mmol), which was used directly in the next step without purification.

### Step 4: Preparation of 1-(3,5-bis(2-(methylthio)pyrimidin-5-yl)phenyl)-1-oxo-5,8,11,14,17,20,23,26-octaoxa-2-azanonacosan-29-oic acid (INT-2-5)

Tert-butyl 1-(3,5-bis(2-(methylthio)pyrimidin-5-yl)phenyl)-1-oxo-5,8,11,14,17,20,23,26-octaoxa-2-azanonacosan-29-oate (3.60 g, 4.24 mmol) was dissolved in dichloromethane (30 mL), TFA (15.3 g, 134 mmol, 10 mL) was added, and the reaction system was stirred at 25° C. for 6 hours. Water (60 mL) and ethyl acetate (40 mL×3) were added to the reaction solution for extraction. The organic phases were combined, dried over anhydrous sodium sulfate, filtered, and concentrated to obtain a crude product, which was purified by preparative HPLC and freeze-dried to obtain 1-(3,5-bis(2-(methylthio)pyrimidin-5-yl)phenyl)-1-oxo-5,8,11,14,17,20,23,26-octaoxa-2-azanonacosan-29-oic acid (2.93 g, 3.63 mmol).

Its structural characterization data are as follows:

ESI-MS (m/z): 794.3 [M+H]⁺.

The purification method was follows:
Chromatographic column: Phenomenex luna C18 (250 mm*70 mm*10 μm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 15.00 | 60 | 40 | 30 |

### Step 5: Preparation of 1-(3,5-bis(2-(methylsulfonyl)pyrimidin-5-yl)phenyl)-1-oxo-5,8,11,14,17,20,23,26-octaoxa-2-azanonacosan-29-oic acid (INT-2)

1-(3,5-Bis(2-(methylthio)pyrimidin-5-yl)phenyl)-1-oxo-5,8,11,14,17,20,23,26-octaoxa-2-az anonacosan-29-oic acid (148 mg, 0.186 mmol) was added to acetonitrile (15 mL) and water (7.5 mL), then sodium periodate (398.71 mg, 1.86 mmol) and ruthenium trichloride hydrate (15.47 mg, 74.56 μmol) were added to the reaction system, and the reaction was carried out under stirring at 25° C. for 30 minutes. The reaction system was extracted with water and ethyl acetate, and concentrated to obtain 1-(3,5-bis(2-(methylsulfonyl)pyrimidin-5-yl)phenyl)-1-oxo-5,8,11,14,17,20,23,26-octaoxa-2-azanonacosan-29-oic acid (155 mg).

Its structural characterization data are as follows:

ESI-MS (m/z): 858.3 [M+H]⁺.

### Intermediate Preparation Example 2: Preparation of allyl (5-amino-2-(((tert-butyldiphenylsilyl)oxy)methyl)benzyl)(methyl)carbamate (INT-3)

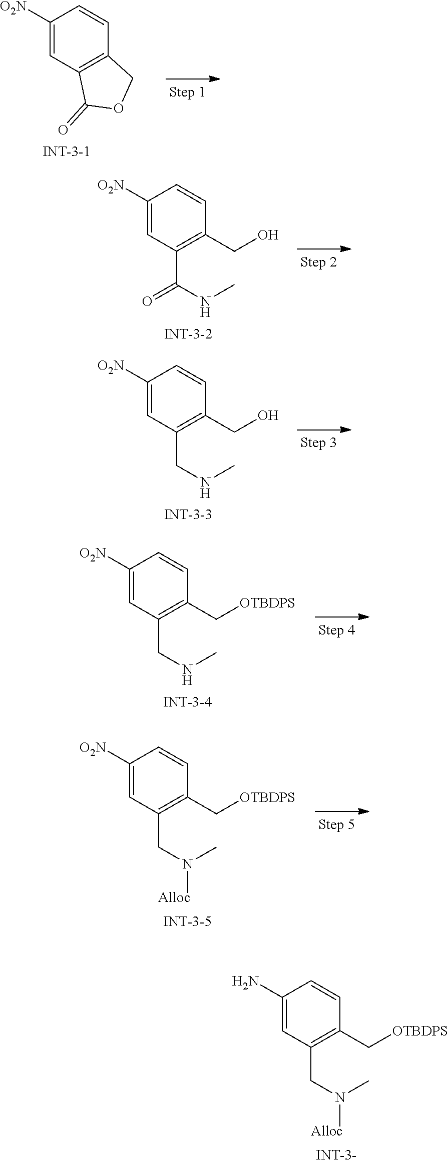

### Step 1: Preparation of 2-hydroxymethyl-N-methyl-5-nitrobenzamide (INT-3-2)

6-Nitroisobenzofuran-1 (3H)-one (50.0 g, 279 mmol) was added to a tetrahydrofuran solution of methylamine (2 M, 500 mL), heated to 75° C. and stirred for 5 hours. The reaction solution was directly concentrated to obtain the crude product of the title compound (58.0 g, crude), which was used directly in the next step without purification.

Its structural characterization data are as follows:

ESI-MS (m/z): 211.0 [M+H]⁺.

### Step 2: Preparation of (2-((methylamino)methyl)-4-nitrophenyl)methanol (INT-3-3)

2-Hydroxymethyl-N-methyl-5-nitrobenzamide (25.0 g, 119 mmol) was dissolved in tetrahydrofuran (500 mL), and cooled to 0° C. Then borane-dimethyl sulfide solution (10 M, 89.3 mL) was added dropwise to the reaction system. After the addition was completed, the reaction system was heated to 70° C. and stirred for 5 hours. The above reaction solution was cooled to 0° C. again, added dropwise with hydrogen chloride-methanol solution (2 M, 100 mL), then heated to 65° C. and stirred for 12 hours. The reaction solution was filtered, and the filtrate was directly concentrated to obtain the crude product of the title compound (44.3 g, crude), which was used directly in the next step without purification.

Its structural characterization data are as follows:

ESI-MS (m/z): 197.0 [M+H]⁺.

### Step 3: Preparation of 1-(2-(((tert-butyldiphenylsilyl)oxy)methyl)-5-nitrophenyl)-N-methylmethylamine (INT-3-4)

(2-((Methylamino)methyl)-4-nitrophenyl)methanol (40.3 g, 205 mmol) was dissolved in dichloromethane (800 mL), cooled to 0° C., imidazole (55.9 g, 822 mmol) and tert-butyldiphenylsilyl chloride (84.0 g, 308 mmol, 78.8 mL) were added, then warmed to 25° C. and stirred for 1 hour. Water (200 mL) was added to the reaction system to quench the reaction and extracted with dichloromethane 3 times (300 mL×3). The organic phases were combined and washed with saturated sodium chloride aqueous solution (200 mL.), dried over anhydrous sodium sulfate, filtered and concentrated to obtain a crude product, which was purified by silica gel column (dichloromethane/methanol=20/1 to 5/1), and concentrated again to obtain the title compound (59.7 g, 137 mmol).

Its structural characterization data are as follows:

ESI-MS (m/z): 435.1 [M+H]⁺.

### Step 4: Preparation of allyl(2-(((tert-butyldiphenylsilyl)oxy)methyl)-5-nitrobenzyl)(methyl)carbamate (INT-3-5)

1-(2-(((Tert-butyldiphenylsilyl)oxy)methyl)-5-nitrophenyl)-N-methylmethylamine (54.9 g, 126 mmol) was dissolved in dichloromethane (550 mL), cooled to 0° C., DIPEA (48.9 g, 379 mmol, 66.0 mL) and allyl chloroformate (30.5 g, 253 mmol, 26.8 mL) were added, warmed to 25° C. and stirred continuously for 1 hour. Water (300 mL) was added to the reaction system to quench the reaction and extracted with dichloromethane 3 times (200 mL×3). The organic phases were combined and washed with saturated sodium chloride aqueous solution (300 mL), dried over anhydrous sodium sulfate, filtered and concentrated to obtain a crude product, which was purified by silica gel column (petroleum ether/ethyl acetate=20/1 to 3/1) and concentrated again to obtain the title compound (65.5 g, 126 mmol).

Its structural characterization data are as follows:

ESI-MS (m/z): 519.1 [M+H]⁺.

### Step 5: Preparation of allyl (5-amino-2-(((tert-butyldiphenylsilyl)oxy)methyl)benzyl)(methyl)carbamate (INT-3)

Allyl(2-(((tert-butyldiphenylsilyl)oxy)methyl)-5-nitrobenzyl)(methyl)carbamate (62.8 g, 121 mmol) was dissolved in a mixed solvent of ethanol (300 mL) and water (300 mL), iron powder (33.8 g, 605 mmol) and ammonium chloride (64.8 g, 1.21 mol) were added, then heated to 80° C. and stirred for 2 hours. The reaction solution was filtered, and the filtrate was extracted with dichloromethane 3 times (100 mL×3). The organic phases were combined and washed with saturated sodium chloride aqueous solution (300 mL), dried over anhydrous sodium sulfate, filtered and concentrated to obtain the crude product of the title compound (49.3 g).

Its structural characterization data are as follows:

ESI-MS (m/z): 511.7 [M+H]⁺.

### Intermediate Preparation Example 3: Preparation of allyl(S)-(5-(2-((((9H-fluoren-9-yl) methoxy)carbonyl)amino)propionamido)-2-((((4-nitrophenoxy)carbonyl)oxy)methyl)benzyl)(me thyl)carbamate (INT-4)

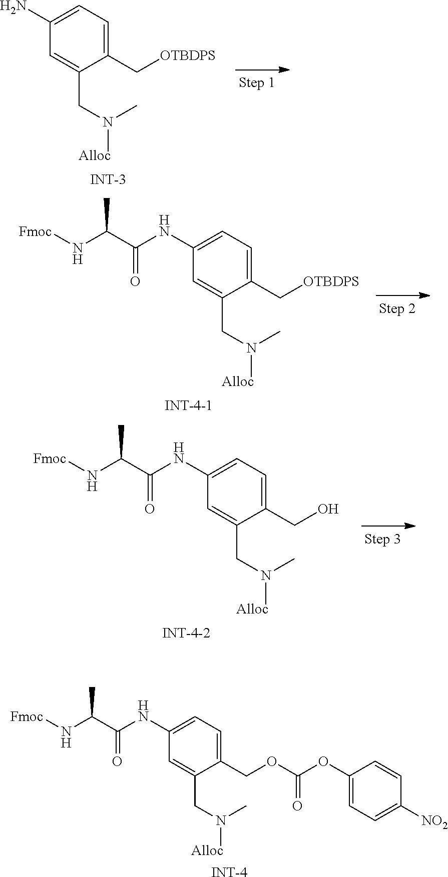

### Step 1: Preparation of allyl(S)-(5-(2-((((9H-fluoren-9-yl)methoxy)carbonyl)amino) propionamido)-2-(((tert-butyldiphenylsilyl)oxy)methyl)benzyl)(methyl)carbamate (INT-4-1)

Allyl (5-amino-2-(((tert-butyldiphenylsilyl)oxy)methyl)benzyl)(methyl)carbamate (150.0 mg, 0.31 mmol) and (((9H-fluoren-9-yl)methoxy)carbonyl)-L-alanine (115.1 mg, 0.37 mmol) were dissolved in DCM 8 mL) and MeOH (2 mL), 2-ethoxy-1-ethoxycarbonyl-1,2-dihydroquinoline (229.7 mg, 0.93 mmol) was added, and stirred at room temperature for 15 hours. The reaction solution was concentrated under reduced pressure to obtain a crude product, which was purified by silica gel column chromatography (ethyl acetato/petroleum ether=0~90%) and concentrated under reduced pressure again to obtain the title compound (194.2 mg, 0.25 mmol)

The structural characterization data are as follows:

MS m/z (ESI): 782.2 [M+H]⁺.

### Step 2: Preparation of(S)-(5-(2-((((9H-fluoren-9-yl)methoxy)carbonyl)amino) propionamido)-2~ (hydroxymethyl)benzyl)(methyl)carbamate (INT-4-2)

Allyl(S)-(5-(2-((((9H-fluoren-9-yl)methoxy)carbonyl)amino)propionamido)-2-(((tert-butyldiphenylsilyl)oxy)methyl)benzyl)(methyl)carbamate (194.2 mg, 0.25 mmol) was dissolved in DMF (5 mL), pyridine hydrofluoride (390.2 mg, 3.93 mmol) was added, and stirred at room temperature for 15 hours. After the reaction was completed, 20 mL of water was added to the reaction solution, extracted with ethyl acetate 3 times (10 mL×3), washed with 10 mL of brine, dried over anhydrous sodium sulfate, and concentrated under reduced pressure to obtain the crude product of the title compound, which was purified by flash column chromatography (C18, water/acetonitrile=2/1) and freeze-dried to obtain the title compound (109.3 mg, 0.21 mmol).

Its structural characterization data are as follows:

MS m/z (ESI): 566.1 [M+Na]⁺.

### Step 3: Preparation of allyl(S)-(5-(2-((((9H-fluoren-9-yl)methoxy)carbonyl)amino) propionamido)-2-((((4-nitrophenoxy)carbonyl)oxy)methyl)benzyl)(methyl)carbamate (INT-4)

(S)-(5-(2-((((9H-Fluoren-9-yl)methoxy)carbonyl)amino)propionamido)-2-(hydroxymethyl)benzyl)(methyl)carbamate (109.3 mg, 0.21 mmol) was dissolved in DMF (5 mL), DIPEA (81.3 mg, 0.63 mmol) was added, followed by the addition of p-nitrophenyl chloroformate (50.8 mg, 0.25 mmol), and stirred at room temperature for 2 hours. After the reaction was completed, the reaction solution was purified by flash column chromatography (C18, water/acetonitrile=2/1) and freeze-dried to obtain the title compound (134.50 mg, 0.19 mmol).

Its structural characterization data are as follows:

MS m/z (ESI): 731.2 [M+Na]⁺.

### Intermediate Preparation Example 4: Preparation of 1-(3,5-bis(2-(methylsulfonyl) pyrimidin-5-yl)phenyl)-1-oxo-5,8,11,14,17-pentaoxa-2-azaeicosan-20-oic acid (INT-5)

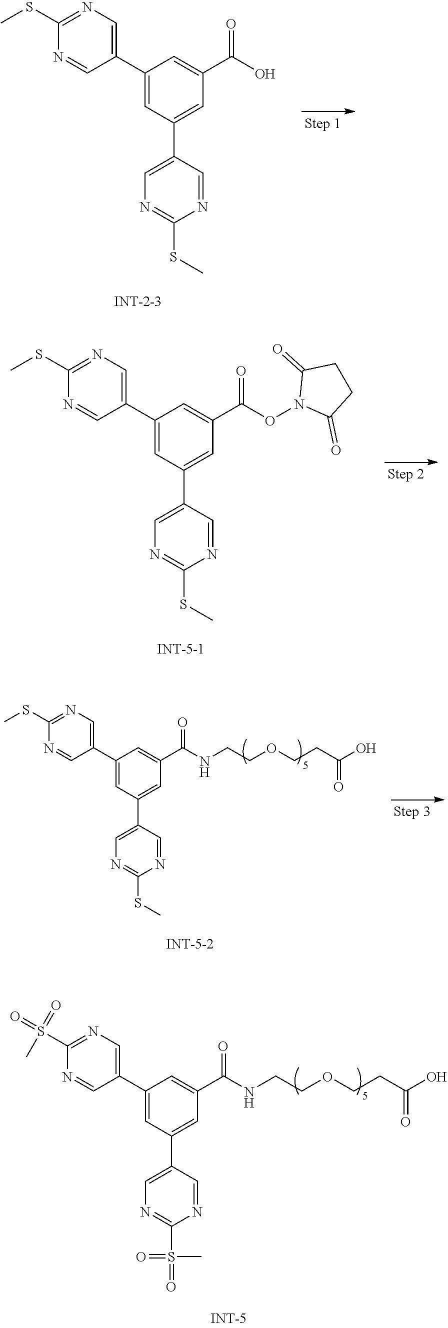

### Step 1: Preparation of 2,5-dioxopyrrolidin-1-yl 3,5-bis(2-(methylthio) pyrimidin-5-yl)benzoate (INT-5-1)

3,5-Bis(2-(methylthio)pyrimidin-5-yl)benzoic acid (0.6 g, 1.62 mmol) was dissolved in THF (15 mL), N-hydroxysuccinimide (278.89 mg, 2.43 mmol) was added, followed by the addition of dicyclohexylcarbodiimide (0.4 g, 1.94 mmol), and stirred at room temperature for 2 hours. After the reaction was completed, the filtrate was collected by suction filtration and concentrated under reduced pressure to obtain the crude product of the title compound (1.5 g, 3.42 mmol), which was directly used in the next reaction without purification.

### Step 2: Preparation of 1-(3,5-bis(2-(methylthio)pyrimidin-5-yl)phenyl)-1-oxo-5,8,11,14,17-pentaoxa-2-azaeicosan-20-oic acid (INT-5-2)

2,5-Dioxopyrrolidin-1-yl 3,5-bis(2-(methylthio)pyrimidin-5-yl)benzoate (331.5 mg, 0.99 mmol) was dissolved in DMF (4 mL), 1-amino-3,6,9,12,15-pentaoxaoctadecan-18-oic acid (0.3 g, 0.67 mmol) and DIPEA (588.24 mg, 4.56 mol) were added, and stirred at room temperature for 2 hours. After the reaction was completed, the reaction solution was purified by flash column chromatography (C18, water/acetonitrile=0.5) to obtain the title compound (315.50 mg, 0.44 mmol).

Its structural characterization data are as follows:

MS m/z (ESI): 662.2 [M+H]⁺

### Step 3: Preparation of 1-(3,5-bis(2-(methylsulfonyl)pyrimidin-5-yl)phenyl)-1-oxo-5,8,11,14,17-pentaoxa-2-azaeicosan-20-oic acid (INT-5)

1-(3,5-Bis(2-(methylthio)pyrimidin-5-yl)phenyl)-1-oxo-5,8,11,14,17-pentaoxa-2-azaeicosan-20-oleic acid (315.50 mg, 0.44 mmol) was dissolved in acetonitrile (3 mL) and water (1.5 mL), sodium periodate (470.50 mg, 2.20 mmol) and ruthenium trichloride monohydrate (9.11 mg, 0.04 mmol) were added, and stirred at room temperature for 0.5 hours. After the reaction was completed, the reaction solution was purified by flash column chromatography (C18, water/acetonitrile=2/1) and freeze-dried to obtain the title compound (115.50 mg, 0.16 mmol).

Its structural characterization data are as follows:

MS m/z (ESI): 726.1 [M+H]⁺

### Intermediate Preparation Example 5: Preparation of (((9H-fluoren-9-yl)methoxy)carbonyl)-D-alanyl-L-proline (INT-6)

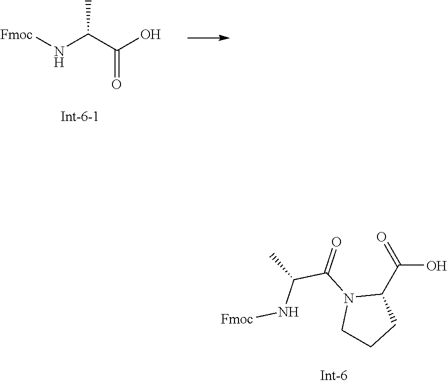

(2R)-2-(9H-Fluoren-9-ylmethoxycarbonylamino)propionic acid (2.00 g, 6.42 mmol), 1-hydroxypyrrolidin-2,5-dione (887.2 mg, 7.71 mmol), and DCC (1.59 g, 7.71 mmol) were added to DMF (30 mL) in sequence, and reacted at 25° C. for 2 hours; L-proline (738.6 mg, 6.42 mmol) was added in one batch, then added dropwise with DIPEA (829.1 mg, 6.42 mmol), and reacted at 25° C. for 4 hours. The reaction solution was poured into water (90 mL), filtered to remove insoluble solids. The filtrate was extracted with ethyl acetate (50 ml+30 ml) to remove impurities. The aqueous phase was adjusted to pH 3-4 with hydrochloric acid and extracted twice with ethyl acetate (100 ml+50 ml). The obtained organic phases were combined, washed with saturated sodium chloride aqueous solution, dried over anhydrous sodium sulfate, filtered and concentrated to obtain the title compound (2.40 g, 4.70 mmol).

Its structural characterization data are as follows:

MS m/z (ESI): 409.2 [M+H]⁺

### Intermediate Preparation Example 6: Preparation of N²-(3,5-bis(2-(methylsulfonyl) pyrimidin-5-yl)benzoyl)-N⁶-(tert-butoxycarbonyl)-L-lysine (INT-8)

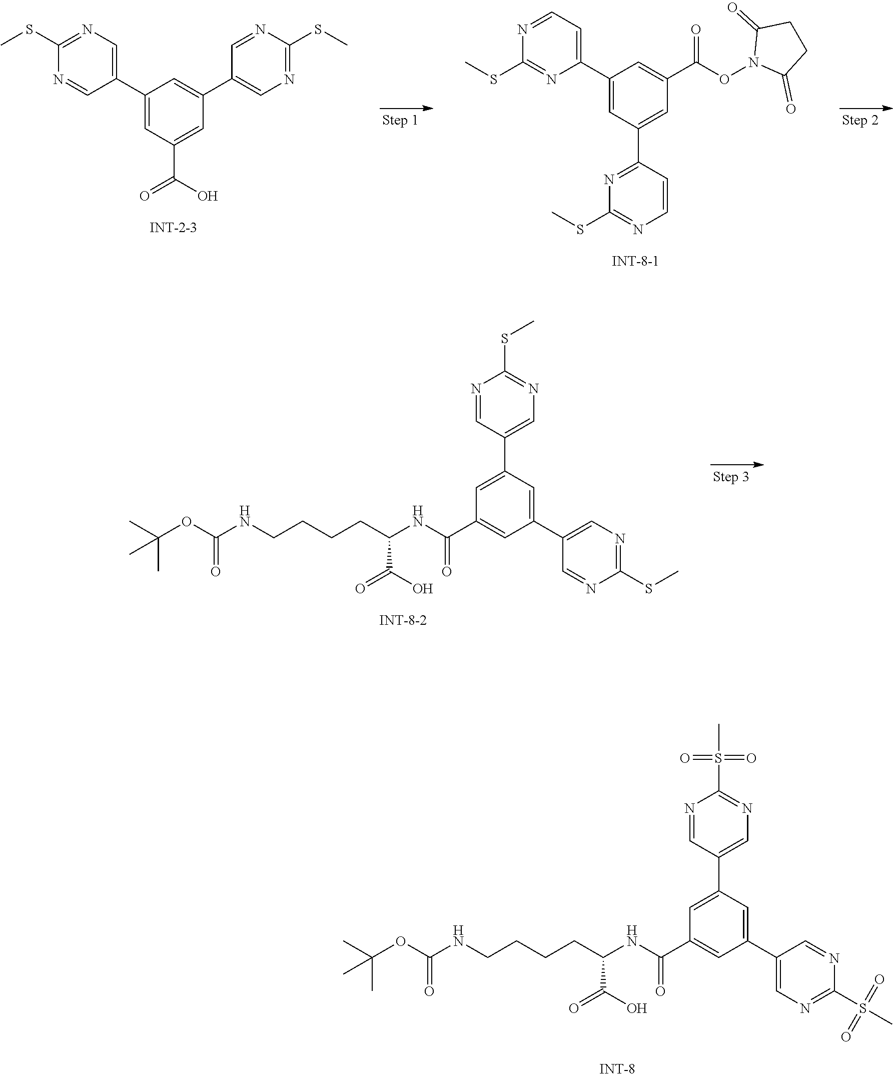

### Step 1: Preparation of 2,5-dioxopyrrolidine-1-yl 3,5-bis(2-(methylthio) pyrimidin-4-yl)benzoate (INT-8-1)

3,5-Bis(2-(methylthio)pyrimidin-4-yl)benzoic acid (5 g, 13.50 mmol) and 1-hydroxypyrrolidin-2,5-dione (1.55 g, 13.50 mmol) were added to a mixed solvent of acetonitrile (75 mL) and DMF (75 mL), followed by an addition of EDCI (5.17 g, 26.99 mmol). The reaction was carried out at 25° C. for 1.5 hours as a pilot test. The reaction solution was filtered. The filter cake was collected, and dried to obtain the title compound (2.41 g, 5.15 mmol).

Its structural characterization data are as follows:

MS m/z (ESI): 468.3 [M+H]⁺

### Step 2: Preparation of N²-(3,5-bis(2-(methylthio)pyrimidin-5-yl)benzoyl)-N⁶-(tert-butoxycarbonyl)-L-lysine (INT-8-2)

N⁶-(Tert-butoxycarbonyl)-L-lysine (895.61 mg, 3.64 mmol) and 2,5-dioxopyrrolidin-1-yl 3,5-bis(2-(methylthio)pyrimidin-4-yl)benzoate (1.7 g, 3.64 mmol) were added to DMSO (50 mL), heated to 60° C. and reacted for 14 hours. The reaction solution was poured into water, precipitating a large amount of white solid. The system was adjusted to pH of about 2 with 1N dilute hydrochloric acid and filtered. The filter cake was collected, and dried to obtain the title compound (2.16 g, 3.61 mmol).

Its structural characterization data are as follows:

MS m/z (ESI): 526.2 [M+H−56]⁺

### Step 3: Preparation of N²-(3,5-bis(2-(methylsulfonyl)pyrimidin-5-yl)benzoyl)-N⁶-(tert-butoxycarbonyl)-L-lysine (INT-8)

N²-(3,5-Bis(2-(methylthio)pyrimidin-5-yl)benzoyl)-N⁶-(tert-butoxycarbonyl)-L-lysine (1.06 g, 1.77 mmol) was dissolved in a mixed solution of water (25 mL) and acetonitrile (50 mL), sodium periodate (2.46 g, 11.51 mmol) and hydrated ruthenium trichloride (36.72 mg, 177.04 μmol) were added, and reacted at 25° C. for 20 minutes. Water was added to the reaction system, adjusted to pH of about 2 with 1N dilute hydrochloric acid, and extracted with EA. Concentration was conducted to obtain a crude product. The crude product was dissolved in acetonitrile, purified by reverse phase column (ACN/H₂O=0-45%, 0.05% formic acid), and freeze-dried to obtain the title compound (825 mg, 1.24 mmol).

Its structural characterization data are as follows:

MS m/z (ESI): 680.2 [M+H₂O]⁺

### Intermediate Preparation Example 7: Preparation of N⁶-(((9H-fluoren-9-yl)methoxy) carbonyl)-N²-(3,5-bis(2-(methylsulfonyl)pyrimidin-5-yl)benzoyl)-L-lysine (INT-9)

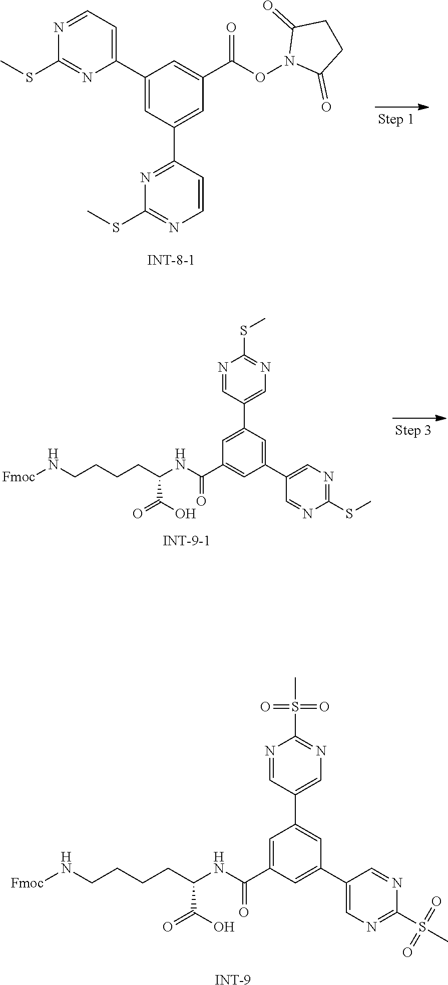

### Step 1: Preparation of N⁶-(((9H-fluoren-9-yl)methoxy)carbonyl)-N²-(3,5-bis(2-(methylthio)pyrimidin-5-yl)benzoyl)-L-lysine (INT-9-1)

N⁶-(((9H-fluoren-9-yl)methoxy)carbonyl)-L-lysine trifluoroacetate (2.8 g, 5.80 mmol) and DIPEA (1.50 g, 11.61 mmol) were added to DMSO (20 mL). The resulting system was reacted under stirring at room temperature for 10 minutes, after which it became clear, followed by an addition of 2,5-dioxypyrrolidin-1-yl 3,5-bis(2-(methylthio)pyrimidin-4-yl)benzoate (2.71 g, 5.80 mmol). After the addition, the system was stirred at room temperature. Solids gradually precipitated during the reaction, and the temperature was raised to 37° C. for further reaction. The reaction solution was purified by reverse phase column (ACN/H₂O=0-45%, 0.05% formic acid) and freeze-dried to obtain the title compound (2.8 g, 3.88 mmol).

Its structural characterization data are as follows:

MS m/z (ESI): 721.9 [M+H]⁺

### Step 2 Preparation of N⁶-(((9H-fluoren-9-yl)methoxy)carbonyl)-N²-(3,5-bis(2-(methylsulfonyl)pyrimidin-5-yl)benzoyl)-L-lysine (INT-9)

N⁶-(((9H-Fluoren-9-yl)methoxy)carbonyl)-N²-(3,5-bis(2-(methylthio)pyrimidin-5-yl)benzoy 1)-L-lysine (580 mg, 804.60 μmol), sodium periodate (1.38 g, 6.44 mmol) and RuCl₃·H₂O (36.28 mg, 160.92 μmol) were added to a mixed solvent of acetonitrile (80 mL) and water (40 mL). After the addition was completed, the reaction was carried out under stirring at room temperature for 40 minutes. Water (150 mL) was added to the reaction solution, extracted with ethyl acetate three times (100 mL×3), and filtered through celite. The liquids were separated. The organic phase was washed once with water (50 mL) and then once with saturated sodium chloride aqueous solution (50 mL), dried over anhydrous sodium sulfate, filtered and concentrated. The residue was dissolved in acetonitrile and water, and freeze-dried to obtain the title compound (590 mg, 676.56 μmol).

Its structural characterization data are as follows:

MS m/z (ESI): 785.9 [M+H]⁺.

### Example 1:4-((31S,34S)-1-(3,5-bis(2-(methylsulfonyl)pyrimidin-5-yl)phenyl)-31-isopropyl-1,29,32-trioxo-34-(3-ureidopropyl)-5,8,11,14,17,20,23,26-octaoxa-2,30,33-triaza-35-a mino)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxoheptan-4-yl)(methyl)amino)-3-methyl-1-oxobutan-2-yl)amino)-3-methyl-1-oxobutan-2-yl)(methyl)carbamate (F-1)

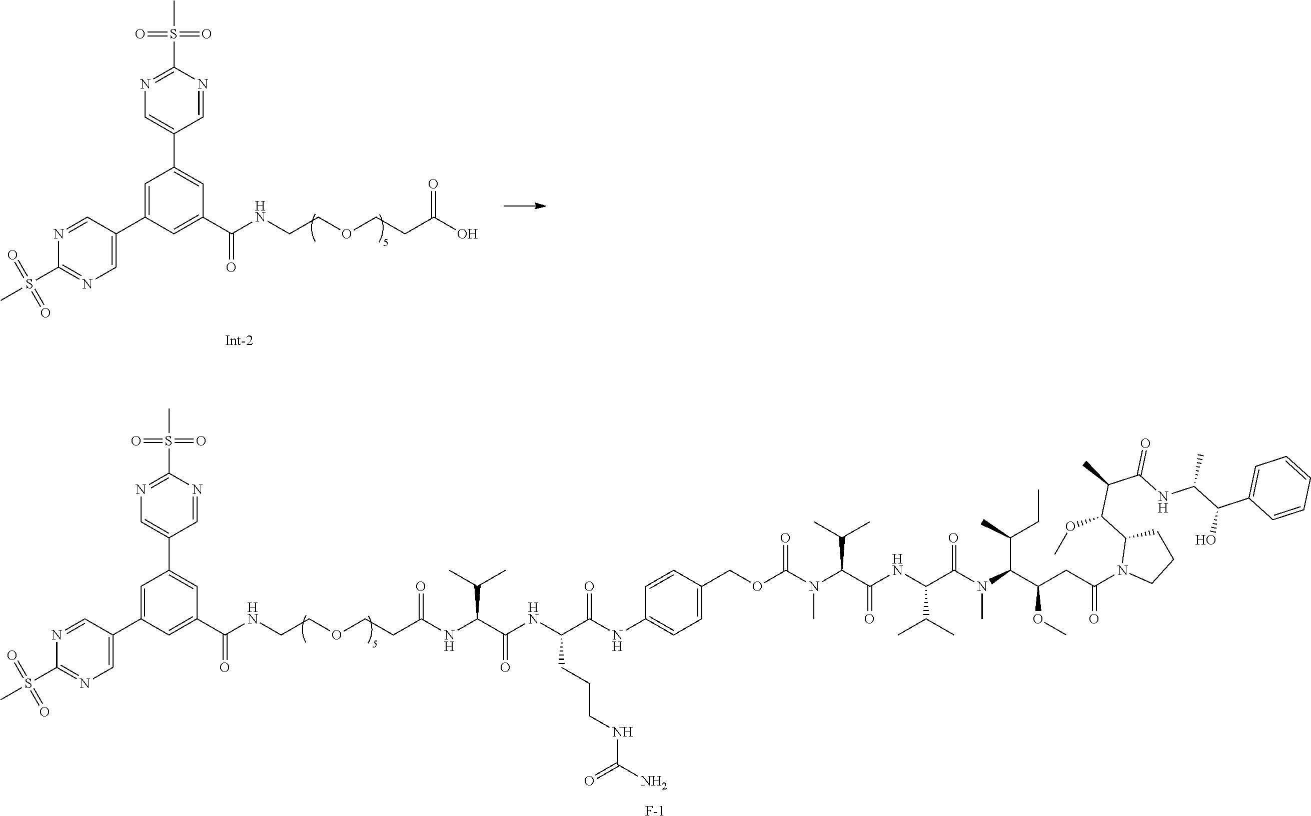

1-(3,5-Bis(2-(methylsulfonyl)pyrimidin-5-yl)phenyl)-1-oxo-5,8,11,14,17,20,23,26-octaoxa-2-azanonacosan-29-oic acid (Int-2, 35 mg, 40.80 μmol) was dissolved in DMF (2 mL), and DIPEA (16 mg), HATU (23 mg) and VC-MMAE (CAS: 644981-35-1; 45 mg, 40.80 μmol) were added in sequence. After the addition was completed, the reaction was carried out at 25° C. for 2 hours. The reaction solution was directly purified by preparative HPLC and freeze-dried to obtain the title compound (31.45 mg, 15.54 μmol).

The structural characterization data are as follows:

MS m/z (ESI): 1963.1 [M+H]⁺

The preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 40 | 60 | 28 |
| 8.00 | 40 | 60 | 28 |
| 24.00 | 70 | 30 | 28 |

### Example 2: Pentafluorophenyl 1-(N-((6S,9S)-14-amino-9-((4-((5S,8S,11S, 12R)-11-((S)-sec-butyl)-12-(2-((S)-2-((1R,2R)-3-(((1S,2R)-1-hydroxy-1-phenylpropan-2-yl)amino)-1-met hoxy-2-methyl-3-oxopropyl)pyrrolidin-1-yl)-2-oxoethyl)-5,8-diisopropyl-4,10-dimethyl-3,6,9-tri oxo-2,13-dioxo-4,7,10-triazatetradecyl)phenyl)carbamoyl)-6-isopropyl-4,7,14-trioxo-2-oxo-5,8,1 3-triazatetradecyl)aminosulfonyl)piperidine-4-carboxylate (L-1)

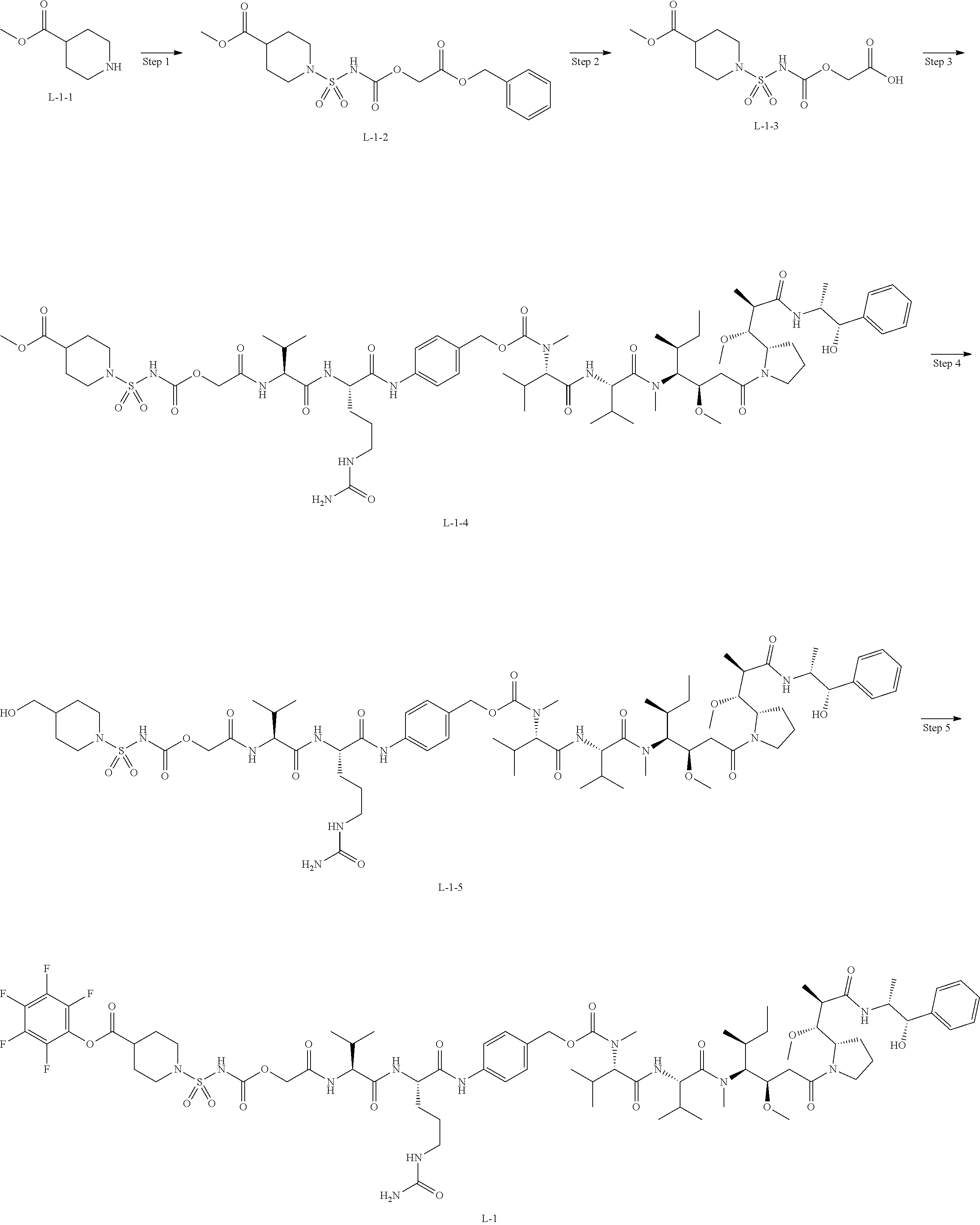

### Step 1

Chlorosulfonyl isocyanate (500 mg, 3.53 mmol, 307 μL) was dissolved in dichloromethane (10.0 mL) at 0° C., benzyl glycolate (533 mg, 3.21 mmol, 455 μL) was added, and stirred continuously for 1 hour. Then, a solution of methyl 4-piperidinecarboxylate L-1-1 (459 mg, 3.21 mmol) and triethylamine (974 mg, 9.63 mmol, 1.34 mL) in dichloromethane (5.00 mL) was added to the above reaction solution, heated to 25° C. and stirred continuously for 1 hour. Water (100 mL) was added to the reaction solution and extracted with dichloromethane 3 times (50.0 mL×3). The combined organic phases were washed with cooled 1N dilute hydrochloric acid (20.0 mL), dried over anhydrous sodium sulfate, filtered and concentrated to obtain the crude product L-1-2 (1.30 g, 3.14 mmol), which was used directly in the next step without purification.

The structural characterization data are as follows:

ESI-MS (m/z): 414.9 [M+H]⁺

### Step 2

Under nitrogen atmosphere, 10% Pd/C (0.70 g) was added to a solution of L-1-2 (700 mg, 1.69 mmol) in methanol (20.0 mL). The reaction system was subjected to hydrogen replacement three times and reacted at 25° C. for 3 hours (15 PSI). The reaction solution was filtered. The filter cake was rinsed with methanol three times (100 mL×3). The filtrate was concentrated to obtain the crude product of L-1-3 (590 mg).

### Step 3

VC-MMAE (CAS: 644981-35-1; 60 mg, 53.4 μmol) and L-1-3 (17.3 mg, 53.4 μmol) were dissolved in DMF (10.0 mL), HOBt (36.0 mg, 267 μmol), DIPEA (34.5 mg, 267 μmol, 46.5 μL) and EDCI (30.7 mg, 160 μmol) were added in sequence, and reacted at 25° C. for 2 hours. Water (20.0 mL) was added to the reaction solution and extracted with dichloromethane 3 times (20.0 mL×3). The organic phases were combined, dried over anhydrous sodium sulfate, filtered and concentrated to obtain the crude product of L-1-4 (60.0 mg), which was used directly in the next step without purification.

The structural characterization data are as follows:

ESI-MS (m/z): 715.4 [M/2+H]⁺

### Step 4

L-1-4 (50.0 mg, 34.9 μmol) was dissolved in tetrahydrofuran (2.50 mL), water (1.00 mL) and MeOH (1.00 mL), lithium hydroxide monohydrate (2.94 mg, 69.9 μmol) was added, and stirred at 30° C. for 1 hour. The reaction solution was adjusted to pH 6 with 1N dilute hydrochloric acid aqueous solution, and then directly concentrated to obtain crude L-1-5 (50.0 mg), which was used directly in the next step without purification.

The structural characterization data are as follows:

ESI-MS (m/z): 1416.6 [M+H]⁺

### Step 5

L-1-5 (50.0 mg, 35.3 μmol) and pentafluorophenol (13.0 mg, 70.6 μmol) were dissolved in DCM (10.0 mL), EDCI (30.4 mg, 158 μmol) was added, and then stirred at 30° C. for 2 hours. The reaction solution was concentrated at 25° C. to obtain a crude product, which was purified by HPLC and freeze-dried to obtain L-1 (4.70 mg, 2.86 μmol).

The structural characterization data are as follows:

ESI-MS (m/z): 1582.1 [M+H]⁺

Separation and purification methods are as follows:
Chromatographic column: Phenomenex luna C18 (10 μm*25 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 56 | 44 | 30 |
| 10.00 | 86 | 14 | 30 |

### Example 3: Pentafluorophenyl 1-(N-((2-(((S)-1-(((R)-1-((S)-2-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-met hoxy-2-methyl-3-oxypropyl)pyrrolidin-1-yl)-2-oxoethyl)-5,8-diisopropyl-4,10-dimethyl-3,6,9-tri oxo-2,13-dioxa-4,7,10-triazatetradecyl)phenyl)carbamoyl)pyrrolidin-1-yl)-1-oxopropan-2-yl) ami no)-3-hydroxy-1-oxopropan-2-yl)amino)-2-oxoethoxy)carbonyl)sulfamoyl)piperidine-4-carboxy late (L-4)

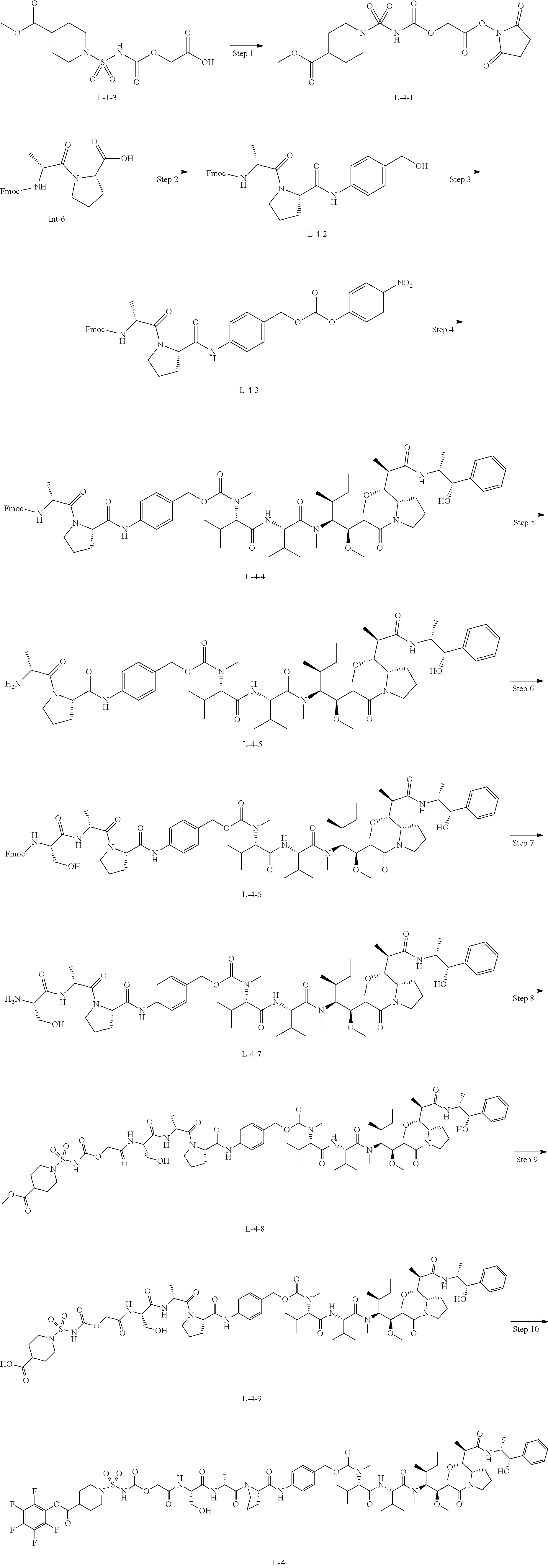

### Step 1: Preparation of methyl 1-(N-((2-((2,5-dioxopyrrolidin-1-yl)oxy)-2-oxoethoxy) carbonyl) sulfamoyl) piperidine-4-carboxylate (L-4-1)

2-((((4-(Methoxycarbonyl) piperidin-1-yl) sulfonyl)carbamoyl)oxy) acetic acid (50 mg, 0.169 mmol), HOSU (23.3 mg, 0.202 mmol), and DCC (41.8 mg, 0.202 mmol) were added to DMF (1 mL), and reacted at 25° C. for 2 hours; the reaction solution was poured into ethyl acetate (20 ml) and water (5 ml), stirred and filtered to separate the organic phase. The organic phase was washed with brine and concentrated to obtain the crude product of the title compound (35 mg, 0.066 mmol), which was used directly in the next step without purification.

### Step 2: Preparation of (9H-fluoren-9-yl)methyl ((R)-1-((S)-2-((4-(hydroxymethyl)phenyl) carbamoyl) pyrrolidin-1-yl)-1-oxopropan-2-yl)carbamate (L-4-2)

(((9H-Fluoren-9-yl)methoxy)carbonyl)-D-alanyl-L-proline (Int-6, 400.0 mg, 0.979 mmol) and 4-aminobenzyl alcohol (144.7 mg, 1.18 mmol) were dissolved in a mixed solvent of dichloromethane (4 mL) and MeOH (2 mL), EEDQ (484.4 mg, 1.96 mmol) was added, heated to 40° C. and reacted for 4 hours; the reaction solution was concentrated to obtain a crude product, which was purified by column chromatography (first with EA/PE=40~60%, then with MeOH/DCM=5~15%) to obtain the title compound (225 mg, 0.394 mmol).

Its structural characterization data are as follows:

MS m/z (ESI): 514 [M+H]⁺

### Step 3: (9H-Fluoren-9-yl)methyl ((R)-1-((S)-2-((4-(((4-nitrophenoxy)carbonyl)oxy)methyl)phenyl) carbamoyl) pyrrolidin-1-yl)-1-oxopropan-2-yl)carbamate (L-4-3)

(9H-Fluoren-9-yl)methyl ((R)-1-((S)-2-((4~ (hydroxymethyl)phenyl)carbamoyl) pyrrolidin-1-yl)-1-oxopropan-2-yl)carbamate (225 mg, 0.438 mmol) and bis(4-nitrophenyl)carbonate (266.6 mg, 0.876 mmol) were dissolved in DMF (3 mL), DIPEA (84.9 mg, 0.657 mmol) was added, and reacted at 25° C. for 2 hours. The reaction solution was added to ethyl acetate (20 ml) and water (10 ml). The organic phase was separated. The aqueous phase was then extracted with ethyl acetate (10 ml) once more. The organic phases were combined, was washed with brine, and concentrated to obtain a crude product. The crude product was purified by column chromatography (MeOH/DCM=5~10%) and concentrated again to obtain the title compound (190 mg, 0.280 mmol).

Its structural characterization data are as follows:

MS m/z (ESI): 701.3 [M+Na]⁺

### Step 4: Preparation of 4-((S)-1-((((9H-fluoren-9-yl)methoxy)carbonyl)-D-propylamino) pyrrolidine-2-carboxamido)benzyl ((S)-1-(((S)-1-(((3R,4S,5 S)-1-((S)-2-((1R,2R)-3-(((1S,2R-1-hydroxy-1-phenylpropan-2-yl)amino)-1-methoxy-2-methyl-3-oxopropyl) pyrrolidin-1-yl)-3-meth oxy-S-methyl-1-oxoheptan-4-yl)(methyl)amino)-3-methyl-1-oxobutan-2-yl)amino)-3-methyl-1-0 xobutan-2-yl)(methyl)carbamate (L-4-4)

MMAE (CAS: 474645-27-7; 150 mg, 0.209 mmol) and (9H-fluoren-9-yl)methyl ((R)-1-((S)-2-((4-(((4-nitrophenoxy)carbonyl)oxy)methyl)phenyl)carbamoyl) pyrrolidin-1-yl)-1-0 xopropan-2-yl)carbamate (170.2 mg, 0.251 mmol) were dissolved in DMF (3 mL), then HOBT (5.65 mg, 0.042 mmol) and DIPEA (27.00 mg, 0.209 mmol) were added, and reacted at 25° C. for 2 hours. The reaction solution was poured into ethyl acetate (20 ml) and water (10 ml). The organic phase was separated. The aqueous phase was extracted once with ethyl acetate (10 ml). The organic phases were combined, washed with brine, and concentrated to obtain a crude product, which was purified by column chromatography (MeOH/DCM=5~10%) and concentrated again to obtain the title compound (60 mg, 0.048 mmol).

Its structural characterization data are as follows:

MS m/z (ESI): 1257.8 [M+H]⁺

### Step 5: Preparation of 4-((S)-1-(D-propylamino)pyrrolidin-2-carboxamido)benzyl ((S)-1-(((S)-1-(((3R,4S,5S)-1-((S)-2-((1R,2R)-3-(((1S,2R-1-hydroxy-1-phenylpropan-2-yl)amino)-1-met hoxy-2-methyl-3-oxopropyl) pyrrolidin-1-yl)-3-methoxy-5-methyl-1-oxoheptan-4-yl)(methyl) am ino)-3-methyl-1-oxobutan-2-yl)amino)-3-methyl-1-oxobutan-2-yl)(methyl)carbamate (L-4-5)

4-((S)-1-((((9H-fluoren-9-yl)methoxy)carbonyl)-D-propylamino)pyrrolidin-2-carboxamido)benzyl ((S)-1-(((S)-1-(((3R,4S,5S)-1-((S)-2-((1R,2R)-3-(((1S,2R-1-hydroxy-1-phenylpropan-2-yl) amino)-1-methoxy-2-methyl-3-oxopropyl) pyrrolidin-1-yl)-3-methoxy-5-methyl-1-oxoheptan-4-y l)(methyl)amino)-3-methyl-1-oxobutan-2-yl)amino)-3-methyl-1-oxobutan-2-yl)(methyl)carbama the (60 mg, 0.048 mmol) was dissolved in DMF (1 mL), diethylamine (17.4 mg, 0.238 mmol) was added, and reacted at 25° C. for 1 hour. The reaction solution was purified by preparative HPLC and freeze-dried to obtain the title compound (36 mg, 0.032 mmol, formate).

The structural characterization data are as follows:

MS m/z (ESI): 1035.7 [M+H]⁺

The separation and purification method is as follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 28 |
| 2.00 | 10 | 90 | 28 |
| 18.00 | 90 | 10 | 28 |

### Step 6: Preparation of 4-((S)-1-(((9H-fluoren-9-yl)methoxy)carbonyl)-L-serine-D-propylamino)pyrrolidine-2-carboxamido)benzyl ((S)-1-(((S)-1-(((3R,4S,5 S)-1-((S)-2-((1R,2R)-3-(((1S,2R-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (L-4-6)

4-((S)-1-(D-propylamino)pyrrolidine-2-carboxamido)benzyl ((S)-1-(((S)-1-(((3R,4S,5S)-1-((S)-2-((1R,2R)-3-(((1S,2R-1-hydroxy-1-phenylpropan-2-yl)amino)-1-methoxy-2-methyl-3-oxop ropyl) pyrrolidin-1-yl)-3-methoxy-5-methyl-1-oxoheptan-4-yl)(methyl)amino)-3-methyl-1-oxobu tan-2-yl)amino)-3-methyl-1-oxobutan-2-yl)(methyl)carbamate (36 mg, 0.032 mmol, formate) and (((9H-fluoren-9-yl)methoxy)carbonyl)-L-serine (13.1 mg, 0.040 mmol) were dissolved in DMF (1 mL), HATU (25.32 mg, 0.066 mmol) and DIPEA (12.9 mg, 0.10 mmol) were added, and reacted at 25° C. for 1 hour. The reaction solution was purified by preparative HPLC and freeze-dried to obtain the title compound (19 mg, 0.013 mmol).

The structural characterization data are as follows:

MS m/z (ESI): 1344.8 [M+H]⁺

The preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 30 |
| 2.00 | 40 | 60 | 30 |
| 15.00 | 90 | 10 | 30 |
| 18.00 | 90 | 10 | 30 |

### Step 7: Preparation of 4-((S)-1-(L-serine-D-propylamino)pyrrolidine-2-carboxamido)benzyl ((S)-1-(((S)-1-(((3R,4S,5S)-1-((S)-2-((1R,2R)-3-(((1S,2R-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(met hyl)amino)-3-methyl-1-oxobutan-2-yl)amino)-3-methyl-1-oxobutan-2-yl)(methyl)carbamate (L-4-7)

4-((S)-1-(((9H-Fluoren-9-yl)methoxy)carbonyl)-L-serine-D-propylamino)pyrrolidine-2-carb oxamido)benzyl ((S)-1-(((S)-1-(((3R,4S,5S)-1-((S)-2-((1R,2R)-3-(((1S,2R-1-hydroxy-1-phenylpropan-2-yl)amino)-1-methoxy-2-methyl-3-oxopropyl) pyrrolidin-1-yl)-3-methoxy-5-meth yl-1-oxoheptan-4-yl)(methyl)amino)-3-methyl-1-oxobutan-2-yl)amino)-3-methyl-1-oxobutan-2-yl)(methyl)carbamate (19 mg, 0.013 mmol) was dissolved in DMF (1 mL), diethylamine (5.2 mg, 0.071 mmol) was added, and reacted at 25° C. for 1 hour. The reaction solution was purified by preparative HPLC and freeze-dried to obtain the title compound (14 mg, 0.011 mmol, formate).

Its structural characterization data are as follows:

MS m/z (ESI): 1122.7 [M+H]⁺

Its preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 10 | 90 | 24 |
| 2.00 | 10 | 90 | 24 |
| 20.00 | 90 | 10 | 24 |

### Step 8: Preparation of 1-(N-((2-(((S)-1-(((R)-1-((S)-2-((4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl) pyrrolidin-1-yl)-1-oxopropan-2-yl)amino)-3-hydroxy-1-oxopropan-2-yl)amino)-2-oxoethoxy)carbonyl) sulfamoyl) piperidine-4-carboxylate (L-4-8)

4-((S)-1-(Z-Serine-D-propylamino)pyrrolidine-2-carboxamido)benzyl ((S)-1-(((S)-1-(((3R,4S,5S)-1-((S)-2-((1R,2R)-3-(((1S,2R-1-hydroxy-1-phenylpropan-2-yl)amino)-1-methoxy-2-meth yl-3-oxopropyl) pyrrolidin-1-yl)-3-methoxy-5-methyl-1-oxoheptan-4-yl)(methyl)amino)-3-methy 1-1-oxobutan-2-yl)amino)-3-methyl-1-oxobutan-2-yl)(methyl)carbamate (8.0 mg, 0.0071 mmol), compound L-4-1 (6.01 mg, 0.014 mmol) and DIPEA (4.6 mg, 0.036 mmol) were added to DMF (0.5 mL), and reacted at 25° C. for 1 hour. The reaction solution was purified by preparative HPLC and freeze-dried to obtain the title compound (8.0 mg, 0.0056 mmol).

Its structural characterization data are as follows:

MS m/z (ESI): 1428 [M+H]⁺

The preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 30 |
| 4.00 | 30 | 70 | 30 |
| 24.00 | 90 | 10 | 30 |

### Step 9: Preparation of 1-(N-((2-(((S)-1-(((R)-1-((S)-2-((4-((5S,8S,11S, 12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxypropyl) pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl) pyrrolidin-1-yl)-1-oxopropan-2-yl)amino)-3-hydroxy-1-oxopropan-2-yl)amino)-2-oxoethoxy)carbonyl) sulfamoyl) piperidine-4-carboxylic acid (L-4-9)

Methyl 1-(N-((2-(((S)-1-(((R)-1-((S)-2-((4-((5S,8S,11S, 12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxypropyl) pyrrolidin-1-yl)-2-oxoethyl)-5,8-diisopropyl-4,10-dimethyl-3,6,9-trioxo-2,13-dioxa-4,7,10-triaza tetradecyl)phenyl)carbamoyl) pyrrolidin-1-yl)-1-oxopropan-2-yl)amino)-3-hydroxy-1-oxopropan-2-yl)amino)-2-oxoethoxy)carbonyl) sulfamoyl) piperidine-4-carboxylate (8.0 mg, 0.0056 mmol) was dissolved in a mixed solvent of MeOH (1 mL) and H₂O (0.5 mL), LiOH·H₂O (2.4 mg, 0.056 mmol) was added, and reacted at 25° C. for 2 hours. The reaction solution was purified by preparative HPLC and freeze-dried to obtain the title compound (5.0 mg, 0.0035 mmol).

Its structural characterization data are as follows:

MS m/z (ESI): 1414 [M+H]⁺

Its preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 30 | 70 | 28 |
| 4.00 | 30 | 70 | 28 |
| 24.00 | 90 | 10 | 28 |

### Step 10: Preparation of pentafluorophenyl 1-(N-((2-(((S)-1-(((R)-1-((S)-2-((4-((5S,8S,11S,12R)-11-((S)-sec-butyl)-12-(2-((S)-2-((1R,2R)-3-(((1S,2R)-1-hydroxy-1-phenylpropan-2-yl)amin o)-1-methoxy-2-methyl-3-oxypropyl) pyrrolidin-1-yl)-2-oxoethyl)-5,8-diisopropyl-4,10-dimethyl-3,6,9-trioxo-2,13-dioxa-4,7,10-triazatetradecyl)phenyl)carbamoyl) pyrrolidin-1-yl)-1-oxopropyl-2-yl)amino)-3-hydroxy-1-oxopropyl-2-yl)amino)-2-oxoethoxy)carbonyl) sulfamoyl) piperidine-4-carboxylic acid (L-4)

1-(N-((2-(((S)-1-(((R)-1-((S)-2-((4-((5S,8S,11S, 12R)-11-((S)-Sec-butyl)-12-(2-((S)-2-((1R,2R)-3-(((1S,2R)-1-hydroxy-1-phenylpropan-2-yl)amino)-1-methoxy-2-methyl-3-oxypropyl) pyrrol idin-1-yl)-2-oxoethyl)-5,8-diisopropyl-4,10-dimethyl-3,6,9-trioxo-2,13-dioxa-4,7,10-triazatetrad ecyl)phenyl)carbamoyl) pyrrolidin-1-yl)-1-oxopropan-2-yl)amino)-3-hydroxy-1-oxopropan-2-yl) amino)-2-oxoethoxy)carbonyl) sulfamoyl) piperidine-4-carboxylic acid (5.0 mg, 0.0035 mmol) and 2,3,4,5,6-pentafluorophenol (2.0 mg, 0.011 mmol) were dissolved in DMF (0.5 mL), then EDCI (3.4 mg, 0.018 mmol) was added, and reacted at 25° C. for 1 hour. The reaction solution was purified by preparative HPLC and freeze-dried to obtain the title compound (2.1 mg, 0.0013 mmol).

Its structural characterization data are as follows:

MS m/z (ESI): 1580 [M+H]⁺

Its preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 40 | 70 | 32 |
| 4.00 | 40 | 70 | 32 |
| 24.00 | 90 | 10 | 32 |

### Example 4: Preparation of 2,2′,2″-(10-(2-((5-((S) 2-((S)-2-(3,5-bis(2-(methylsulfonyl) pyrimidin-5-yl)benzamido)-3-methylbutyramido) propionamido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-0 xopropyl) pyrrolidin-1-yl)-2-oxoethyl)-5,8-diisopropyl-4,10-dimethyl-3,6,9-trioxo-2,13-dioxa-4,7, 10-triazatetradecyl)benzyl)(methyl)amino)-2-oxoethyl)-1,4,7,10-tetraazacyclododecan-1,4,7-triy l)triacetic acid (F-13)

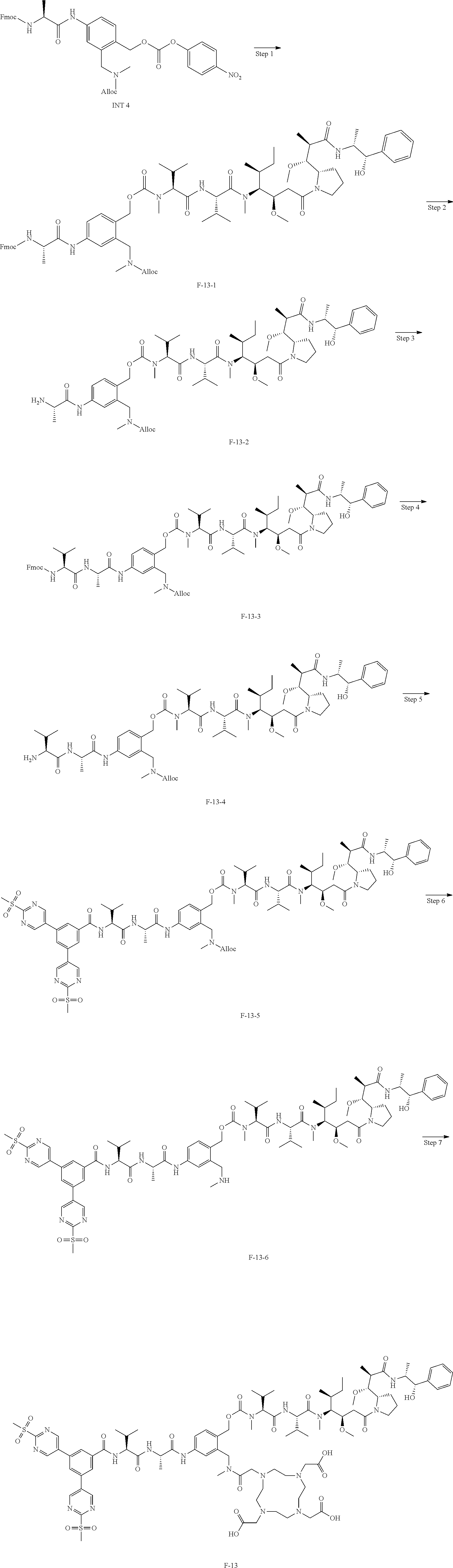

### Step 1: Preparation of 4-((S)-2-((((9H-fluoren-9-yl)methoxy)carbonyl)amino) propionamido)-2-((((allyloxy)carbonyl)(methyl)amino)methyl)benzyl (1-((1-(((3R,5S)-1-((S)-2-((1R,2R)-3-(((1S,2R)-1-hydroxy-1-phenylpropan-2-yl))amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (F-13-1)

Allyl(S)-(5-(2-((((9H-fluoren-9-yl)methoxy)carbonyl)amino)propionamido)-2-((((4-nitrophenoxy)carbonyl)oxy)methyl)benzyl)(methyl)carbamate (1.0 g, 1.41 mmol) and MMAE (CAS: 474645-27-7; 1.52 g, 2.12 mmol) were dissolved in DMF (6 mL), DIPEA (820.6 mg) and 1-hydroxybenzotriazole (190.6 mg, 1.41 mmol) were added. After the addition, the resulting mixture was reacted at 25° C. for 2 hours. After the reaction was completed, the reaction solution was purified by flash column chromatography (C18, water/acetonitrile=0.6) and freeze-dried to obtain the title compound (1.18 g, 0.92 mmol).

Its structural characterization data are as follows:

MS m/z (ESI): 1287.3 [M+H]⁺

### Step 2 Preparation of 2-((((allyloxy)carbonyl)(methyl)amino)methyl)-4-((S)-2-aminopropionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)amino)-3-methyl-1-oxobutan-2-yl)(methyl)carbamate (F-13-2)

4-((S)-2-((((9H-Fluoren-9-yl)methoxy)carbonyl)amino)propionamido)-2-((((allyloxy)carbo nyl)(methyl)amino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-met hyl-1-oxohept-4-yl)(methyl)amino)-3-methyl-1-oxobutan-2-yl)amino)-3-methyl-1-oxobutan-2-yl)(methyl)carbamate (1.18 g, 91.65 μmol) was dissolved in DMF (10 mL), diethylamine (201.6 mg) was added. After the addition, the resulting mixture was reacted at 25° C. for 0.5 hours. After the reaction was completed, the reaction solution was evaporated under reduced pressure to remove solvent, a mixed solution of ethyl acetate/petroleum ether (100 mL, ethyl acetate/petroleum ether=1/3) was added, stirred for 2 hours, filtered and dried to obtain the title compound (878.5 mg, 0.83 mmol).

Its structural characterization data are as follows:

MS m/z (ESI): 1065.3 [M+H]⁺

### Step 3: Preparation of 4-((S)-2-((S)-2-((((9H-fluoren-9-yl)methoxy)carbonyl)amino)-3-methylbutyramido) propionamido)-2-((((allyloxy)carbonyl)(methyl)amino)methyl)benzyl (1-((1-(((3R,5S)-1-((S)-2-((1R,2R))-3-(((1S,2R)-1-hydroxy-1-phenylpropan-2-yl)amino)-1-metho xy-2-methyl-3-oxypropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-13-3)

2-((((Allyloxy)carbonyl)(methyl)amino)methyl)-4-((S)-2-aminopropionamido)benzyl (1-((1-(((3R,5S)-1-((S)-2-((1R,2R)-3-(((1S,2R)-1-hydroxy-1-phenylpropan-2-yl)amino)-1-methox y-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (878.5 mg, 82.5 μmol) and (((9H-fluoren-9-yl)methoxy)carbonyl)-L-valine (308.1 mg, 90.75 μmol) were dissolved in DMF (5 mL), DIPEA (320 mg) and HATU (470 mg) were added in sequence. After the addition, the resulting mixture was reacted at 25° C. for 2 hours. The reaction solution was purified by flash column chromatography (C18, water/acetonitrile=0.7) and freeze-dried to obtain the title compound (800.2 mg, 0.58 mmol).

Its structural characterization data are as follows:

MS m/z (ESI): 1386.9 [M+H]⁺

### Step 4: Preparation of 2-((((allyloxy)carbonyl)(methyl)amino)methyl)-4-((S)-2-((S)-2-amino-3-methylbutyramido) propionamido)benzyl (1-((1-(((3R,5S)-1-(S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methy 1-1-oxobutan-2-yl)(methyl)carbamate (F-13-4)

4-((S)-2-((S)-2-((((9H-Fluoren-9-yl)methoxy)carbonyl)amino)-3-methylbutyramido) propion amido)-2-((((allyloxy)carbonyl)(methyl)amino)methyl)benzyl 2R))-3-(((1S,2R)-1-hydroxy-1-phenylpropan-2-yl)amino)-1-methoxy-2-methyl-3-oxopropyl) pyrr olidin-1-yl)-3-methoxy-5-methyl-1-oxohept-4-yl)(methyl)amino)-3-methyl-1-oxobutan-2-yl) ami no)-3-methyl-1-oxobutan-2-yl)(methyl)carbamate (800.2 mg, 57.7 μmol) was dissolved in DMF (5 mL), diethylamine (126.6 mg) was added. After the addition, the resulting mixture was reacted at 25° C. for 0.5 hours. After the reaction was completed, the reaction solution was evaporated under reduced pressure to remove solvent, a mixed solution of ethyl acetate/petroleum ether (60 mL, ethyl acetate/petroleum ether=1/3) was added, stirred for 2 hours, filtered and dried to obtain the title compound (604.5 mg, 0.52 mmol).

Its structural characterization data are as follows:

MS m/z (ESI): 1164.7 [M+H]⁺

### Step 5: Preparation of 2-((((allyloxy)carbonyl)(methyl)amino)methyl)-4-((S)-2-((S)-2-(3,5-bis(2-(methylsulfonyl)pyrimidin-5-yl)benzamido)-3-methylbutyramido) propionamido)benz yl(1-((1-(((3R,5S)-1-((S)-2-((1R,2R)-3-(((1S,2R)-1-hydroxy-1-phenylpropan-2-yl)amino)-1-methoxyl-2-methyl-3-oxopropyl) pyrrolidin-1-yl)-3-methoxy-5-methyl-1-oxohept-4-yl)(methyl) a mino)-3-methyl-1-oxobutan-2-yl)amino)-3-methyl-1-oxobutan-2-yl)(methyl)carbamate (F-13-5)

2-((((Allyloxy)carbonyl)(methyl)amino)methyl)-4-((S)-2-((S)-2-amino-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxyl-2-methyl-3-oxopropyl) pyrrolidin-1-yl)-3-methoxy-5-met hyl-1-oxohept-4-yl)(methyl)amino)-3-methyl-1-oxobutan-2-yl)amino)-3-methyl-1-oxobutan-2-yl)(methyl)carbamate (100 mg, 85.9 μmol) was dissolved in DMF (3 mL), DIPEA (35 mg) and 2,5-dioxopyrrolidin-1-yl 3,5-bis(2-(methylsulfonyl)pyrimidin-5-yl)benzoate (54.7 mg, 103.1 μmol) were added. After the addition, the resulting mixture was reacted at 25° C. for 1 hour. After the reaction was completed, the reaction solution was purified by flash column chromatography (C18, water/acetonitrile=0.6) and freeze-dried to obtain the title compound (90.2 mg, 56.9 μmol).

Its structural characterization data are as follows:

MS m/z (ESI): 1580.8 [M+H]⁺

### Step 6: Preparation of 4-((S)-2-((S)-2-(3,5-bis(2-(methylsulfonyl)pyrimidin-5-yl)benzamido)-3-methylbutyramido) propionamido)-2-((methylamino)methyl)benzyl (1-((1-(((3R,5S)-1-((S)-2-((1R,2R)-3-(((1S,2R)-1-hydroxy-1-phenylpropan-2-yl)amino)-1-methox y-2-methyl-3-oxopropyl) pyrrolidin-1-yl)-3-methoxy-5-methyl-1-oxoheptan-4-yl)amino)-3-meth yl-1-oxobutan-2-yl)amino)-3-methyl-1-oxobutan-2-yl)(methyl)carbamate (F-13-6)

2-((((Allyloxy)carbonyl)(methyl)amino)methyl)-4-((S)-2-((S)-2-(3,5-bis(2-(methylsulfonyl) pyrimidin-5-yl)benzamido)-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxo propyl) pyrrolidin-1-yl)-3-methoxy-5-methyl-1-oxoheptan-4-yl)amino)-3-methyl-1-oxobutan-2-y 1)amino)-3-methyl-1-oxobutan-2-yl)(methyl)carbamate (90.2 mg, 56.9 μmol) was dissolved in DMF (2 mL), tetrakis(triphenylphosphine) palladium (13.2 mg, 11.4 μmol) was added, then 35 μL of formic acid and 70 μL of N-methylmorpholine were added, and stirred at room temperature for 1 hour under nitrogen protection. After the reaction was completed, the reaction solution was purified by flash column chromatography (C18, water/acetonitrile=0.6) and freeze-dried to obtain the title compound (69.5 mg, 46.4 μmol).

Its structural characterization data are as follows:

MS m/z (ESI): 1496.8 [M+H]⁺

### Step 7: Preparation of 2,2′,2″-(10-(2-((5-((S)-2-((S)-2-(3,5-bis(2-(methylsulfonyl) pyrimidin-5-yl)benzamido)-3-methylbutyramido) propionamido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-0 xopropyl) pyrrolidin-1-yl)-2-oxoethyl)-5,8-diisopropyl-4,10-dimethyl-3,6,9-trioxo-2,13-dioxa-4,7, 10-triazatetradecyl)benzyl)(methyl)amino)-2-oxoethyl)-1,4,7,10-tetraazacyclododecan-1,4,7-triy 1)triacetic acid (F-13)

4-((S)-2-((S)-2-(3,5-Bis(2-(methylsulfonyl)pyrimidin-5-yl)benzamido)-3-methylbutyramido) propionamido)-2-((methylamino)methyl)benzyl (1-((1-(((3R,5S)-1-((S)-2-((1R,2R)-3-(((1S,2R)-1-hydroxy-1-1-phenylpropan-2-yl)amino)-1-methoxy-2-methyl-3-oxopropyl) pyrrolidin-1-yl)-3-methoxy-5-methyl-1-oxoheptan-4-yl)amino)-3-methyl-1-oxobutan-2-yl)amino)-3-methyl-1-oxob utan-2-yl)(methyl)carbamate (69.5 mg, 46.4 μmol) was dissolved in DMF (2 mL), and DIPEA (30 mg) was added, then 2,2′,2″-(10-(2-((2,5-dioxopyrrolidin-1-yl)oxy)-2-oxoethyl)-1,4,7,10-tetraazacyclododecan-1,4,7-triyl)triacetic acid (70.1 mg, 0.14 mmol) was added. After the reaction was completed, the reaction solution was directly purified by preparative HPLC and freeze-dried to obtain the title compound (6.86 mg, 3.6 μmol).

Its structural characterization data are as follows:

MS m/z (ESI): 1883.1 [M+H]⁺

Its preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 4.00 | 20 | 80 | 30 |
| 24.00 | 90 | 10 | 30 |

### Example 5: Preparation of 2,2′,2″-(10-(2-((5-((S)-2-((S)-2-(3,5-bis(2-(methylsulfonyl) pyrimidin-5-yl)benzamido)-3-methylbutyramido)-5-ureidopentanamido)-2-((12R)-11-((S)-sec-bu tyl)-12-(2-((S)-2-((1R,2R)-3-(((1S,2R)-1-hydroxy-1-phenylpropan-2-yl)amino)-1-methoxy-2-met hyl-3-oxopropyl) pyrrolidin-1-yl)-2-oxoethyl)-5,8-diisopropyl-4,10-dimethyl-3,6,9-trioxo-2,13-di oxa-4,7,10-triazatetradecyl)benzyl)(methyl)amino)-2-oxoethyl)-1,4,7,10-tetraazacyclododecan~1, 4,7-triyl)triacetic acid (F-14)

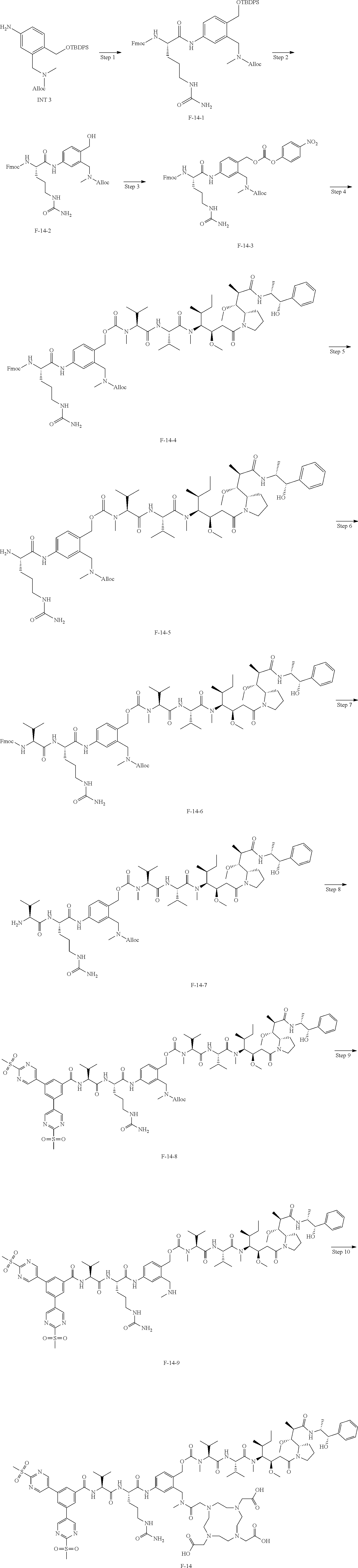

### Step 1: Preparation of allyl(S)-(5-(2-((((9H-fluoren-9-yl)methoxy)carbonyl)amino)-5-ureidopentanamido)-2-(((tert-butyldiphenylsilyl)oxy)methyl)benzyl)(methyl)carbamate (F-14-1)

Allyl (5-amino-2-(((tert-butyldiphenylsilyl)oxy)methyl)benzyl)(methyl)carbamate (800 mg, 1.64 mmol) and(S)-2-((((9H-fluoren-9-yl)methoxy)carbonyl)amino)-5-ureidopentanic acid (649.2 mg, 1.64 mmol) were dissolved in DCM (30 mL) and MeOH (6 mL), then 2-ethoxy-1-ethoxycarbonyl-1,2-dihydroquinoline (1.22 g, 4.92 mmol) was added. After the addition, the resulting mixture was reacted at 25° C. for 16 hours. After the reaction was completed, the reaction solution was purified by flash column chromatography (silica gel, ethyl acetate/petroleum ether=1) and further concentrated to obtain the title compound (995.5 mg, 1.15 mmol).

Its structural characterization data are as follows:

MS m/z (ESI): 885.3 [M+H₂O]⁺

### Step 2: Preparation of allyl(S)-(5-(2-((((9H-fluoren-9-yl)methoxy)carbonyl)amino)-5-ureidopentanamido)-2-(hydroxymethyl)benzyl)(methyl)carbamate (F-14-2)

Allyl(S)-(5-(2-((((9H-fluoren-9-yl)methoxy)carbonyl)amino)-5-ureidopentanamido)-2-(((tert-butyldiphenylsilyl)oxy)methyl)benzyl)(methyl)carbamate (995.5 mg, 1.15 mmol) was dissolved in DMF (10 mL), added dropwise with pyridinium hydrofluoride (3.5 mL) under ice bath. After the addition, the resulting mixture was reacted at 25° C. for 16 hours. After the reaction was completed, the reaction solution was purified by flash column chromatography (silica gel, ethyl acetate/petroleum ether=1) and further concentrated to obtain the title compound (506.5 mg, 0.81 mmol).

Its structural characterization data are as follows:

MS m/z (ESI): 647.3 [M+H₂O]⁺

### Step 3: Preparation of allyl(S)-(5-(2-((((9H-fluoren-9-yl)methoxy)carbonyl)amino)-5-ureidopentanamido)-2-((((4-nitrophenoxy)carbonyl)oxy)methyl)(methyl)carbamate (F-14-3)

Allyl(S)-(5-(2-((((9H-fluoren-9-yl)methoxy)carbonyl)amino)-5-ureidopentanamido)-2-(hydroxymethyl)benzyl)(methyl)carbamate (506.5 mg, 0.81 mmol) was dissolved in DMF (3 mL), DIPEA (209 mg) was added, then bis(4-nitrophenyl)carbonate (295.5 mg, 0.972 mmol) was added. After the addition, the resulting mixture was reacted at 25° C. for 16 hours. After the reaction was completed, the reaction solution was purified by flash column chromatography (silica gel, ethyl acetate/petroleum ether=1) and further concentrated to obtain the title compound (515.2 mg, 0.65 mmol).

Its structural characterization data are as follows:

MS m/z (ESI): 795.3 [M+H]⁺

### Step 4: Preparation of 4-((S)-2-((((9H-fluoren-9-yl)methoxy)carbonyl)amino)-5-ureidopentanamido)-2-((((allyloxy)carbonyl)(methyl)amino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxo propyl) pyrrolidin-1-yl)-3-methoxy-5-methyl-1-oxohept-4-yl)(methyl)amino)-3-methyl-1-oxobut an-2-yl)amino)-3-methyl-1-oxobutan-2-yl)(methyl)carbamate (F-14-4)

Allyl(S)-(5-(2-((((9H-fluoren-9-yl)methoxy)carbonyl)amino)-5-ureidopentanamido)-2-((((4-nitrophenoxy)carbonyl)oxy)methyl)(methyl)carbamate (515.2 mg, 0.65 mmol) and MMAE (CAS: 474645-27-7; 699.1 mg, 0.98 mmol) were dissolved in DMF (3 mL), DIPEA (251.6 mg) and 1-hydroxybenzotriazole (87.8 mg, 0.65 mmol) were added. After the addition, the resulting mixture was reacted at 25° C. for 2 hours. After the reaction was completed, the reaction solution was purified by flash column chromatography (C18, water/acetonitrile=0.6) and freeze-dried to obtain the title compound (624.7 mg, 0.46 mmol).

The structural characterization data are as follows:

MS m/z (ESI): 1373.8 [M+H]⁺

### Step 5: Preparation of 2-((((allyloxy)carbonyl)(methyl)amino)methyl)-4-((S)-2-amino-5-ureidopentanamido)benzyl (1-((1-(((3R,5S)-1)-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)amino)-3-methyl-1-oxobutan-2-yl) (methyl)carbamate (F-14-5)

4-((S)-2-((((9H-Fluoren-9-yl)methoxy)carbonyl)amino)-5-ureidopentanamido)-2-((((allylox y) carbonyl)(methyl)amino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-meth oxy-5-methyl-1-oxoheptan-4-yl)(methyl)amino)-3-methyl-1-oxobutan-2-yl)amino)-3-methyl-1-0 xobutan-2-yl)(methyl)carbamate (624.7 mg, 0.46 mmol) was dissolved in DMF (5 mL), diethylamine (120 mg) was added. After the addition, the resulting mixture was reacted at 25° C. for 0.5 hours. After the reaction was completed, the reaction solution was evaporated under reduced pressure to remove the solvent, then a mixed solution of ethyl acetate/petroleum ether (60 mL, ethyl acetate/petroleum ether=1/3) was added, stirred for 2 hours, filtered and dried to obtain the title compound (450.1 mg, 0.39 mmol).

Its structural characterization data are as follows:

MS m/z (ESI): 1151.8 [M+H]⁺

### Step 6: Preparation of 4-((S)-2-((S)-2-((((9H-fluoren-9-yl)methoxy)carbonyl)amino)-3-methylbutyramido)-S-ureidopentanamido)-2-((((allyloxy)carbonyl)(methyl)amino)methyl)benzyl (1-((1-(((3R,5S)-1-((S)-2-((1R,2R)-3-(((1S,2R)-1-hydroxy-1-phenylpropan-2-yl)amino)-1-methox y-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-14-6)

2-((((Allyloxy)carbonyl)(methyl)amino)methyl)-4-((S)-2-amino-5-ureidopentanamido)benz yl (1-((1-(((3R,5S)-1)-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl) a mino)-3-methyl-1-oxobutan-2-yl)amino)-3-methyl-1-oxobutan-2-yl)(methyl)carbamate (450.1 mg, 0.39 mmol) and (((9H-fluoren-9-yl)methoxy)carbonyl)-L-valine (164.8 mg, 0.47 mmol) were dissolved in DMF (5 mL), DIPEA (151 mg) and HATU (223.1 mg) were added in sequence. After the addition, the resulting mixture was reacted at 25° C. for 1 hour. The reaction solution was purified by flash column chromatography (C18, water/acetonitrile=0.8) and freeze-dried to obtain the title compound (459.3 mg, 0.31 mmol).

Its structural characterization data are as follows:

MS m/z (ESI): 1472.9 [M+H]⁺

### Step 7: Preparation of 2-((((allyloxy)carbonyl)(methyl)amino)methyl)-4-((S)-2-((S)-2-amino-3-methylbutyramido)-5-ureidopentanamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-14-7)

((S)-2-((S)-2-((((9H-Fluoren-9-yl)methoxy)carbonyl)amino)-3-methylbutyramido)-5-ureido pentanamido)-2-((((allyloxy)carbonyl)(methyl)amino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl) amino)-3-methyl-1-oxobutan-2-yl)(methyl)carbamate (459.3 mg, 0.31 mmol) was dissolved in DMF (5 mL), diethylamine (90 mg) was added. After the addition, the resulting mixture was reacted at 25° C. for 0.5 hours. After the reaction was completed, the reaction solution was evaporated under reduced pressure to remove the solvent, a mixed solution of ethyl acetate/petroleum ether (50 mL, ethyl acetate/petroleum ether=1/3) was added, stirred for 2 hours, filtered and dried to obtain the title compound (348.8 mg, 0.28 mmol).

Its structural characterization data are as follows:

MS m/z (ESI): 1250.8 [M+H]⁺

### Step 8: Preparation of 2-((((allyloxy)carbonyl)(methyl)amino)methyl)-4-((S)-2-((S)-2-(3,5-bis(2-(methylsulfonyl)pyrimidin-5-yl)benzamido)-3-methylbutyramido)-5-ureidopentanami do)benzyl (1-((1-(((3R,5S)-1-((S)-2-((1R,2R)-3-(((1S,2R)-1-hydroxy-1-phenylpropan-2-yl) amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1- oxobutan-2-yl)(methyl)carbamate (F-14-8)

2-((((Allyloxy)carbonyl)(methyl)amino)methyl)-4-((S)-2-((S)-2-amino-3-methylbutyramido)-5-ureidopentanamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)amino)-3-methyl-1-oxobutan-2-yl) (methyl)carbamate (100 mg, 80 μmol) was dissolved in DMF (3 mL), DIPEA (31 mg) and 2,5-dioxopyrrolidin-1-yl 3,5-bis(2-(methylsulfonyl)pyrimidin-5-yl)benzoate (63.7 mg, 0.12 mmol) were added. After the addition, the resulting mixture was reacted at 25° C. for 1 hour. After the reaction was completed, the reaction solution was purified by flash column chromatography (C18, water/acetonitrile=0.7) and freeze-dried to obtain the title compound (90.3 mg, 54.1 μmol).

Its structural characterization data are as follows:

MS m/z (ESI): 1666.8 [M+H]⁺

### Step 9: Preparation of 4-((S)-2-((S)-2-(3,5-bis(2-(methylsulfonyl)pyrimidin-5-yl)benzamido)-3-methylbutyramido)-5-ureidopentanamido)-2-((methylamino)methyl)benzyl (1-((1-(((3R,5S)-1-((S)-2-((1R,2R)-3-(((1S,2R)-1-hydroxy)-1-phenylpropan-2-yl)amino)-1-metho xy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-14-9)

2-((((Allyloxy)carbonyl)(methyl)amino)methyl)-4-((S)-2-((S)-2-(3,5-bis(2-(methylsulfonyl) pyrimidin-5-yl)benzamido)-3-methylbutyramido)-5-ureidopentanamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-0 xobutan-2-yl)amino)-3-methyl-1-oxobutan-2-yl)(methyl)carbamate (90.3 mg, 54.1 μmol) was dissolved in DMF (2 mL), tetrakis(triphenylphosphine) palladium (12.5 mg, 10.8 μmol) was added, followed by an addition of 40 μL of formic acid and 80 μL of N-methylmorpholine, and reacted under stirring and nitrogen protection at room temperature for 1 hour. After the reaction was completed, the product was purified by flash column chromatography (C18, water/acetonitrile=0.5) and freeze-dried to obtain the title compound (59.9 mg, 37.9 μmol).

The structural characterization data are as follows:

MS m/z (ESI): 1582.9 [M+H]⁺

### Step 10: Preparation of 2,2′,2″-(10-(2-((5-((S)-2-((S)-2-(3,5-bis(2-(methylsulfonyl) pyrimidin-5-yl)benzamido)-3-methylbutyramido)-5-ureidopentanamido)-2-((12R)-11-((S)-sec-bu tyl)-12-(2-((S)-2-((1R,2R)-3-(((1S,2R)-1-hydroxy-1-phenylpropan-2-yl)amino)-1-methoxy-2-met hyl-3-oxopropyl) pyrrolidin-1-yl)-2-oxoethyl)-5,8-diisopropyl-4,10-dimethyl-3,6,9-trioxo-2,13-di oxa-4,7,10-triazatetradecyl)benzyl)(methyl)amino)-2-oxoethyl)-1,4,7,10-tetraazacyclododecan-1, 4,7-triyl)triacetic acid (F-14)

4-((S)-2-((S)-2-(3,5-bis(2-(methylsulfonyl)pyrimidin-5-yl)benzamido)-3-methylbutyramido)-5-ureidopentanamido)-2-((methylamino)methyl)benzyl (1-((1-(((3R.5S)-1-((S)-2-((1R,2R)-3-(((1S,2R)-1-hydroxy)-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (59.9 mg, 37.9 μmol) was dissolved in DMF (2 mL), DIPEA (25 mg) was added, followed by an addition of 2,2′,2″-(10-(2-((2,5-dioxopyrrolidin-1-yl)oxy)-2-oxoethyl)-1,4,7,10-tetraazacyclododecan-1,4,7-triyl)triacetic acid (56.9 mg, 113.6 μmol). After the reaction was completed, the reaction solution was directly purified by preparative HPLC and freeze-dried to obtain the title compound (14.6 mg, 7.4 μmol).

Its structural characterization data are as follows:

MS m/z (ESI): [M+H]⁺=1969.1

Its preparation method was follows:
Chromatographic column: Waters SunFire Prep C18 OBD (5 μm*19 mm*150 mm)
Mobile phase A: acetonitrile; Mobile phase B: water (0.05% trifluoroacetic acid)

| Time | Mobile | Mobile | Flow rate |
|---|---|---|---|
| [min] | phase A [%] | phase B [%] | [mL/min] |
| 0.00 | 30 | 70 | 30 |
| 4.00 | 30 | 70 | 30 |
| 24.00 | 90 | 10 | 30 |

### Example 6: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-a mido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-5,8-diisopropyl-4,1 0-dimethyl-3,6,9-trioxo-2,13-dioxa-4,7,10-triazatetradecyl)benzyl)(methyl)amino)-2-oxoethyl)-1, 4,7,10-tetraazacy clododecan-1,4,7-triyl)triacetic acid (F-15)

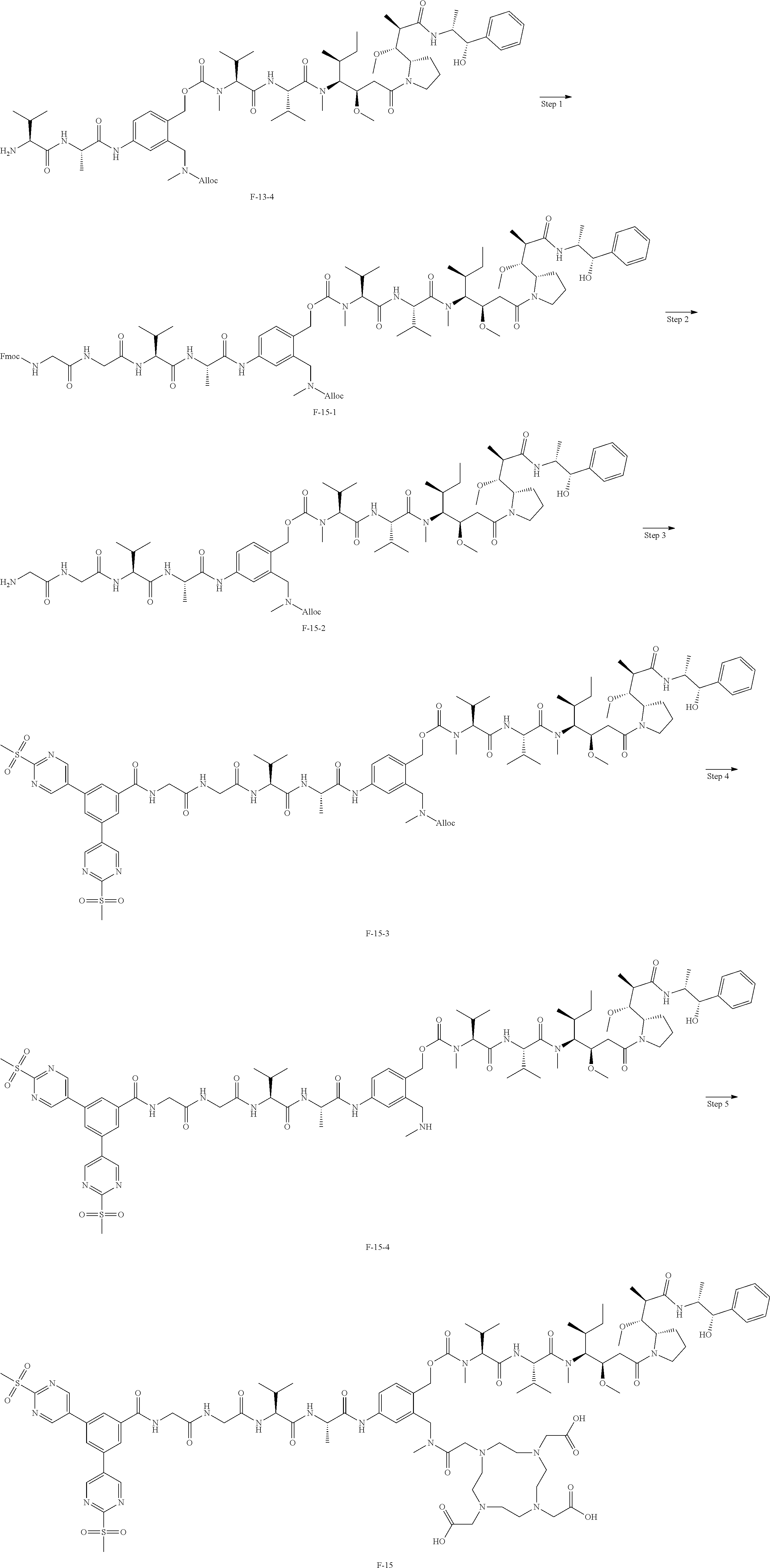

### Step 1: Preparation of 2-((((allyloxy)carbonyl)(methyl)amino)methyl)-4-((S)-2-((S)-2-(2-(2-aminoacetamido) acetamido)-3-methylbutyramido) propionamido)benzyl (1-((1-(((3R,5S)-1-(S)-2-((1R,2R)-3-(((1S,2R)-1-hydroxy-1-phenylpropan-2-yl)amino)-1-methoxy-2-methyl-3-ox opropyl) pyrrolidin-1-yl)-3-methoxy-5-methyl-1-oxoheptan-4-yl)(methyl)amino)-3-methyl-1-oxo butan-2-yl)amino)-3-methyl-1-oxobutan-2-yl)(methyl)carbamate (F-15-1)

2-((((Allyloxy)carbonyl)(methyl)amino)methyl)-4-((S)-2-((S)-2-amino-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohepta n-4-yl)(methyl)amino)-3-methyl-1-oxobutan-2-yl)amino)-3-methyl-1-oxobutan-2-yl)(methyl) car bamate (120 mg, 104.2 μmol) and (((9H-fluoren-9-yl)methoxy)carbonyl)-L-valine (44.1 mg, 125.4 μmol) were dissolved in DMF (3 mL), DIPEA (42 mg) and HATU (60.1 mg) were added in sequence. After the addition, the resulting mixture was reacted at 25° C. for 1 hour. The product was purified by flash column chromatography (C18, water/acetonitrile=0.8) and then freeze-dried to obtain the title compound (125.2 mg, 93.7 μmol).

Its structural characterization data are as follows:

MS m/z (ESI): 1501.1 [M+H]⁺

### Step 2: Preparation of 2-((((allyloxy)carbonyl)(methyl)amino)methyl)-4-((S)-2-((S)-2-(2-(2-aminoacetamido) acetamido)-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-ox opropyl) pyrrolidin-1-yl)-3-methoxy-5-methyl-1-oxohept-4-yl)(methyl)amino)-3-methyl-1-oxobu tan-2-yl)amino)-3-methyl-1-oxobutan-2-yl)(methyl)carbamate (F-15-2)

2-((((Allyloxy)carbonyl)(methyl)amino)methyl)-4-((S)-2-((S)-2-(2-(2-aminoacetamido) acet amido)-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-met hyl-1-oxobutan-2-yl)(methyl)carbamate (125.2 mg, 93.7 μmol) was dissolved in DMF (3 mL), diethylamine (100 mg) was added. After the addition, the resulting mixture was reacted at 25° C. for 0.5 hours. After the reaction was completed, the reaction solution was evaporated under reduced pressure to remove the solvent, a mixed solution of ethyl acetate/petroleum ether (30 mL, ethyl acetate/petroleum ether=1/3) was added, stirred for 2 hours, filtered and dried to obtain the title compound (97.3 mg, 75.8 μmol).

Its structural characterization data are as follows:

MS m/z (ESI): 1278.8 [M+H]⁺

### Step 3: Preparation of 2-((((allyloxy)carbonyl)(methyl)amino)methyl)-4-((9S,12S)-1-(3,5-bis(2-(methylsulfonyl)pyrimidin-5-yl)phenyl)-9-isopropyl-12-methyl-1,4,7,10-tetraoxo-2,5, 8,11-tetraazatridecan-13-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-meth oxy-5-methyl-1-oxohept-4-yl)(methyl)amino)-1-oxobutan-2-yl)amino)-3-methyl-1-oxobutan-2-yl)(methyl)carbamate (F-15-3)

2-((((Allyloxy)carbonyl)(methyl)amino)methyl)-4-((S)-2-((S)-2-(2-(2-aminoacetamido) acet amido)-3-methylbutyramido) propionamido)benzyl (1-((1-(((3R,5S)-1-(S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(metbyl)amino)-3-methyl-1-oxobutan-2-yl)amino)-3-methy 1-1-oxobutan-2-yl)(methyl)carbamate (48.3 mg, 37.8 μmol) was dissolved in DMF (2 mL), DIPEA (35 mg) and 2,5-dioxopyrrolidin-1-yl 3,5-bis(2-(methylsulfonyl)pyrimidin-5-yl)benzoate (72.6 mg, 0.15 mmol) were added. After the addition, the resulting mixture was reacted at 25° C. for 1 hour. After the reaction was completed, the product was purified by flash column chromatography (C18, water/acetonitrile=0.6) and freeze-dried to obtain the title compound (57.2 mg, 33.8 μmol).

Its structural characterization data are as follows:

MS m/z (ESI): 1694.9 [M+H]⁺

### Step 4: Preparation of 4-((9S,12S)-1-(3,5-bis(2-(methylsulfonyl)pyrimidin-5-yl)phenyl)-9-isopropyl-12-methyl-1,4,7,10-tetraoxo-2,5,8,11-tetraazatridecan-13-amido)-2-((methylamino) me thyl)benzyl (1-((1-(((3R,5S)-1-((S)-2-((1R,2R)-3-(((1S,2R)-1-hydroxy-1-phenylpropan-2-yl) amino)-1-methoxy-2-methyl-3-oxopropyl) pyrrolidin-1-yl)-3-methoxy-5-methyl-1-oxoheptan-4-y l)(methyl)amino)-1-oxobutan-2-yl)amino)-3-methyl-1-oxobutan-2-yl)(methyl)carbamate (F-15-4)

2-((((allyloxy)carbonyl)(methyl)amino)methyl)-4-((9S,12S)-1-(3,5-bis(2-(methylsulfonyl) p yrimidin-5-yl)phenyl)-9-isopropyl-12-methyl-1,4,7,10-tetraoxo-2,5,8,11-tetraazatridecan-13-ami do)benzyl (1-((1-(((3R,5S)-1-((S)-2-((1R,2R)-3-(((1S,2R)-1-hydroxy-1-phenylpropan-2-yl) amino)-1-methoxy-2-methyl-3-oxopropyl) pyrrolidin-1-yl)-3-methoxy-5-methyl-1-oxohept-4-yl)(methyl)amino)-1-oxobutan-2-yl)amino)-3-methyl-1-oxobutan-2-yl)(methyl)carbamate (57.2 mg, 33.8 mol) was dissolved in DMF (2 mL), tetrakis(triphenylphosphine) palladium (7.8 mg, 6.8 μmol) was added, followed by an addition of 30 μL of formic acid and 60 μL of N-methylmorpholine, and stirred at room temperature for 1 hour under nitrogen protection. After the reaction was completed, the product was purified by flash column chromatography (C18, water/acetonitrile=0.55) and freeze-dried to obtain the title compound (43.5 mg, 27.0 μmol).

Its structural characterization data are as follows:

MS m/z (ESI): 1610.8 [M+H]⁺

### Step 5: Preparation of 2,2′,2″-(10-(2-((5-((9S,12S)-1-(3,5-bis(2-(methylsulfonyl) pyrimidin-5-yl)phenyl)-9-isopropyl-12-methyl-1,4,7,10-tetraoxo-2,5,8,11-tetraazatridecan-13-a mido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-5,8-diisopropyl-4,1 0-dimethyl-3,6,9-trioxo-2,13-dioxa-4,7,10-triazatetradecyl)benzyl)(methyl)amino)-2-oxoetbyl)-1, 4,7,10-tetraazacyclododecan-1,4,7-triyl)triacetic acid (F-15)

4-((9S,12S)-1-(3,5-Bis(2-(methylsulfonyl)pyrimidin-5-yl)phenyl)-9-isopropyl-12-methyl-1, 4,7,10-tetraoxo-2,5,8,11-tetraazatridecan-13-amido)-2-((methylamino)methyl)benzyl (1-((1-(((3R,5S)-1-((S)-2-((1R,2R)-3-(((1S,2R)-1-hydroxy-1-phenylpropan-2-yl)amino)-1-methoxy-2-m ethyl-3-oxopropyl) pyrrolidin-1-yl)-3-methoxy-5-methyl-1-oxohept-4-yl)(methyl)amino)-1-oxob utan-2-yl)amino)-3-methyl-1-oxobutan-2-yl)(methyl)carbamate (43.5 mg, 27.0 μmol) was dissolved in DMF (2 mL), DIPEA (18 mg) was added, followed by an addition of 2,2′,2″-(10-(2-((2,5-dioxopyrrolidin-1-yl)oxy)-2-oxoethyl)-1,4,7,10-tetraazacyclododecan-1,4,7-t riyl)triacetic acid (40.6 mg, 81.0 μmol). After the reaction was completed, the reaction solution was directly purified by preparative HPLC and freeze-dried to obtain the title compound (12.61 mg, 6.3 μmol)

Its structural characterization data are as follows:

MS m/z (ESI): 1997.1 [M+H]⁺

Its preparation method was follows:
Chromatographic column: Waters SunFire Prep C18 OBD (5 μm*19 mm*150 mm)
Mobile phase A: acetonitrile; Mobile phase B: water (0.05% trifluoroacetic acid)

| Time | Mobile | Mobile | Flow rate |
|---|---|---|---|
| [min] | phase A [%] | phase B [%] | [mL/min] |
| 0.00 | 25 | 75 | 30 |
| 4.00 | 25 | 75 | 30 |
| 24.00 | 90 | 10 | 30 |

### Example 7: Preparation of 2,2′,2″-(10-(2-((5-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-tr iazahexacosan-26-amido)-2-((12R)-11-((S)-sec-butyl)-12-(2-((S)-2-((1R,2R)-3-(((1S,2R)-1-hydro xy-1-phenylpropan-2-yl)amino)-1-methoxy-2-methyl-3-oxopropyl) pyrrolidin-1-yl)-2-oxoethyl)-5,8-diisopropyl-4,10-dimethyl-3,6,9-trioxo-2,13-dioxa-4,7,10-triazacyclododecan-1,4,7-triyl)tria cetic acid (F-16)

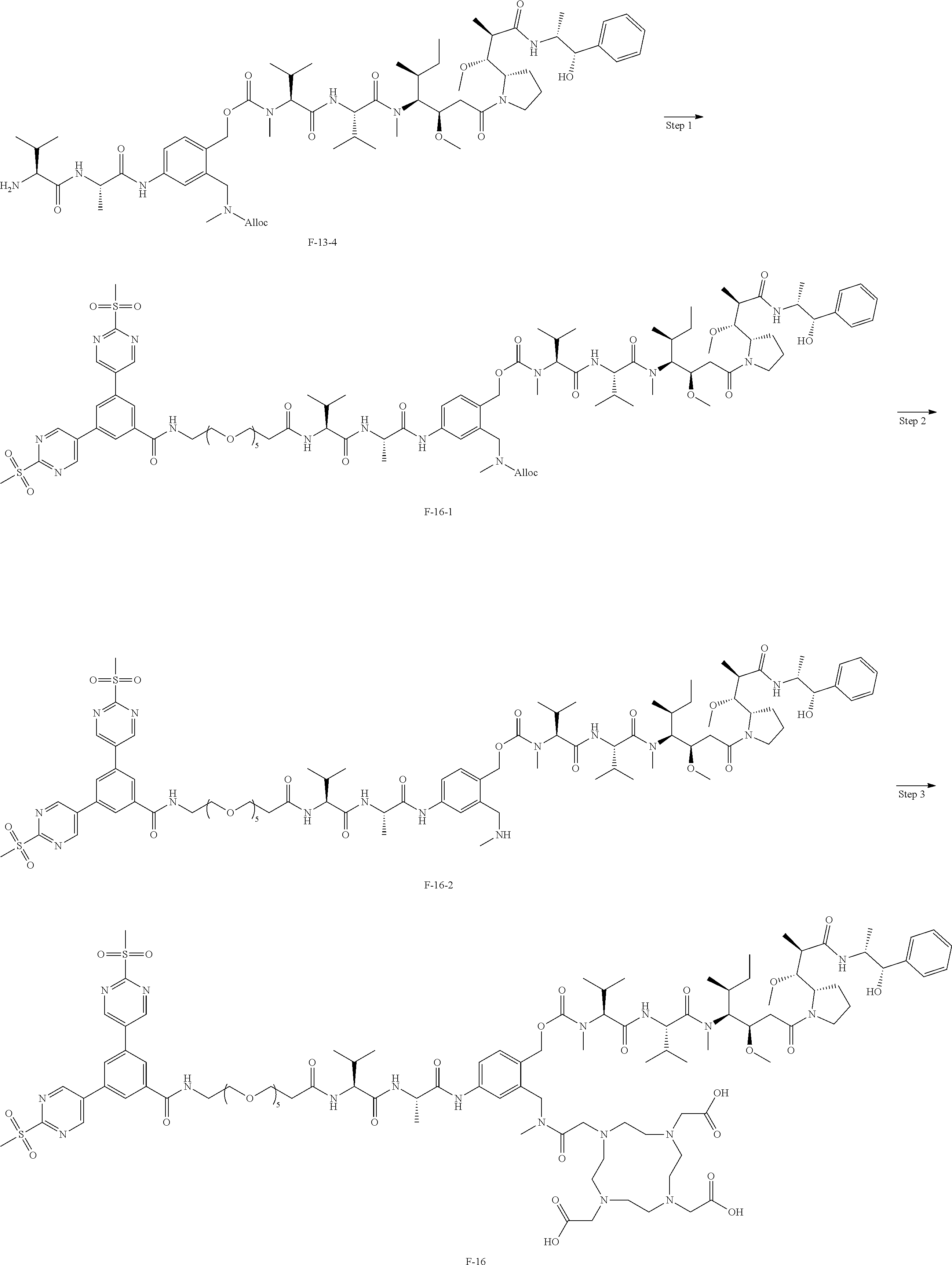

### Step 1: Preparation of 2-((((allyloxy)carbonyl)(methyl)amino)methyl)-4-((22S,25S)-1-(3,5-bis(2-(methylsulfonyl)pyrimidin-5-yl)phenyl)-22-isopropyl-25-methyl-1,20,23-trioxo-5,8,1 1,14,17-pentaoxa-2,21,24-triazahexacosan-26-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidi n-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-16-1)

2-((((Allyloxy)carbonyl)(methyl)amino)methyl)-4-((S)-2-((S)-2-amino-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carba mate (30.0 mg, 25.8 μmol) and 1-(3,5-bis(2-(methylsulfonyl)pyrimidin-5-yl)phenyl)-1-oxo-5,8,11,14,17-pentaoxa-2-azaeicosan-20-oic acid (Int-5, 28.1 mg, 38.7 μmol) were dissolved in DMF (2 mL), DIPEA (15 mg) and HATU (15.1 mg) were added in sequence. After the addition, the resulting mixture was reacted at 25° C. for 1 hour. The product was purified by flash column chromatography (C18, water/acetonitrile=0.6) and freeze-dried to obtain the title compound (33.8 mg, 18.1 μmol).

Its structural characterization data are as follows:

MS m/z (ESI): 1872.1 [M+H]⁺

### Step 2: Preparation of 4-((22S,25S)-1-(3,5-bis(2-(methylsulfonyl)pyrimidin-5-yl)phenyl)-22-isopropyl-25-methyl-1,20,23-trioxo-5,8,11,14,17-pentaoxa-2,21,24-triazahexacosan-26-amid 0)-2-((methylamino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxoheptan-4-yl)(methyl)amino)-3-methyl-1-oxobutan-2-yl)amino)-3-methyl-1-oxobutan-2-yl)(methyl)carbamate (F-16-2)

2-((((Allyloxy)carbonyl)(methyl)amino)methyl)-4-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-tr iazahexacosan-26-amido)benzyl (1-((1-(((3R,5S)-1-(S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)(methyl)carbamate (33.8 mg, 18.1 μmol) was dissolved in DMF (2 mL), tetrakis(triphenylphosphine) palladium (4.2 mg, 3.6 μmol) was added, then 20 μL of formic acid and 40 μL of N-methylmorpholine was added, and stirred at room temperature for 1 hour under nitrogen protection. After the reaction was completed, the product was purified by flash column chromatography (C18, water/acetonitrile=0.55) and freeze-dried to obtain the title compound (22.6 mg, 12.7 μmol).

Its structural characterization data are as follows:

MS m/z (ESI): 1788.1 [M+H]⁺

### Step 3: Preparation of 2,2′,2″-(10-(2-((5-((22S,25S)-1-(3,5-bis(2-(methylsulfonyl) pyrimidin-5-yl)phenyl)-22-isopropyl-25-methyl-1,20,23-trioxo-5,8,11,14,17-pentaoxa-2,21,24-tr iazahexacosan-26-amido)-2-((12R)-11-((S)-sec-butyl)-12-(2-((S)-2-((1R,2R)-3-(((1S,2R)-1-hydro xy-1-phenylpropan-2-yl)amino)-1-methoxy-2-methyl-3-oxopropyl) pyrrolidin-1-yl)-2-oxoethyl)-5,8-diisopropyl-4,10-dimethyl-3,6,9-trioxo-2,13-dioxa-4,7,10-triazacyclododecan-1,4,7-triyl)tria cetic acid (F-16)

4-((22S,25S)-1-(3,5-Bis(2-(methylsulfonyl)pyrimidin-5-yl)phenyl)-22-isopropyl-25-methyl-1,20,23-trioxo-5,8,11,14,17-pentaoxa-2,21,24-triazahexadecan-26-amido)-2-((methylamino) met hyl)benzyl (1-((1-(((3R,5S)-1-((S)-2-((1R,2R)-3-(((1S,2R)-1-hydroxy-1-phenylpropan-2-yl) amino)-1-methoxy-2-methyl-3-oxopropyl) pyrrolidin-1-yl)-3-methoxy-5-methyl-1-oxoheptan-4-y l)(methyl)amino)-3-methyl-1-oxobutan-2-yl)amino)-3-methyl-1-oxobutan-2-yl)(methyl)carbama the (22.6 mg, 12.7 μmol) was dissolved in DMF (2 mL), DIPEA (10 mg) was added, then 2.2′,2″-(10-(2-((2,5-dioxopyrrolidin-1-yl)oxy)-2-oxoethyl)-1,4,7,10-tetraazacyclododecan-1,4,7-t riyl)triacetic acid (19.1 mg, 38.1 μmol) was added. After the reaction was completed, the reaction solution was directly purified by preparative high performance liquid chromatography and freeze-dried to obtain the title compound (5.17 mg, 2.4 μmol).

Its structural characterization data are as follows:

MS m/z (ESI): [M/2+H]⁺=1087.1

Its preparation method was follows:
Chromatographic column: Waters SunFire Prep C18 OBD (5 μm*19 mm*150 mm)
Mobile phase A: acetonitrile; Mobile phase B: water (0.05% trifluoroacetic acid)

| Time | Mobile | Mobile | Flow rate |
|---|---|---|---|
| [min] | phase A [%] | phase B [%] | [mL/min] |
| 0.00 | 20 | 80 | 30 |
| 4.00 | 20 | 80 | 30 |
| 24.00 | 90 | 10 | 30 |

### Example 8: Preparation of 2,2′,2″-(10-(2-((5-((22S,25S)-1-(3,5-bis(2-(methylsulfonyl) pyrimidin-5-yl)phenyl)-22-isopropyl-1,20,23-trioxo-25-(3-ureidopropyl)-5,8,11,14,17-pentaoxa-2,21,24-triazahexacosan-26-amido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-5,8-diisopropyl-4,10-dimethyl-3,6,9-trioxo-2,13-dioxa-4,7,10-triazatetradecyl)benzyl) (methyl)amino)-2-oxoethyl)-1,4,7,10-tetraazacyclododecan-1,4,7-triyl)triacetic acid (F-17)

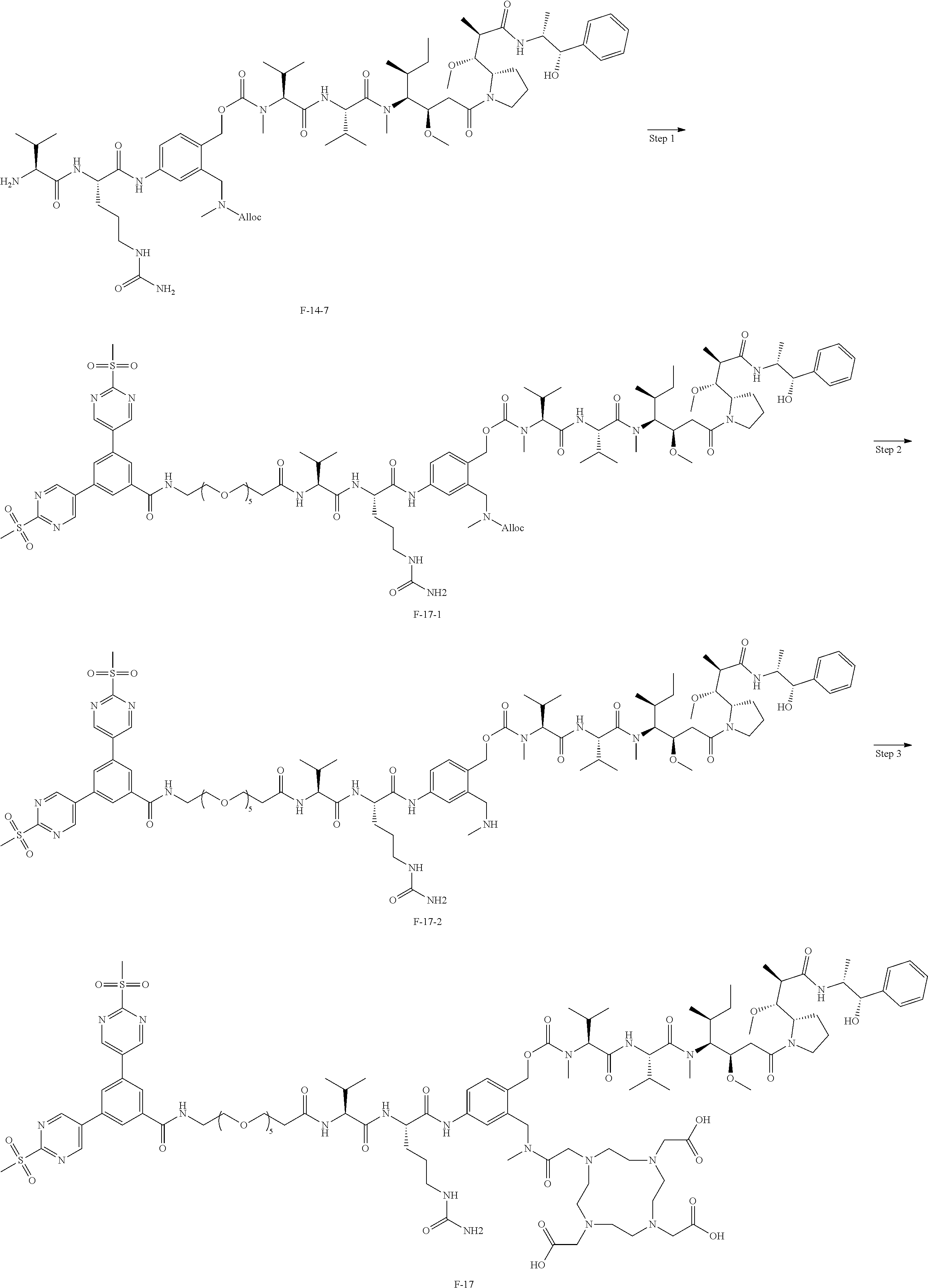

### Step 1: Preparation of 2-((((allyloxy)carbonyl)(methyl)amino)methyl)-4-((22S,25S)-1-(3,5-bis(2-(methylsulfonyl)pyrimidin-5-yl)phenyl)-22-isopropyl-1,20,23-trioxo-25-(3-ureidoprop yl)-5,8,11,14,17-pentaoxa-2,21,24-triazahexadecan-26-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl) amino)-3-methyl-1-oxobutan-2-yl)(methyl)carbamate (F-17-1)

2-((((Allyloxy)carbonyl)(methyl)amino)methyl)-4-((S)-2-((S)-2-amino-3-methylbutyramido)-5-ureidopentanamido)benzyl (1-((1-(((3R,5S)-1-((S)-2-((1R,2R)-3-(((1S,2R)-1-hydroxy-1-phenylpropan-2-yl)amino))-1-methoxy-2-methyl-3-oxopropyl) pyrrolidin-1-yl)-3-methoxy-5-met hyl-1-oxoheptan-4-yl)(methyl)amino)-3-methyl-1-oxobutan-2-yl)amino)-3-methyl-1-oxobutan-2-yl)(methyl)carbamate (100.0 mg, 80.0 μmol) and 1-(3,5-bis(2-(methylsulfonyl)pyrimidin-5-yl)phenyl)-1-oxo-5,8,11,14,17-pentaoxa-2-azaeicosan-20-oleic acid (87.2 mg, 120.0 μmol) were dissolved in DMF (3 mL), DIPEA (31 mg) and HATU (60.8 mg) were added in sequence. After the addition, the resulting mixture was reacted at 25° C. for 1 hour. After the reaction was completed, the product was purified by flash column chromatography (C18, water/acetonitrile=0.6) and freeze-dried to obtain the title compound (93.8 mg, 47.9 μmol).

Its structural characterization data are as follows:

MS m/z (ESI): 1958.1 [M+H]⁺

### Step 2: Preparation of methyl 4-((22S,25S)-1-(3,5-bis(2-(methylsulfonyl)pyrimidin-5-yl)phenyl)-22-isopropyl-1,20,23-trioxo-25-(3-ureidopropyl)-5,8,11,14,17-pentaoxa-2,21,24-triazah exacosan-26-amido)-2-((methylamino)methyl)benzyl (1-((1-(((3R,5S)-1-((S)-2-((1R,2R)-3-(((1S,2R)-1-hydroxy-1-phenylpropan-2-yl)amino)-1-metho xy-2-methyl-3-oxopropyl) pyrrolidin-1-yl)-3-methoxy-5-methyl-1-oxoheptan-4-yl)(methyl) amin 0)-3-methyl-1-oxobutan-2-yl)amino)-3-methyl-1-oxobutan-2-yl)(methyl)carbamate (F-17-2)

Methyl(2-(((allyloxy)carbonyl)(methyl)amino)methyl)-4-((22S,25S)-1-(3,5-bis(2-(methylsulfonyl)pyrimidin-5-yl)phenyl)-22-isopropyl-1,20,23-trioxo-25-(3-ureidopropyl)-5,8,11, 14,17-pentaoxa-2,21,24-triazahexadecan-26-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (93.8 mg, 47.9 μmol) was dissolved in DMF (2 mL), tetrakis(triphenylphosphine) palladium (11.1 mg, 9.6 μmol) was added, followed by an addition of 40 μL of formic acid and 80 μL of N-methylmorpholine, and stirred at room temperature for 1 hour under nitrogen protection. After the reaction was completed, the product was purified by flash column chromatography (C18, water/acetonitrile=0.5) was purified and freeze-dried to obtain the title compound (62.8 mg, 33.5 μmol).

Its structural characterization data are as follows:

MS m/z (ESI): 1874.1 [M+H]⁺

### Step 3: Preparation of 2,2′,2″-(10-(2-((5-((22S,25S)-1~ (3,5-bis(2-(methylsulfonyl) pyrimidin-5-yl)phenyl)-22-isopropyl-1,20,23-trioxo-25-(3-ureidopropyl)-5,8,11,14,17-pentaoxa-2,21,24-triazahexacosan-26-amido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoethyl)-5,8-diisopropyl-4,10-dimethyl-3,6,9-trioxo-2,13-dioxa-4,7,10-triazatetradecyl)benzyl)(methyl)amino)-2-oxoethyl)-1,4,7,10-tetraazacyclododecan-1,4,7-triyl)triacetic acid (F-17)

Methyl 4-((22S,25S)-1-(3,5-bis(2-(methylsulfonyl)pyrimidin-5-yl)phenyl)-22-isopropyl-1, 20,23-trioxo-25-(3-ureidopropyl)-5,8,11,14,17-pentaoxa-2,21,24-triazahexacosan-26-amido)-2-((methylamino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxoheptan-4-yl)(methyl)amino)-3-methyl-1-oxobutan-2-yl)amino)-3-methyl-1-oxobutan-2-yl)(methyl)carbamate (62.8 mg, 33.5 μmol) was dissolved in DMF (2 mL), DIPEA (22 mg) was added, followed by an addition of 2,2′,2″-(10-(2-((2,5-dioxopyrrolidin-1-yl)oxy)-2-oxoethyl)-1,4,7,10-tetraazacyclododecan-1,4,7-triyl)triacetic acid (50.4 mg, 100.6 μmol). After the reaction was completed, the reaction solution was directly purified by preparative HPLC and freeze-dried to obtain the title compound (20.47 mg, 7.4 μmol).

Its structural characterization data are as follows:

MS m/z (ESI): [M/2+H]⁺=1130.1

Its preparation method was follows:

### Chromatographic column: Waters SunFire Prep C18 OBD (5 μm*19 mm*150 mm)

### Mobile phase A: acetonitrile: Mobile phase B: water (0.05% trifluoroacetic acid)

| Time | Mobile | Mobile | Flow rate |
|---|---|---|---|
| [min] | phase A [%] | phase B [%] | [mL/min] |
| 0.00 | 30 | 70 | 30 |
| 4.00 | 30 | 70 | 30 |
| 24.00 | 70 | 30 | 30 |

### Example 9: Preparation of 2,2′,2″-(10-(2-((5-((28S,31S)-1-(3,5-bis(2-(methylsulfonyl) pyrimidin-5-yl)phenyl)-28-isopropyl-31-methyl-1,20,23,26,29-pentaoxo-5,8,11,14,17-pentaoxa-2,21,24,27,30-pentaazadotriacontan-32-amido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolid in-1-yl)-2-oxoethyl)-5,8-diisopropyl-4,10-dimethyl-3,6,9-trioxo-2,13-dioxa-4,7,10-triazatetradec yl)benzyl)(methyl)amino)-2-oxoethyl)-1,4,7,10-tetraazacyclododecan-1,4,7-triyl)triacetic acid (F-18)

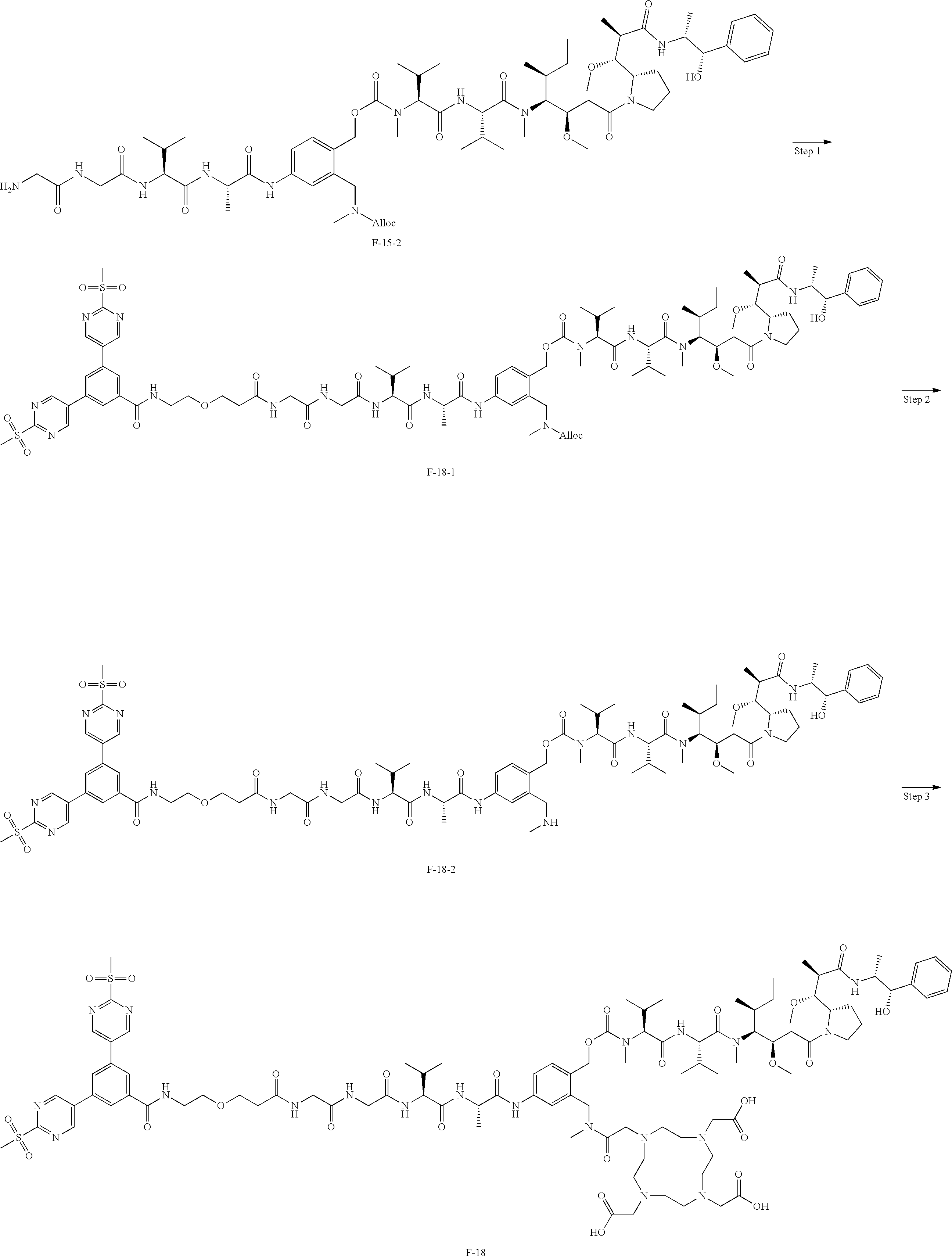

### Step 1: Preparation of 2-((((allyloxy)carbonyl)(methyl)amino)methyl)-4-((28S,31S)-1-(3,5-bis(2-(methylsulfonyl)pyrimidin-5-yl)phenyl)-28-isopropyl-31-methyl-1,20,23,26,29-pent aoxo-5,8,11,14,17-pentaoxa-2,21,24,27,30-pentaazadotriacontan-32-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-0 xobutan-2-yl)amino)-3-methyl-1-oxobutan-2-yl)(methyl)carbamate (F-8-1)

Methyl 2-((((allyloxy)carbonyl)(methyl)amino)methyl)-4-((S)-2-((S)-2-(2-(2-aminoacetamido) acetamido)-3-methylbutyramido) propionamido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxoprop yl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (48.0 mg, 56.4 μmol) and 1-(3,5-bis(2-(methylsulfonyl)pyrimidin-5-yl)phenyl)-1-oxo-5,8,11,14,17-pentaoxa-2-azaeicosan-20-oic acid (41.1 mg, 56.4 μmol) were dissolved in DMF (3 mL), DIPEA (20 mg) and HATU (21.4 mg) were added in sequence, and then reacted at 25° C. for 1 hour. The product was purified by flash column chromatography (C18, water/acetonitrile=0.6) and freeze-dried to obtain the title compound (52.2 mg, 26.3 μmol).

Its structural characterization data are as follows:

MS m/z (ESI): 1986.1 [M+H]⁺

### Step 2: Preparation of 4-((28S,31S)-1-(3,5-bis(2-(methylsulfonyl)pyrimidin-5-yl)phenyl)-28-isopropyl-31-methyl-1,20,23,26,29-pentaoxo-5,8,11,14,17-pentaoxa-2,21,24,27,30-pentaazad otriacontan-32-amido)-2-((methylamino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-18-2)

2-((((Allyloxy)carbonyl)(methyl)amino)methyl)-4-((28S,31S)-1-(3,5-bis(2-(methylsulfonyl) pyrimidin-5-yl)phenyl)-28-isopropyl-31-methyl-1,20,23,26,29-pentaoxo-5,8,11,14,17-pentaoxa-2,21,24,27,30-pentaazadotriacontan-32-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methy 1-1-oxobutan-2-yl)(methyl)carbamate (52.2 mg, 26.3 μmol) was dissolved in DMF (2 mL), tetrakis(triphenylphosphine) palladium (7.8 mg, 6.8 μmol) was added, then 30 μL of formic acid and 60 μL of N-methylmorpholine was added, and stirred at room temperature for 1 hour under nitrogen protection. After the reaction was completed, the product was purified by flash column chromatography (C18, water/acetonitrile=0.55) and freeze-dried to obtain the title compound (35.1 mg, 18.4 μmol).

Its structural characterization data are as follows:

MS m/z (ESI): 1902.1 [M+H]⁺

### Step 3: Preparation of 2,2′,2″-(10-(2-((5-((28S,31S)-1-(3,5-bis(2-(methylsulfonyl) pyrimidin-5-yl)phenyl)-28-isopropyl-31-methyl-1,20,23,26,29-pentaoxo-5,8,11,14,17-pentaoxa-2,21,24,27,30-pentaazadotriacontan-32-amido)-2-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolid in-1-yl)-2-oxoethyl)-5,8-diisopropyl-4,10-dimethyl-3,6,9-trioxo-2,13-dioxa-4,7,10-triazatetradec yl)benzyl)(methyl)amino)-2-oxoethyl)-1,4,7,10-tetraazacyclododecan-1,4,7-triyl)triacetic acid (F-18)

4-((28S,31S)-1-(3,5-Bis(2-(methylsulfonyl)pyrimidin-5-yl)phenyl)-28-isopropyl-31-methyl-1,20,23,26,29-pentaoxo~5,8,11,14,17-pentaoxa-2,21,24,27,30-pentaazadotriacontan-32-amido)-2-((methylamino)methyl)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)amino)-3-methyl-1-oxobutan-2-yl) (methyl)carbamate (35.1 mg, 18.4 μmol) was dissolved in DMF (2 mL), DIPEA (18 mg) was added, then 2,2′,2″-(10-(2-((2,5-dioxopyrrolidin-1-yl)oxy)-2-oxoethyl)-1,4,7,10-tetraazacyclododecan-1,4,7-triyl)triacetic acid (46.3 mg, 92.0 μmol) was added. After the reaction was completed, the reaction solution was directly purified by preparative high performance liquid chromatography and freeze-dried to obtain the title compound (23.17 mg, 10.1 μmol)

Its structural characterization data are as follows:

MS m/z (ESI): [M/2+H]⁺=1144.1

Its preparation method was follows:
Chromatographic column: Waters SunFire Prep C18 OBD (5 μm*19 mm*150 mm)
Mobile phase A: acetonitrile; Mobile phase B: water (0.05% trifluoroacetic acid)

| Time | Mobile | Mobile | Flow rate |
|---|---|---|---|
| [min] | phase A [%] | phase B [%] | [mL/min] |
| 0.00 | 25 | 75 | 30 |
| 4.00 | 25 | 75 | 30 |
| 24.00 | 90 | 10 | 30 |

### Example 10: Preparation of 2,2′,2″-(10-((6S,9S,12S)-1-amino-12-(3,5-bis(2-(methylsulfonyl) pyrimidin-5-yl)benzamido)-6-((4-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoe thyl)-5,8-diisopropyl-4,10-dimethyl-3,6,9-trioxo-2,13-dioxa-4,7,10-triazatetradecyl)phenyl)carba moyl)-9-isopropyl-1,8,11,18-tetraoxo-2,7,10,17-tetraazanonadecan-19-yl)-1,4,7,10-tetraazacyclo dodecan-1,4,7-triyl)triacetic acid (F-19)

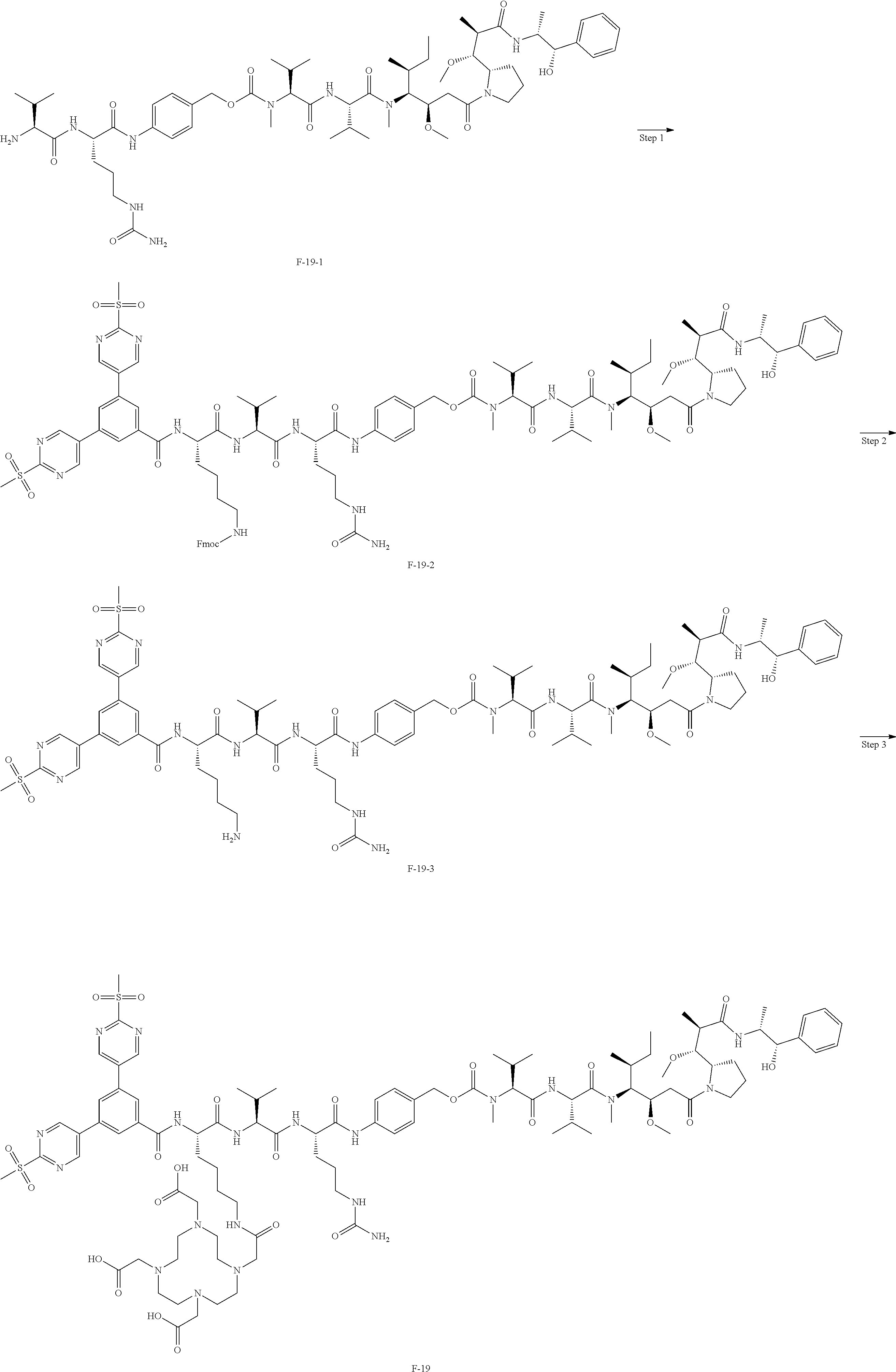

### Step 1: Preparation of 4-((9S,12S,15S)-9-(3,5-bis(2-(methylsulfonyl)pyrimidin-5-yl)benzamido)-1-(9H-fluoren-9-yl)-12-isopropyl-3,10,13-trioxo-15-(3-ureidopropyl)-2-oxa-4,11,14-triazahexadecan-16-amido)benzyl (1-((1-(((3R,5S)-1-((S)-2-((1R,2R)-3-(((1S,2R)-1-hydroxy-1-phenylpropan-2-yl)amino)-1-methoxy-2-methyl-3-oxopropyl) pyrrolidin-1-yl)-3-methoxy-5-meth yl-1-oxohept-4-yl)(methyl)amino)-3-methyl-1-oxobutan-2-yl)amino)-3-methyl-1-oxobutan-2-yl) (methyl)carbamate (F-19-2)

N⁶-(((9H-Fluoren-9-yl)methoxy)carbonyl)-N²-(3,5-bis(2-(methylsulfonyl)pyrimidin-5-yl) be nzoyl)-L-lysine (25 mg, 31.90 μmol) and VC-MMAE (CAS: 644981-35-1; 42.5 mg, 38.30 μmol) were dissolved in DMF (2 mL), DIPEA (16 mg) and HATU (19 mg) were added, and then reacted at 25° C. for 1 hour. After the reaction was completed, the product was purified by flash column chromatography (C18, water/acetonitrile=0.7) and freeze-dried to obtain the title compound (36.2 mg, 19.1 μmol).

Its structural characterization data are as follows:

MS m/z (ESI): 1890.1 [M+H]⁺

### Step 2: Preparation of 4-((S)-2-((S)-2-((S)-6-amino-2-(3,5-bis(2-(methylsulfonyl) pyrimidin-5-yl)benzamido) hexanamido)-3-methylbutyramido)-5-ureidopentanamido)benzyl (1-((1-(((3R,5S)-1-((S)-2-((1R,2R)-3-(((1S,2R))-1-hydroxy-1-phenylpropan-2-yl)amino)-1-metho xy-2-methyl-3-oxopropyl) pyrrolidin-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (F-19-3)

4-((9S,12S,15S)-9-(3,5-Bis(2-(methylsulfonyl)pyrimidin-5-yl)benzamido)-1-(9H-fluoren-9-yl)-12-isopropyl-3,10,13-trioxo-15-(3-ureidopropyl)-2-oxa-4,11,14-triazahexadecan-16-amido) b enzyl (1-((1-(((3R,5S)-1-((S)-2-((1R,2R)-3-(((1S,2R)-1-hydroxy-1-phenylpropan-2-yl)amino)-1~ methoxy-2-methyl-3-oxopropyl) pyrrolidin-1-yl)-3-methoxy-5-methyl-1-oxohept-4-yl)(methyl) a mino)-3-methyl-1-oxobutan-2-yl)amino)-3-methyl-1-oxobutan-2-yl)(methyl)carbamate (36.2 mg, 19.1 μmol) was dissolved in DMF (2 mL), diethylamine (50 mL) was added. After the addition, the resulting mixture was reacted at 25° C. for 0.5 hours. After the reaction was completed, the product was purified by flash column chromatography (C18, water/acetonitrile=0.5) and freeze-dried to obtain the title compound (25.5 mg, 15.3 μmol).

Its structural characterization data are as follows:

MS m/z (ESI): 1667.9 [M+H]⁺

### Step 3: Preparation of 2,2′,2″-(10-((6S,9S,12S)-1-amino-12-(3,5-bis(2-(methylsulfonyl) pyrimidin-5-yl)benzamido)-6-((4-((12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl) pyrrolidin-1-yl)-2-oxoe thyl)-5,8-diisopropyl-4,10-dimethyl-3,6,9-trioxo-2,13-dioxa-4,7,10-triazatetradecyl)phenyl)carba moyl)-9-isopropyl-1,8,11,18-tetraoxo-2,7,10,17-tetraazanonadecan-19-yl)-1,4,7,10-tetraazacyclo dodecan-1,4,7-triyl)triacetic acid (F-19)

4-((S)-2-((S)-2-((S)-6-Amino-2-(3,5-bis(2-(methylsulfonyl)pyrimidin-5-yl)benzamido) hexa namido)-3-methylbutyramido)-5-ureidopentanamido)benzyl (1-((1-(((3R,5S)-1-((S)-2-((1R,2R)-3-(((1S,2R))-1-hydroxy-1-phenylpropan-2-yl)amino)-1-methoxy-2-methyl-3-oxopropyl) pyrrolidi n-1-yl)-3-methoxy-5-methyl-1-oxohept-4-yl)(methyl)amino)-3-methyl-1-oxobutan-2-yl)amino)-3-methyl-1-oxobutan-2-yl)(methyl)carbamate (25.5 mg, 15.3 μmol) was dissolved in DMF (2 mL), DIPEA (10 mg) was added, and then 2,2′,2″-(10-(2-((2,5-dioxopyrrolidin~1-yl)oxy)-2-oxoethyl)-1,4,7,10-tetraazacyclododecan-1,4,7-triyl)triacetic acid (23.1 mg, 45.9 μmol) was added. After the reaction was completed, the reaction solution was directly purified by preparative HPLC and freeze-dried to obtain the title compound (12.09 mg, 5.9 μmol).

Its structural characterization data are as follows:

MS m/z (ESI): [M/2+H]⁺=1027.1

Its preparation method was follows:
Chromatographic column: Waters SunFire Prep C18 OBD (5 μm*19 mm*150 mm)
Mobile phase A: acetonitrile; Mobile phase B: water (0.05% formic acid)

| Time | Mobile | Mobile | Flow rate |
|---|---|---|---|
| [min] | phase A [%] | phase B [%] | [mL/min] |
| 0.00 | 20 | 80 | 30 |
| 4.00 | 20 | 80 | 30 |
| 24.00 | 90 | 10 | 30 |

### II. Preparation of Antibodies

Based on the amino acid sequence of Trastuzumab (IMGT/mAb-DB ID: 97) and the amino acid sequence of Pertuzumab (IMGT/mAb-DB ID: 80) retrieved from IMGT database, codon optimization was carried out. Subsequently, the coding gene was synthesized and cloned into an expression vector. The recombinant expression vector was then transfected into CHO cells. Pressure-screening was performed to establish stable expression cell lines. After expression, the supematants were collected, and the corresponding antibodies, Trastuzumab and Pertuzumab, were purified using Protein A affinity chromatography. The information of amino acid sequences of Trastuzumab and Pertuzumab is presented above.

### III. Conjugation of Compounds Containing Cell Bioactive Molecules and Linkers with Antibodies

The antibodies Trastuzumab and Pertuzumab involved in the antibody-drug conjugates prepared in the following examples were Trastuzumab and Pertuzumab described in the second part above.

### 1. Preparation of Trastuzumab-L-1 (DAR 2)

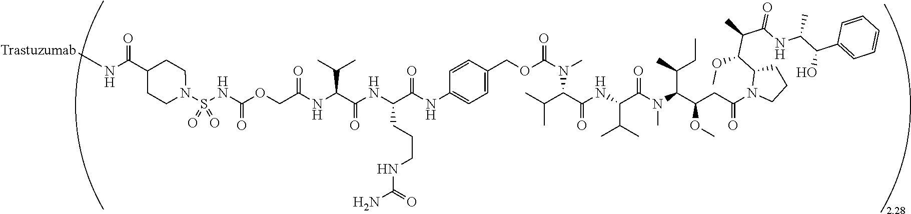

0.31 mL of Trastuzumab antibody (16.2 mg/mL) was taken, adjusted to pH 7.40 with 1M Na₂HPO₄ solution, L-1 (18.15 μL, 10 mM) in dimethylsulfoxide solution was added at a 5-fold molar ratio to the antibody. The resulting mixture was mixed well, allowed to stand at room temperature for 18 hours, and then subjected to buffer exchange using a NAP-5 gel column (Cytiva) equilibrated with 20 mM histidine buffer solution at pH 6.0 to obtain the antibody-drug conjugate (i.e., Trastuzumab-L-1). The average drug-to-antibody ratio (DAR value) determined by mass spectrometry was 2.28, with DAR2 accounting for 60.28%.

### 2. Preparation of Trastuzumab-L-4 (DAR 2)

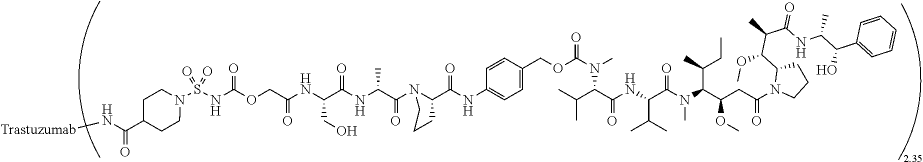

0.123 mL of Trastuzumab antibody (16.2 mg/mL) was taken, diluted with 200 μL of 20 mM PB (pH 7.60), and then adjusted to pH 7.40 with 1M Na₂HPO₄ solution, followed by an addition of L-4 (7.10 μL, 10 mM) in dimethylsulfoxide solution at a 5-fold molar ratio to the antibody. The resulting mixture was mixed well, allowed to stand at room temperature for 18 hours, and then subjected to buffer exchange using a NAP-5 gel column (Cytiva) equilibrated with 20 mM histidine buffer solution at pH 6.0 to obtain the antibody-drug conjugate (i.e., Trastuzumab-L-4). The DAR value determined by mass spectrometry was 2.35, with DAR2 accounting for 57.97%.

### 3. Preparation of Trastuzumab-F-15

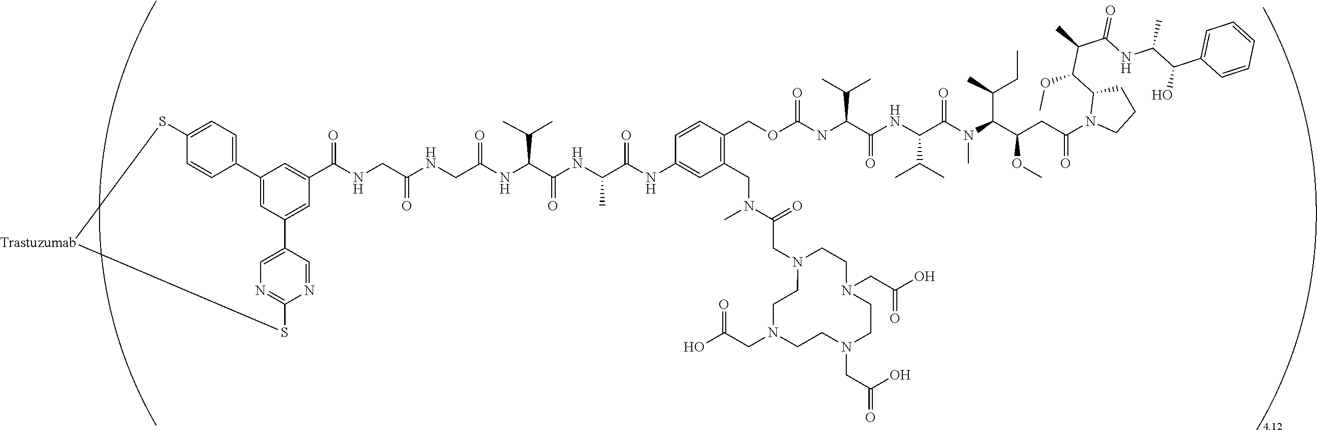

2.027 mL of Trastuzumab antibody (14.8 mg/mL) was taken, diluted with 101 μL of 20 mM PB+0.1M EDTA (pH 7.60), and then adjusted to pH 7.60 with 1M Na₂HPO₄ solution, followed by an addition of 10 mM TCEP (tri (2-carboxyethyl) phosphine, 113.6 μL, pH 7.60) solution, mixed well, and allowed to stand at room temperature for 1.5 hours. Then F-15 (135.7 μL, 10 mM) in dimethylsulfoxide solution was added at a 6-fold molar ratio to the antibody. The resulting mixture was mixed well, allowed to stand at room temperature for 18 hours, and then subjected to buffer exchange using a NAP gel column (Cytiva) equilibrated with 20 mM histidine buffer solution (pH 6.0) to obtain the antibody-drug conjugate (i.e., Trastuzumab-F-15). The DAR value determined by mass spectrometry was 4.12, with DAR4 accounting for 88.43%.

### 4. Preparation of Trastuzumab-F-16

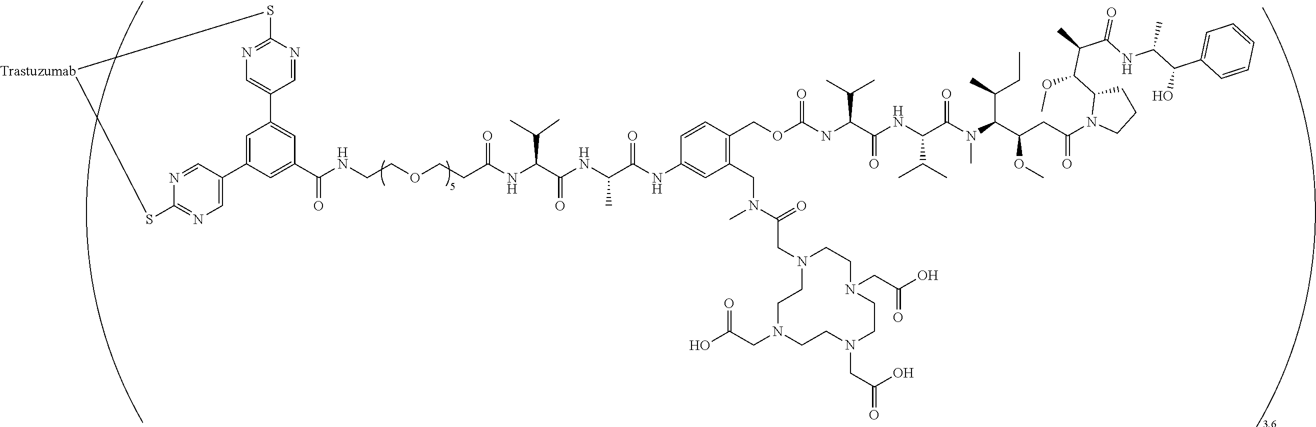

1.7 mL of Trastuzumab antibody (14.8 mg/mL) was taken, diluted with 85 μL of 20 mM PB+0.1M EDTA (pH 7.60), and then adjusted to pH 7.60 with 1M Na₂HPO₄ solution, followed by an addition of 10 mM TCEP (tris(2-carboxyethyl) phosphine, 94.7 μL, pH 7.60) solution, mixed well, and allowed to stand at room temperature for 1.5 hours. Then F-16 (110.5 μL, 10 mM) dissolved in dimethyl sulfoxide was added at a 6-fold molar ratio to the antibody. The resulting mixture was mixed well, allowed to stand at room temperature for 18 hours, and then subjected to buffer exchange using a NAP gel column (Cytiva) equilibrated with 20 mM histidine buffer solution at pH 6.0 to obtain the antibody-drug conjugate (i.e., Trastuzumab-F-16). The DAR value determined by mass spectrometry was 3.6, with DAR4 accounting for 72.01%.

### 5. Preparation of Trastuzumab-F-18

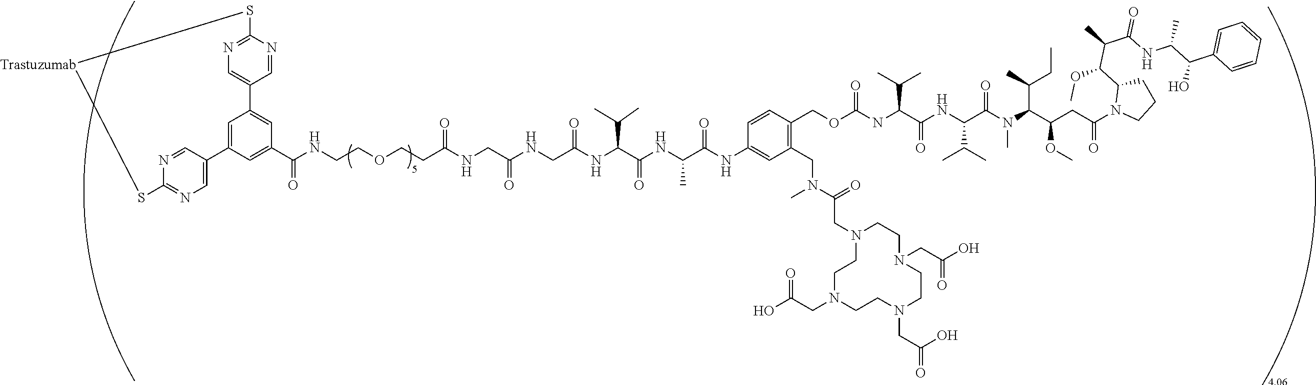

2.027 mL of Trastuzumab antibody (14.8 mg/mL) was taken, diluted with 101 μL of 20 mM PB+0.1M EDTA (pH 7.60), and then adjusted to pH 7.60 with 1M Na₂HPO₄ solution, followed by an addition of 10 mM TCEP (tris(2-carboxyethyl) phosphine, 113.6 μL, pH 7.60) solution, mixed well, and allowed to stand at room temperature for 1.5 hours. Then F-18 (131.8 μL, 10 mM) dissolved in dimethyl sulfoxide was added at 6-fold molar ratio to the antibody. The resulting mixture was mixed well, allowed to stand at room temperature for 18 hours, and subjected to buffer exchange using a NAP gel column (Cytiva) equilibriated with 20 mM histidine buffer solution at pH 6.0 to obtain the antibody-drug conjugate (i.e., Trastuzumab-F-18). The DAR value determined by mass spectrometry was 4.08, with DAR4 accounting for 92.32%.

### IV. Detection of Inhibitory Effect of Compounds on Tumor Cell Proliferation

### 1. Detection of Efficacy of Anti-Human Her2 Antibody-Drug Conjugates in the NCI-N87 Model

NCI-N87 cells were cultured in RPMI 1640 medium supplemented with 10% fetal bovine serum at 37° C. and 5% CO₂. NCI-N87 cells in the exponential growth phase were harvested, resuspended in PBS to an appropriate concentration, and then subcutaneously inoculated into female Balb/c-Nude mice to establish a gastric cancer model. When the average tumor volume reached approximately 150 mm³, the mice were randomly grouped according to tumor size, including the following groups in sequence: a vehicle control group (negative control, Vehicle group), a Trastuzumab-F-16 group at 1 mg/kg, a Trastuzumab-F-15 group at 1 mg/kg, a Trastuzumab-F-18 group at 1 mg/kg, and a control DS8201 group at 1 mg/kg (Note: DS8201 was an ADC targeting human HER2 developed by Daiichi Sankyo, and the sample used in this experiment was prepared in-house by Kelun Biotech). Each group received the administration via tail vein injection (i.v.). The conjugates of the present invention were administered on Day 0 and Day 7, for a total of two doses. The control DS8201 was administered on Day 0, Day 7 and Day 14, for a total of three doses. After administration, tumor diameters were measured twice a week using a vernier caliper, and tumor volume was calculated according to the following calculation formula: V=0.5 a×b², where a and b represent the long diameter and short diameter of the tumor, respectively. Animal mortality was observed and recorded daily.

The tumor growth inhibition rate TGI (%) was calculated using the following formula to evaluate the tumor-inhibitory efficacy of the ADCs:

$$If {V}_{T end} > {V}_{T 0} , TGI ( \% ) = [ 1 - ( {V}_{T end} - {V}_{T 0} ) / ( {V}_{C end} - {V}_{C 0} ) ] * 100 \% , or \\ If {V}_{T end} \leq {V}_{T 0} , TGI ( \% ) = [ 1 - ( {V}_{T end} - {V}_{T 0} ) / {V}_{T 0} ] * 100 \% .$$

where,
V_{T end}: mean tumor volume of the treatment group at the end of experiment
V_{T0}: mean tumor volume of the treatment group at the beginning of administration
V_{C end}: mean tumor volume of the negative control group at the end of experiment
V_{C0}: mean tumor volume of the negative control group at the beginning of administration

The following formula was used to calculate the tumor relative proliferation rate T/C (%), which was used to evaluate the tumor-inhibitory efficacy of the ADCs:

T/C(%)=(V_{T end}/V_{T0})/(V_{C end}/V_{C0})*100%.

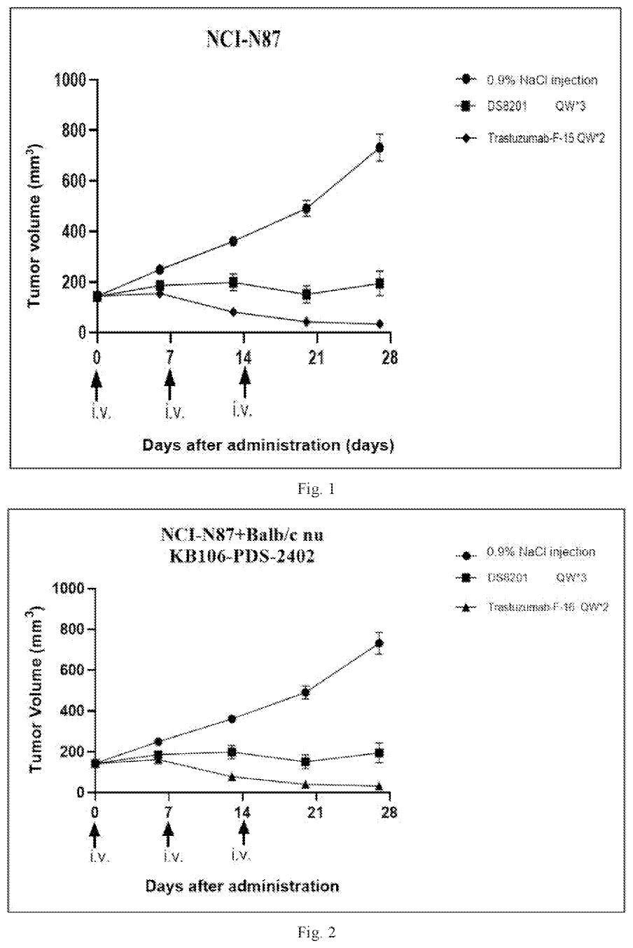

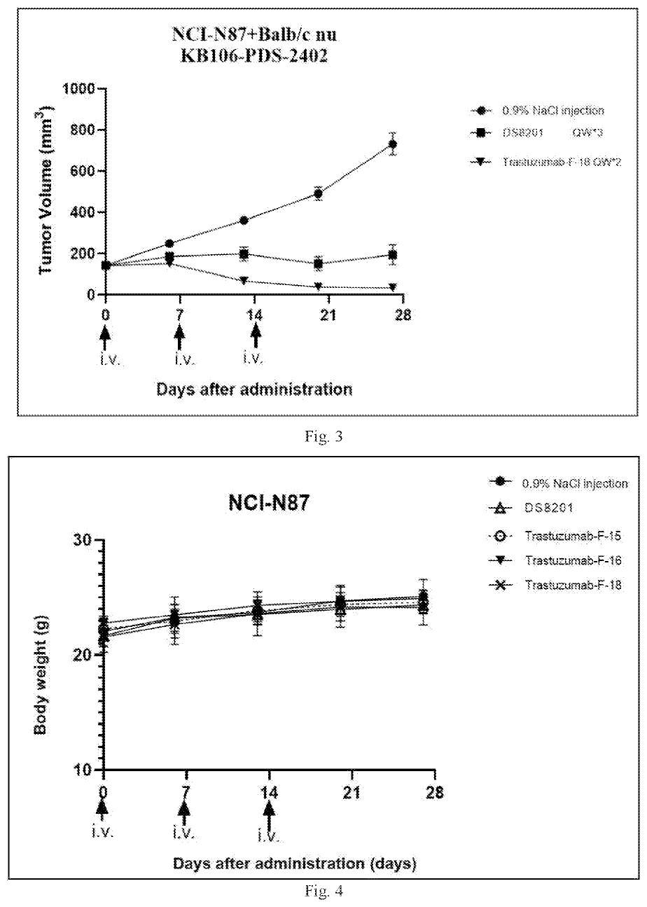

The ADCs of the present invention exhibited significant inhibitory effects on tumor growth in the NCI-N87 gastric cancer xenograft model. Compared with the Vehicle group, the tumor growth inhibition rate (TGI) for the Trastuzumab-F-16 at 1 mg/kg, Trastuzumab-F-15 at 1 mg/kg, Trastuzumab-F-18 at 1 mg/kg and the DS8201 group were 177.42%, 176.60%, 176.53% and 91.25%, respectively. By Day 27, no animal death or significant weight loss was observed in the treatment groups, and no obvious drug toxicity reaction was noted. The mice had good tolerance to the ADCs of the present invention during the treatment period. Specific results are shown in Table 11, FIG. 1, FIG. 2, FIG. 3 and FIG. 4.

TABLE 11
Human gastric cancer cell NCI-N87 CDX model

| | | Day 27 | | | |
|---|---|---|---|---|---|
| | Dose | Tumor | | | P value |
| | (mg/ | volume (mm³) | TGI | T/C | (vs. |
| Group | kg) | (x̅ ± SEM) | (%) | (%) | Vehicle) |
| Vehicle | — | 732.45 ± 53.51 | — | — | — |
| DS8201 | 1 | 194.7 ± 48.78 | 91.25 | 26.66 | <0.001 |
| Trastuzumab-F-15 | 1 | 33.73 ± 3.30 | 176.60 | 4.59 | <0.001 |
| Trastuzumab-F-16 | 1 | 32.15 ± 3.19 | 177.42 | 4.43 | <0.001 |
| Trastuzumab-F-18 | 1 | 33.37 ± 4.93 | 176.53 | 4.60 | <0.001 |

Although specific embodiments of the present invention have been described in details, those skilled in the art will understand that various modifications and substitutions can be made to those details based on all the teachings disclosed, and these changes are within the scope of protection of the present invention. The full scope of the present invention is given by the appended claims and any equivalents thereof.

What is claimed is:

1. An antibody-drug conjugate, which has the formula Ab-[M-L-E-D]ₓ, wherein:
Ab represents an antibody or antigen-binding fragment thereof capable of specifically binding to epidermal growth factor receptor 2 (Her2), a member of the ErbB family of receptor tyrosine kinases:
M represents a structural fragment connected to the antibody or antigen-binding fragment thereof;
L represents a structural fragment connecting the linking site M and E;
E represents a structural fragment connecting L and D;
D represents a cytotoxic drug fragment;
x is 1 to 10.

2. The antibody-drug conjugate according to claim 1, wherein M is selected from the group consisting of the following structural fragments:

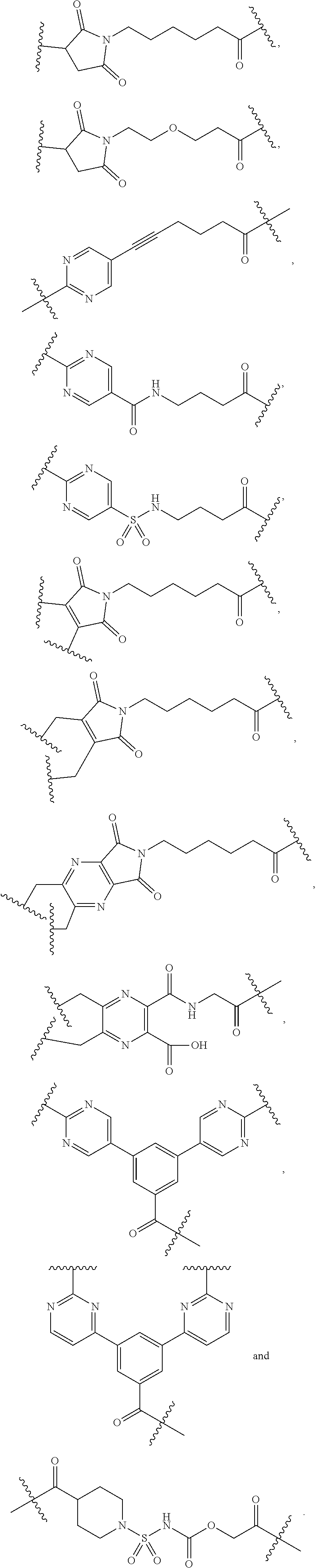

3. The antibody-drug conjugate according to claim 1, wherein M is selected from the group consisting of the following structural fragments:

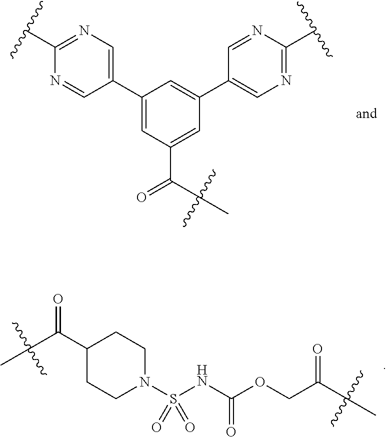 [SMILES: *C(=O)c1cc(-c2cnc(*)nc2)cc(-c2cnc(*)nc2)c1 ; *C(=O)COC(=O)NS(=O)(=O)N1CCC(C(*)=O)CC1]

4. The antibody-drug conjugate according to any one of claims 1 to 3, wherein L is selected from the group consisting of divalent structural fragments composed of one or more of the following groups: C₁₋₆ alkylene, 6- to 10-membered arylene, 5- to 6-membered heteroarylene, —N(R′)—, carbonyl group, —O—, a residue of an amino acid or an analog thereof (the amino acid is a natural amino acid or a non-natural amino acid, such as Ala, Arg, Asn, Asp, Cit, Cys, Gln, Glu, Gly, His, Ile, Leu, Lys, Met, Phe, Pro, Ser, Thr, Trp, Tyr, Val, D-Val, D-Leu, D-Ala or Lys (COCH₂CH₂(OCH₂CH₂)ᵣOCH₃)), a fragment of a short peptide consisting of two or more amino acids (the short peptide is, for example, selected from the group consisting of Ala-Ala, Ala-Lys, Ala-Lys(Ac), Ala-Pro, Gly-Glu, Gly-Gly, Phe-Lys, Phe-Lys(Ac), Val-Ala, Val-Cit, Val-Lys, Val-Lys(Ac), Ala-Ala-Ala, Ala-D-Ala-Ala, Ala-Ala-Asn, Ala-Ala-Gly, D-Leu-Ala-Glu, Gly-Gly-Arg, Gly-Glu-Gly, Gly-Gly-Gly, Gly-Ser-Lys, Glu-Val-Ala, Glu-Val-Cit, Ser-D-Ala-Pro, Val-Leu-Lys, Val-Lys-Ala, Val-Lys-Gly, Gly-Gly-Phe-Gly, Gly-Gly-Val-Ala, Gly-Phe-Leu-Gly, Glu-Ala-Ala-Ala and Gly-Gly-Gly-Gly-Gly), Lys (R′), 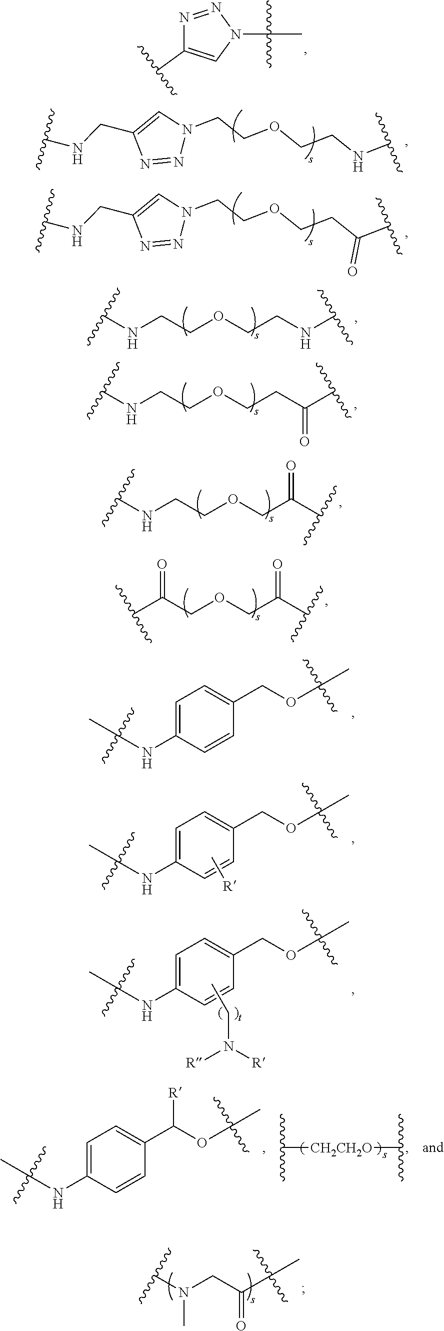
wherein R′ and R″ each independently represent hydrogen, C₁₋₆ alkyl, glucosyl, galactosyl, glucuronic acid group, galacturonic acid group, —CH₂N(C₁₋₆ alkyl)-C(═O)—(CH₂CH₂O)ᵣ—C₁₋₆ alkyl, —CH₂N(C₁₋₆ alkyl)-C(═O)—O—(CH₂CH₂O)ᵣ—C₁₋₆ alkyl, —(CH₂N(Me)-C(═O))ᵣ—C₁₋₆ alkyl, a polyethylene glycol fragment containing 1 to 10 EO units (i.e., —(CH₂CH₂O)ᵣ—C₁₋₆ alkyl), DOTA (1,4,7,10-tetraazacyclododecane-1,4,7,10-tetraacetic acid residue), DOTAGA (1,4,7,10-tetraazacyclododecane-1,4,7,10-tetraacetic acid-α-propionyl), or NOTA (1,4,7-triazacyclononane-N,N′,N″-triacetic acid residue), wherein r is an integer selected from 1 to 20, such as 1, 2, 3, 4, 5, 6, 7, 8, 9, 10, 11, 12, 13, 14, 15, 16, 17, 18, 19, 20; s is an integer selected from 1 to 20, such as 1, 2, 3, 4, 5, 6, 7, 8, 9, 10, 11, 12, 13, 14, 15, 16, 17, 18, 19, 20, preferably 5, 8 or 9; t is an integer selected from 1 to 5; preferably, tis 1;
preferably, L is selected from the group consisting of divalent structural fragments composed of one or more of the following groups: 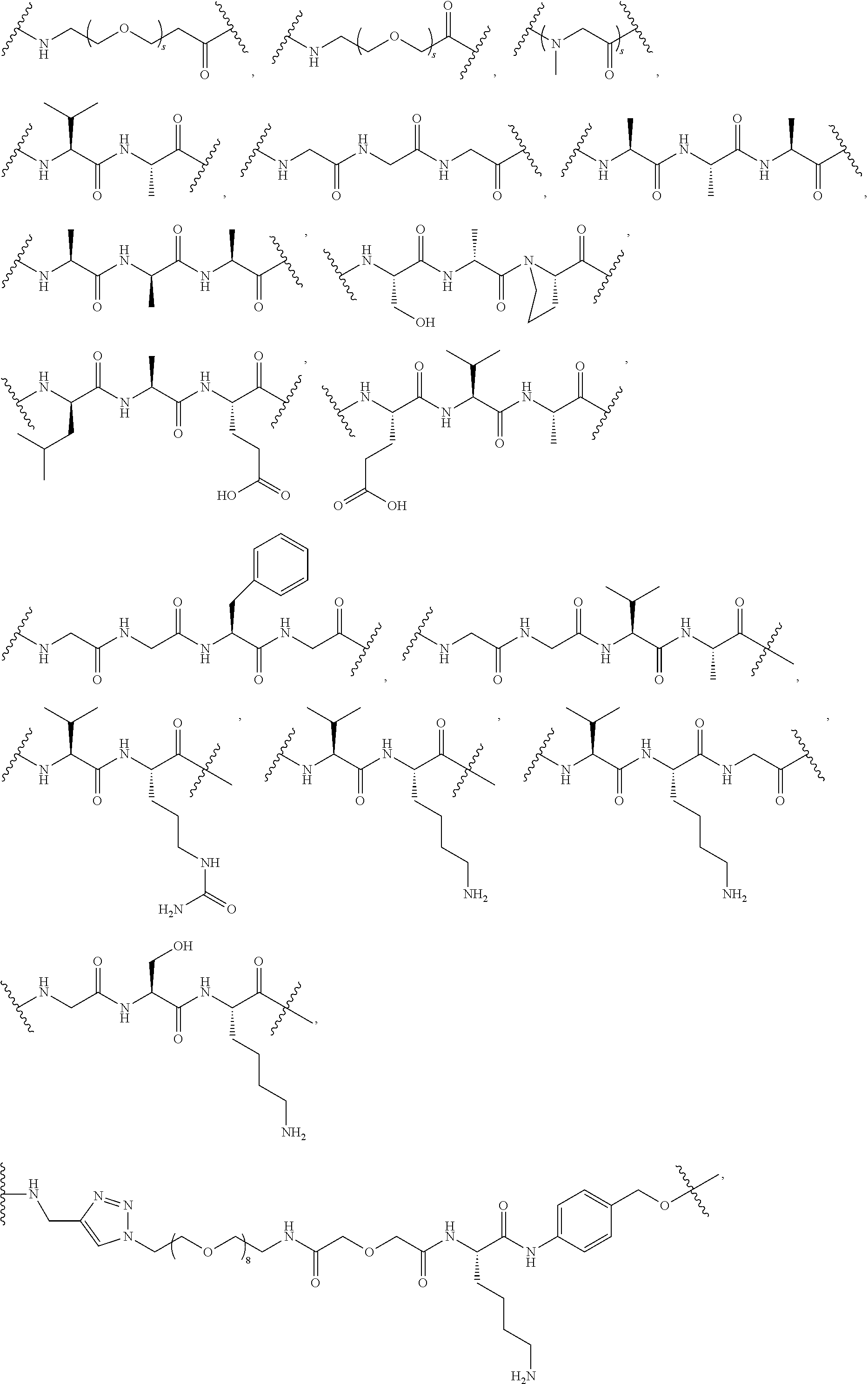 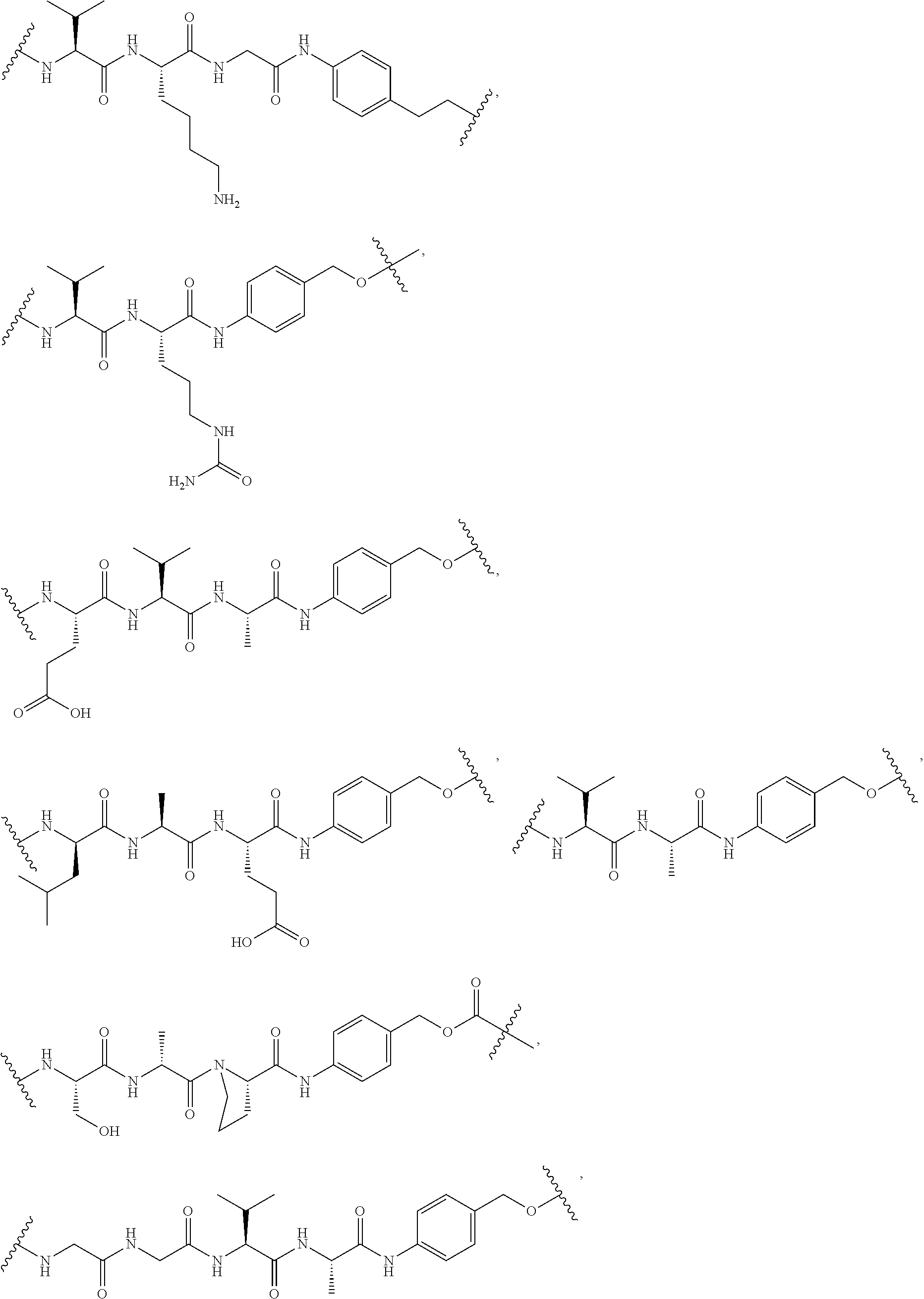 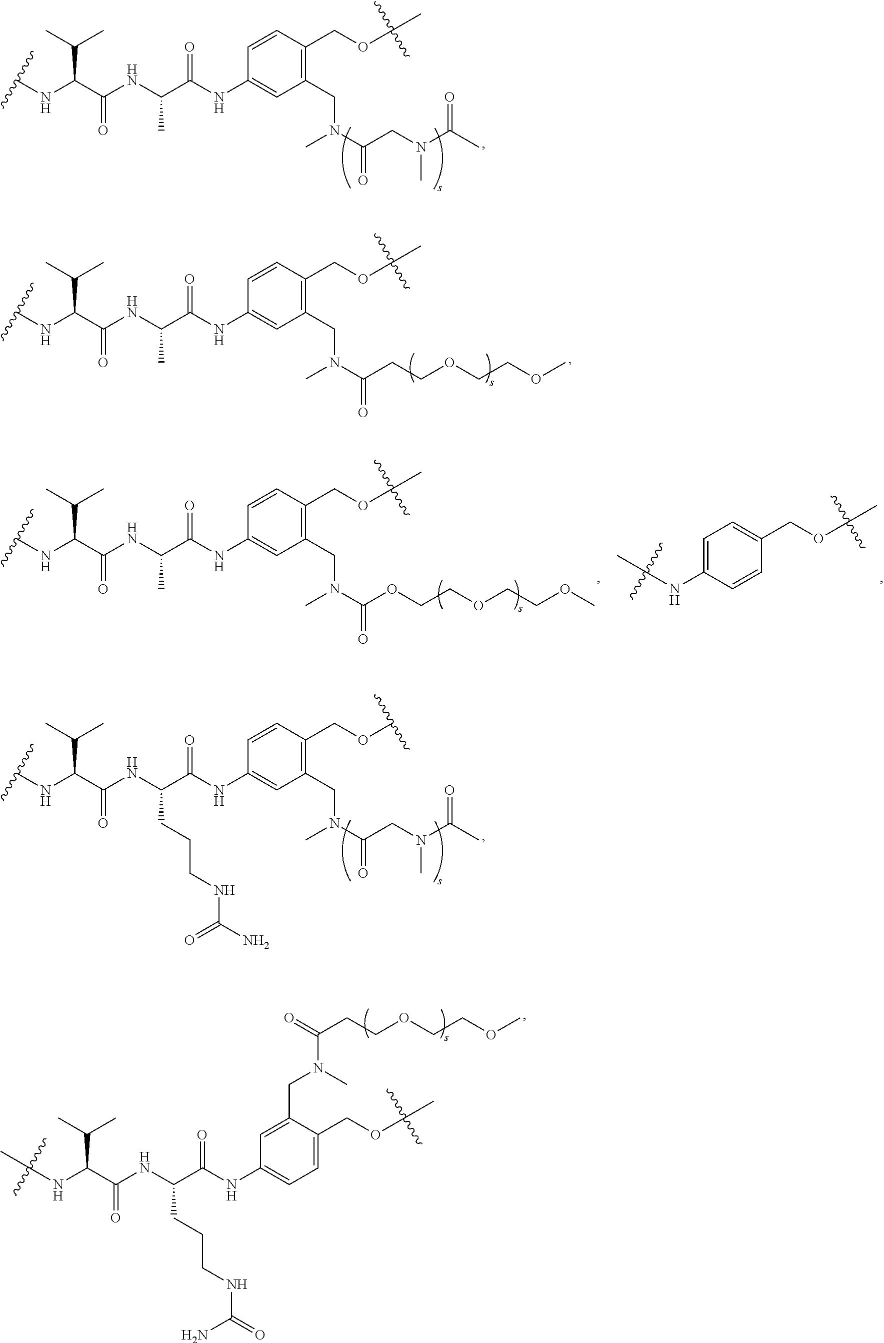 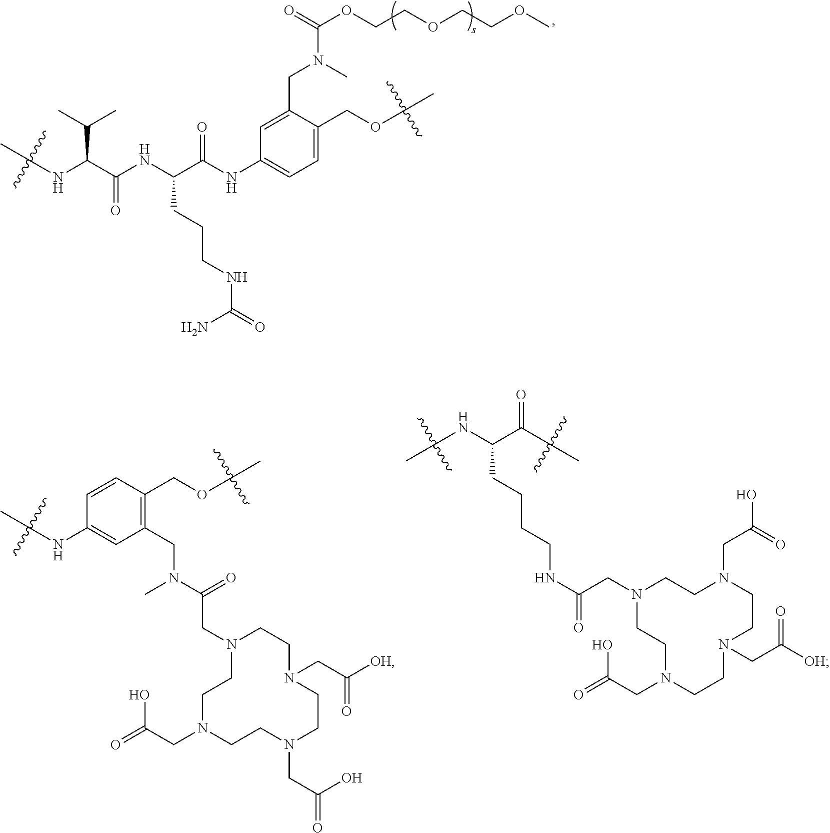
s is an integer selected from 1 to 20;
preferably, L is selected from the group consisting of: 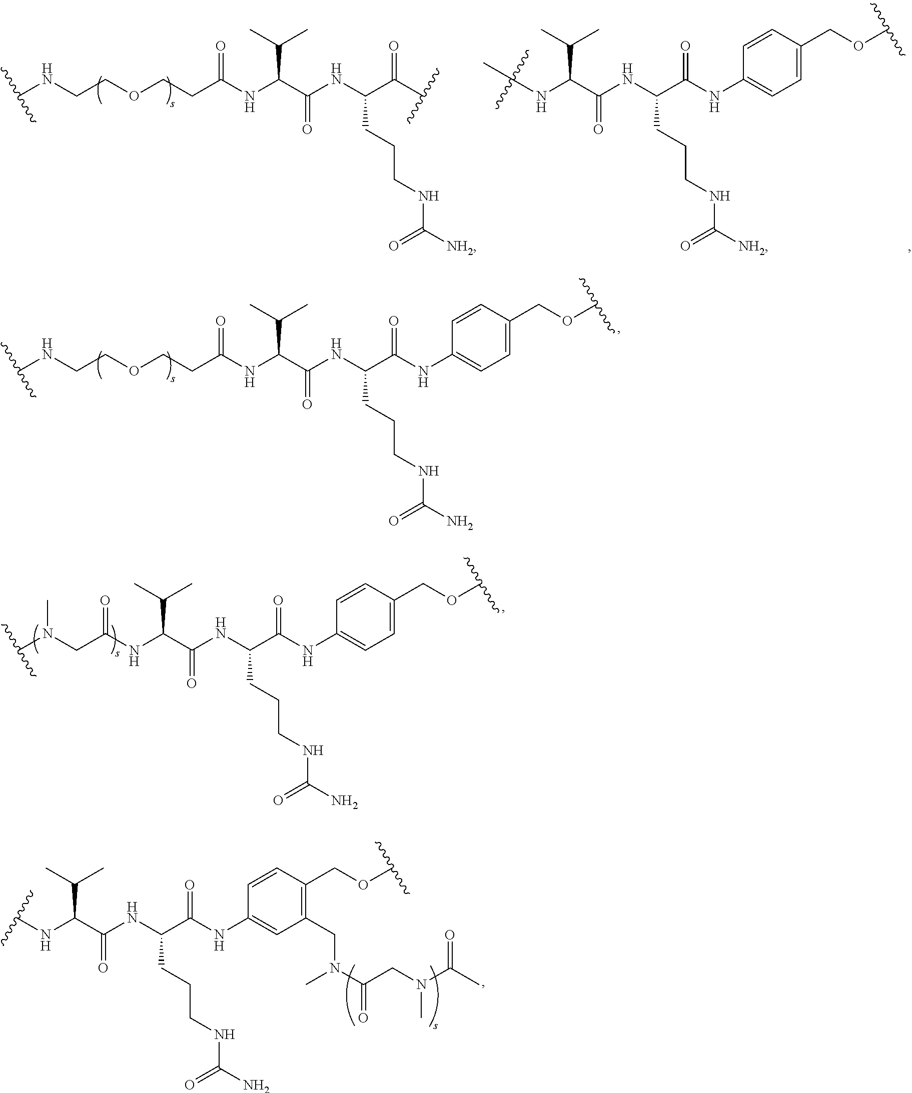 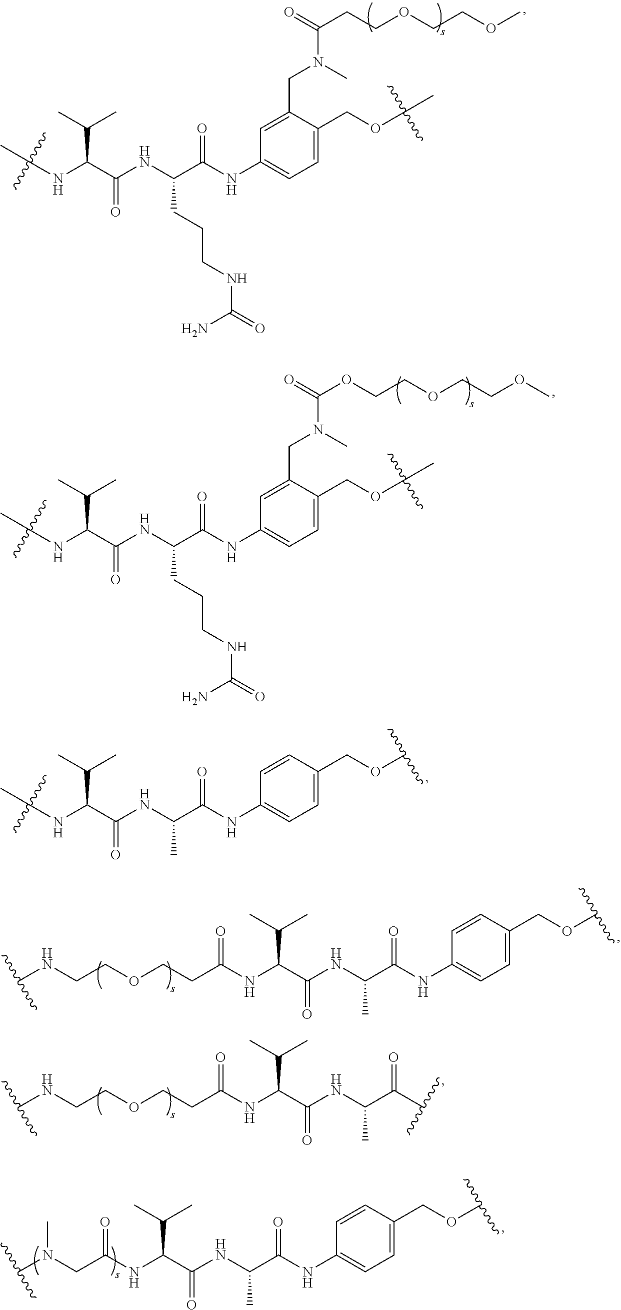 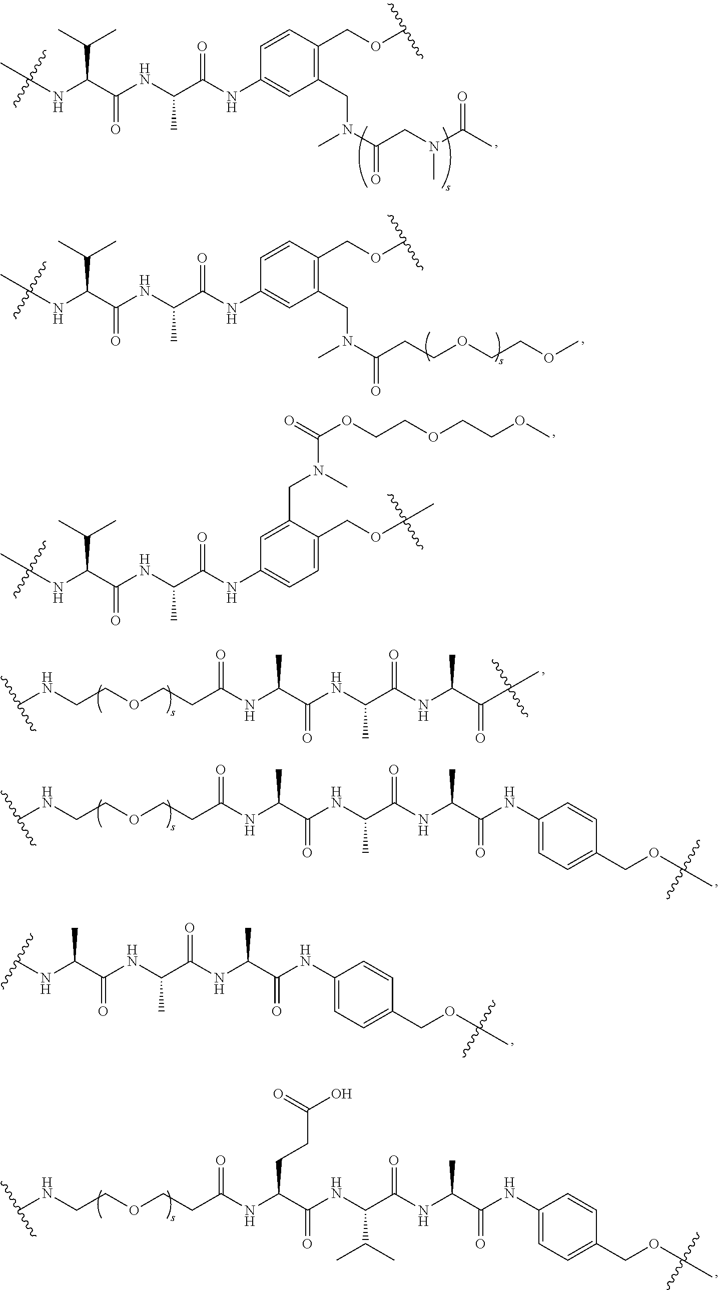 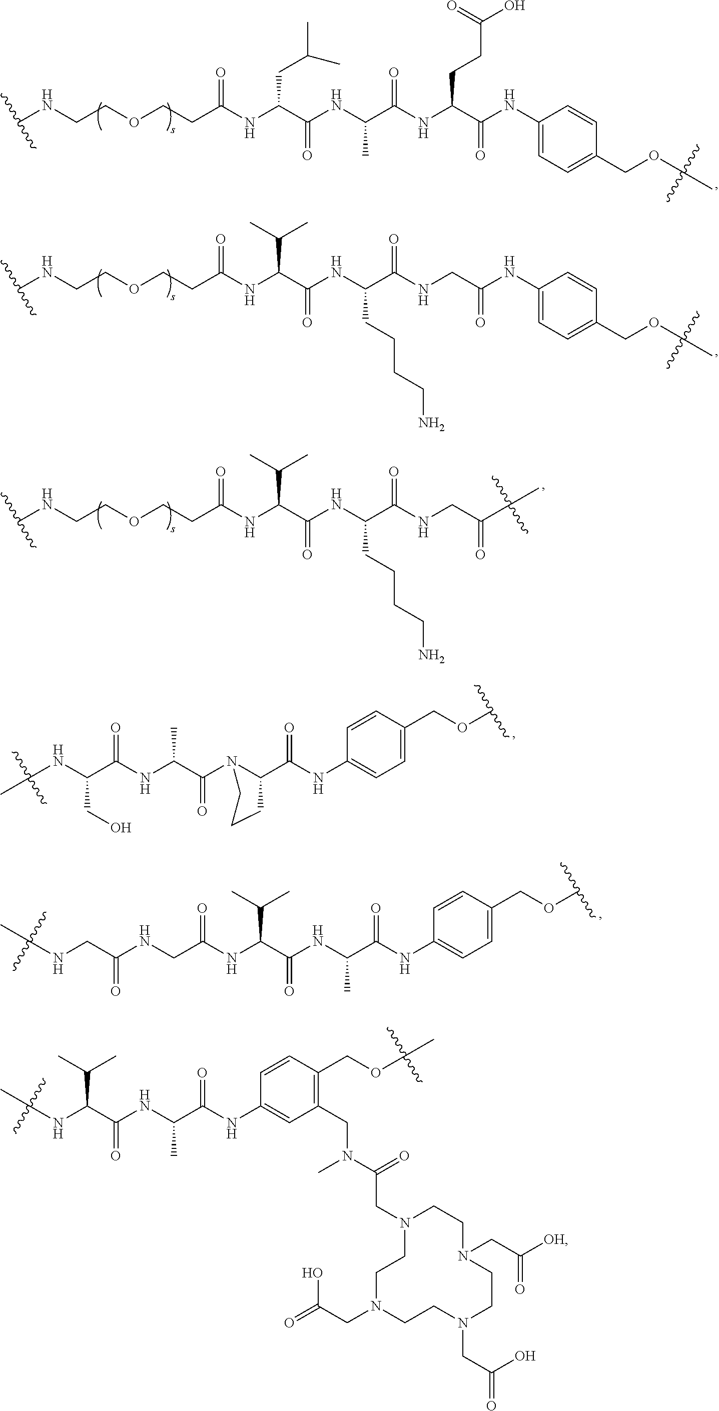 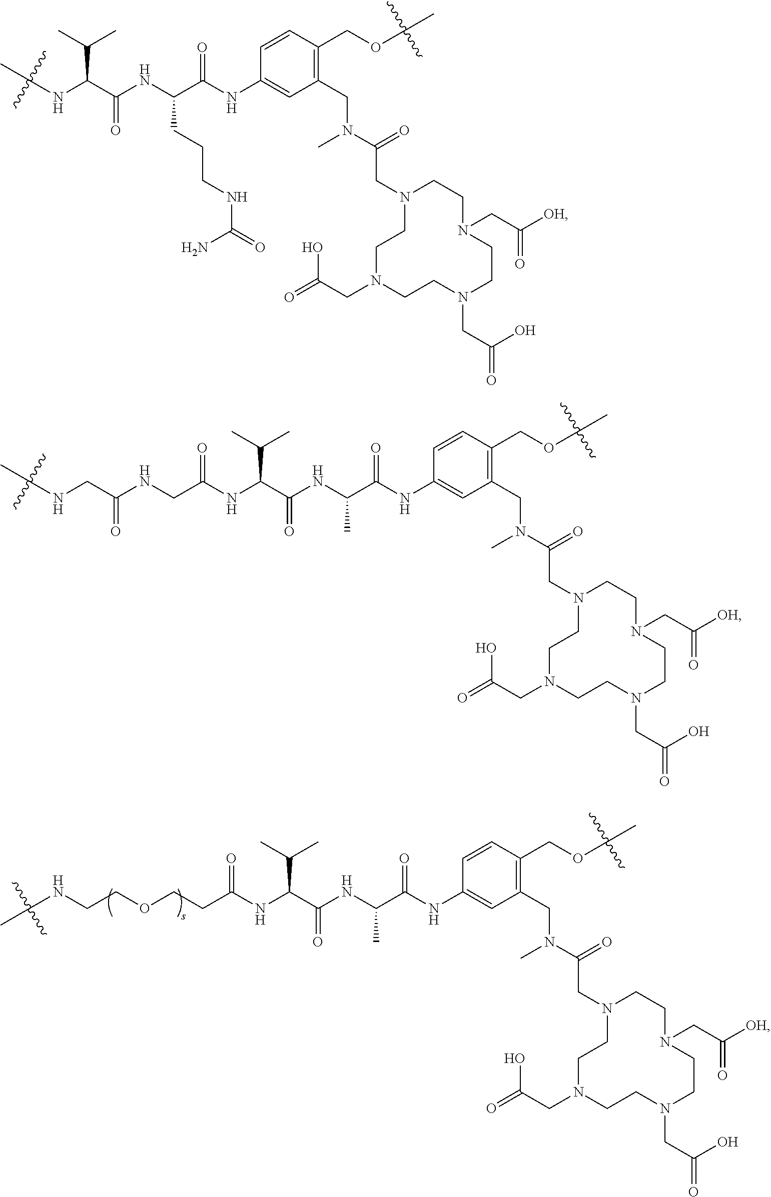 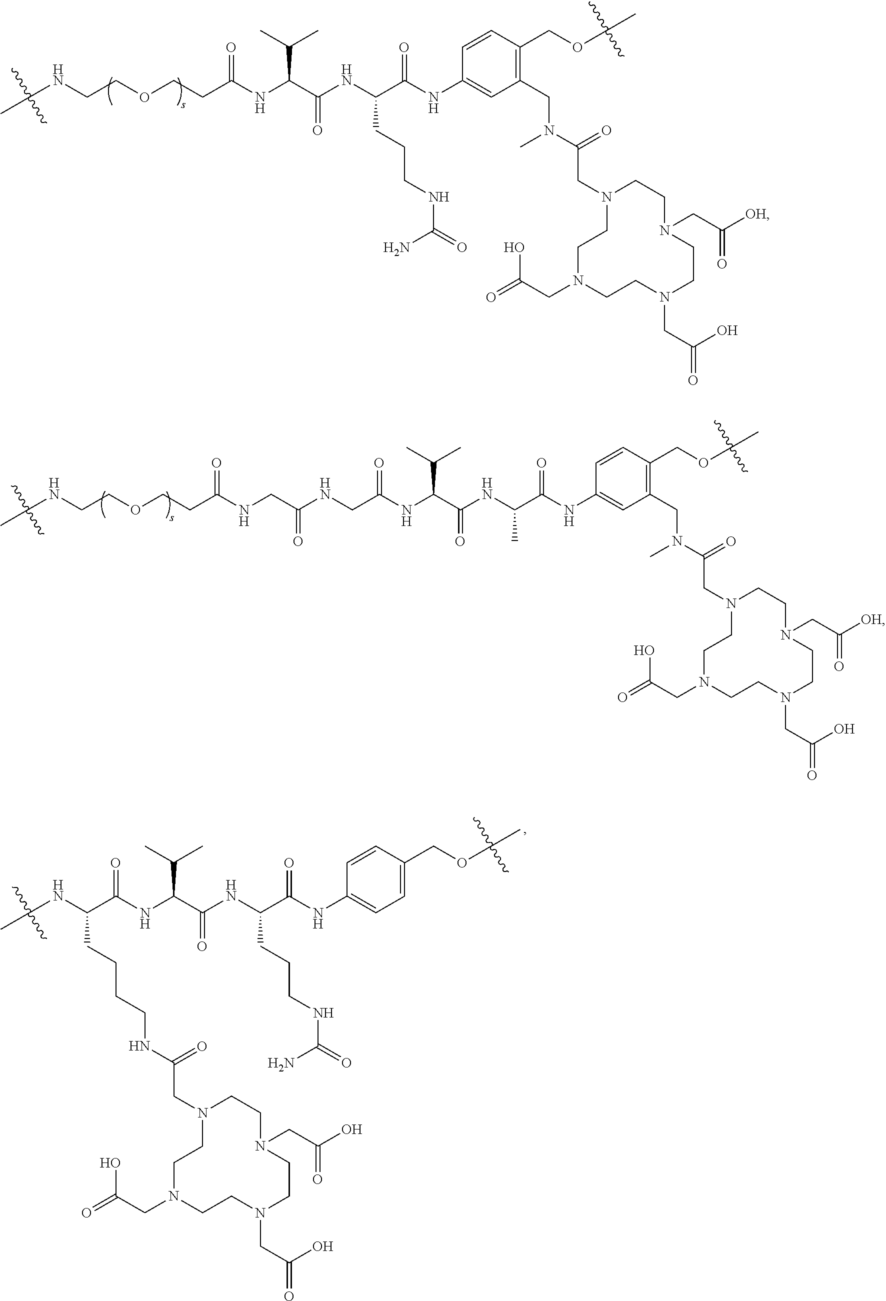 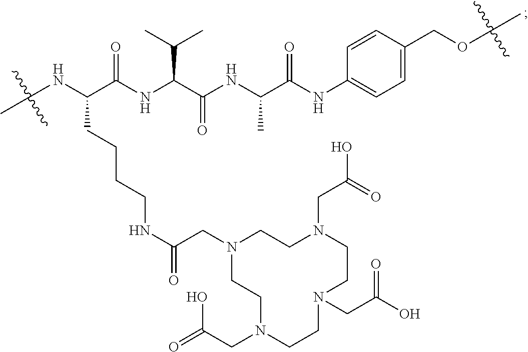 [SMILES: *N[C@@H](CCCCNC(=O)CN1CCN(CC(=O)O)CCN(CC(=O)O)CCN(CC(=O)O)CC1)C(=O)N[C@H](C(=O)N[C@@H](C)C(=O)Nc1ccc(CO*)cc1)C(C)C]
s is an integer selected from 1 to 20.

5. The antibody-drug conjugate according to any one of claims 1 to 4, wherein E is a single bond, —NH—CH₂—, or is selected from the group consisting of the following structural fragments: 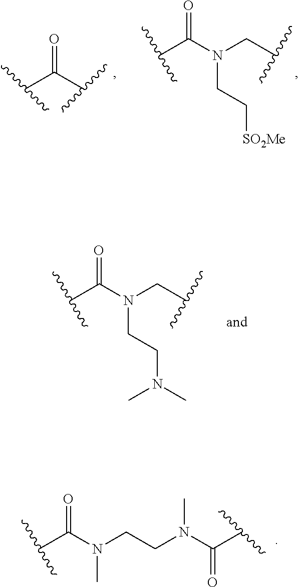
preferably, E is —NH—CH₂— or 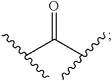 [SMILES: *C(*)=O]
further preferably, E is

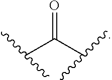 [SMILES: *C(*)=O]

6. The antibody-drug conjugate according to any one of claims 1 to 5, wherein the cytotoxic drug is an anti-tubulin agent;
preferably, the tubulin inhibitor is an auristatin compound;
preferably, the auristatin compound is selected from the group consisting of the following compounds or isotopically labeled compounds thereof: 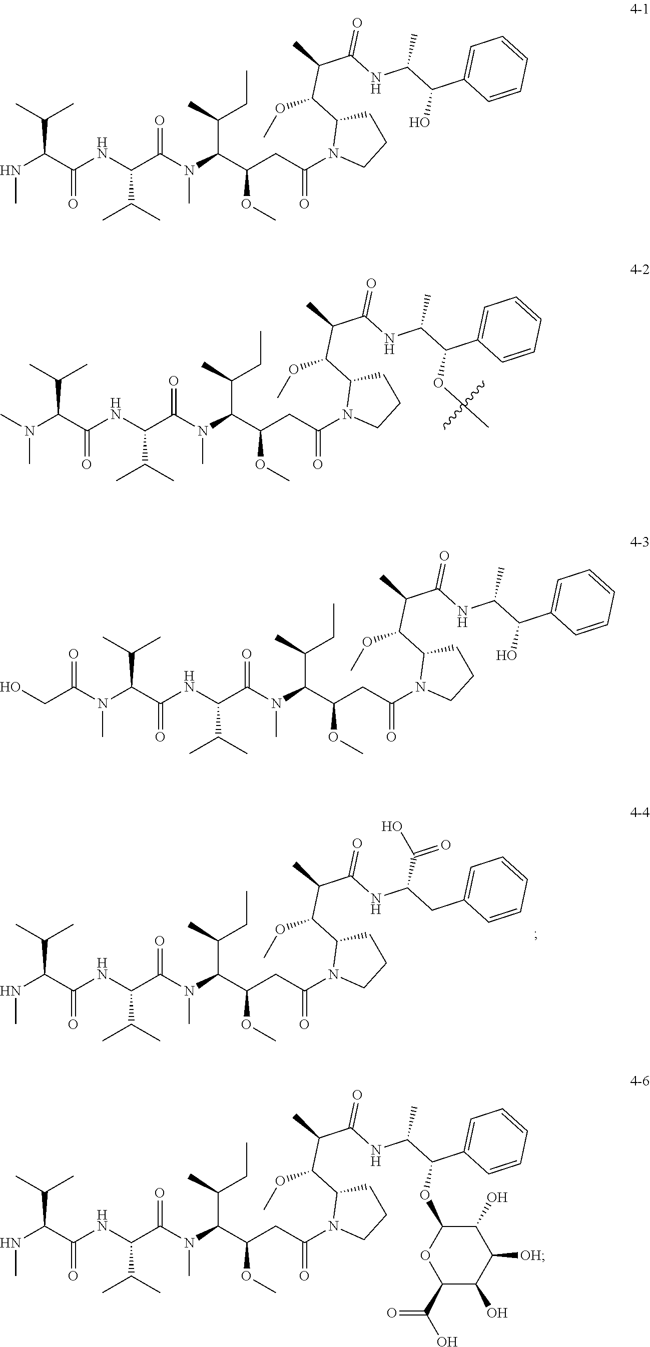
preferably, the cytotoxic drug is connected via —OH, primary amino, secondary amino or tertiary amino thereon to E in the antibody-drug conjugate;
preferably, the cytotoxic drug fragment D is selected from the group consisting of:

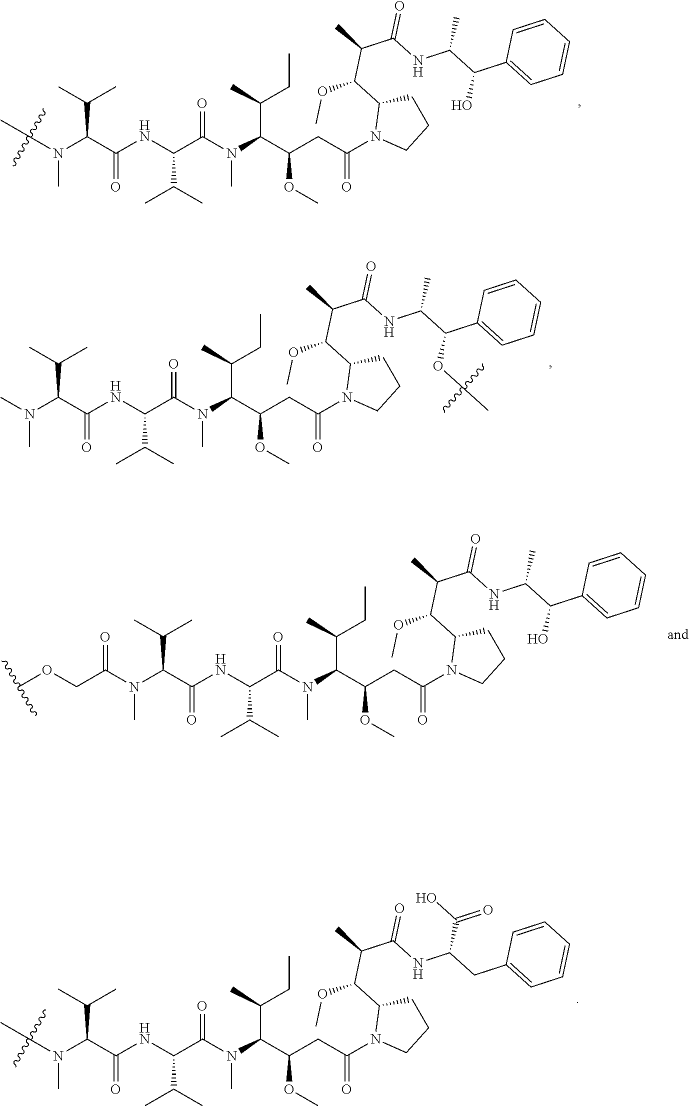

7. The antibody-drug conjugate according to any one of claims 1 to 6, wherein -M-L-E-D is formed from a drug-linker selected from the group consisting of F′-1 to F′-12, F′-16 to F-18, L′-6 to L′-11, F-1 to F-20 and L-1 to L-11, preferably selected from the group consisting of F-1 to F-20 and L-1 to L-11, for example, by a substitution reaction (for example, removal of a methylsulfonyl or a pentafluorophenoxy thereon) or by an addition reaction: 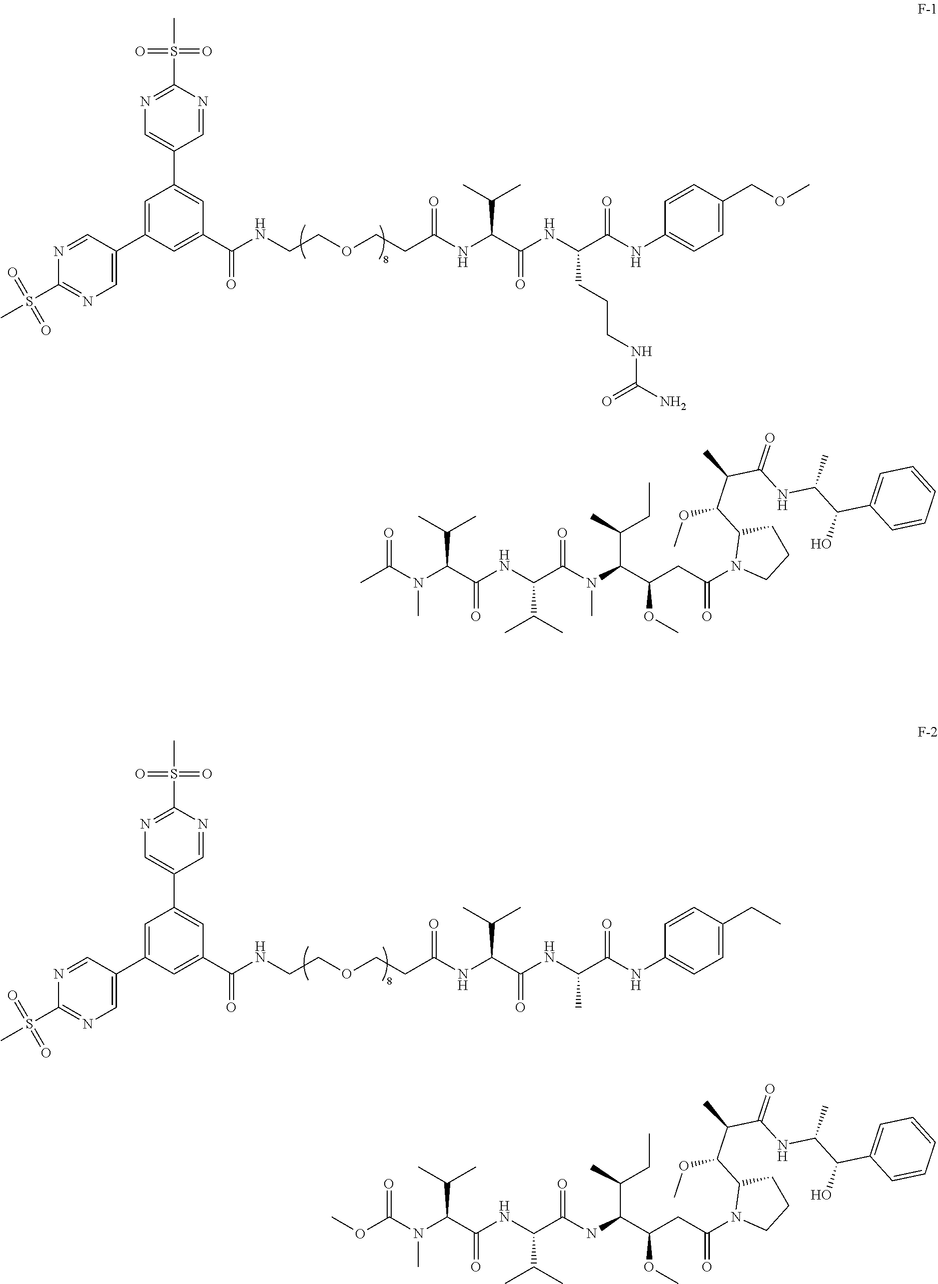 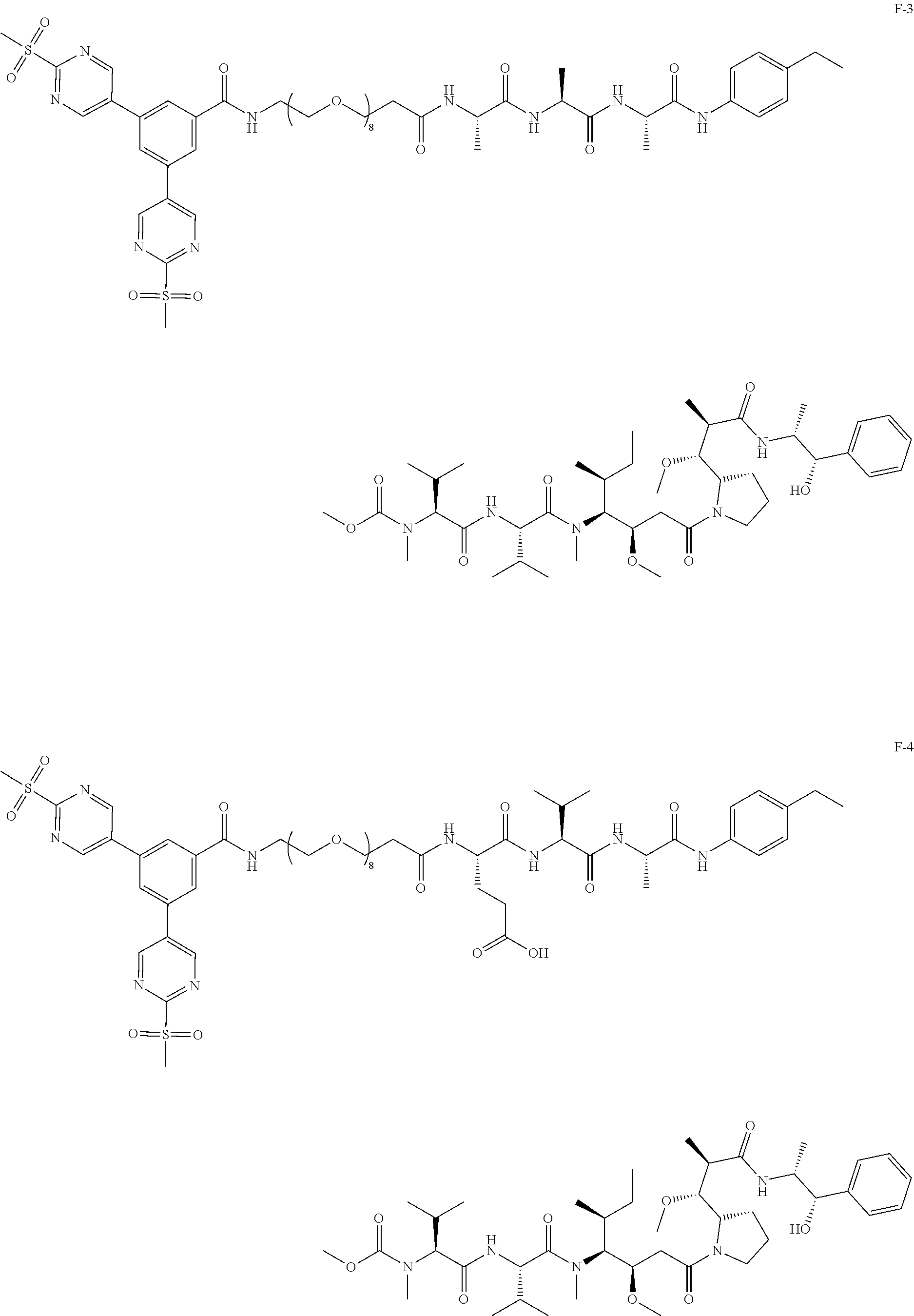 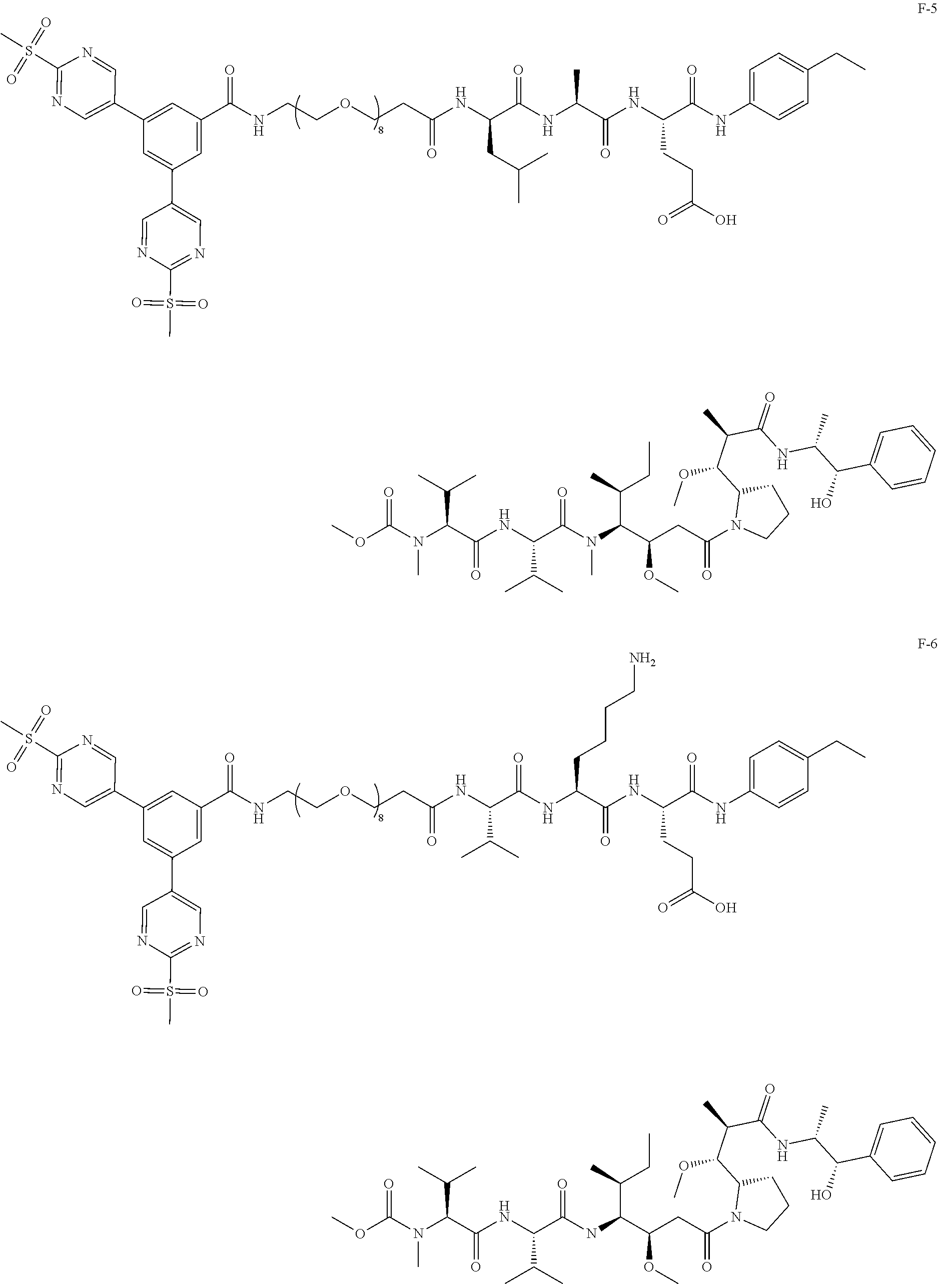 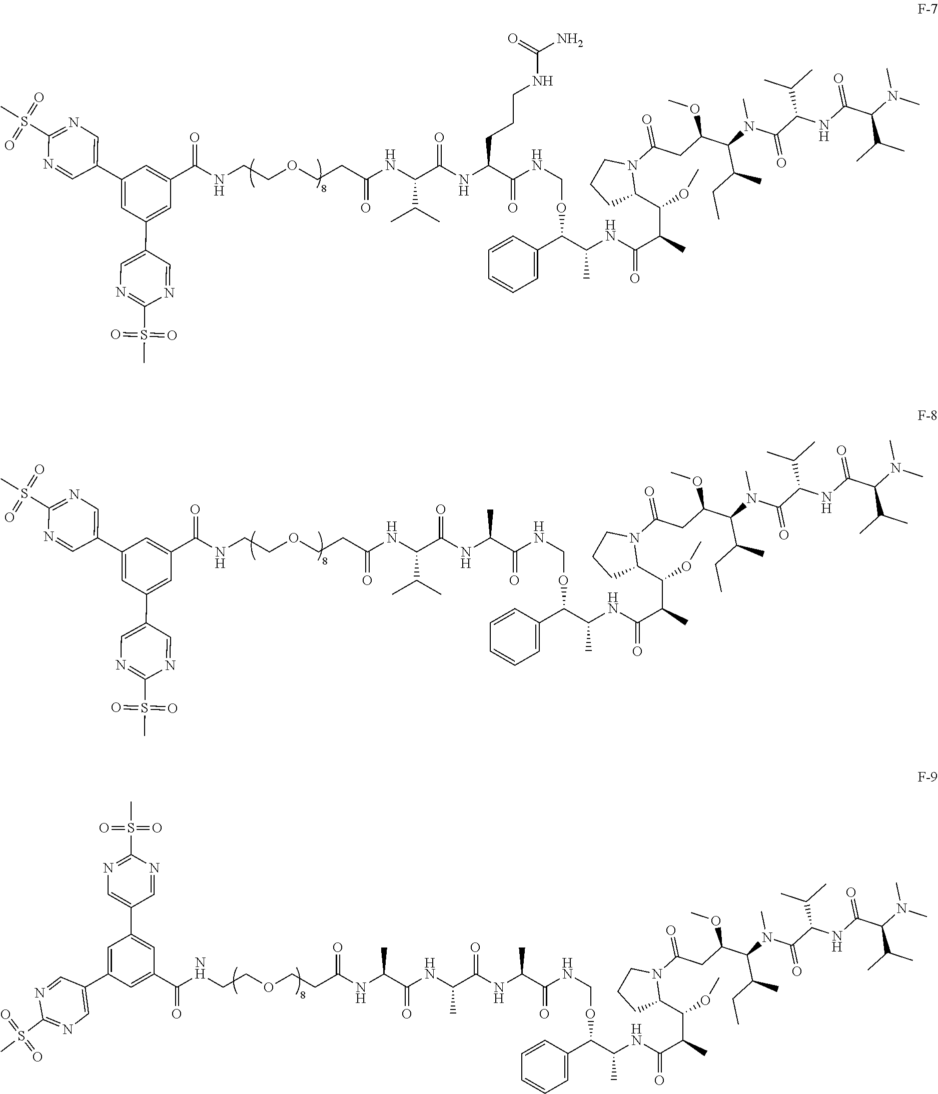 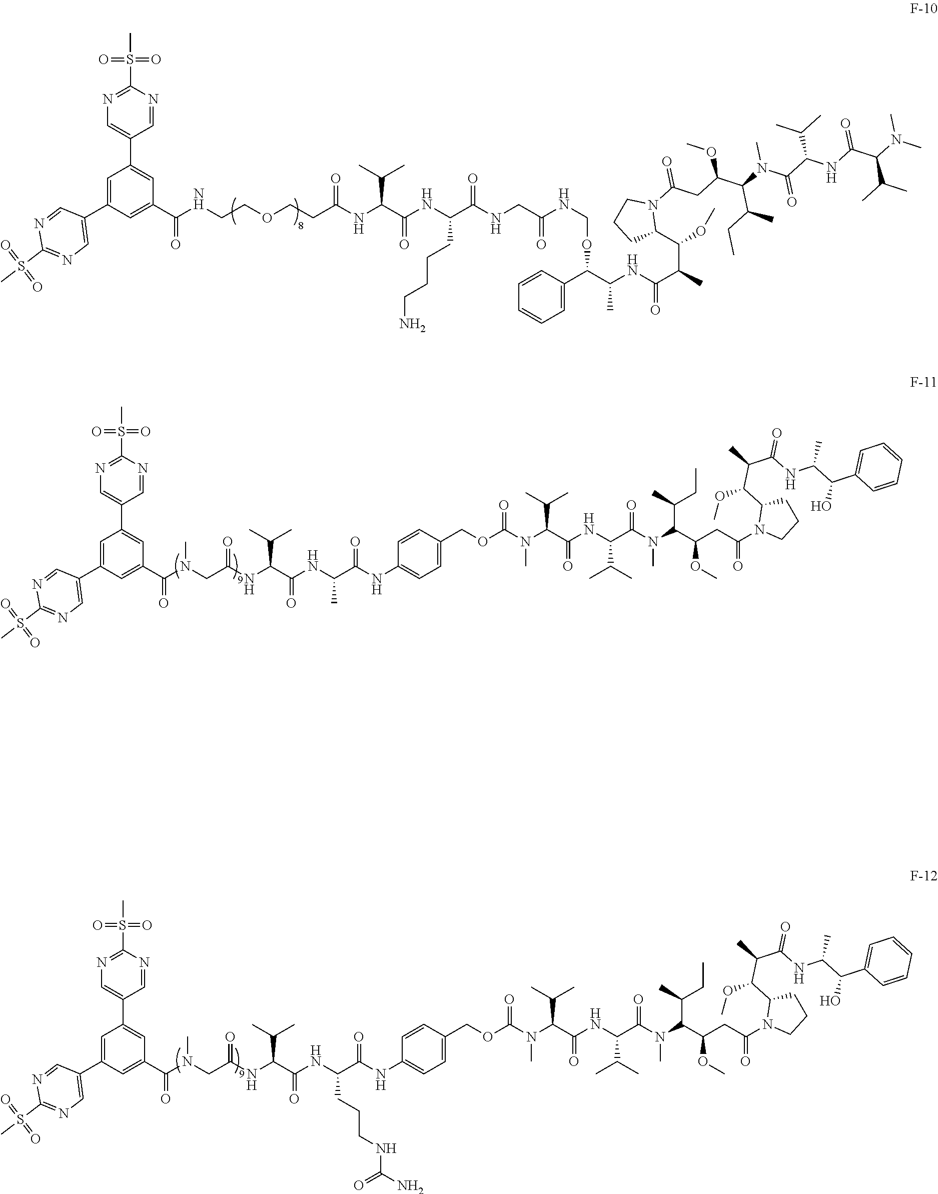 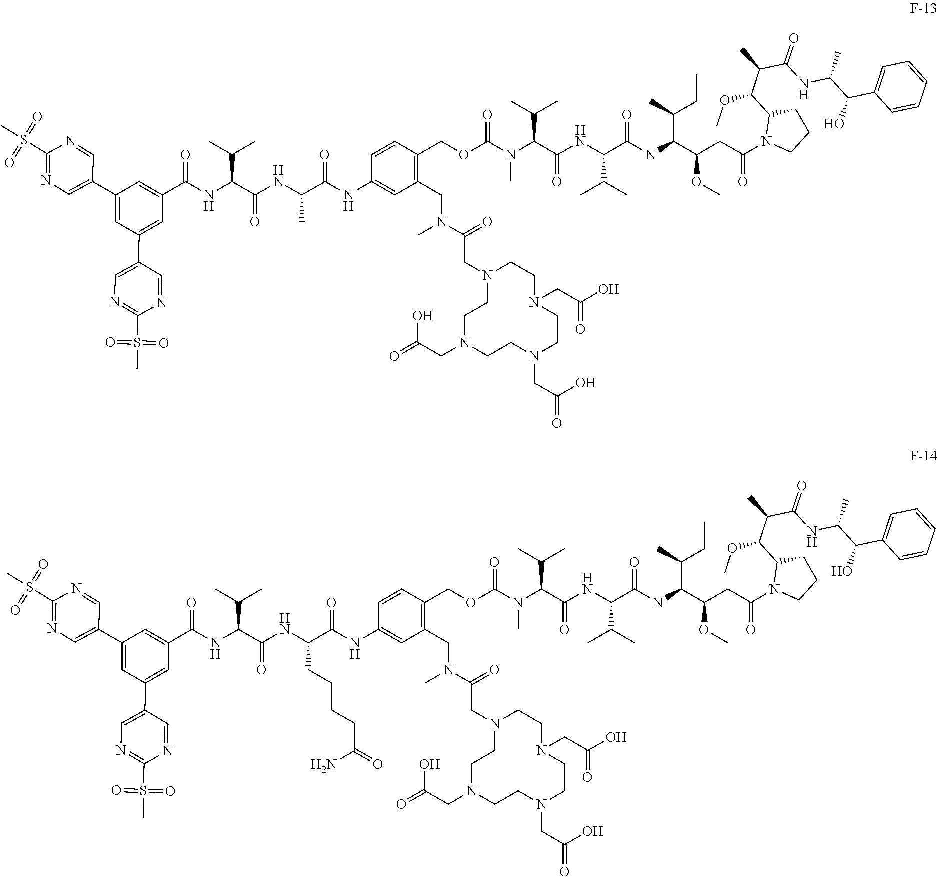 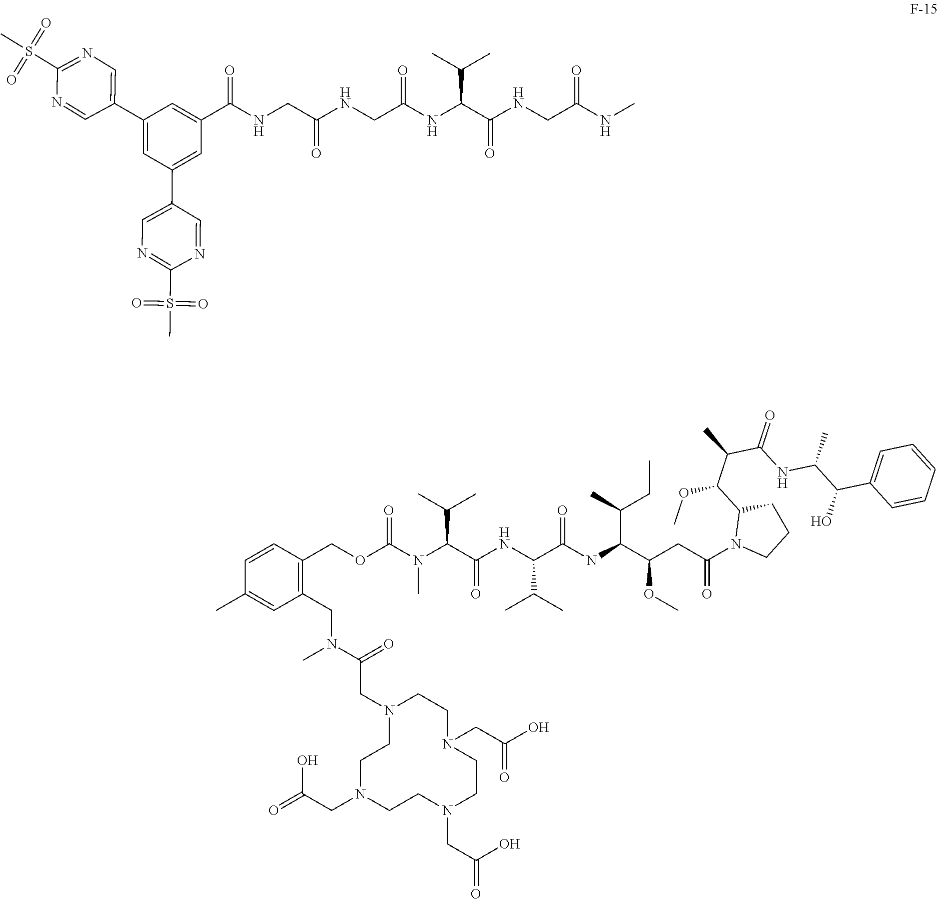 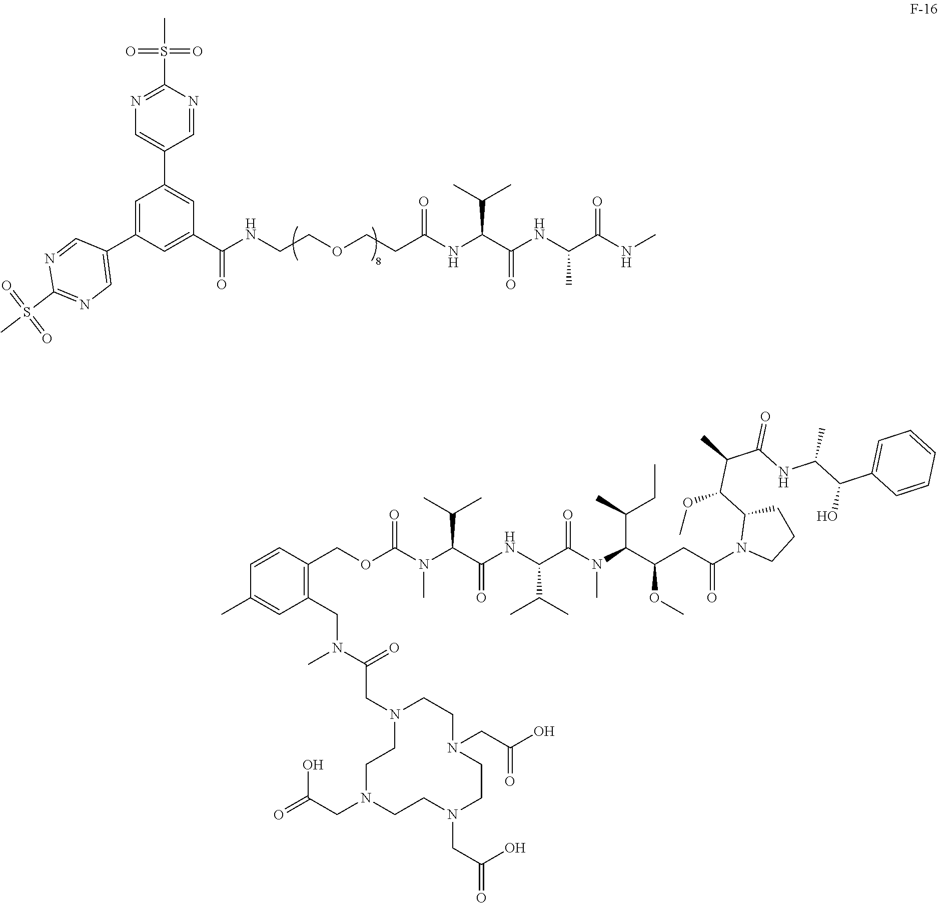 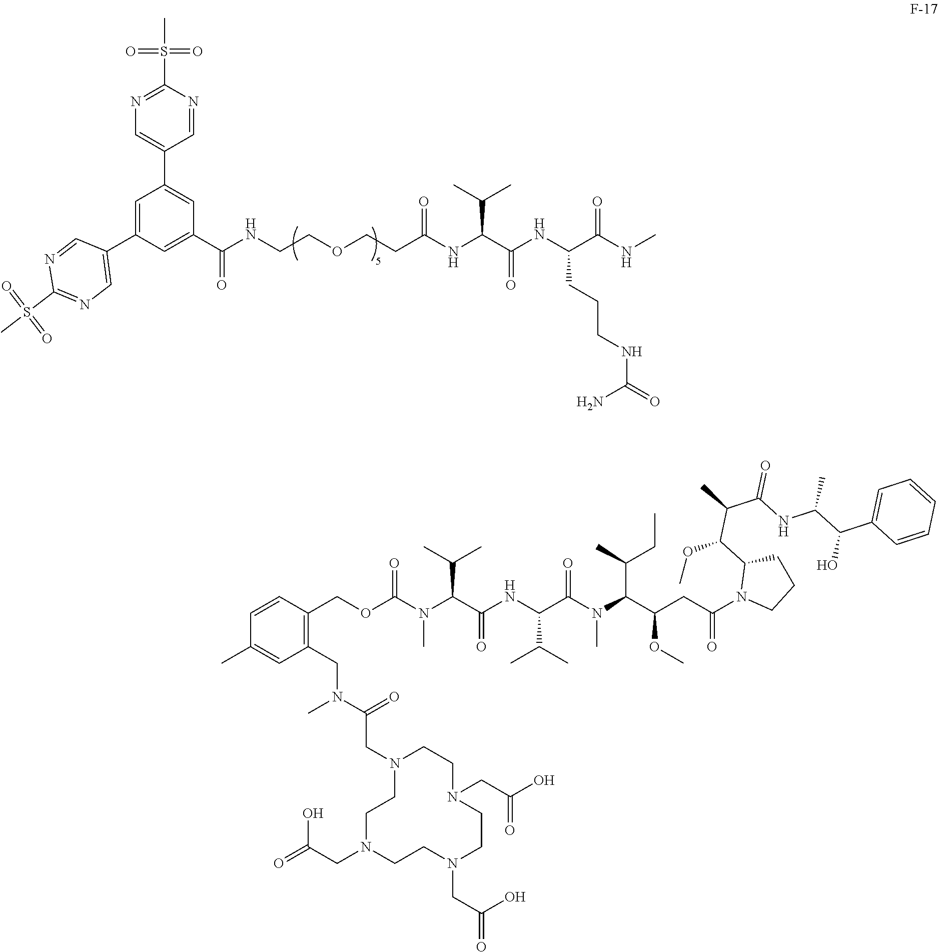 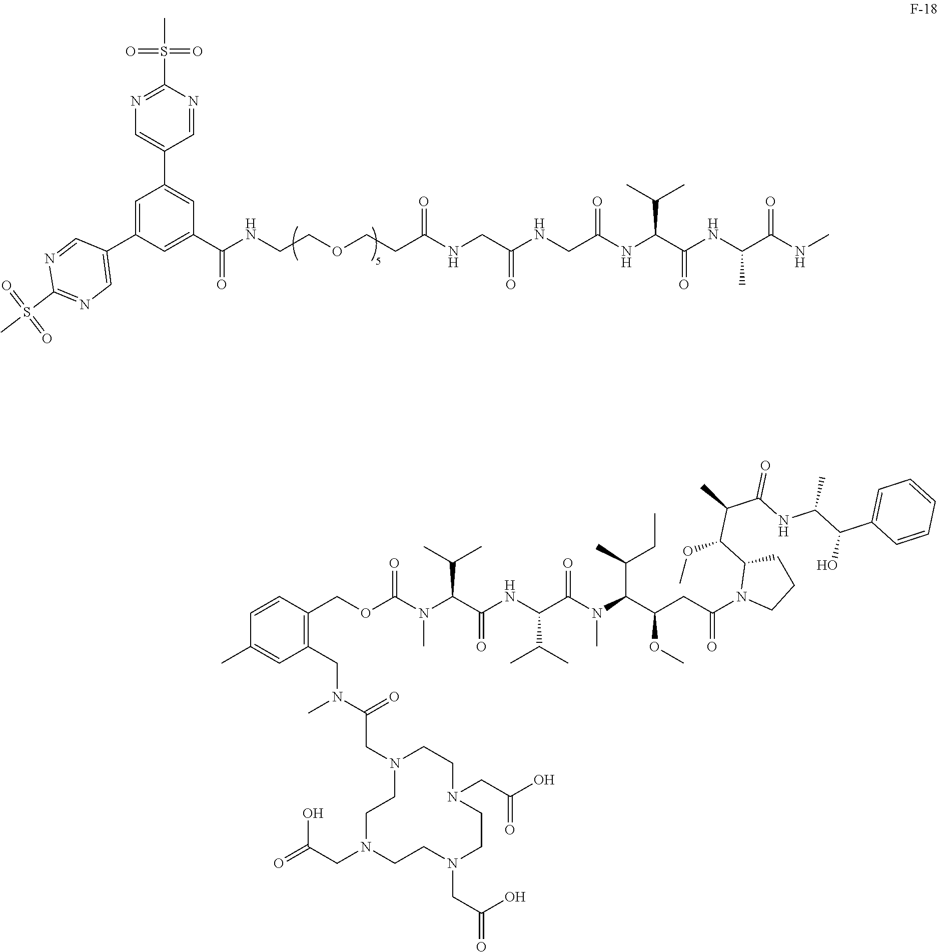 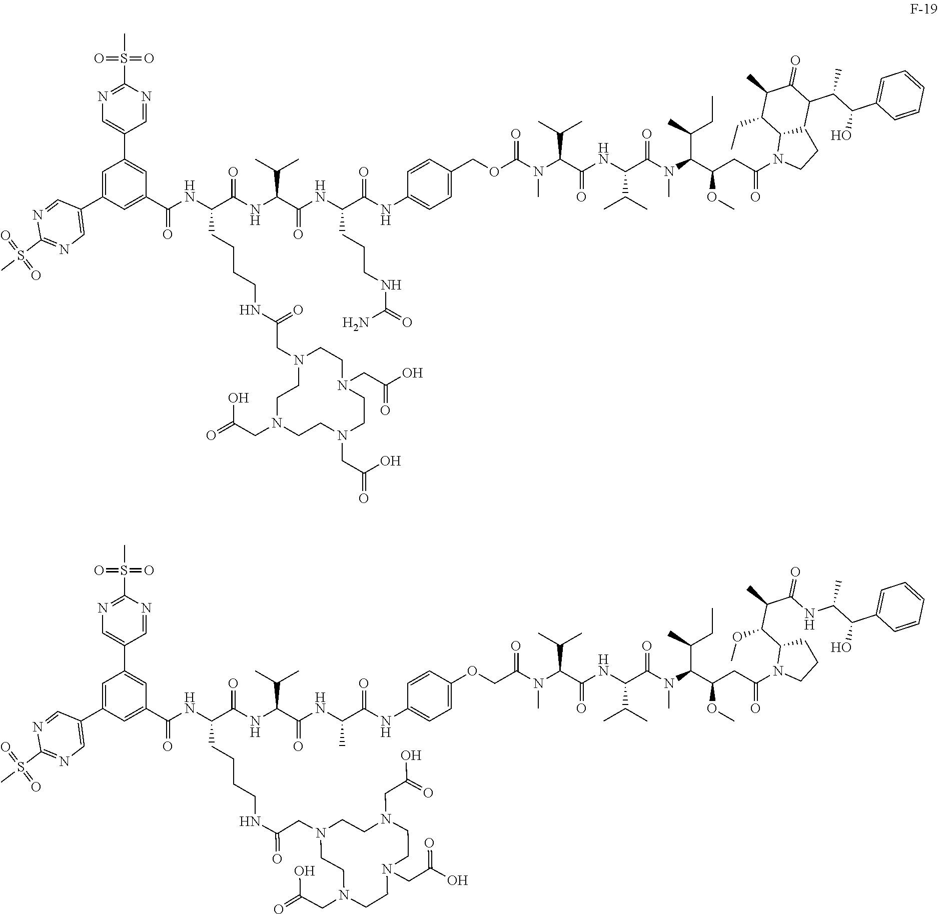 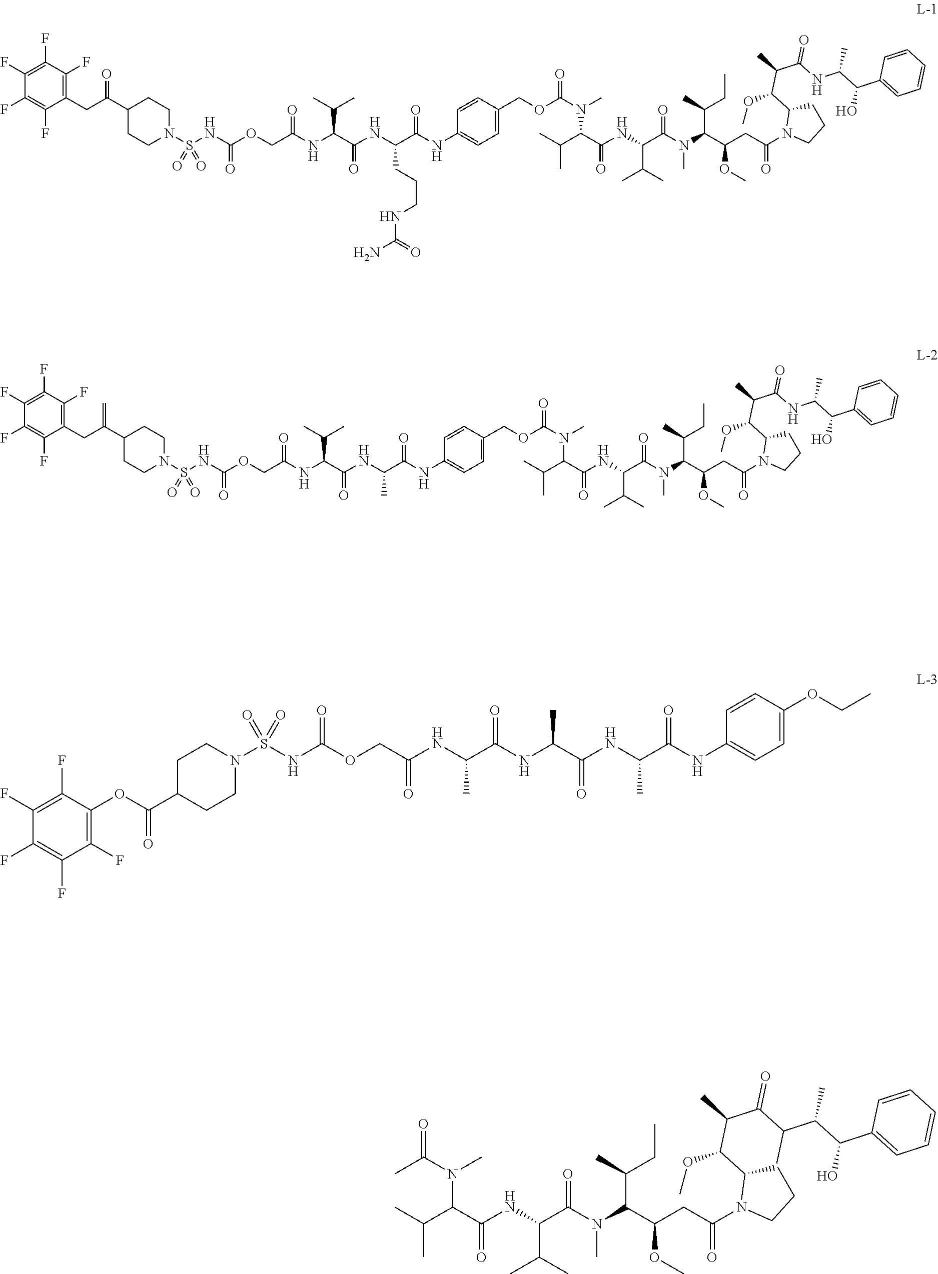 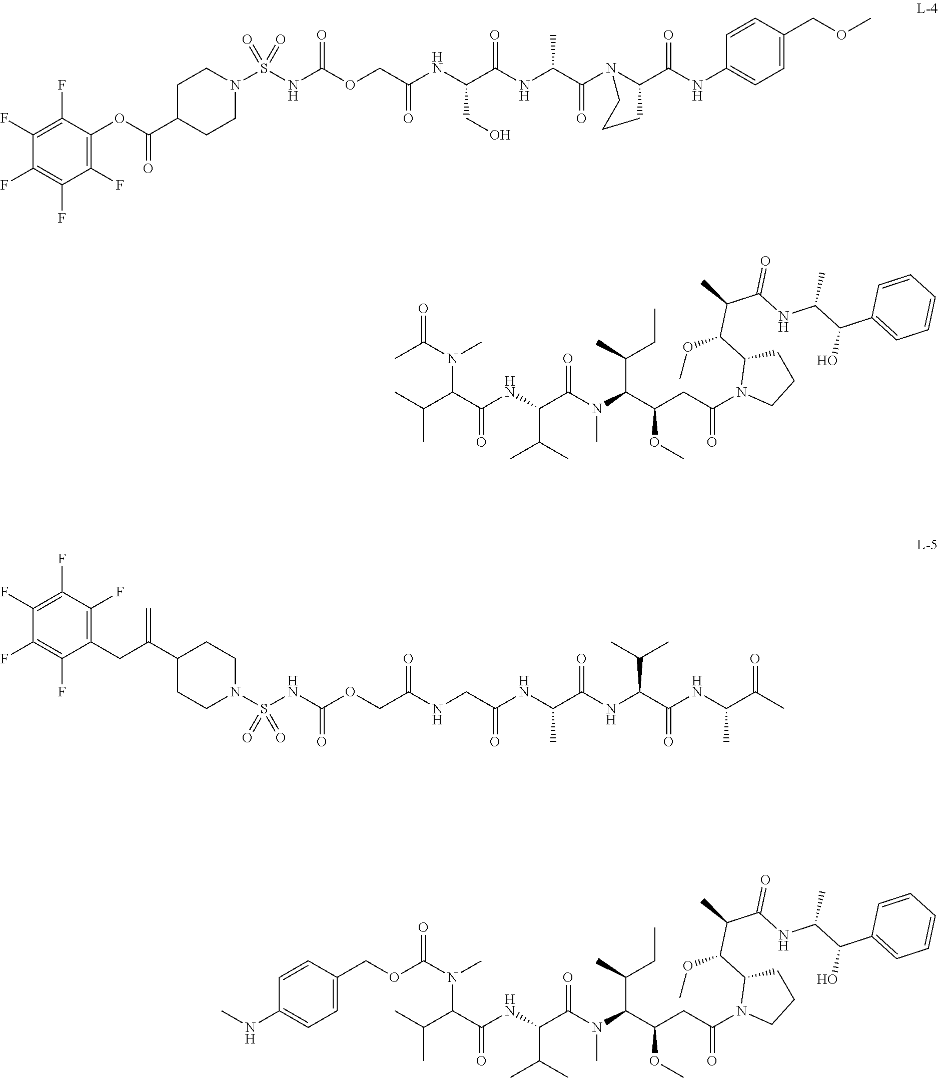 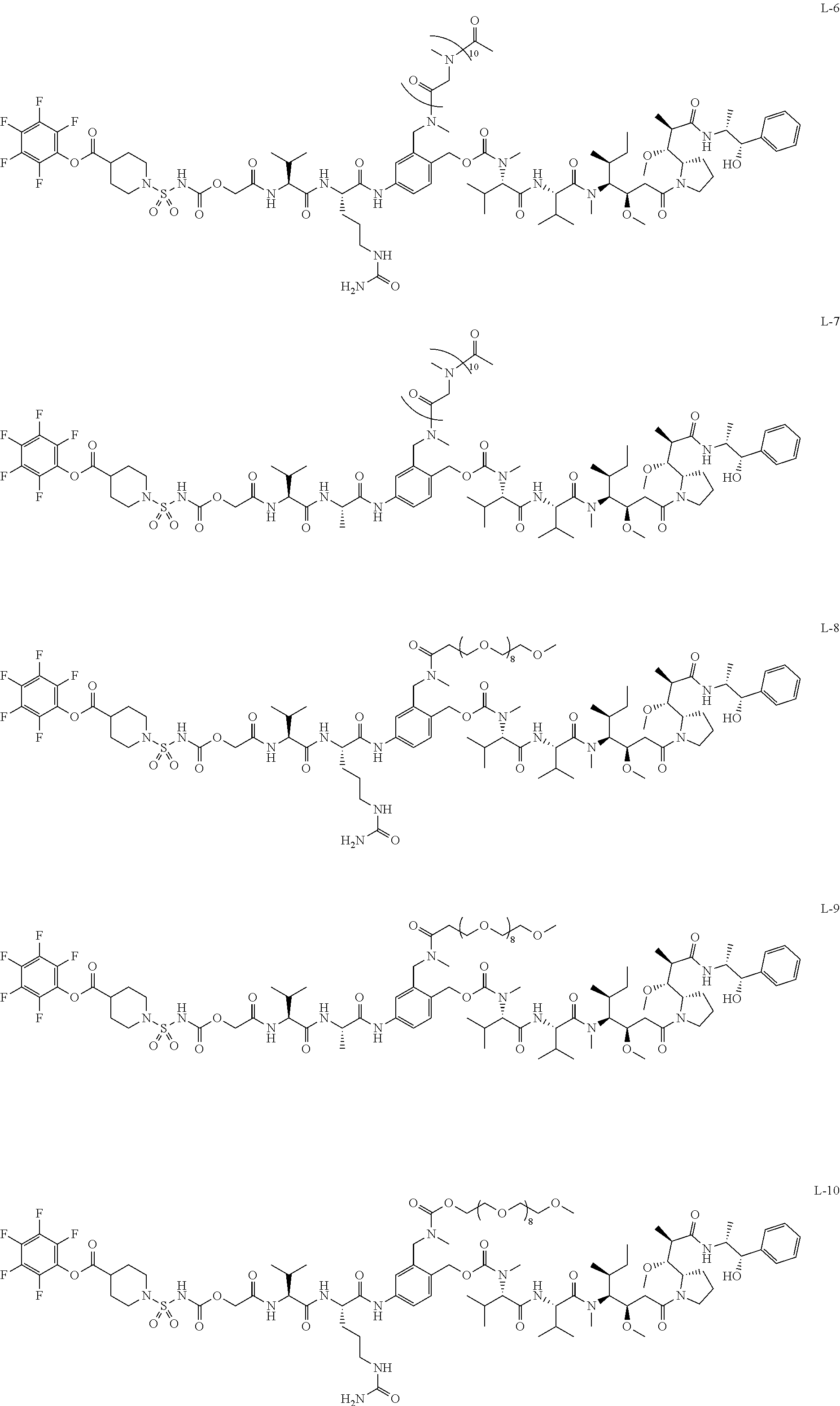 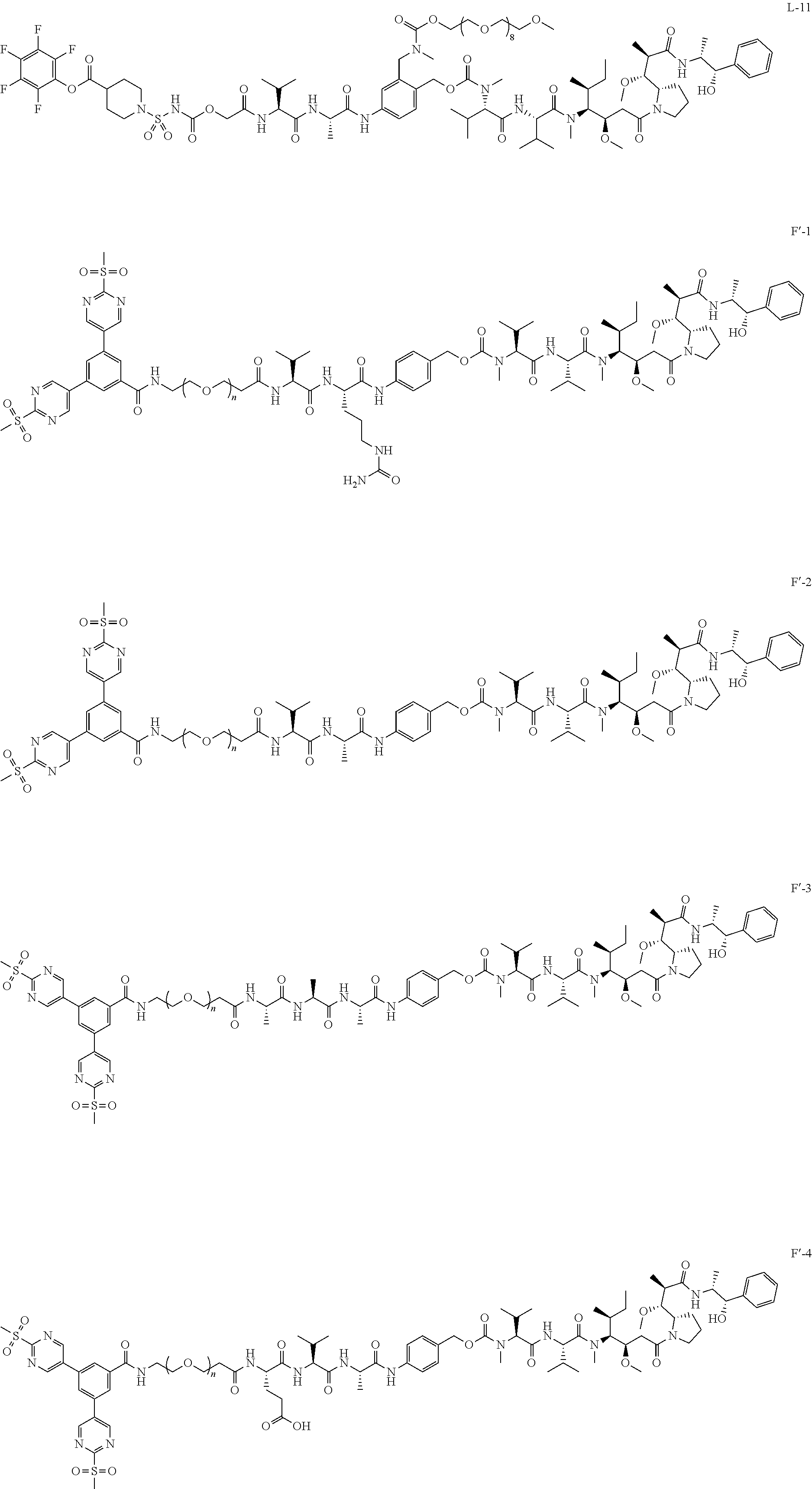 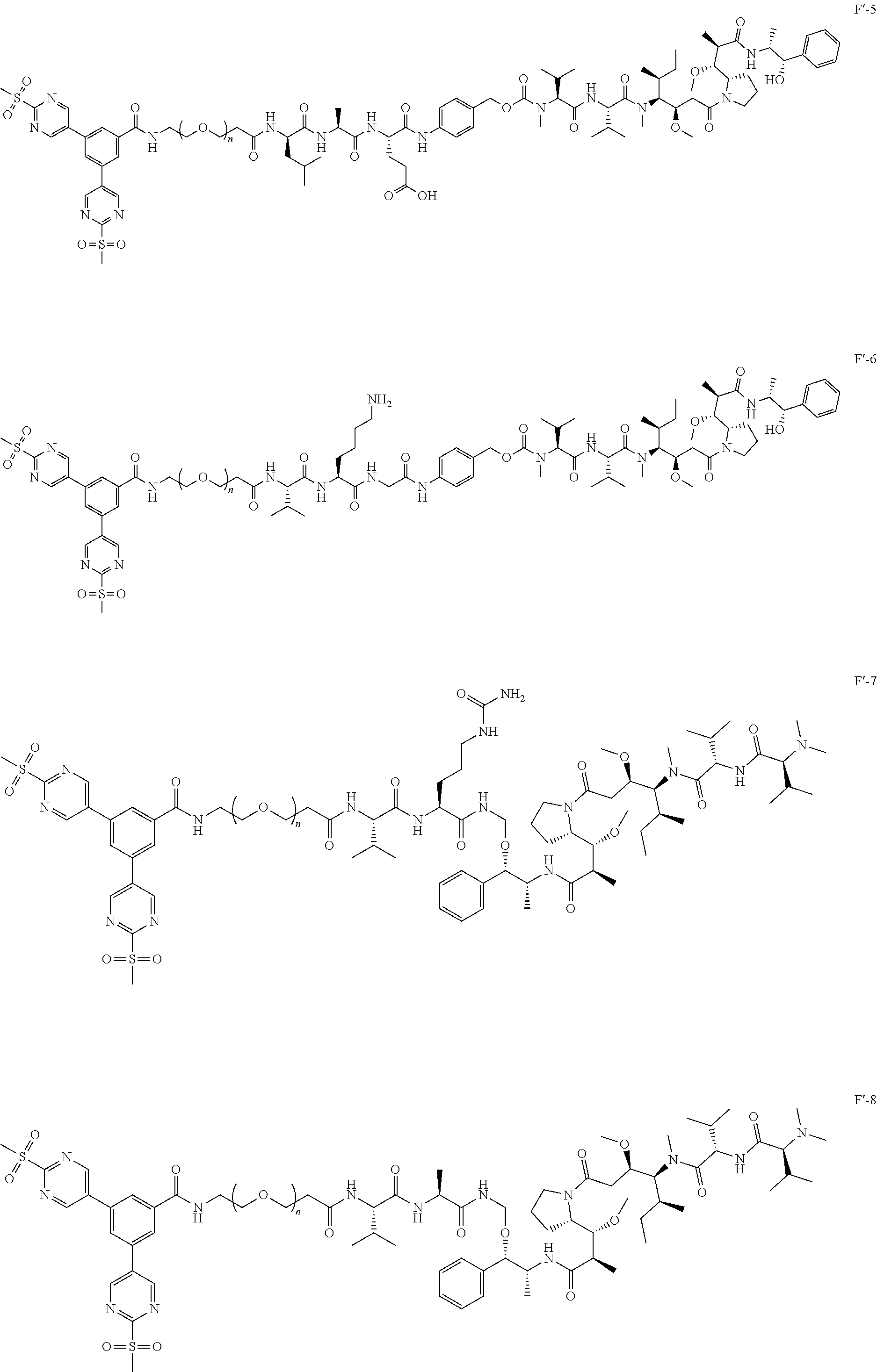 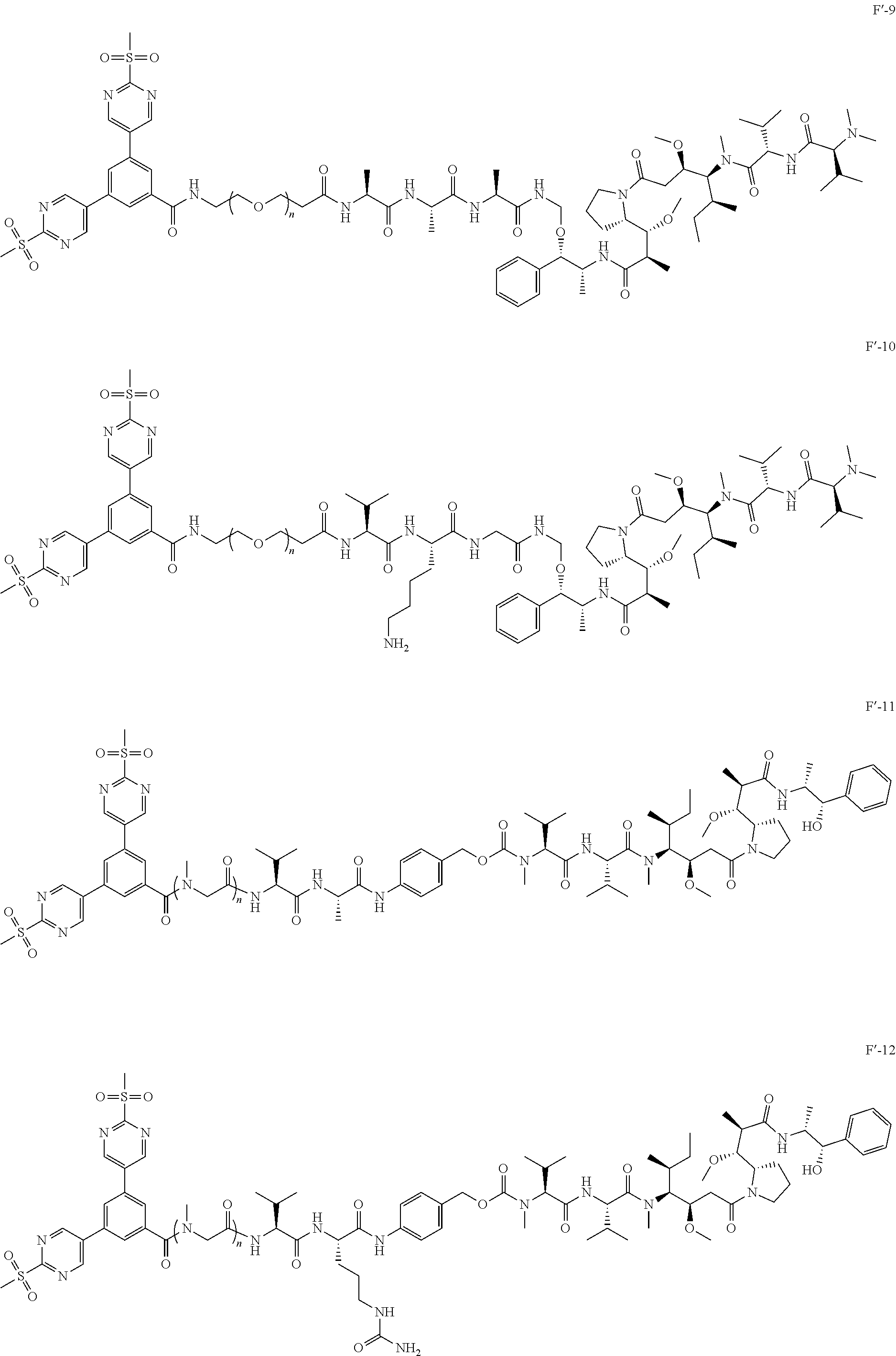 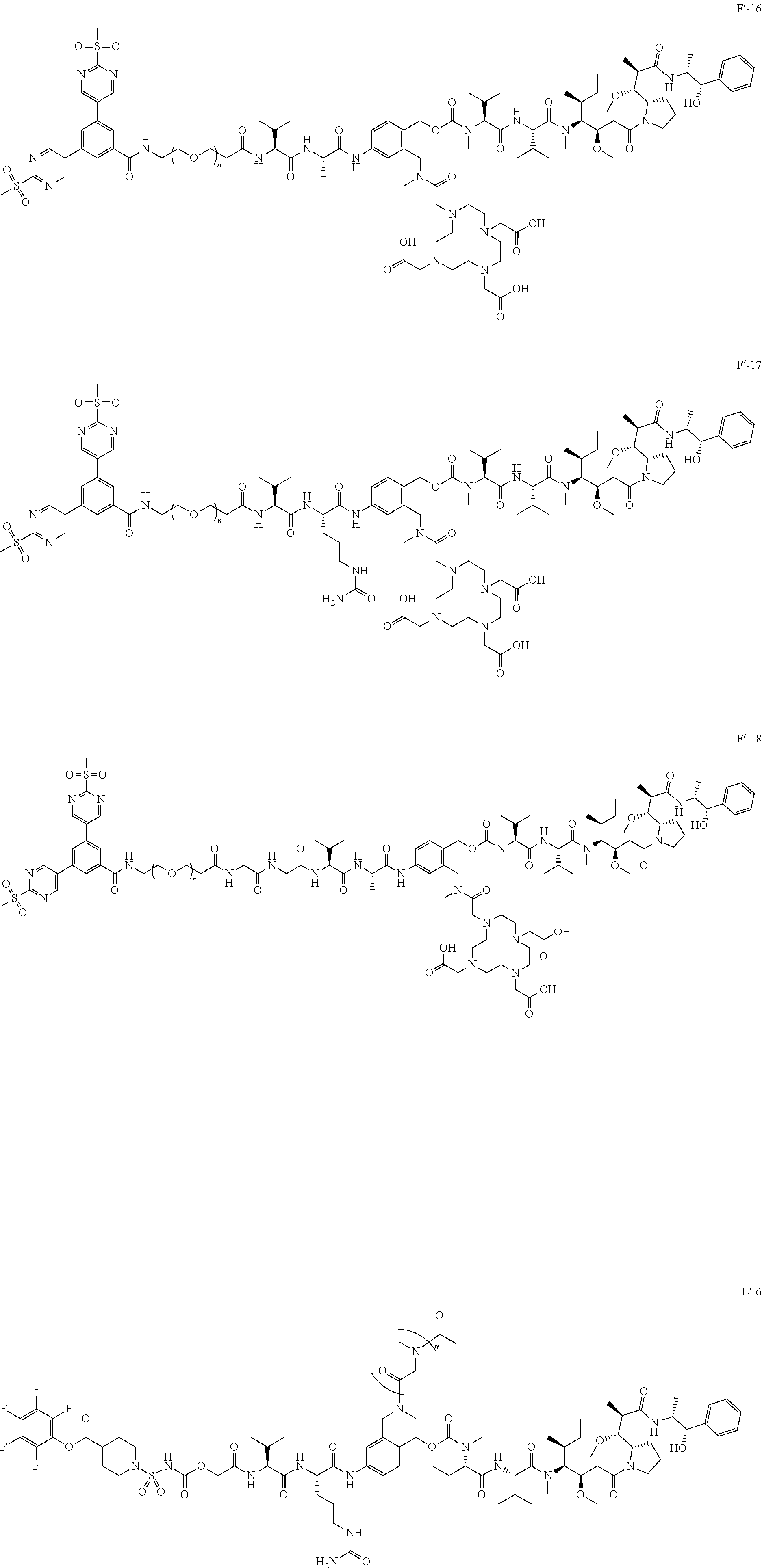 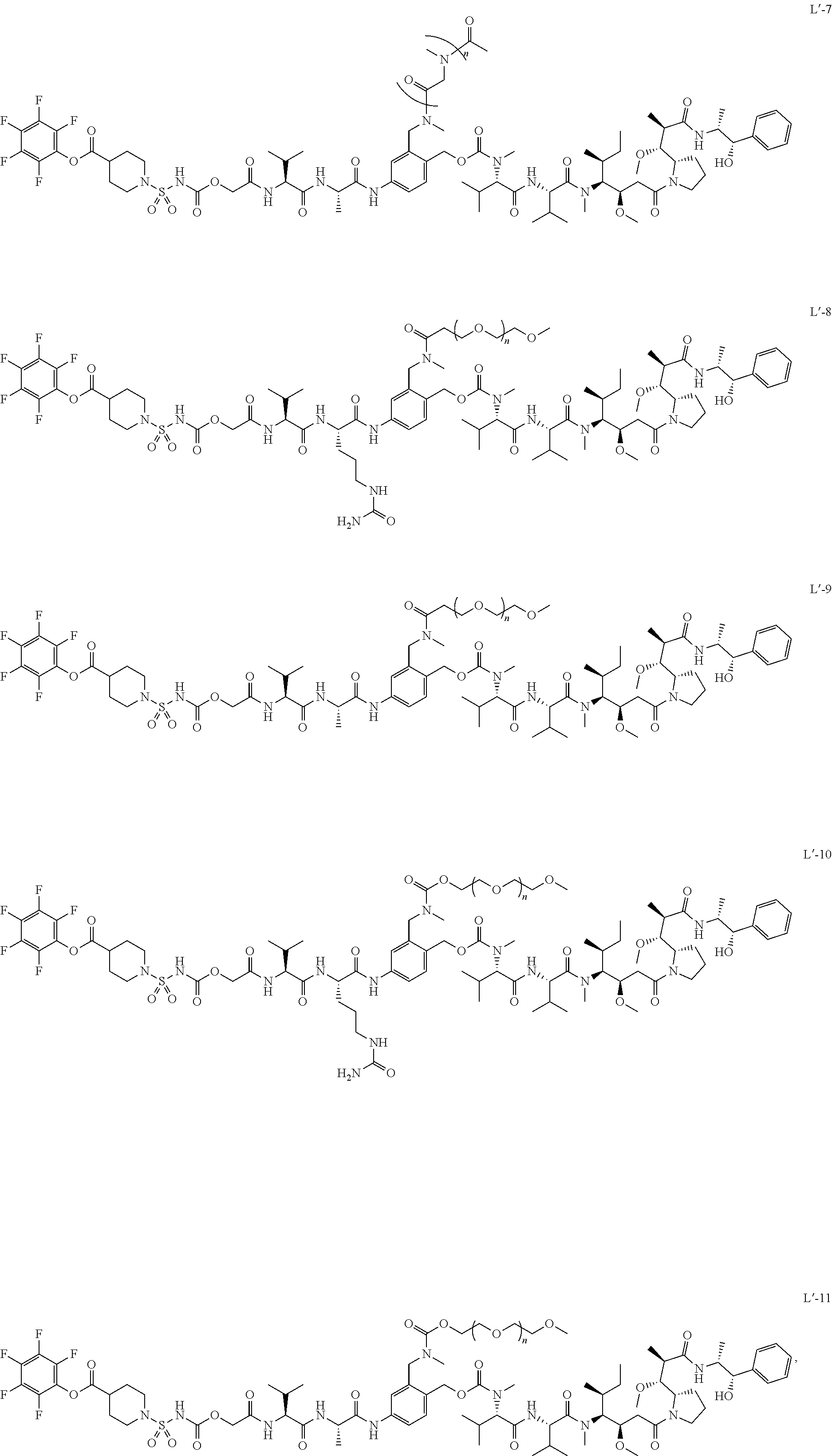
wherein n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 5, 8, 9 or 10.

8. The antibody-drug conjugate according to any one of claims 1 to 7, wherein the antibody or antigen-binding fragment thereof comprises:
(1) the following heavy chain variable region (VH) and/or light chain variable region (VL):
(1a) having a sequence as set forth in SEQ ID NO: 5 or variant thereof, a CDR-H2 having a sequence as set forth in SEQ ID NO: 6 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(1b) a heavy chain variable region (VH) comprising the following three CDRs: a CDR-H1 having a sequence as set forth in SEQ ID NO: 20 or variant thereof, a CDR-H2 having a sequence as set forth in SEQ ID NO: 21 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (1a) and (1b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(2) the following heavy chain variable region (VH) and/or light chain variable region (VL):
(2a) a heavy chain variable region (VH) comprising the following three CDRs: a CDR-H1 having a sequence as set forth in SEQ ID NO: 18 or variant thereof, a CDR-H2 having a sequence as set forth in SEQ ID NO: 19 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(2b) a heavy chain variable region (VH) comprising the following three CDRs: a CDR-H1 having a sequence as set forth in SEQ ID NO: 33 or variant thereof, a CDR-H2 having a sequence as set forth in SEQ ID NO: 34 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (2a) and (2b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(3) the following heavy chain variable region (VH) and/or light chain variable region (VL):
(3a) a heavy chain variable region (VH) comprising the following three CDRs: a CDR-H1 having a sequence as set forth in SEQ ID NO: 11 or variant thereof, a CDR-H2 having a sequence as set forth in SEQ ID NO: 12 or variant thereof, and a CDR-H3 having a sequence as set forth in SEQ ID NO: 7 or variant thereof; and/or, a light chain variable region (VL) comprising the following three CDRs: a CDR-L1 having a sequence as set forth in SEQ ID NO: 8 or variant thereof, a CDR-L2 having a sequence as set forth in SEQ ID NO: 9 or variant thereof, and a CDR-L3 having a sequence as set forth in SEQ ID NO: 10 or variant thereof; or,
(3b) a heavy chain variable region (VH) comprising the following three CDRs: a CDR-H1 having a sequence as set forth in SEQ ID NO: 26 or variant thereof, a CDR-H2 having a sequence as set forth in SEQ ID NO: 27 or variant thereof, and a CDR-H3 having a sequence as set forth in SEQ ID NO: 22 or variant thereof; and/or, a light chain variable region (VL) comprising the following three CDRs: a CDR-L1 having a sequence as set forth in SEQ ID NO: 23 or variant thereof, a CDR-L2 having a sequence as set forth in SEQ ID NO: 24 or variant thereof, and a CDR-L3 having a sequence as set forth in SEQ ID NO: 25 or variant thereof;
wherein, the variant described in any one of (3a), (3b), and (3c) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution;
or,
(4) the following heavy chain variable region (VH) and/or light chain variable region (VL):
(4a) a heavy chain variable region (VH) comprising the following three CDRs: a CDR-H1 having a sequence as set forth in SEQ ID NO: 13 or variant thereof, a CDR-H2 having a sequence as set forth in SEQ ID NO: 14 or variant thereof, and a CDR-H3 having a sequence as set forth in SEQ ID NO: 15 or variant thereof; and/or, a light chain variable region (VL) comprising the following three CDRs: a CDR-L1 having a sequence as set forth in SEQ ID NO: 16 or variant thereof, a CDR-L2 having a sequence as set forth in SEQ ID NO: 17 or variant thereof, and a CDR-L3 having a sequence as set forth in SEQ ID NO: 10 or variant thereof; or,
(4b) a heavy chain variable region (VH) comprising the following three CDRs: a CDR-H1 having a sequence as set forth in SEQ ID NO: 28 or variant thereof, a CDR-H2 having a sequence as set forth in SEQ ID NO: 29 or variant thereof, and a CDR-H3 having a sequence as set forth in SEQ ID NO: 30 or variant thereof; and/or, a light chain variable region (VL) comprising the following three CDRs: a CDR-L1 having a sequence as set forth in SEQ ID NO: 31 or variant thereof, a CDR-L2 having a sequence as set forth in SEQ ID NO: 32 or variant thereof, and a CDR-L3 having a sequence as set forth in SEQ ID NO: 25 or variant thereof;
wherein, the variant described in any one of (4a) and (4b) has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2 or 3 amino acids) as compared with the sequence from which it is derived; preferably, the substitution is a conservative substitution.

9. The antibody-drug conjugate according to any one of claims 1 to 8, wherein the antibody or antigen-binding fragment thereof comprises:
(a) a VH as set forth in SEQ ID NO: 1 or variant thereof, and/or, a VL or variant thereof as set forth in SEQ ID NO: 2; or
(b) a VH as set forth in SEQ ID NO: 3 or variant thereof, and/or, a VL or variant thereof as set forth in SEQ ID NO: 4;
wherein, the variant has a sequence identity of at least 70%, at least 80%, at least 85%, at least 90%, at least 91%, at least 92%, at least 93%, at least 94%, at least 95%, at least 96%, at least 97%, at least 98%, at least 99%, or 100% as compared with the sequence from which it is derived, or the variant has a substitution, deletion or addition of one or more amino acids (e.g., a substitution, deletion or addition of 1, 2, 3, 4 or 5 amino acids) as compared with the sequence from which it is derived; preferably, the substitution is a conservative substitution.

10. The antibody-drug conjugate according to claim 8 or 9, wherein the antibody or antigen-binding fragment thereof further comprises:
(a) a human immunoglobulin heavy chain constant region (CH) or variant thereof, wherein the variant has a substitution, deletion or addition of one or more amino acids (e.g., a substitution, deletion or addition of up to 20, up to 15, up to 10, or up to 5 amino acids; e.g., a substitution, deletion or addition of 1, 2, 3, 4 or 5 amino acids) as compared with the wild-type sequence from which it is derived; and
(b) a human immunoglobulin light chain constant region (CL) or variant thereof, wherein the variant has a substitution, deletion or addition of one or more amino acids (e.g., a substitution, deletion or addition of up to 20, up to 15, up to 10, or up to 5 amino acids; for example, a substitution, deletion or addition of 1, 2, 3, 4 or 5 amino acids) as compared with the wild-type sequence from which it is derived;
preferably, the heavy chain constant region is an IgG heavy chain constant region, such as an IgG1, IgG2, IgG3 or IgG4 heavy chain constant region, such as a human IgG1 heavy chain constant region or a human IgG4 heavy chain constant region;
preferably, the antibody or antigen-binding fragment thereof comprises a heavy chain constant region (CH) as set forth in SEQ ID NO: 35 or variant thereof, wherein the variant has a conservative substitution of up to 20 amino acids as compared with SEQ ID NO: 35 (e.g., a conservative substitution of up to 15, up to 10, or up to 5 amino acids; for example, a conservative substitutions of 1, 2, 3, 4 or 5 amino acids);
preferably, the antibody or antigen-binding fragment thereof comprises a heavy chain constant region (CH) as set forth in SEQ ID NO: 35 or variant thereof, wherein the variant has a conservative substitution of up to 20 amino acids as compared with SEQ ID NO: 35 (e.g., a conservative substitution of up to 15, up to 10, or up to 5 amino acids; for example, a conservative substitution of 1, 2, 3, 4 or 5 amino acids);
preferably, the antibody or antigen-binding fragment thereof comprises a light chain constant region (CL) as set forth in SEQ ID NO: 36 or variant thereof, wherein the variant has a conservative substitution of up to 20 amino acids (e.g., a conservative substitution of up to 15, up to 10, or up to 5 amino acids; e.g., a conservative substitution of 1, 2, 3, 4 or 5 amino acids) as compared with SEQ ID NO: 36;
preferably, the antibody or antigen-binding fragment thereof comprises a heavy chain constant region (CH) as set forth in SEQ ID NO: 35 and a light chain constant region (CL) as set forth in SEQ ID NO: 36.

11. The antibody-drug conjugate according to any one of claims 1 to 10, wherein the antibody or antigen-binding fragment thereof comprises:
(1) a heavy chain comprising a VH having a sequence as set forth in SEQ ID NO: 1 and a heavy chain constant region (CH) as set forth in SEQ ID NO: 35, and a light chain comprising a VL having a sequence as set forth in SEQ ID NO: 2 and a light chain constant region (CL) as set forth in SEQ ID NO: 36; or
(2) a heavy chain comprising a VH having a sequence as set forth in SEQ ID NO: 3 and a heavy chain constant region (CH) as set forth in SEQ ID NO: 35, and a light chain comprising a VL having a sequence as set forth in SEQ ID NO: 4 and a light chain constant region (CL) as set forth in SEQ ID NO: 36.

12. The antibody-drug conjugate according to any one of claims 1 to 11, wherein M is connected to a sulfhydryl (—SH) or amino group (—NH₂) on Ab.

13. The antibody-drug conjugate according to any one of claims 1 to 12, wherein the antibody or antigen-binding fragment thereof is selected from the antibody or antigen-binding fragment thereof according to claim 11; -M-L-E-D is as defined in claim 7; x is 1 to 10; preferably, x is 1 to 8, or x is 1 to 4.

14. The antibody-drug conjugate according to any one of claims 1 to 13, which is selected from the group consisting of: 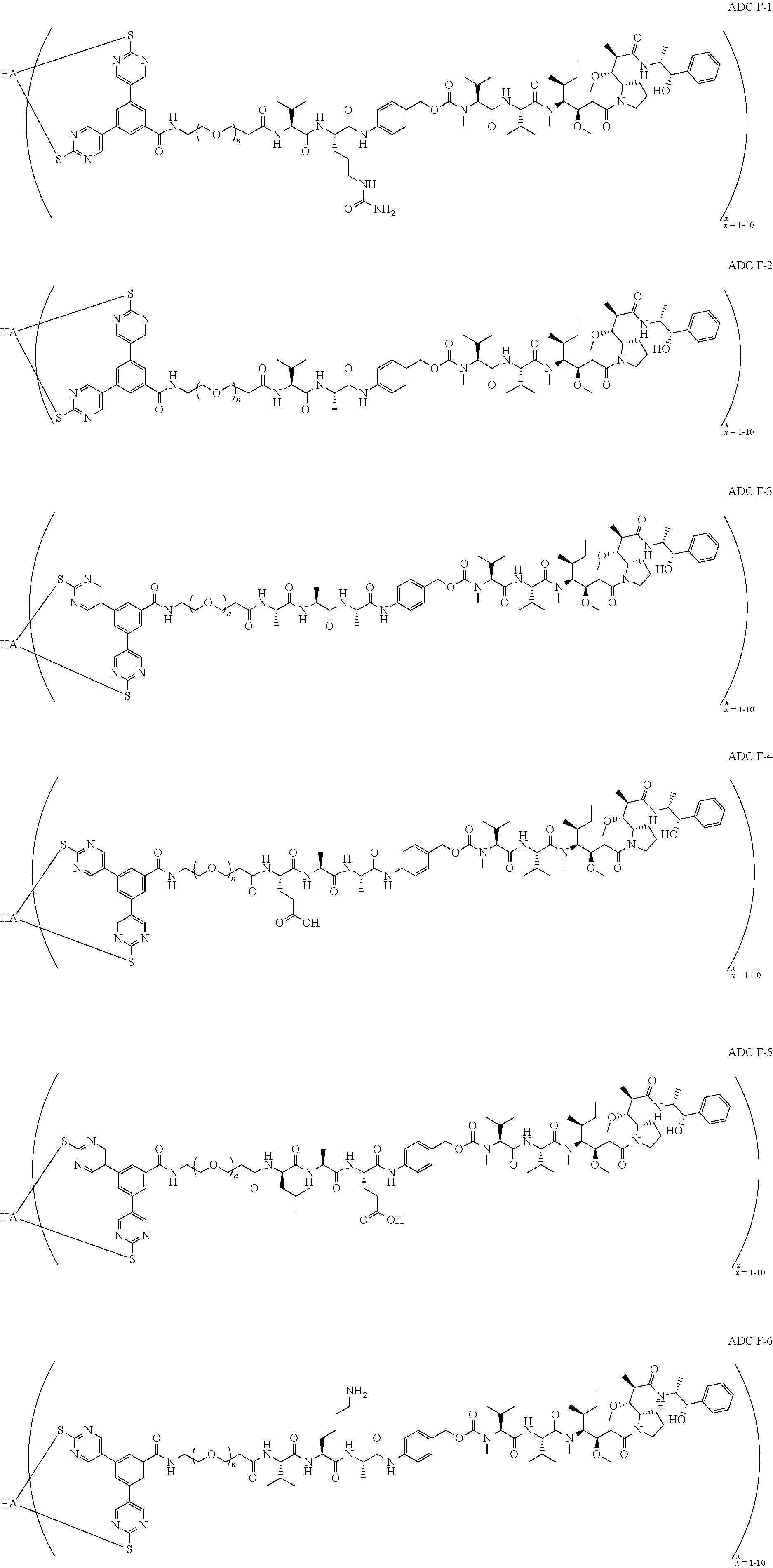 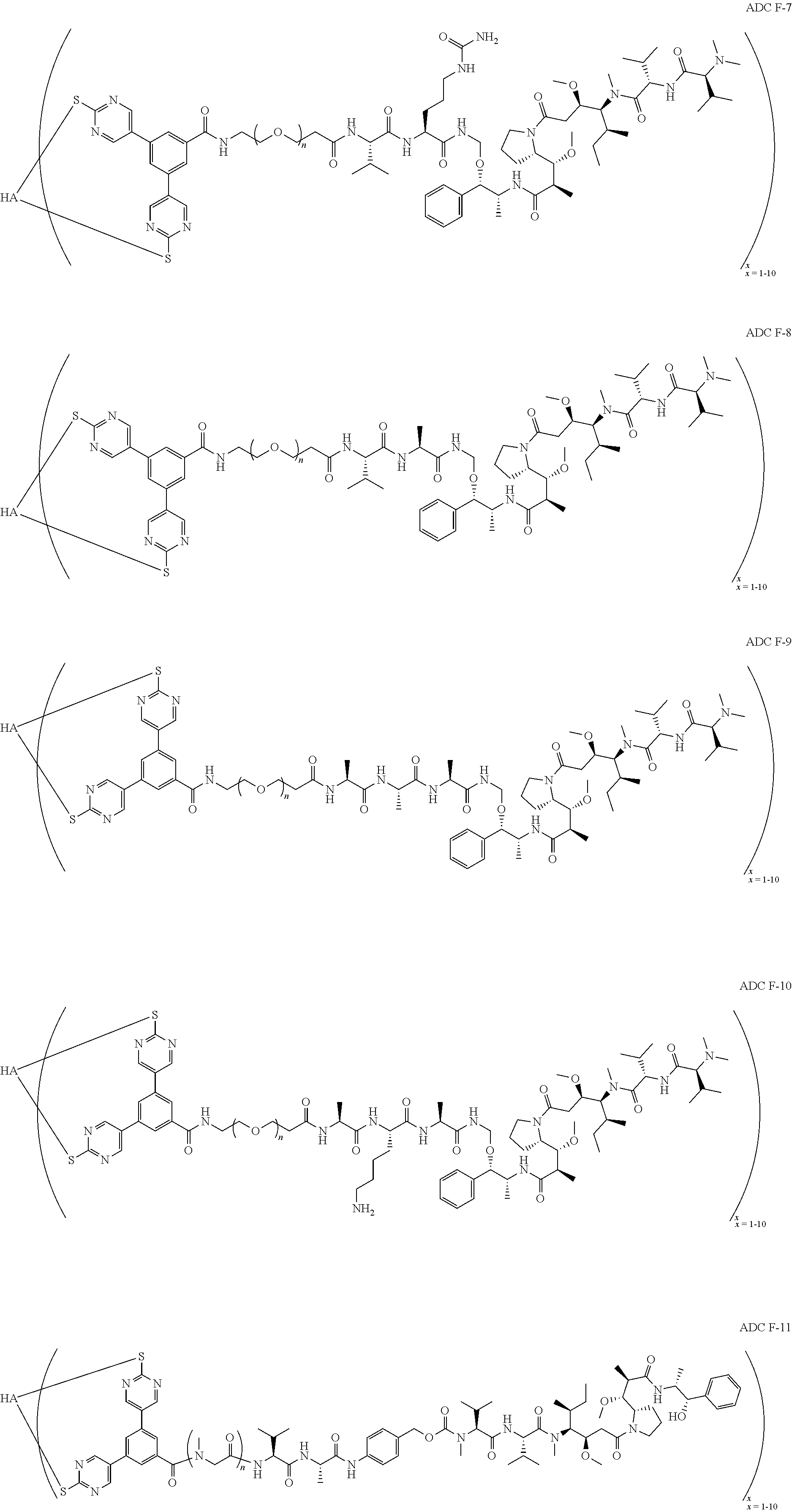 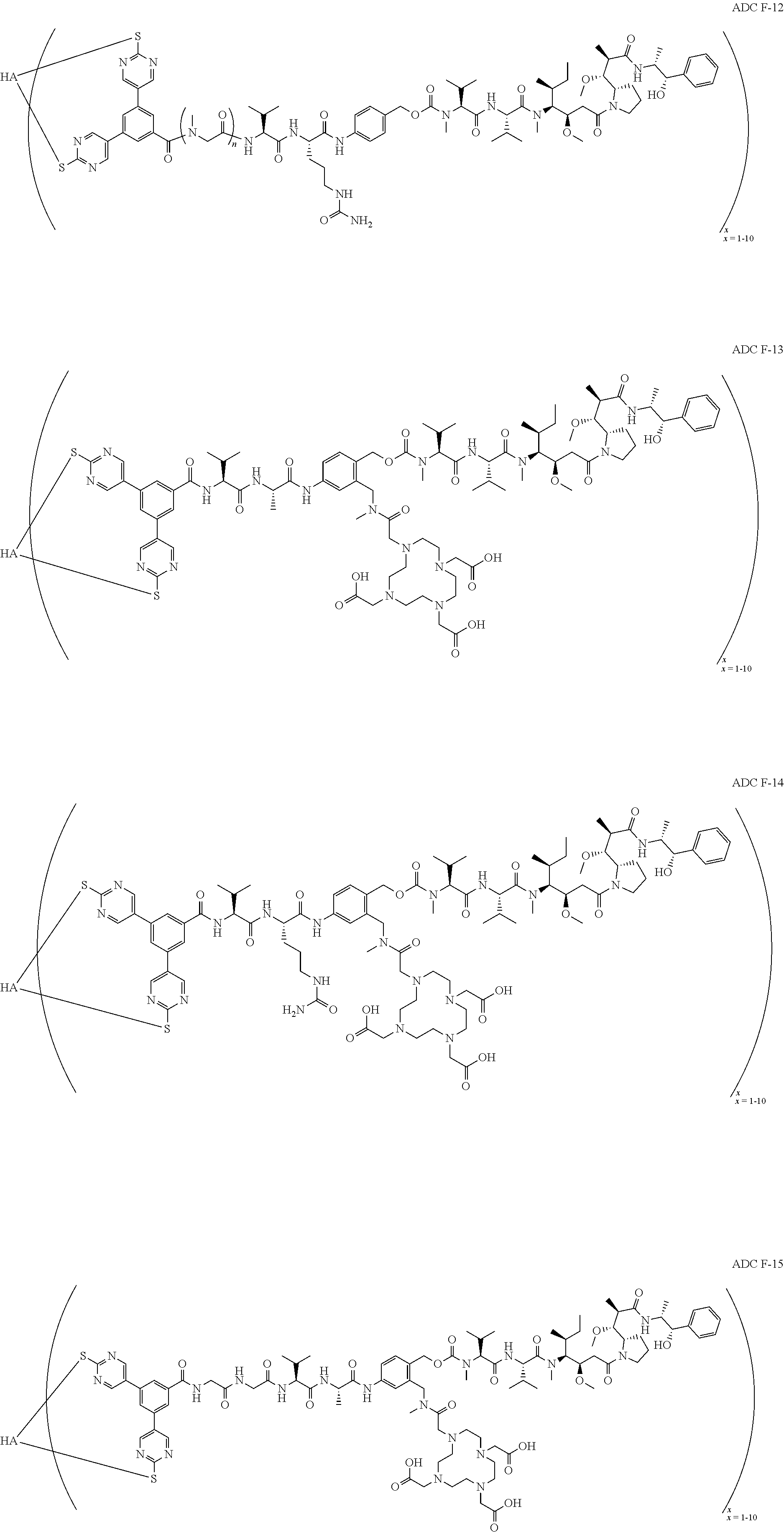 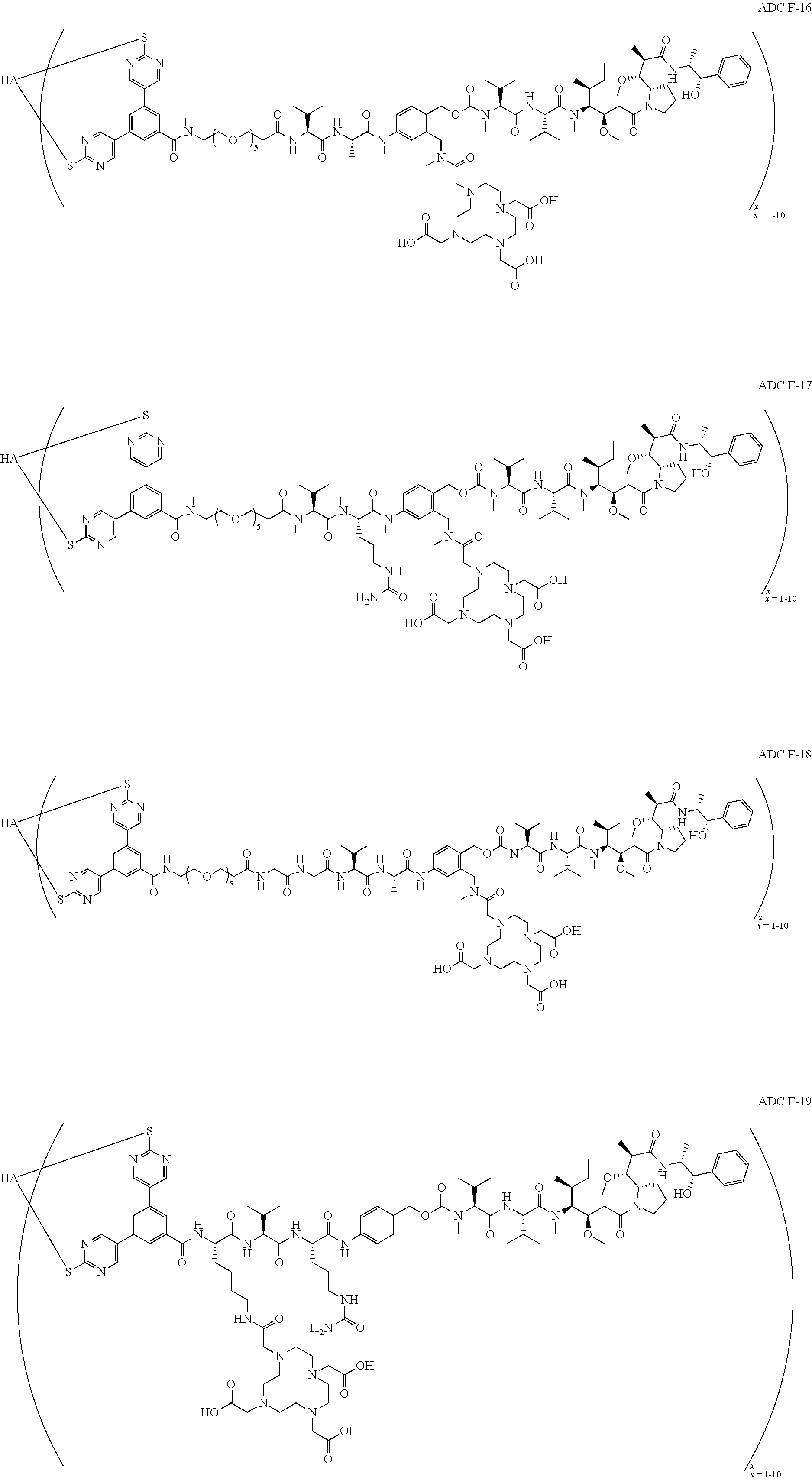 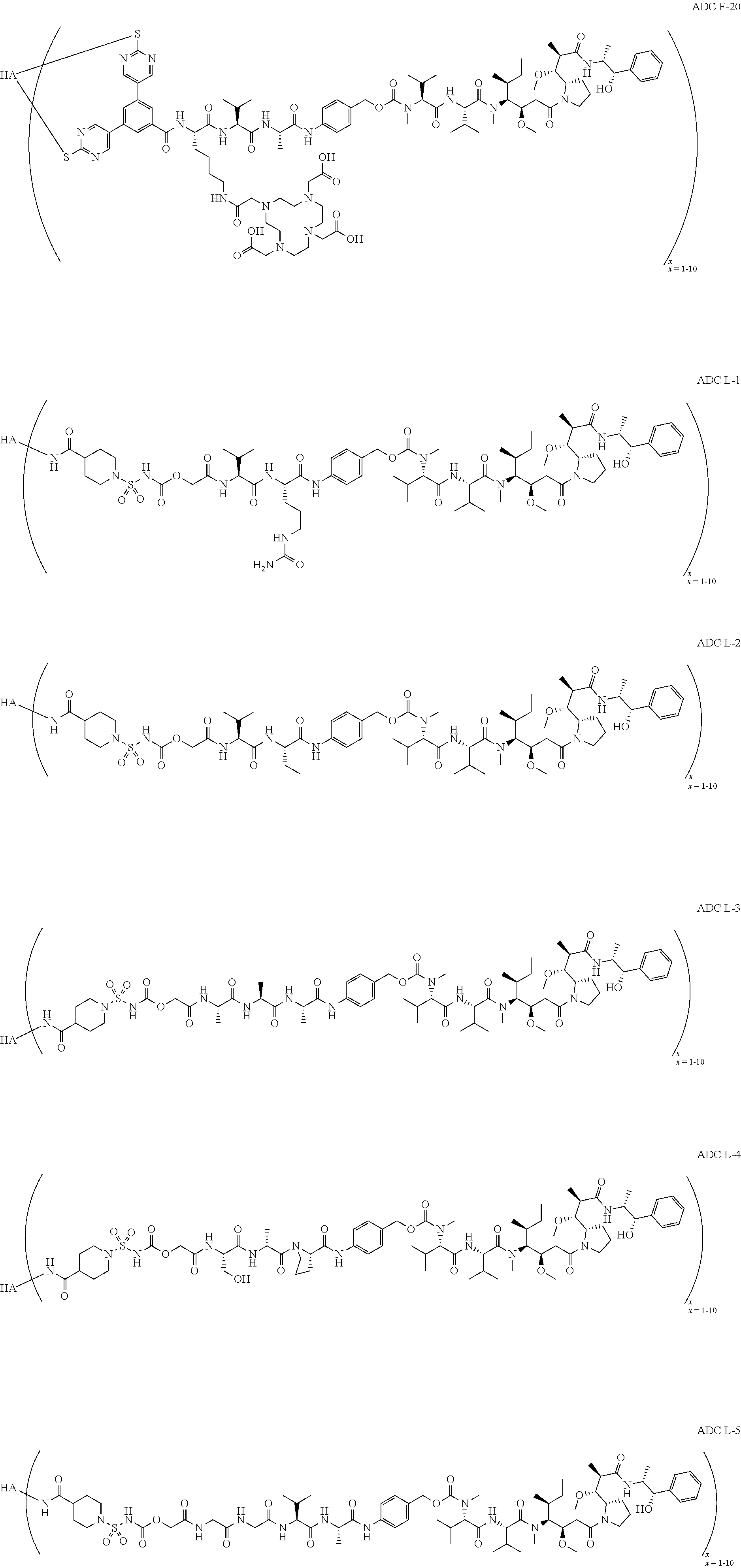 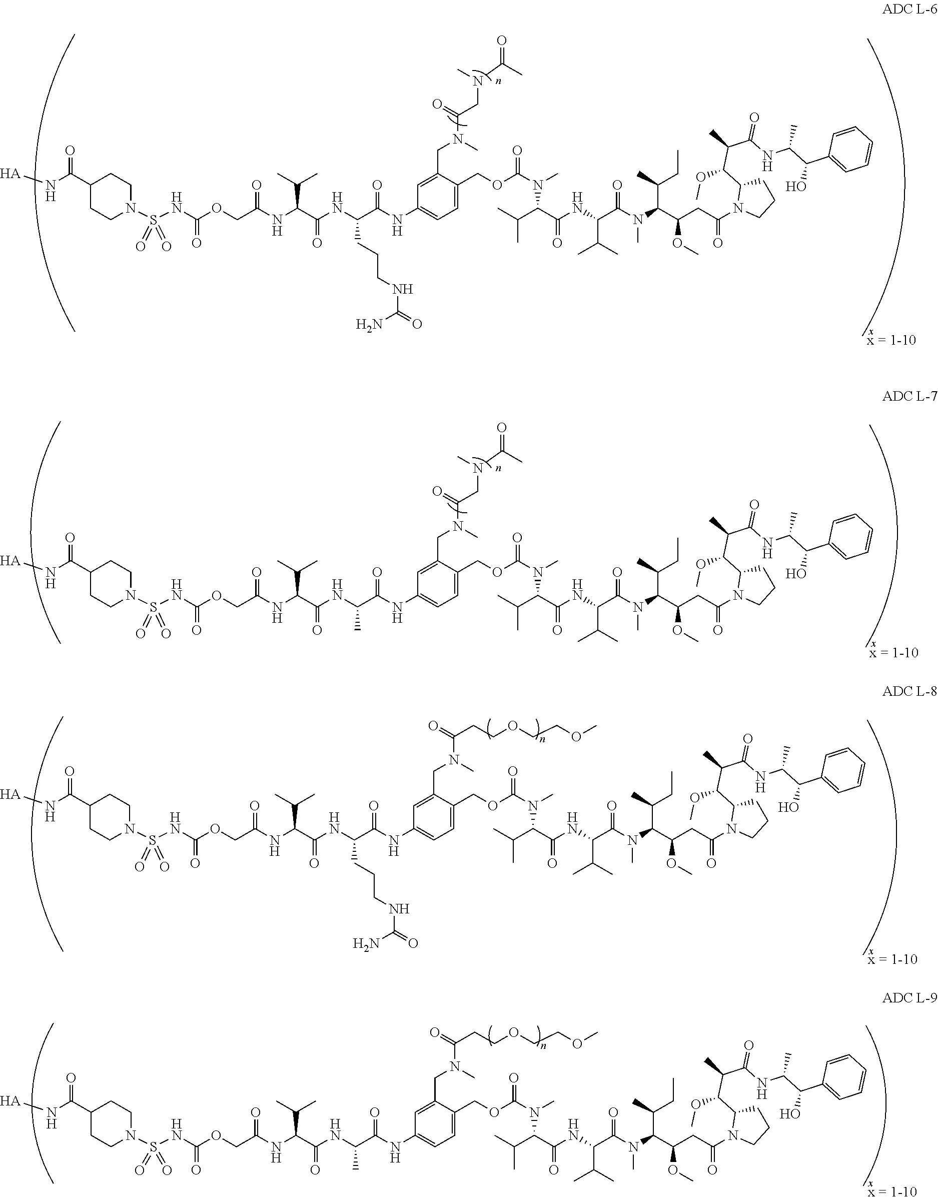 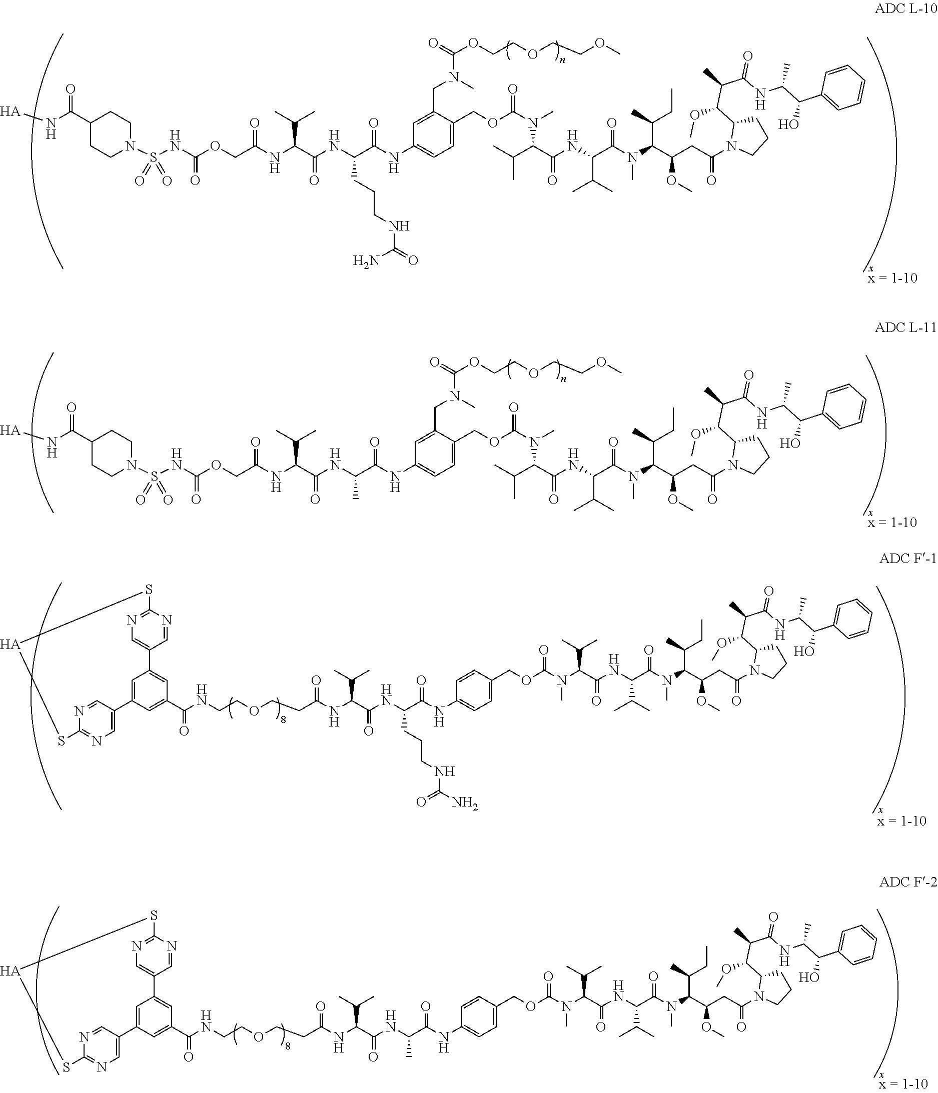 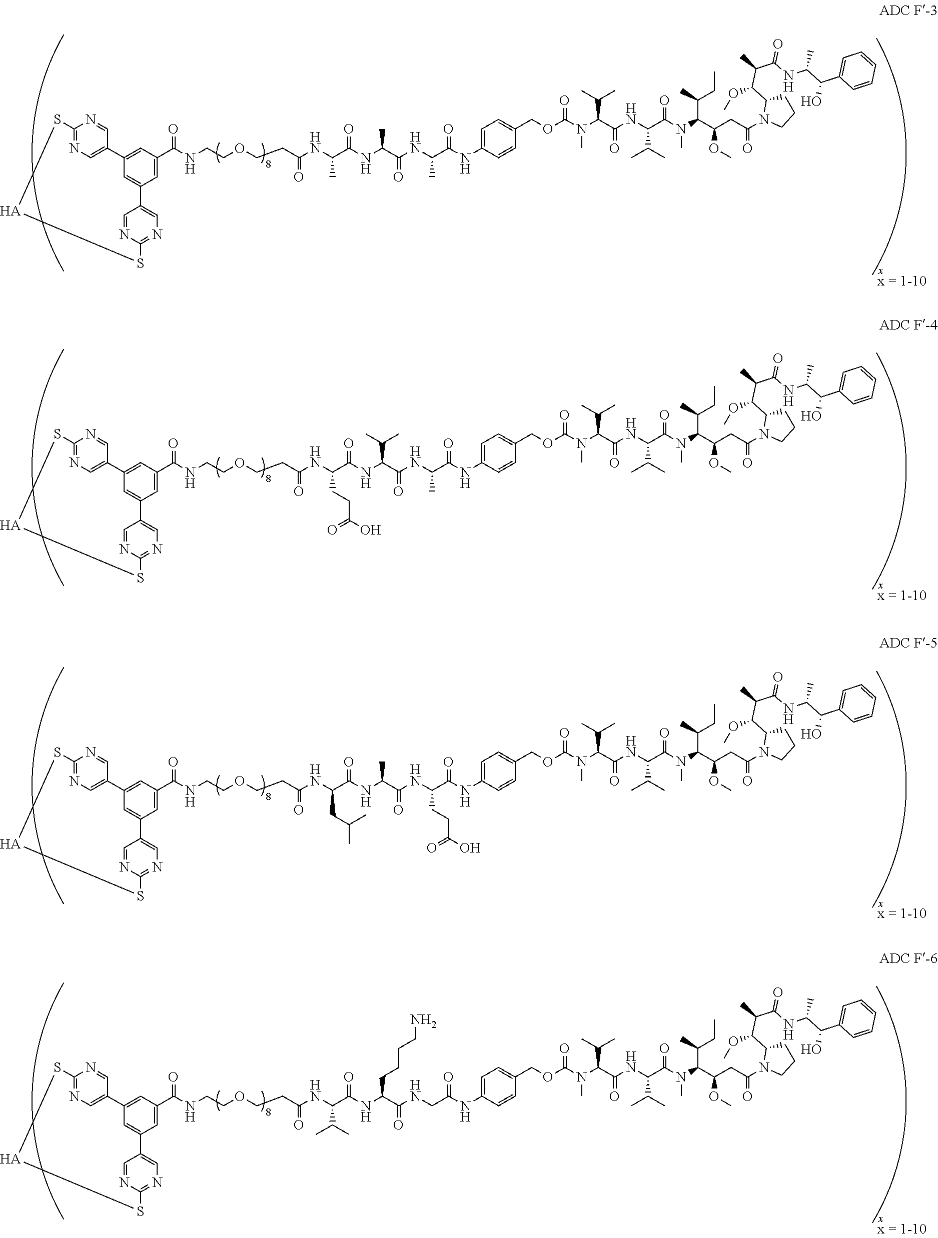 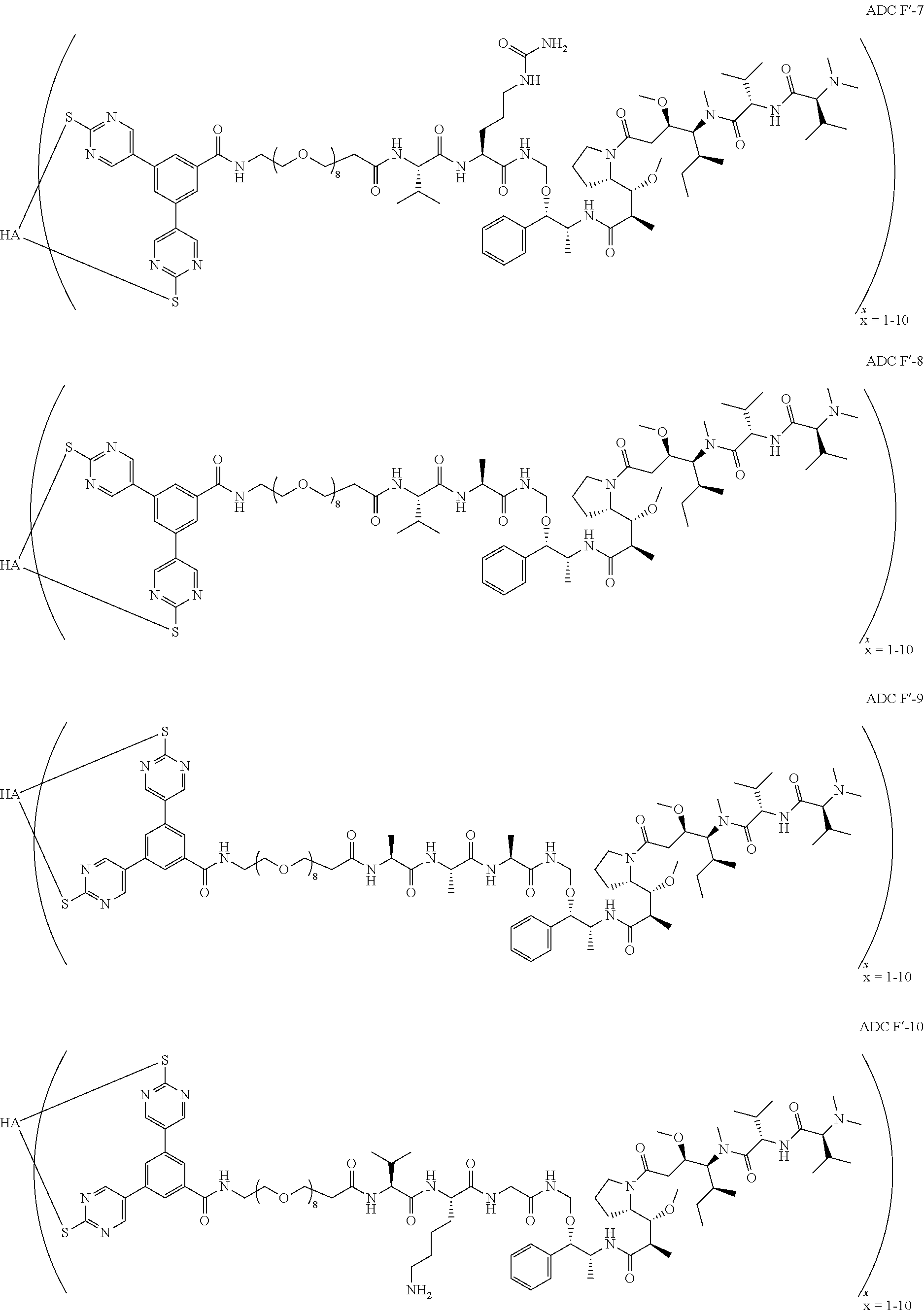 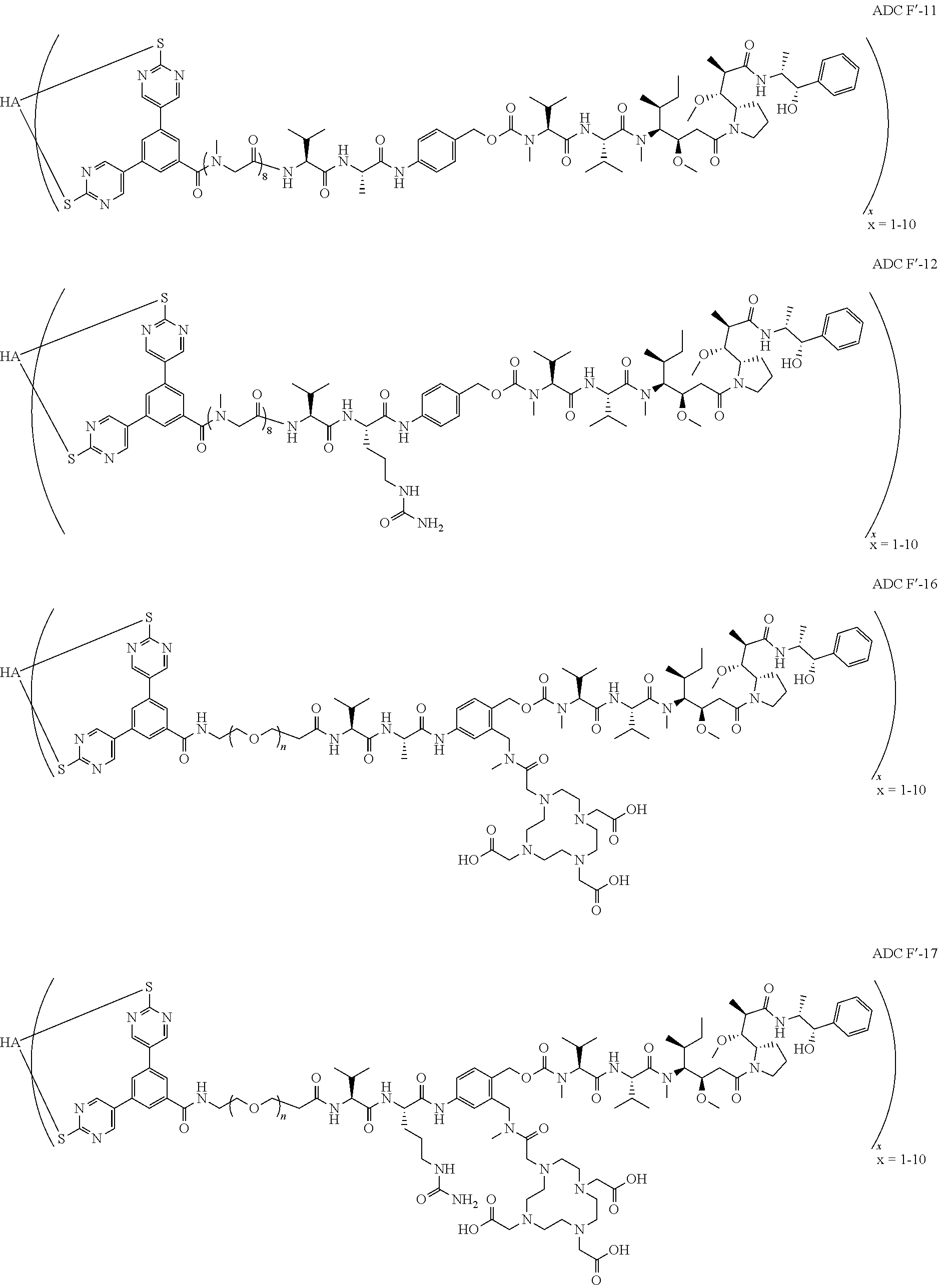 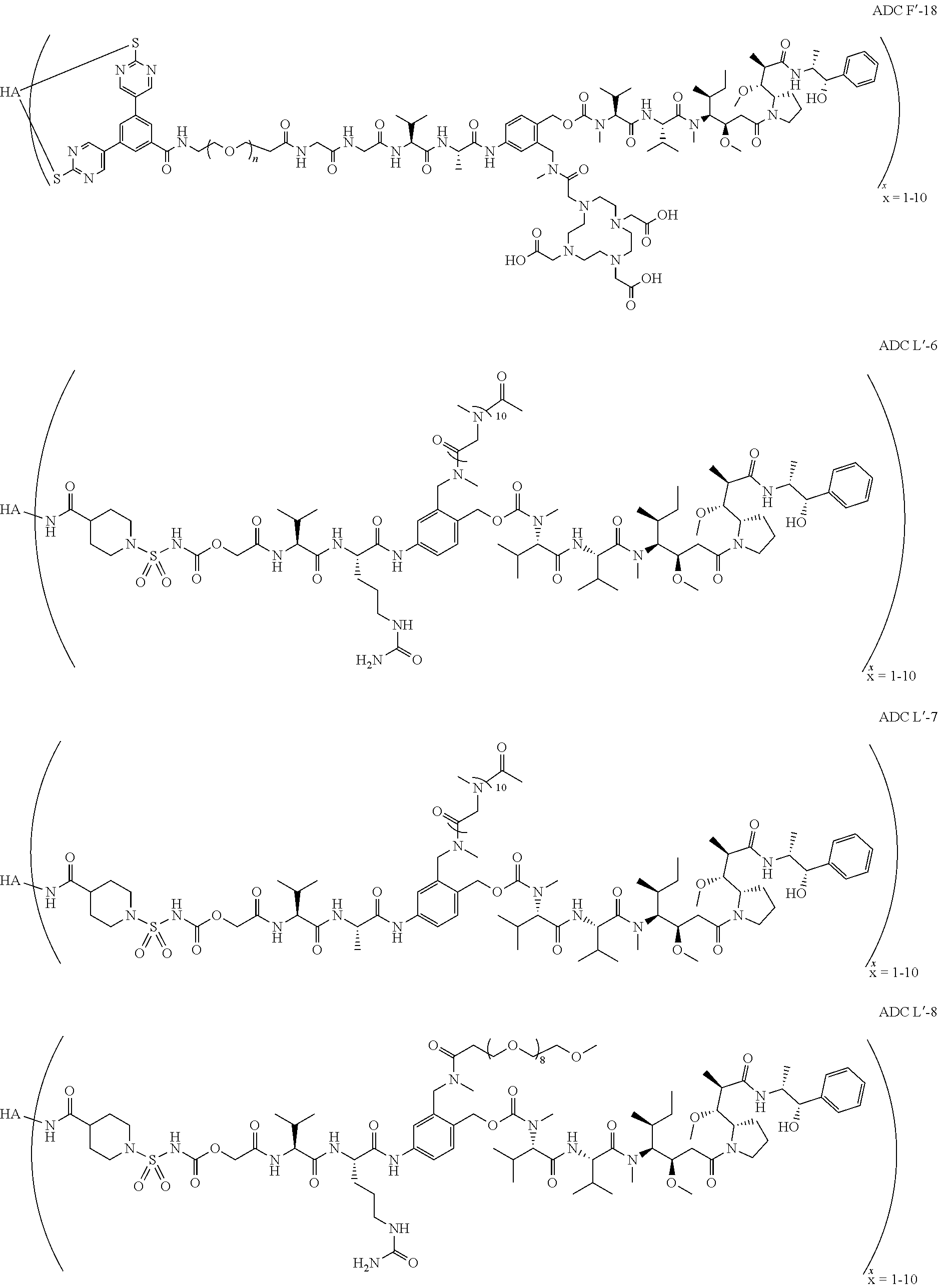 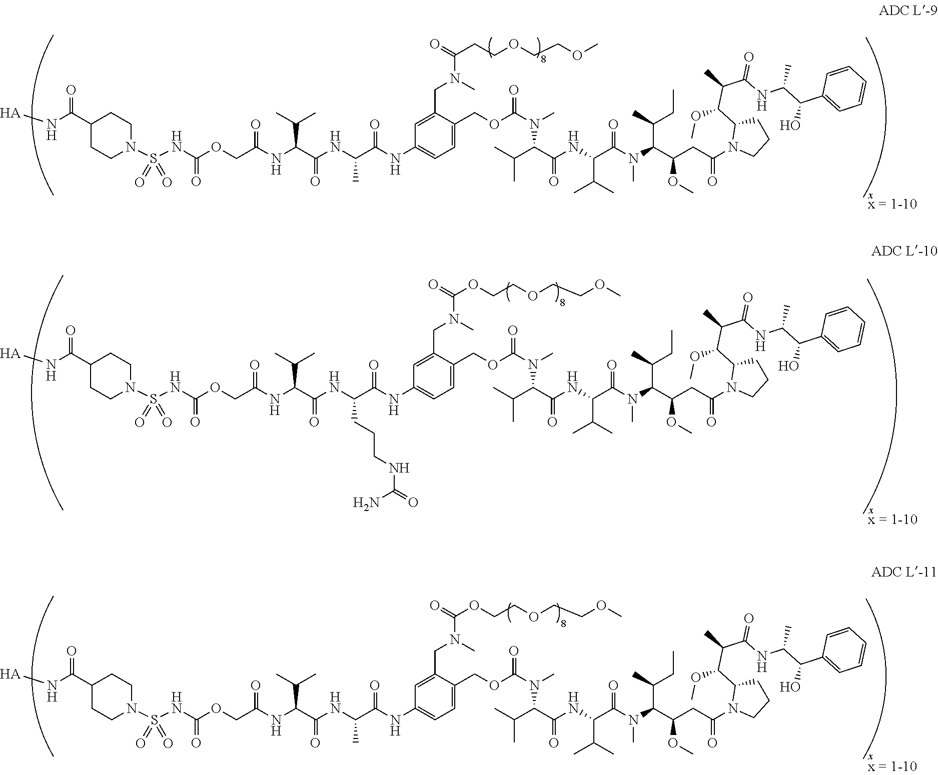
wherein, HA in each antibody-drug conjugate represents an antibody or antigen-binding fragment thereof capable of specifically binding to epidermal growth factor receptor 2 (Her2), a member of the ErbB family of receptor tyrosine kinases; preferably, the antibody or antigen-binding fragment thereof is as defined in any one of claims 8 to 11; n is an integer selected from 1 to 20, preferably n is an integer selected from 1 to 15, such as an integer of 1 to 12, 3 to 12, 5 to 15, 5 to 10, such as 1, 2, 3, 4, 5, 6, 7, 8, 9, 10, 11, 12, 13, 14 or 15, preferably 5, 8, 9 or 10;
wherein, 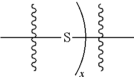 [SMILES: *S*]
represents a specific linkage between the sulfhydryl group in the antibody or antigen-binding fragment thereof and the linker; 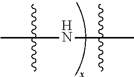 [SMILES: *N*]
represents a specific linkage between the amino group in the antibody or antigen-binding fragment thereof and the linker.

15. A composition, wherein the composition comprises one or more of the antibody-drug conjugate according to any one of claims 1 to 14, wherein the DAR value (drug-to-antibody ratio) of the composition is 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 8, for example, 3.0 to 3.5, 3.0 to 4.0, 3.0 to 4.5, 3.0 to 5.0, 6.0 to 6.5, 6.0 to 7.0, 6.0 to 7.5, 6.0 to 8.0, 6.0 to 8.5, 6.5 to 7.0, 6.5 to 7.5, 6.5 to 8.0, 6.5 to 8.5, 7.0 to 7.5, 7.0 to 8.0 or 7.5 to 8.0.

16. A pharmaceutical composition, which comprises the antibody-drug conjugate according to any one of claims 1 to 14 or the composition according to claim 15, and one or more pharmaceutical excipients.

17. Use of the antibody-drug conjugate according to any one of claims 1 to 14, the composition according to claim 15, or the pharmaceutical composition according to claim 15 in the manufacture of a medicament for treating an Her2-expressing cancer.

18. The use according to claim 17, wherein the cancer is solid tumors or hematological malignancies, such as gastric cancer, breast cancer, lung cancer (e.g., non-small cell lung cancer, specifically lung adenocarcinoma) or urothelial carcinoma.

19. The antibody-drug conjugate according to any one of claims 1 to 14, the composition according to claim 15, or the pharmaceutical composition according to claim 16, for use in treating an Her2-expressing cancer.

20. The antibody-drug conjugate, composition, or pharmaceutical composition according to claim 19, wherein the cancer is solid tumors or hematological malignancies, such as gastric cancer, breast cancer, lung cancer (e.g., non-small cell lung cancer, specifically lung adenocarcinoma) or urothelial carcinoma.

21. A method for treating Her2-expressing cancer, comprising a step of administering to a subject in need thereof an effective amount of the antibody-drug conjugate according to any one of claims 1 to 14, the composition according to claim 15, or the pharmaceutical composition according to claim 16.

22. The method according to claim 21, wherein the cancer is solid tumors or hematological malignancies, such as gastric cancer, breast cancer, lung cancer (e.g., non-small cell lung cancer, specifically lung adenocarcinoma) or urothelial carcinoma.
